(19) United States
(12) Patent Application Publication
(10) Pub. No.: US 2024/0263171 A1
(43) Pub. Date: Aug. 8, 2024

(54) ADVANCED GENOME EDITING

(71) Applicant: MBP TITAN, LLC, South San Francisco, CA (US)

(72) Inventors: Xuezi LI, Hayward, CA (US); James KEALEY, Sebastopol, CA (US); Kevin DIETZEL, Pacifica, CA (US)

(73) Assignee: MBP TITAN, LLC, South San Francisco, CA (US)

(21) Appl. No.: 18/408,520

(22) Filed: Jan. 9, 2024

Related U.S. Application Data
(62) Division of application No. 16/755,513, filed on Apr. 10, 2020, now Pat. No. 11,905,513.
(60) Provisional application No. 62/572,012, filed on Oct. 13, 2017.

(51) Int. Cl. C12N 15/11; C12N 1/20; C12N 9/22

(52) U.S. Cl. CPC C12N 15/11; C12N 1/20; C12N 9/22; C12N 2310/20; C12N 2800/80

(57) ABSTRACT

Methods of genetically modifying microorganisms using advanced genome editing are disclosed. Methods of modifying the genome of these microorganisms for point mutations, deletions, and DNA insertions are also disclosed. Further, inhibiting expression of genes without manipulating the genome of the microorganism is disclosed. In some cases, the microorganism can be a methylotroph, e.g., a methanotroph.

### SEQUENCE LISTING

This application contains a Sequence Listing, which has been submitted electronically in ASCII format and is hereby incorporated by reference in its entirety. The ASCII copy was created on Oct. 11, 2017, is named INX00399_SL.txt and is 208,080 bytes in size.

### BACKGROUND OF THE INVENTION

Genomic editing is the key to molecular biology. Genome editing uses specific nucleases to create site-specific double-strand breaks (DSBs) at desired locations within the genome of a cell in order to insert, delete, or replace one or more nucleotides. The DSBs are then repaired, which result in the desired modifications.

Currently there are four families of engineered nucleases that are used for genome edits, including meganucleases, zinc finger nucleases (ZFN), transcription activator-like effector-based nucleases (TALEN), and clustered regularly interspaced short palindromic repeats (CRISPR)-Cas systems.

In general, the meganucleases method of gene editing is the least efficient of the methods mentioned above. Due to the nature of its DNA-binding element and the cleaving element, it is limited to recognizing one potential target every 1,000 nucleotides. Boglioli, E., Richard, M., “Rewriting the book of life: a new era in precision genome editing”. Boston Consulting Group, September 2015. ZFN was developed to overcome the limitations of meganucleases. The number of possible targets ZFN can recognize was increased to one in every 140 nucleotides. Boglioli, E., 2015. However, both methods are unpredictable due to the ability of their DNA-binding elements affecting each other. As a result, high degrees of expertise and lengthy and costly validation processes are required.

TALE nucleases are the most precise and specific method and yield a higher efficiency than the previous two methods using meganucleases and ZFN. TALEN achieves higher efficiency because the DNA-binding element contains an array of TALE subunits, each of them having the capability of recognizing a specific DNA nucleotide chain independent from others, resulting in a higher number of target sites with high precision. New TALEN take about one week and a few hundred dollars to create, with specific expertise in molecular biology and protein engineering. Boglioli, E., 2015.

CRISPR nucleases are slightly less precise compared to TALENs. However, the CRISPR method has been shown to be the quickest and cheapest method. CRISPR also requires the least amount of expertise in molecular biology as the design lays in the guide RNA instead of the proteins. One major advantage that CRISPR has over the ZFN and TALEN methods is that it can be directed to target different DNA sequences using its ˜80 nt CRISPR single guide ribonucleic acids (sgRNAs), while both ZFN and TALEN methods required construction and testing of the proteins created for targeting each DNA sequence. Barrangou. R., and Doudna, J.A., “Applications of CRISPR technologies in research and beyond”. Nature Biotechnology. 34:933-941, 2016.

The subject matter of the present invention relates to microorganisms, such as methanotrophs, and methods to genomically edit their DNA.

### INCORPORATION BY REFERENCE

All publications, patents, and patent applications cited herein are hereby incorporated by reference in their entireties to the same extent as if each individual publication, patent, or patent application was specifically and individually indicated to be incorporated by reference in its entirety. In the event of a conflict between a term herein and a term in an incorporated reference, the term herein controls.

### SUMMARY

Disclosed herein is a method of genetic engineering comprising: (a) contacting a microorganism capable of converting a C₁ carbon to a multicarbon product with a polynucleotide encoding for a Cas enzyme and a guide ribonucleic acid (gRNA); and (b) growing the microorganism until genetic modification occurs.

In some cases, the microorganism capable of converting a C₁ carbon to a multicarbon product is a methylotroph. For example, the methylotroph can be a methanotroph. If the microorganism is a methanotroph, it can be from the genera Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, or Methyloacidophilum. Particular methanotrophs that can be used are methanotrophs from the genus Methylococcus, such as Methylococcus capsulatus.

In some cases, the C₁ carbon is carbon monoxide (CO), carbon dioxide (CO₂), methane (CH₄), or any combination thereof. For example, the C₁ carbon used can be CH₄.

In some cases, the Cas enzyme is Cas1, Cas1B, Cas2, Cas3, Cas4, Cas5, Cas5d, Cas5t, Cas5h, Cas5a, Cas6, Cas7, Cas8, Cas9, Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof. For example, the Cas enzyme use can be Cas9.

In some cases, the polynucleotide encoding for a gRNA used can be at least partially homologous to a promoter, intron, or coding sequence of an RNA polymerase beta-subunit (rpoB) gene or a gene within the 2,3-butanediol (2,3-BDO), 1,4-butanediol (1,4-BDO), isobutyraldehyde, or isobutanol pathway. For example, the polynucleotide encoding for a gRNA can be at least partially homologous to a promoter, intron, or coding sequence of rpoB. Additionally, the polynucleotide encoding for a gRNA can be directed to a promoter, intron, or coding sequence of gene within the 2,3-BDO pathway. If targeting the promoter, intron, or coding sequence of a gene within the 2,3-BDO pathway, the promoter, intron or coding sequence can be from the genes encoding an acetoin reductase, alpha-acetolactate decarboxylase, or acetolactate synthase.

In some cases the polynucleotide encoding for a gRNA can be directed to a promoter, intron, or coding sequence of a gene within the 1,4-BDO pathway. If targeting the promoter, intron, or coding sequence of a gene within the 1,4-BDO pathway, the promoter, intron or coding sequence can be from the genes encoding a pyruvate dehydrogenase (aceEF), citrate synthase (gltA), aconitate hydratase 1 (acnA), isocitrate dehydrogenase (icdA), citrate synthase (gltA), succinyl-CoA synthetase (SucC), CoA-dependent succinate semialdehyde dehydrogenase (SucD). 4-hyrobutyrate dehydrogenase (4hbD), 4-hydroxybutyryl-CoA transferase (Cat2), aldehyde dehydrogenase (Ald), alcohol dehydrogenase (Adh), or α-ketoglutarate decarboxylase (kgd).

In some cases, the polynucleotide encoding for a gRNA can be directed to a promoter, intron, or coding sequence of a gene within the isobutyraldehyde pathway. If targeting the promoter, intron, or coding sequence of a gene within the isobutyraldehyde pathway, the promoter, intron or coding sequence can be from the genes encoding an acetolactate synthase (AlsS), ketol-acid reductoisomerase (IlvC), dihydroxy-acid dehydratase (IlvD), and 2-keto acid decarboxylase (KDC).

In some cases, the polynucleotide encoding for a gRNA can be directed to a promoter, intron, or coding sequence of a gene within the isobutanol pathway. If targeting the promoter, intron, or coding sequence of a gene within the isobutanol pathway, the promoter, intron or coding sequence can be from the genes encoding an AlsS, IlvC, IlvD, KDC, or Adh.

In some cases, the microorganism used has a lower transformation efficiency compared to an E. coli bacterium. The transformation efficiency of the microorganism is increased prior to trying to transform the microorganism with any nucleic acids.

In some cases, the polynucleotide encoding for the gRNA can be transformed prior to the polynucleotide encoding for a Cas enzyme. Additionally, the method can further comprise contacting the microorganism with a donor polynucleotide. In some cases, the microorganism is contacted with the donor polynucleotide prior to being contacted with a polynucleotide encoding for a Cas enzyme. In some cases, the microorganism is contacted concurrently with the donor polynucleotide and the polynucleotide encoding for a gRNA. In some cases, the donor polynucleotide and the polynucleotide encoding for a gRNA are on a single plasmid. The donor polynucleotides used in the methods can be less than 1000 bases. For example, the donor polynucleotide can be less than 600 bases. In some cases, the donor polynucleotide can be less than 100 bases.

In some cases, the polynucleotide encoding for a Cas enzyme can be within a plasmid. The plasmid in some cases does not comprise a strong promoter. In some cases, the plasmid can comprise a mutated promoter. In some cases, the promoter can be a pMxaF promoter.

Also disclosed is a genetically modified microorganism capable of converting a C₁ carbon to a multicarbon product comprising a nucleic acid encoding a heterologous Cas enzyme. The genetically modified microorganism can be a methylotroph, such as a methanotroph or any of the genus and/or species described throughout. The C₁ carbon is carbon monoxide (CO), carbon dioxide (CO₂), methane (CH₄), any combination thereof, or any C₁ described throughout

In some cases, the genetically modified microorganism comprises a heterologous Cas enzyme. Any of the Cas enzymes described throughout can be used. The Cas enzymes can be expressed in a plasmid. The plasmids can also include a stronger, mutated, and/or pMxaF promoter.

In some cases, the genetically modified microorganism can further comprise a polynucleotide encoding for a gRNA. In some cases, the genetically modified microorganism can comprise a polynucleotide encoding for a gRNA that is at least partially homologous to a portion of a promoter, intron, or coding sequence of an rpoB gene or a gene within the 2,3-BDO, 1,4-BDO, isobutyraldehyde, or isobutanol pathway. For example, the polynucleotide encoding for a gRNA can be at least partially homologous to a promoter, intron, or coding sequence of rpoB.

In some cases, the genetically modified microorganism can have a lower transformation efficiency compared to an E. coli bacteria. However, before transformation with nucleic acids, the transformation efficiency of the microorganism can be increased.

In some cases, the microorganism can comprise a point mutation compared to a wild-type microorganism of the same species. For example, the point mutation can be within a promoter, intron, or coding sequence of an rpoB gene or a gene within the 2,3-butanediol (2,3-BDO), 1,4-butanediol (1,4-BDO), isobutyraldehyde, or isobutanol pathway.

In some cases, the microorganism can comprise a deletion of one or more nucleotides compared to a wild-type microorganism of the same species. For example, the deletion of one or more nucleotides can be within a promoter, intron, or coding sequence of an rpoB gene or a gene within the 2,3-butanediol (2,3-BDO), 1,4-butanediol (1,4-BDO), isobutyraldehyde, or isobutanol pathway.

In some cases, the microorganism can comprise an addition of one or more nucleotides compared to a wild-type microorganism of the same species. For example, the addition of one or more nucleotides can be within a promoter, intron, or coding sequence of an rpoB gene or a gene within the 2,3-butanediol (2,3-BDO), 1,4-butanediol (1,4-BDO), isobutyraldehyde, or isobutanol pathway.

Also disclosed herein is a method of replacing a single nucleotide within the genome of a microorganism that is capable of converting a C₁ carbon to a multicarbon product comprising: (a) contacting the microorganism with a polynucleotide encoding for i) a Cas enzyme and ii) a polynucleotide encoding for a gRNA; and (b) growing the microorganism until a single nucleotide is replaced within the genome of the microorganism.

In some cases, the microorganism can be a methylotroph, such as a methanotroph or any microorganism described throughout. In some cases, the C₁ carbon can be any C₁ carbon described throughout. In some cases, the Cas enzyme can be any described throughout, such as a Cas9 enzyme. The Cas enzymes can be expressed in a plasmid. The plasmids can also include a stronger, mutated, and/or pMxaF promoter. In some cases, the polynucleotide encoding for a gRNA is at least partially complementary to a polynucleotide that is within a promoter, intron, or coding sequence of an rpoB gene or a gene within the 2,3-butanediol (2,3-BDO), 1,4-butanediol (1,4-BDO), isobutyraldehyde, or isobutanol pathway.

In some cases, the polynucleotide encoding for a gRNA is transformed prior to the polynucleotide encoding for a Cas enzyme. In some instances, the method can further comprise contacting the microorganism with a donor polynucleotide. For example, the microorganism can be contacted with the donor polynucleotide prior to being contacted with a polynucleotide encoding for a Cas enzyme. In some cases, the microorganism can be contacted concurrently with the donor polynucleotide and polynucleotide encoding for a gRNA. In some cases, the donor polynucleotide and polynucleotide encoding for a gRNA are contained on a single plasmid. In some cases, the donor polynucleotide can be less than 1000 bases. For example, the donor polynucleotide can be less than 600 bases. In some cases, the donor polynucleotide is can be less than 100 bases.

In some cases, the replacement of a single nucleotide can result in a different nucleotide. In some cases, the replacement occurs at a single nucleotide within a promoter, intron, or coding sequence of an rpoB gene or a gene within the 2,3-butanediol (2,3-BDO), 1,4-butanediol (1,4-BDO), isobutyraldehyde, or isobutanol pathway.

In some cases, as a result of the single nucleotide replacement, the expression of one or more genes can be changed. Additionally, in some cases, the replacement can change the activity of one or more enzymes.

Also disclosed herein is a method of removing one or more nucleotides from the genome of a genetically modified microorganism that is capable of converting a C₁ carbon to a multicarbon product comprising: (a) contacting the microorganism with a polynucleotide encoding for i) a Cas enzyme and ii) a polynucleotide encoding for a gRNA; and (b) growing the microorganism until one or more nucleotides within the genome of the microorganism is removed.

In some cases, the microorganism can be a methylotroph, such as a methanotroph or any microorganism described throughout. In some cases, the C₁ carbon can be any C₁ carbon described throughout. In some cases, the Cas enzyme can be any described throughout, such as a Cas9 enzyme. The Cas enzymes can be expressed in a plasmid. The plasmids can also include a stronger, mutated, and/or pMxaF promoter. In some cases, the polynucleotide encoding for a gRNA is at least partially complementary to a polynucleotide that is within a promoter, intron, or coding sequence of an rpoB gene or a gene within the 2,3-butanediol (2,3-BDO), 1,4-butanediol (1,4-BDO), isobutyraldehyde, or isobutanol pathway.

In some cases, two or more nucleotides are removed from the targeted nucleic acid. For example, the removal of two or more nucleotides can occur within a promoter, intron, or coding sequence of an rpoB gene or a gene within the 2,3-butanediol (2,3-BDO), 1,4-butanediol (1,4-BDO), isobutyraldehyde, or isobutanol pathway.

In some cases, the polynucleotide encoding for a gRNA is transformed prior to the polynucleotide encoding for a Cas enzyme. In some instances, the method can further comprise contacting the microorganism with a donor polynucleotide. For example, the microorganism can be contacted with the donor polynucleotide prior to being contacted with a polynucleotide encoding for a Cas enzyme. In some cases, the microorganism can be contacted concurrently with the donor polynucleotide and polynucleotide encoding for a gRNA. In some cases, the donor polynucleotide and polynucleotide encoding for a gRNA are contained on a single plasmid. In some cases, the donor polynucleotide can be less than 1500 bases. In some cases, the donor polynucleotide can be less than 1000 bases. For example, the donor polynucleotide can be less than 600 bases. In some cases, the donor polynucleotide is can be less than 100 bases.

In some cases, the removal of one or more nucleotides can change the expression of one or more genes. In some cases, the removal of one or more nucleotides can change the activity of one or more enzymes.

Disclosed herein is a method of adding one or more nucleotides to the genome of a microorganism capable of converting a C₁ carbon to a multicarbon product comprising: (a) contacting the microorganism with a polynucleotide encoding for i) a Cas enzyme and ii) a polynucleotide encoding for a gRNA; and (b) growing the microorganism until one or more nucleotides is added to the genome of the microorganism.

In some cases, the microorganism can be a methylotroph, such as a methanotroph or any microorganism described throughout. In some cases, the C₁ carbon can be any C₁ carbon described throughout. In some cases, the Cas enzyme can be any described throughout, such as a Cas9 enzyme. The Cas enzymes can be expressed in a plasmid. The plasmids can also include a stronger, mutated, and/or pMxaF promoter. In some cases, the polynucleotide encoding for a gRNA is at least partially complementary to a polynucleotide that is within a promoter, intron, or coding sequence of an rpoB gene or a gene within the 2,3-butanediol (2,3-BDO), 1,4-butanediol (1,4-BDO), isobutyraldehyde, or isobutanol pathway.

In some cases, two or more nucleotides can be added to a target nucleic acid. For example, the addition of one or more nucleotides can occur within a promoter, intron, or coding sequence of an rpoB gene or a gene within the 2,3-butanediol (2,3-BDO), 1,4-butanediol (1,4-BDO), isobutyraldehyde, or isobutanol pathway.

In some cases, the polynucleotide encoding for a gRNA is transformed prior to the polynucleotide encoding for a Cas enzyme. In some instances, the method can further comprise contacting the microorganism with a donor polynucleotide. For example, the microorganism can be contacted with the donor polynucleotide prior to being contacted with a polynucleotide encoding for a Cas enzyme. In some cases, the microorganism can be contacted concurrently with the donor polynucleotide and polynucleotide encoding for a gRNA. In some cases, the donor polynucleotide and polynucleotide encoding for a gRNA are contained on a single plasmid. In some cases, the donor polynucleotide can be less than 1500 bases. In some cases, the donor polynucleotide can be less than 1000 bases. For example, the donor polynucleotide can be less than 600 bases. In some cases, the donor polynucleotide is can be less than 100 bases.

In some cases, the addition of one or more nucleotides can change the expression of one or more genes. In some cases, the addition of one or more nucleotides can change the activity of one or more enzymes.

Also disclosed herein is a method of inhibiting expression of a gene within a methylotroph comprising contacting the methylotroph with a polynucleotide encoding for i) a modified Cas enzyme and ii) a polynucleotide encoding for a gRNA, where the modified Cas enzyme does not cleave nucleic acids.

In some cases, the microorganism can be a methylotroph, such as a methanotroph or any microorganism described throughout. In some cases, the modified Cas enzyme can be any described throughout, such as a modified Cas9 enzyme. In some cases, the Cas enzyme can be expressed within a plasmid. In some cases, the polynucleotide encoding for a gRNA is at least partially complementary to a polynucleotide that is within a promoter, intron, or coding sequence of an rpoB gene or a gene within the 2,3-butanediol (2,3-BDO), 1,4-butanediol (1,4-BDO), isobutyraldehyde, or isobutanol pathway.

In some cases, the inhibition of gene expression is greater than 10% compared to a wild-type microorganism of the same species. For example, the inhibition of gene expression is greater than 50% compared to a wild-type microorganism of the same species.

Also disclosed herein is a vector comprising a polynucleotide encoding for a Cas9 enzyme, where the Cas9 enzyme is capable of being expressed in a methylotroph, such as a methanotroph (e.g., a methanotroph from the genus Methylococcus).

Further disclosed herein is a method of screening for genome editing in a methylotroph comprising contacting the methylotroph with a first polynucleotide encoding for a gRNA, and subsequently with a second polynucleotide encoding a Cas9 enzyme, where the first polynucleotide encoding for a gRNA is at least partially complementary to a polynucleotide that is within a promoter, intron, or coding sequence of an rpoB gene. In some cases, the methylotroph can be a methanotroph, for example from the genus Methylococcus.

In some cases, the Cas enzyme can be any Cas enzyme disclosure throughout, such as a Cas9 enzyme. In some cases, the Cas enzyme can be expressed within a plasmid.

The method used herein can produce colonies when plated. These can be referred to as colony forming units (CFU). In some cases, the method described herein can produce CFU that are decreased by at least 1.1 fold. In some cases, the CFU can be decreased by at least 2 fold. In some cases, the CFU can be decreased by at least 3 fold. In some cases, the CFU can be decreased by at least 4 fold.

### BRIEF DESCRIPTION OF THE DRAWINGS

FIG. 1 depicts plates of Methylococcus capsulatus colonies. Methylococcus capsulatus were transformed with a plasmid expressing: 1) Cas9 only; 2) Cas9+gRNA2; 3) Cas9+gRNA5; or 4) Cas9+gRNA2+double stranded DNA (dsDNA). Methylococcus capsulatus that were transformed with all three (i.e., group 4) did not produce any colonies, indicating a lack of gene editing. The other groups, which are negative controls, grew colonies indicating an off target effect.

FIG. 2 depicts a two plasmid approach for advanced genome editing (AGE). In one plasmid, Cas9 is expressed with a promoter, such as a weak promoter. In the second plasmid, a gRNA and a donor dsDNA are expressed.

FIG. 3 depicts the number of colony forming units (CFU) for gene editing of the gene rpoB. As shown, the targeting of the rpoB gene resulted in four orders of magnitude drop of CFUs.

FIG. 4 depicts a colony polymerase chain reaction (cPCR) verification of a ppdK deletion of about 600 bases. As shown in the gel, approximately 72% of the clones were successfully edited.

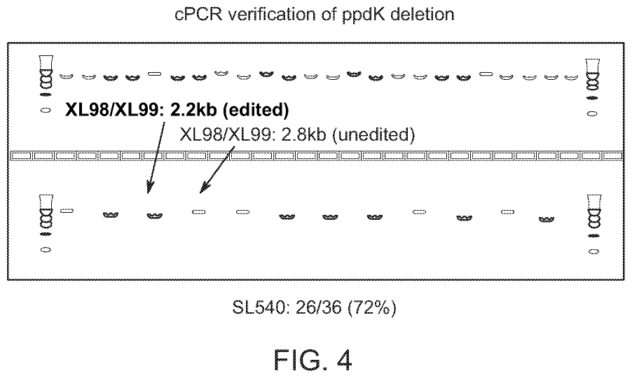

FIG. 5 depicts a cPCR verification of a ppdK addition of about 400 bases. As shown in the gel, several clones were successfully edited.

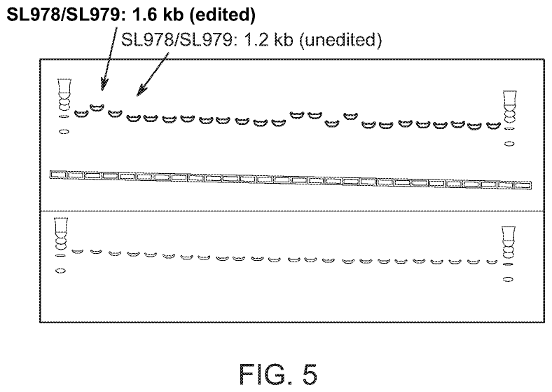

FIG. 6 shows that the expression of LacZ was successfully inhibited by the introduction of dCas9 in combination of a LacZ specific gRNA after 24, 30, 48, and 72 hours. The inhibition of LacZ in some strains was up to 8-fold (30 hours−Ptrc=LacZ+dCas9_gRNA3).

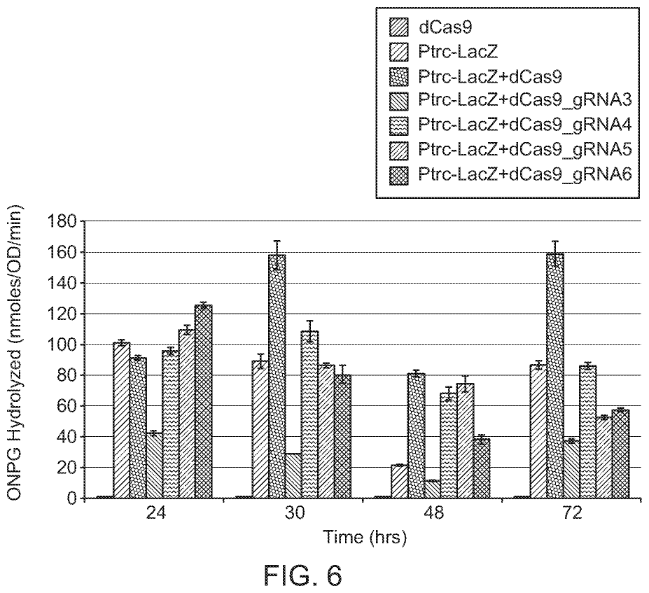

### DETAILED DESCRIPTION OF THE INVENTION

As summarized above, aspects of the invention include genetically modified microorganisms that are produced using advanced genomic editing tools. The genetically modified microorganisms include methylotrophs, such as methanotrophs, which are capable of using a C₁ carbon source, such as methane, as the primary carbon source for the organism. Additionally, as summarized above, advanced genome editing tools can be used to inhibit the expression of a gene.

Advanced genome editing can be used in many ways to alter the genome of a microorganism. For example, advanced genome editing can be used to generate a point mutation at any sequence within the genome. Additionally, advanced genome editing can be used to add one or more nucleotides to any sequence within a genome.

The precision, accuracy, and efficacy of the advanced genome editing is very high compared to that of traditional methods of genetic engineering.

Before the present invention is described in greater detail, it is to be understood that this invention is not limited to particular cases described, as such can, of course, vary. It is also to be understood that the terminology used herein is for the purpose of describing particular cases only, and is not intended to be limiting, since the scope of the present invention will be limited only by the appended claims.

### I. Definitions

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. In some cases, the numerical disclosed throughout can be “about” that numerical value even without specifically mentioning the term “about.”

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The phrases “recombinant host cell,” “genetically engineered host cell,” “engineered host cell,” “genetically modified host cell,” and their grammatical equivalents as used herein may be used interchangeably and can refer to host cells that have been genetically modified to: (a) express one or more exogenous polynucleic acids; (b) over-express one or more endogenous and/or one or more exogenous polynucleic acids, such as those included in a vector, or which have an alteration in expression of an endogenous gene; or (c) knock-out or down-regulate an endogenous gene. In addition, certain genes may be physically removed from the genome (e.g., knock-outs) or they may be engineered to have reduced, altered or enhanced activity. The phrases “recombinant host cell,” “genetically engineered host cell,” “engineered host cell,” and “genetically modified host cell” refer not only to the particular subject host cell, but to the progeny or potential progeny of such a cell. Because certain modifications may occur in succeeding generations due to either mutation or environmental influences, such progeny may not, in fact, be identical to the parent cell, but are still included within the scope of the term(s) as used herein.

The terms “engineer,” “genetically engineer,” “modify,” “genetically modify,” and their grammatical equivalents as used herein can refer to any manipulation of a microorganism that results in a detectable change in the microorganism, where the manipulation includes, but is not limited to, introducing non-native metabolic functionality via heterologous (exogenous) polynucleic acids or removing native-functionality via polynucleic acid deletions, mutations or knock-outs. The term “metabolically engineered” generally involves rational pathway design and assembly of biosynthetic genes (or open reading frames), genes associated with operons, and control elements of such polynucleic acids, for the production of a desired metabolite. “Metabolically engineered” may further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate leading to a desired pathway.

As used herein, the terms “genetic modification,” “genetically modified” and their grammatical equivalents can refer to any modification of a polynucleic acid and/or polypeptide that results in an altered nucleic acid or polypeptide (i.e., relative to the wild-type nucleic acid or polypeptide sequence). Genetic modification includes, for example, point mutations, substitutions, deletions, or insertions of single or multiple residues in a polynucleic acid (or the encoded polypeptide), which includes alterations arising within a protein-encoding region of a gene as well as alterations in regions outside of a protein-encoding sequence, such as, but not limited to, regulatory or promoter sequences. A genetic modification may be an alteration of any type. For instance, the modification may be a deletion, insertion, mutation, rearrangement, or any combination thereof. In certain cases, a portion of a genetically modified microorganism's genome may be replaced with one or more heterologous (exogenous) polynucleic acids. In some cases, the modification is naturally-occurring. In other cases, the modification is the result of artificial selection pressure. In still other cases, the modification is the result of genetic engineering. One form of genetic modification is disruption, such as by knockout. As used herein, the term “introducing,” as used in phrases such as “introducing into the host cell” at least one polynucleic acid includes methods known in the art for introducing polynucleic acids into a cell, including, but not limited to transformation (e.g., calcium chloride, electroporation), transduction, transfection, conjugation and the like.

The term “genetic modification” or “genetically modified” and their grammatical equivalents as used herein can refer to one or more alterations of a nucleic acid, e.g., the nucleic acid within a microorganism's genome. For example, genetic modification can refer to alterations, additions, and/or deletion of nucleic acid (e.g., whole genes or fragments of genes).

The term “disrupting” and its grammatical equivalents as used herein can refer to a process of altering a gene, e.g., by deletion, insertion, mutation, rearrangement, or any combination thereof. For example, a gene can be disrupted by knockout. Disrupting a gene can be partially reducing or completely suppressing expression (e.g., mRNA and/or protein expression) of the gene. Disrupting can also include inhibitory technology, such as shRNA, siRNA, microRNA, dominant negative, or any other means to inhibit functionality or expression of a gene or protein.

The term “gene editing” and its grammatical equivalents as used herein can refer to genetic engineering in which one or more nucleotides are inserted, replaced, or removed from a genome. For example, gene editing can be performed using a nuclease (e.g., a natural-existing nuclease or an artificially engineered nuclease).

As used herein, the term “endogenous,” and its grammatical equivalents when used in reference to polynucleic acids (and the polypeptides encoded therein), can refer to polynucleic acids and polypeptides that are expressed in the organism in which they originated (i.e., they are innate to the organism). In contrast, the terms “heterologous” and “exogenous” are used interchangeably, and as defined herein with reference to polynucleic acids (and the polypeptides encoded therein), indicates polynucleic acids and polypeptides that are expressed in an organism other than the organism from which they (i.e., the polynucleic acid or polypeptide sequences) originated or where derived.

As used herein, the term “homolog” and its grammatical equivalents, as used with respect to an original protein, polypeptide, gene, or polynucleic acid (or ORF encoding the same) of a first family or species, can refer to distinct proteins, genes, or polynucleic acids of a second family or species that correspond (structurally, functionally, and/or genomically) to the original protein, gene, or polynucleic acid of the first family or species. Most often, “homologs” will have functional, structural or genomic similarities. Techniques are known by which homologs of a protein, gene or polynucleic acid can readily be cloned using genetic probes and PCR. Identity of cloned sequences as “homologs” can be confirmed using functional assays and/or by genomic mapping of the genes.

As used herein, the term “strong promoter” and its grammatical equivalents as used herein can refer to a promoter that has the ability to increase the transcription at a high level. For example, pMxaF, J2311, J12100, and J23102 each can be considered a strong promoter. As used herein, the term “weak promoter” and its grammatical equivalents as used herein can refer to a promoter that has the ability to increase the transcription, but at a low level. For example, pBAD, J23110, 1acO, J23116, J23106, J23105, J23108, J23107, J23115, and J23114 can each be considered a weak promoter. Additionally, the term “medium strength promoter” and its grammatical equivalents, as used herein can refer to a promoter that has the ability to increase the transcription at a level that is less than what is considered high but higher than what is considered low. For example, J23118, J23104, J23101, J23119, and uMCA3034, can each be considered a medium strength promoter. In some cases, medium strength promoters can be used in lieu of strong or weak promoters.

The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.”

As used herein, the term “substantially similar” and its grammatical equivalents, when used in reference to the similarity between a sequence and a reference sequence, means that the sequences are at least 50% (but not 100%) identical. In some cases, the sequences are 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, or 99.9999% identical. In some cases, the term substantially similar refers to a sequence that is at least 50% identical. In some instances, the term substantially similar refers to a sequence that is 55% identical. In some instances, the term substantially similar refers to a sequence that is 60% identical. In some instances, the term substantially similar refers to a sequence that is 65% identical. In some instances, the term substantially similar refers to a sequence that is 70% identical. In some instances, the term substantially similar refers to a sequence that is 75% identical. In some instances, the term substantially similar refers to a sequence that is 80% identical. In some instances, the term substantially similar refers to a sequence that is 81% identical. In some instances, the term substantially similar refers to a sequence that is 82% identical. In other instances, the term substantially similar refers to a sequence that is 83% identical. In other instances, the term substantially similar refers to a sequence that is 84% identical. In some instances, the term substantially similar refers to a sequence that is 85% identical. In some instances, the term substantially similar refers to a sequence that is 86% identical. In other instances, the term substantially similar refers to a sequence that is 87% identical. In other instances, the term substantially similar refers to a sequence that is 88% identical. In other instances, the term substantially similar refers to a sequence that is 89% identical. In some instances, the term substantially similar refers to a sequence that is 90% identical. In some instances, the term substantially similar refers to a sequence that is 91% identical. In some instances, the term substantially similar refers to a sequence that is 92% identical. In some instances, the term substantially similar refers to a sequence that is 93% identical. In some instances, the term substantially similar refers to a sequence that is 94% identical. In some instances, the term substantially similar refers to a sequence that is 95% identical. In some instances, the term substantially similar refers to a sequence that is 96% identical. In some instances, the term substantially similar refers to a sequence that is 97% identical. In some instances, the term substantially similar refers to a sequence that is 98% identical. In some instances, the term substantially similar refers to a sequence that is 99% identical. In some instances, the term substantially similar refers to a sequence that is 99.9% identical. In some instances, the term substantially similar refers to a sequence that is 99.99% identical. In some instances, the term substantially similar refers to a sequence that is 99.999% identical. In some instances, the term substantially similar refers to a sequence that is 99.9999% identical. To determine the percentage of identity between two sequences, the two sequences are aligned, using, for example, the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids/nucleotides is determined between the two sequences. Methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et al., J. Molec. Biol., 1990:215:403). A particularly preferred method for determining the percentage identity between two polypeptides involves the Clustal W algorithm (Thompson, J D, Higgines, D G and Gibson T J, 1994, Nucleic Acid Res 22(22): 4673-4680 together with the BLOSUM 62 scoring matrix (Henikoff S & Henikoff, J G, 1992, Proc. Natl. Acad. Sci. USA 89: 10915-10919) using a gap opening penalty of 10 and a gap extension penalty of 0.1, so that the highest order match obtained between two sequences where at least 50% of the total length of one of the two sequences is involved in the alignment.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

As will be apparent to those of skill in the art upon reading this disclosure, each of the individual cases described and illustrated herein has discrete components and features which may be readily separated from or combined with the features of any of the other several cases without departing from the scope or spirit of the present invention. Any recited method can be carried out in the order of events recited or in any other order which is logically possible.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

### II. Genetically Modified Microorganisms and Methods of Making the Same

The present disclosure is directed, in part, to genetically modified microorganisms that have been modified using advanced genome editing techniques.

### Microorganisms

In some cases, the microorganisms can use C₁ carbon substrates, such as CO, CO₂, and CH₄, to synthesize a desired end product. This, however, does not mean that these microorganisms use solely Ci carbons. Some of the microorganisms can be made to utilize additional carbon substrates, including carbon substrates that the microorganism naturally uses. For example, if the microorganism naturally uses sugar for carbon substrates, this microorganism can be made to utilize a different carbon source such as a C₁ carbon.

The microorganisms can be a prokaryote or a eukaryote. In some cases, for example, the microorganisms can be bacteria, yeast, or algae.

Microorganisms that can convert C₁ carbon substrates into desired products include those capable of using natural gas as a carbon substrate. For example, the microorganism can use methane contained within the natural gas as a carbon source to make such desired products. Such microorganisms can include methanotrophs. Methanotrophs that can be particularly useful include those from the genera Methylobacter, Methylomicrobium, Methylomonas, Methylocaldum, Methylococcus, Methylosoma. Methylosarcina, Methylothermus, Methylohalobius, Methylogaea, Methylovulum, Crenothrix, Clonothrix, Methylosphaera, Methylocapsa, Methylocella, Methylosinus, Methylocystis, Methyloacidophilum, or any combinations thereof. In some cases, the methanotroph is from the genus Methylococcus. In one instance, the methanotroph can be a methanotroph from the species Methylococcus capsulatus. In some cases, the methanotroph can be an obligate methanotroph. In other cases, the methanotroph can be a facultative methanotroph.

Some microorganisms are capable of using CO₂ as a substrate. Such microorganisms include methanogens. Microorganisms that are capable of using CO₂ as a substrate can contain chlorophyll. Examples thereof include algae and cyanobacteria.

Some microorganisms are capable of using CO as a substrate. Examples include anaerobic microorganisms such as Clostridium. These microorganism can be genetically modified so as to make substantial amounts of 2,3-BDO

In some cases, the microorganism used in the methods described throughout can be one that does not naturally express any Cas enzymes. In this case, any Cas enzymes that are present within the microorganism are heterologous to that microorganism.

In some cases, the heterologous Cas enzyme that is present within the microorganism can be expressed within a plasmid. The plasmids expressing the heterologous Cas enzyme can comprise a promoter, including but not limited to such promoters as a pMxaF or pBAD promoter. In some cases however, the plasmid does not comprise a strong promoter, for example a weak promoter. In some cases, the plasmid can comprise a mutated promoter. For example, the promoter can be a mutated promoter, such as a mutated pMxaF promoter. In some cases, the mutation contained in the promoter can make the promoter weaker.

### Nucleic Acids Encoding for Enzymes

Certain enzymes can be used to generate useful chemical products. Some useful chemical products can include, but are not limited to, isobutanol, isobutyraldehyde, 2,3-butanediol (2,3-BDO), and 1,4-butanediol (1,4-BDO). In some cases, certain proteins, such as RNA polymerase beta-subunits (EC:2.7.7.6) (encoded by such genes as rpoB or rpoB2) can confer to a microorganism resistance to some antibiotics, such as rifampin. In some cases, the polynucleotide of the promoters or introns of these enzymes can be altered by using the techniques described throughout.

### Isobutanol

In some cases, polynucleotides encoding for enzymes of the isobutanol pathway can be used. For example, the microorganism can contain (either endogenously or heterologous) one or more polynucleotides encoding for an acetolactate synthase (AlsS); ketol-acid reductoisomerase (KARI); dihydroxy-acid dehydratase (DHAD); 2-keto acid decarboxylase (KDC); and alcohol dehydrogenase (ADH). One or more of the polynucleotides can be native to the microorganism. In some cases, one or more of the polynucleotides can be heterologous to the microorganism.

In some cases, the acetolactate synthase (AlsS) can be encoded by a polynucleotide that is substantially similar to a gram positive bacterium AlsS gene. In some cases, the AlsS can be encoded by a polynucleotide that is substantially similar to SEQ ID NO: 1. In some other cases, the AlsS can be encoded by a polynucleotide that is substantially similar to SEQ ID NO: 2.

In some cases, the ketol-acid reductoisomerase can be encoded by a polynucleotide that is substantially similar to a gram negative bacterium ketol-acid reductoisomerase gene. In some cases, the ketol-acid reductoisomerase can be encoded by a polynucleotide that is substantially similar to SEQ ID NO: 3.

In some cases, the dihydroxy-acid dehydratase can be encoded by a polynucleotide that is substantially similar to a gram negative bacterium dihydroxy-acid dehydratase gene. In some cases, the dihydroxy-acid dehydratase can be encoded by a polynucleotide that is substantially similar to SEQ ID NO: 4. In some cases, the dihydroxy-acid dehydratase can be encoded by a polynucleotide that is substantially similar to SEQ ID NO: 5.

In some cases, the 2-keto acid decarboxylase (KDC) can be encoded by a polynucleotide that is substantially similar to a gram positive bacterium KDC gene. In some cases, the KDC can be encoded by a polynucleotide that is substantially similar to any one of SEQ ID NOs: 6 to 29.

In some cases, the alcohol dehydrogenase (ADH) can be encoded by a polynucleotide that is substantially similar to a gram positive or gram negative bacterium ADH gene. In some cases, the ADH can be encoded by a polynucleotide that is substantially similar to any one of SEQ ID NOS: 30 to 48.

In some cases, the promoters and/or the introns of the isobutanol pathway genes can be altered using the advance genome editing tools described herein. This alteration may enhance the expression of the genes that are controlled by the promoters and/or introns. In some cases, the alternation may inhibit the expression of the genes that are controlled by the promoters and/or introns.

In some cases, the codons can be optimized based on the microorganism in which the genes will be provided or the enzymes will be expressed.

### Isobutyraldehyde

In some instances, polynucleotides encoding enzymes of the isobutyraldehyde pathway can be used. For example, the microorganism can contain (either endogenously or heterologous) one or more polynucleotides encoding for an acetolactate synthase (AlsS); ketol-acid reductoisomerase; dihydroxy-acid dehydratase; and 2-keto acid decarboxylase (KDC). One or more of the polynucleotides can be native to the microorganism. In some cases, one or more of the polynucleotides can be heterologous to the microorganism.

In some cases, the acetolactate synthase (AlsS) can be encoded by a polynucleotide that is substantially similar to a gram positive bacterium AlsS gene. In some cases, the AlsS can be encoded by a polynucleotide that is substantially similar to SEQ ID NO: 1. In some other cases, the AlsS can be encoded by a polynucleotide that is substantially similar to SEQ ID NO: 2.

In some cases, the ketol-acid reductoisomerase can be encoded by a polynucleotide that is substantially similar to a gram negative bacterium ketol-acid reductoisomerase gene. In some cases, the ketol-acid reductoisomerase can be encoded by a polynucleotide that is substantially similar to SEQ ID NO: 3.

In some cases, the dihydroxy-acid dehydratase can be encoded by a polynucleotide that is substantially similar to a gram negative bacterium dihydroxy-acid dehydratase gene. In some cases, the dihydroxy-acid dehydratase can be encoded by a polynucleotide that is substantially similar to SEQ ID NO: 4. In some cases, the dihydroxy-acid dehydratase can be encoded by a polynucleotide that is substantially similar to SEQ ID NO: 5.

In some cases, the 2-keto acid decarboxylase (KDC) can be encoded by a polynucleotide that is substantially similar to a gram positive bacterium KDC gene. In some cases, the KDC can be encoded by a polynucleotide that is substantially similar to any one of SEQ ID NOs: 6 to 29.

In some cases, the promoters and/or the introns of the isobutyraldehyde pathway genes can be altered using the advance genome editing tools described herein. This alteration may enhance the expression of the genes that are controlled by the promoters and/or introns. In some cases, the alternation may inhibit the expression of the genes that are controlled by the promoters and/or introns.

In some cases, the codons can be optimized based on the microorganism in which the genes will be provided or the enzymes will be expressed.

### 2,3-BDO

In some instances, polynucleotides encoding enzymes of the 2,3-BDO pathway can be used. For example, the microorganism can contain (either endogenously or heterologous) one or more polynucleotides encoding for an acetolactate synthase (AlsS), alpha-acetolactate decarboxylase (budA), and/or acetoin reductase. One or more of the polynucleotides can be native to the microorganism. In some cases, one or more of the polynucleotides can be heterologous to the microorganism.

In some cases, the acetolactate synthase can be encoded by a polynucleotide that is substantially similar to any one of SEQ ID NOs: 49 to 51.

In some cases, the alpha-acetolactate decarboxylase can be encoded by a polynucleotide that is substantially similar to SEQ ID NO: 52 or 53.

In some cases, the acetoin reductase can be encoded by a polynucleotide that is substantially similar to any one of SEQ ID NOs: 54 to 56. In some cases, the acetoin reductase can be NADPH-dependent. In some cases, the acetoin reductase can be NADH-dependent.

In some cases, the promoters and/or the introns of the 2,3-BDO pathway genes can be altered using the advance genome editing tools described herein.

In some cases, the codons can be optimized based on the microorganism in which the genes will be provided or the enzymes will be expressed.

### 1,4-BDO

In some instances, polynucleotides encoding enzymes of the 1,4-BDO pathway can be used. For example, the microorganism can contain (either endogenously or heterologous) one or more polynucleotides encoding for a pyruvate dehydrogenase (aceEF), citrate synthase (gltA), aconitate hydratase 1 (acnA), isocitrate dehydrogenase (icdA), α-ketoglutarate decarboxylase (kgd), succinyl-CoA synthetase (sucC), CoA-dependent succinate semialdehyde dehydrogenase (sucD), 4-hyrobutyrate dehydrogenase (4hbD), 4-hydroxybutyryl-CoA transferase (cat2), aldehyde dehydrogenase (ald), and/or alcohol dehydrogenase (adh).

In some cases, the α-ketoglutarate decarboxylase (kgd) can be encoded by a polynucleotide that is substantially similar to any one of SEQ ID NOs: 57 to 60.

In some cases, the 4-hydroxybutyrate dehydrogenase (4hbD) can be encoded by a polynucleotide that is substantially similar to SEQ ID NO. 61 or 62.

In some cases, the 4-hydroxybutyrate CoA transferase (Cat2) can be encoded by a polynucleotide that is substantially similar to any one of SEQ ID NOs: 63 to 65.

In some cases, the aldehyde dehydrogenase gene and/or alcohol dehydrogenase can be encoded by a polynucleotide that is substantially similar to any one of SEQ ID NOs: 66 to 73.

In some cases, the succinyl CoA synthease beta subunit (sucC) can be encoded by a polynucleotide that is substantially similar to SEQ ID NO: 74.

In some cases, the succinyl CoA synthease alpha subunit (sucD) can be encoded by a polynucleotide that is substantially similar to any one of SEQ ID NOs: 75 to 77.

In some cases, the promoters and/or the introns of the 1,4-BDO pathway genes can be altered using the advance genome editing tools described herein.

In some cases, the codons can be optimized based on the microorganism in which the genes will be provided or the enzymes will be expressed.

RNA polymerase beta-subunits

In some cases, polynucleotides encoding for RNA polymerase beta-subunits can be used. RNA polymerase beta-subunits (e.g., those with an EC:2.7.7.6) can be used to confer onto a microorganism resistance to some antibiotics, such as rifampin. In some cases, RNA polymerase beta-subunits can be expressed endogenously by a microorganism. Repression or knocking out of the genes encoding RNA polymerase beta-subunits (including but not limited to RNA polymerase beta-subunits encoded by such genes as rpoB or rpoB2) can lead to the loss of resistance to such antibiotics, such as rifampin. In these cases, repressing or knocking out of the genes encoding RNA polymerase beta-subunits can lead to cell death.

The microorganism can contain (either endogenously or heterologous) one or more polynucleotides encoding for RNA polymerase beta-subunits. One or more of the polynucleotides can be native to the microorganism. In some cases, one or more of the polynucleotides can be heterologous to the microorganism.

In some cases, the RNA polymerase beta-subunits can be encoded by a polynucleotide that is substantially similar to SEQ ID NO: 123 or 126. In some cases, the gRNA used can be substantially similar to SEQ ID NO: 124 or 125.

In some cases, the donor DNA can be substantially similar to SEQ ID NO: 126.

In some cases, the promoters and/or the introns of the RNA polymerase beta-subunits gene(s) can be altered using the advance genome editing tools described herein. This alteration may enhance the expression of the genes that are controlled by the promoters and/or introns. In some cases, the alternation may inhibit the expression of the genes that are controlled by the promoters and/or introns.

In some cases, the codons can be optimized based on the microorganism in which the genes will be provided or the enzymes will be expressed.

### Vectors

Since Cas enzymes are not native to some microorganisms, expression vectors can be used to express Cas enzymes within most microorganisms and cells. Methylotrophs such as methanotrophs do not naturally express Cas enzymes. Therefore, in some cases, the Cas enzymes can be expressed using certain expression vectors. Vector constructs prepared for introduction into the host microorganisms described throughout may typically, but not always, comprise a replication system (i.e. vector) recognized by the host. In some cases, the vector includes the intended polynucleotide fragment encoding the desired polypeptide and, optionally, transcription and translational initiation regulatory sequences operably linked to the polypeptide-encoding segment. Expression vectors may include, for example, an origin of replication or autonomously replicating sequence (ARS), expression control sequences, a promoter, an enhancer and necessary processing information sites, such as ribosome-binding sites, RNA splice sites, polyadenylation sites, transcriptional terminator sequences, mRNA stabilizing sequences, polynucleotides homologous to host chromosomal DNA, and/or a multiple cloning site. Signal peptides may also be included where appropriate, preferably from secreted polypeptides of the same or related species, which allow the protein to cross and/or lodge in cell membranes or be secreted from the cell.

The vectors can be constructed using standard methods (see, e.g., Sambrook et al., Molecular Biology: A Laboratory Manual, Cold Spring Harbor, N.Y. 1989; and Ausubel, et al., Current Protocols in Molecular Biology, Greene Publishing, Co. N.Y, 1995).

Manipulation of polynucleotides that encode the enzymes disclosed throughout is typically carried out in recombinant vectors. Vectors which may be employed include bacterial plasmids, bacteriophage, artificial chromosomes, episomal vectors and gene expression vectors. Vectors may be selected to accommodate a polynucleotide encoding a protein of a desired size. Following production of a selected vector, a suitable host cell (e.g., the microorganisms described herein) is transfected or transformed with the vector. Each vector contains various functional components, which generally include a cloning site, an origin of replication and at least one selectable marker gene. A vector may additionally possess one or more of the following elements: an enhancer, promoter, a transcription termination sequence and/or other signal sequences. Such sequence elements may be optimized for the selected host species. Such sequence elements may be positioned in the vicinity of the cloning site, such that they are operatively linked to the gene encoding a preselected enzyme.

Vectors, including cloning and expression vectors, may contain polynucleotides that enable the vector to replicate in one or more selected microorganisms. For example, the sequence may be one that enables the vector to replicate independently of the host chromosomal DNA and may include origins of replication or autonomously replicating sequences. Such sequences are well known for a variety of bacteria, yeast and viruses. For example, the origin of replication from the plasmid pBR322 is suitable for most gram-negative bacteria, the origin of replication for 2 micron plasmid is suitable for yeast, and various viral origins of replication (e.g. SV40, adenovirus) are useful for cloning vectors.

A cloning or expression vector may contain a selection gene, also referred to as a selectable marker. This gene encodes a protein necessary for the survival or growth of transformed microorganisms in a selective culture medium. Microorganisms not transformed with the vector containing the selection gene will therefore not survive in the culture medium. Typical selection genes encode proteins that confer resistance to antibiotics and other toxins, e.g. ampicillin, neomycin, methotrexate, hygromycin, thiostrepton, apramycin or tetracycline, complement auxotrophic deficiencies, or supply critical nutrients not available in the growth media.

The replication of vectors may be performed in E. coli. An example of a E. coli-selectable marker is the β-lactamase gene, which confers resistance to the antibiotic ampicillin. These selectable markers can be obtained from E. coli plasmids, such as pBR322 or a pUC plasmid such as pUC18 or pUC19, or pUC119.

The vectors of the present invention can comprise one or more switches, such as an inducible or repressible switch, e.g., an arabinose or lanthanum switch. The vectors can also comprise one or more different/same promoters.

### Promoters

Vectors may contain a promoter that is recognized by the host microorganism. The promoter may be operably linked to a coding sequence of interest. Such a promoter may be inducible or constitutive. Polynucleotides are operably linked when the polynucleotides are in a relationship permitting them to function in their intended manner.

Different promoters can be used to drive the expression of the genes. For example, if temporary gene expression (i.e., non-constitutively expressed) is desired, expression can be driven by inducible promoters.

In some cases, the desired gene is expressed temporarily. In other words, the desired gene is not constitutively expressed. The expression of the desired gene can be driven by inducible or repressible promoters. Examples of inducible or repressible promoters include, but are not limited to, those promoters inducible or repressible by: (a) sugars such as arabinose and lactose (or non-metabolizable analogs, e.g., isopropyl β-D-1-thiogalactopyranoside (IPTG)); (b) metals such as rare earth metals (e.g., lanthanum or cerium), copper, and calcium; (c) temperature; (d) nitrogen-source; (e) oxygen; (f) cell state (growth or stationary); (g) metabolites such as phosphate; (h) CRISPRi; (i) jun; (j) fos; (k) metallothionein, and/or (1) heat shock. These promoters can be used in a methanotroph system. An example of an inducible promoter that can be used within methanotrophs is a pBAD promoter.

Inducible or repressible promoters that can be particularly useful are sugar and rare earth metal switches. For example, promoters that are sensitive to the sugar arabinose can be used as an inducible switch. In some cases, arabinose switches can be used to drive expression of one or more genes. For example, in the presence arabinose, a desired vector or expression of a gene set can be “turned-on.” The arabinose switch can turn on the expression of a desired gene.

Other particularly useful switches can be rare earth metal switches, such as lanthanum switches. In some cases, the lanthanum switch can be a repressible switch that can be used to repress expression of one or more genes, until the repressor is removed, after which the genes are “turned-on”. For example, in the presence the metal lanthanum, the desired gene set or vector can be “turned-off.” The lanthanum switch can turned off (and expression of the genes induced) by either removing the lanthanum from the media or diluting the lanthanum in the media to levels where its repressible effects are reduced, minimized, or eliminated.

Constitutively expressed promoters can also be used in the vector systems herein. For example, the expression of one or more desired genes can be controlled by constitutively active promoters. Examples of such promoters include but are not limited to pMxaF and p.Bba.J23111.

Promoters suitable for use with prokaryotic hosts may include, for example, the α-lactamase and lactose promoter systems, alkaline phosphatase, the tryptophan (trp) promoter system, the erythromycin promoter, apramycin promoter, hygromycin promoter, methylenomycin promoter and hybrid promoters such as the tac promoter. Promoters for use in bacterial systems will also generally contain a Shine-Dalgarno sequence operably linked to the coding sequence.

Generally, a strong promoter may be employed to provide for high level transcription and expression of the desired product. For example, promoters that can be used include but are not limited to a pMxaF promoter. In some cases, a mutation can increase the strength of the promoter and therefore result in elevated levels of expression.

In some cases however, a weaker promoter is desired. For example, this is the case where too much expression of a certain gene results in a detrimental effect (e.g., the killing of cells). A weak promoter can be used, for example, a pBAD promoter. However, in some cases, a weaker promoter can be made by mutation. For example, the pMxaF promoters can be mutated to be weaker.

One or more promoters of a transcription unit can be an inducible promoter. For example, a green fluorescent protein (GFP) can be expressed from a constitutive promoter while an inducible promoter is used to drive transcription of a gene coding for one or more enzymes as disclosed herein and/or the amplifiable selectable marker.

Some vectors may contain prokaryotic sequences that facilitate the propagation of the vector in bacteria. Thus, the vectors may have other components such as an origin of replication (e.g., a polynucleotide that enables the vector to replicate in one or more selected microorganisms), antibiotic resistance genes for selection in bacteria, and/or an amber stop codon which can permit translation to read through the codon. Additional selectable gene(s) may also be incorporated. Generally, in cloning vectors, the origin of replication is one that enables the vector to replicate independently of the host chromosomal DNA, and includes origins of replication or autonomously replicating sequences. Such sequences can include the ColEl origin of replication in bacteria or other known sequences.

### Genes

The genes described throughout all have a promoter driving their expression. The methods described herein, e.g., genome editing and expression inhibition using Cas, can be used to edit the polynucleotide of the promoters or used to inhibit the effectiveness of the promoters. Inhibition can be done by blocking the transcription machinery (e.g., transcription factors) from binding to the promoter or by altering the promoter in such a way that the transcription machinery no longer recognizing the promoter sequence.

The vectors described throughout can also comprise a polynucleotide encoding for one or more of the genes within the 2,3-butanediol (2,3-BDO), 1,4-butanediol (1,4-BDO), isobutyraldehyde, or isobutanol pathway. The vectors described throughout can also comprise a polynucleotide encoding for an RNA polymerase beta-subunit. These vectors can also contain one or more regulatory elements (inducible and/or repressible promoters) that control the expression of the genes within the vectors. In some cases, the switches that can be used include, but are not limited to, inducible or repressible switches, e.g., an arabinose or lanthanum switches. These genes can be heterologous to the microorganism in which the vector is contacted with (and eventually transformed with).

The genes used in the vectors can be any genes described throughout the application. For example, the genes of the 2,3-BDO, 1,4-BDO, isobutanol, and/or isobutyraldehyde pathways. These enzymes can be encoded by a polynucleotide that is at least 60%, 65%, 70%, 75%, 80%, 85%, 90%, 91%, 92%, 93%, 94%, 95%, 96%, 97%, 98%, 99%, 99.9%, 99.99%, 99.999%, or 99.9999% identical to any one of SEQ ID NOs: 1 to 77. In some cases, the RNA polymerase beta-subunit genes can be used in the vectors. This enzyme can be encoded by a polynucleotide that is at least 60%, 65%, 70%, 75%, 80%, 85%, 90%, 91%, 92%, 93%, 94%, 95%, 96%, 97%, 98%, 99%, 99.9%, 99.99%, 99.999%, or 99.9999% identical to SEQ ID NOs: 123 or 126.

The genes that are inserted into a microorganism can be heterologous to the microorganism itself. For example, if the microorganism is a methanotroph, the inserted genes can, for example, be from yeast, a bacterium, or a different species of methanotroph. Further, the genes can be endogenously part of the genome of the microorganism.

### III. Techniques for Genetic Modification

The microorganisms disclosed herein may be genetically engineered by using classic microbiological techniques. These classical techniques can be in addition to the advanced genome editing techniques. Some of such classical techniques are generally disclosed, for example, in Sambrook et al., 1989, Molecular Cloning: A Laboratory Manual, Cold Spring Harbor Labs Press.

The genetically modified microorganisms disclosed herein may include a polynucleotide that has been inserted, deleted or modified (i.e., mutated; e.g., by insertion, deletion, substitution, and/or inversion of nucleotides), in such a manner that such modifications provide the desired effect of expression (e.g., over-expression or decreased expression) of one or more enzymes as provided herein within the microorganism. Genetic modifications which result in an increase in gene expression or function can be referred to as amplification, overproduction, overexpression, activation, enhancement, addition, or up-regulation of a gene. Addition of a gene to increase expression can include maintaining the gene(s) on replicating plasmids or integrating the cloned gene(s) into the genome of the production microorganism. Furthermore, increasing the expression of desired genes can include operatively linking the cloned gene(s) to native or heterologous transcriptional control elements. Additionally, increasing expression of a desired gene can also include modifying the promoter region of the gene. Genetic modifications which result in a decrease in gene expression or function can be referred to as reduction, repression, underproduction, deactivation, deletion, or down-regulation of a gene. In some cases, the genetic modification which results in a decrease in gene expression or function can be complete elimination of gene expression (knockout) or partial elimination of gene expression (knockdown—e.g., via RNAi).

Where desired, the expression of one or more of the enzymes provided herein is under the control of a regulatory sequence that controls directly or indirectly the enzyme expression in a time-dependent fashion during the fermentation. Inducible promoters can be used to achieve this. As discussed throughout, the methods described herein can be used to alter the polynucleotide of the promoters.

In some cases, a microorganism is transformed or transfected with a genetic vehicle, such as an expression vector comprising a heterologous polynucleotide encoding for the enzymes as provided herein. In some cases, the heterologous polynucleotide encoding for the enzymes throughout can be altered using the techniques described throughout, before or after, the heterologous enzyme is placed within the microorganism.

To facilitate insertion and expression of different genes coding for the enzymes as disclosed herein from the constructs and expression vectors, the constructs may be designed with at least one cloning site for insertion of any gene coding for any enzyme disclosed herein. The cloning site may be a multiple cloning site, e.g., containing multiple restriction sites.

### Transfection

Standard transfection techniques can be used to insert genes into a microorganism. As used herein, the term “transfection” or “transformation” can refer to the insertion of an exogenous nucleic acid or polynucleotide into a host cell. The exogenous nucleic acid or polynucleotide may be maintained as a non-integrated vector, for example, a plasmid, or alternatively, may be integrated into the host cell genome. The term transfecting or transfection is intended to encompass all conventional techniques for introducing nucleic acid or polynucleotide into microorganisms. Examples of transfection techniques include, but are not limited to, calcium phosphate precipitation, DEAE-dextran-mediated transfection, lipofection, electroporation, microinjection, rubidium chloride or polycation mediated transfection, protoplast fusion, and sonication. The transfection method that provides optimal transfection frequency and expression of the construct in the particular host cell line and type is favored. For stable transfectants, the constructs are integrated so as to be stably maintained within the host chromosome. In some cases, the preferred transfection is a stable transfection.

### Transformation

Expression vectors or other nucleic acids may be introduced to selected microorganisms by any of a number of suitable methods. For example, vector constructs may be introduced to appropriate cells by any of a number of transformation methods for plasmid vectors. Standard calcium-chloride-mediated bacterial transformation is still commonly used to introduce naked DNA to bacteria (see, e.g., Sambrook et al., 1989, Molecular Cloning, A Laboratory Manual, Cold Spring Harbor Laboratory Press, Cold Spring Harbor, N.Y.), but electroporation and conjugation may also be used (see, e.g., Ausubel et al., 1988, Current Protocols in Molecular Biology, John Wiley & Sons, Inc., NY, N.Y.).

For the introduction of vector constructs to yeast or other fungal cells, chemical transformation methods may be used (e.g., Rose et al., 1990, Methods in Yeast Genetics, Cold Spring Harbor Laboratory Press, Cold Spring Harbor, N.Y.). Transformed cells may be isolated on selective media appropriate to the selectable marker used. Alternatively, or in addition, plates or filters lifted from plates may be scanned for GFP fluorescence to identify transformed clones.

For the introduction of vectors comprising differentially expressed sequences to certain types of cells, the method used may depend on the form of the vector. Plasmid vectors may be introduced by any of a number of transfection methods, including, for example, lipid-mediated transfection (“lipofection”), DEAE-dextran-mediated transfection, electroporation or calcium phosphate precipitation (see, e.g., Ausubel et al., 1988, Current Protocols in Molecular Biology, John Wiley & Sons, Inc., NY, N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. Many companies offer kits and ways for this type of transfection.

The host cell may be capable of expressing the construct encoding the desired protein, processing the protein and transporting a secreted protein to the cell surface for secretion. Processing includes co- and post-translational modification such as leader peptide cleavage, GPI attachment, glycosylation, ubiquitination, and disulfide bond formation.

Microorganisms can be transformed or transfected with the above-described expression vectors or polynucleotides coding for one or more enzymes as disclosed herein and cultured in nutrient media modified as appropriate for the specific microorganism, inducing promoters, selecting transformants, or amplifying the genes encoding the desired sequences. In some cases, electroporation methods can be used to deliver an expression vector.

Expression of a vector (and the gene contained in the vector) can be verified by an expression assay, for example, qPCR or by measuring levels of RNA. Expression level can be indicative also of copy number. For example, if expression levels are extremely high, this can indicate that more than one copy of a gene was integrated in a genome. Alternatively, high expression can indicate that a gene was integrated in a highly transcribed area, for example, near a highly expressed promoter. Expression can also be verified by measuring protein levels, such as through Western blotting.

### CRISPR Cas

The methods disclosed throughout can involve pinpoint nucleotide replacement, pinpoint insertion of one or more nucleotides (e.g., addition of genes or parts of genes) or the pinpoint deletion of one or more nucleotide (e.g., deletion of genes or parts of genes). Methods described herein can use a CRISPR/cas system. For example, double-strand breaks (DSBs) can be generated using a CRISPR/cas system, e.g., a type II CRISPR/cas system. A Cas enzyme used in the methods disclosed herein can be Cas9, which catalyzes DNA cleavage. Enzymatic action by Cas9 from Streptococcus pyogenes or any closely related Cas9 can generate double stranded breaks at target site sequences which hybridize to 20 nucleotides of a guide sequence and have a protospacer-adjacent motif (PAM) following the 20 nucleotides of the target sequence.

A vector can also encode a Cas enzyme. Cas enzymes that can be used include class 1 and class 2. Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof. An unmodified Cas enzyme can have DNA cleavage activity. A Cas enzyme can direct cleavage of one or both strands at a target sequence, such as within a target sequence and/or within a complement of a target sequence. For example, a Cas enzyme can direct cleavage of one or both strands within 1, 2, 3, 4, 5, 6, 7, 8, 9, 10, 15, 20, 25, 30, 35, 40, 45, 50, 60, 70, 80, 90, 100, 125, 150, 175, 200, 300, 400, 500, or more base pairs from the first or last nucleotide of a target sequence. A vector that encodes a Cas enzyme that is mutated with respect to a corresponding wild-type enzyme such that the mutated Cas enzyme lacks the ability to cleave one or both strands of a target polynucleotide containing a target sequence can be used. Additionally, a modified Cas enzyme that lacks the ability to cleave but has the ability to block binding of the transcriptional machinery can be used.

A vector that encodes a Cas enzyme comprising one or more nuclear localization sequences (NLSs) can be used. For example, there can be 1, 2, 3, 4, 5, 6, 7, 8, 9, 10 NLSs used. A Cas enzyme can comprise the NLSs at or near the ammo-terminus (e.g., 1, 2, 3, 4, 5, 6, 7, 8, 9, 10 NLSs), or at or near the carboxy-terminus (e.g., 1, 2, 3, 4, 5, 6, 7, 8, 9, 10 NLSs), or any combination of these (e.g., one or more NLS at the ammo-terminus and one or more NLS at the carboxy terminus). When more than one NLS is present, each can be selected independently of others, such that a single NLS can be present in more than one copy and/or in combination with one or more other NLSs present in one or more copies.

Cas enzyme used in the methods can comprise at most 6 NLSs. An NLS is considered near the N- or C-terminus when the nearest amino acid to the NLS is within 50 amino acids along a polypeptide chain from the N- or C-terminus, e.g., within 1, 2, 3, 4, 5, 10, 15, 20, 25, 30, 40, or 50 amino acids.

### Guide RNA

As used herein, the term “guide RNA” (gRNA) and its grammatical equivalents can refer to an RNA which can be specific for a target DNA and can form a complex with Cas enzyme. An RNA/Cas complex can assist in “guiding” Cas enzyme to a target DNA.

A method disclosed herein also can comprise introducing into a microorganism at least one guide RNA or other nucleic acid, e.g., DNA encoding at least one guide RNA. A guide RNA can interact with a RNA-guided endonuclease to direct the endonuclease to a specific target site, at which site the 5′ end of the guide RNA base pairs with a specific protospacer sequence in a chromosomal sequence.

A guide RNA can comprise two RNAs, e.g., CRISPR RNA (crRNA) and transactivating crRNA (tracrRNA). A guide RNA can sometimes comprise a single-chain RNA, or single guide RNA (sgRNA) formed by fusion of a portion (e.g., a functional portion) of crRNA and tracrRNA. A guide RNA can also be a dualRNA comprising a crRNA and a tracrRNA. Furthermore, a crRNA can hybridize with a target DNA.

As discussed above, a guide RNA can be an expression product. For example, a DNA that encodes a guide RNA can be a vector comprising a sequence coding for the guide RNA. A guide RNA can be transferred into a microorganism by transfecting the microorganism with an isolated guide RNA or plasmid DNA comprising a sequence encoding for the guide RNA and a promoter. A guide RNA can also be transferred into a microorganism in other ways, such as using virus-mediated gene delivery.

A guide RNA can be isolated. For example, a guide RNA can be transfected in the form of an isolated RNA into a microorganism. A guide RNA can be prepared by in vitro transcription using any in vitro transcription system. A guide RNA can be transferred to a microorganism in the form of isolated RNA rather than in the form of plasmid comprising encoding sequence for a guide RNA.

A guide RNA can comprise three regions: a first region at the 5′ end that can be complementary to a target site in a chromosomal sequence; a second internal region that can form a stem loop structure; and a third 3′ region that can be single-stranded. A first region of each guide RNA can also be different such that each guide RNA guides a fusion protein to a specific target site. Further, second and third regions of each guide RNA can be identical in all guide RNAs.

A first region of a guide RNA can be complementary to sequence at a target site in a chromosomal sequence such that the first region of the guide RNA can base pair with the target site. In some cases, a first region of a guide RNA can comprise from 10 nucleotides to 25 nucleotides (i.e., from 10 nts to 25 nts; or 10 nts to 25 nts; or from 10 nts to 25 nts; or from 10 nts to 25 nts) or more. For example, a region of base pairing between a first region of a guide RNA and a target site in a chromosomal sequence can be 10, 11, 12, 13, 14, 15, 16, 17, 18, 19, 20, 22, 23, 24, 25, or more nucleotides in length. Sometimes, a first region of a guide RNA can be 19, 20, or 21 nucleotides in length.

A guide RNA can also comprises a second region that forms a secondary structure. For example, a secondary structure formed by a guide RNA can comprise a stem (or hairpin) and a loop. A length of a loop and a stem can vary. For example, a loop can range from 3 to 10 nucleotides in length, and a stem can range from 6 to 20 base pairs in length. A stem can comprise one or more bulges of 1 to 10 nucleotides. The overall length of a second region can range from 16 to 60 nucleotides in length. For example, a loop can be 4 nucleotides in length and a stem can be 12 base pairs.

A guide RNA can also comprise a third region at the 3′ end that can be essentially single-stranded For example, a third region is sometimes not complementarity to any chromosomal sequence in a cell of interest and is sometimes not complementarity to the rest of a guide RNA. Further, the length of a third region can vary. A third region can be more than 4 nucleotides in length. For example, the length of a third region can range from 5 to 60 nucleotides in length.

A guide RNA can be introduced into a microorganism as an RNA molecule. For example, a RNA molecule can be transcribed in vitro and/or can be chemically synthesized. An RNA can be transcribed from a synthetic DNA molecule, e.g., a gBlocks® gene fragment. A guide RNA can then be introduced into a microorganism as an RNA molecule. A guide RNA can also be introduced into a microorganism in the form of a non-RNA nucleic acid molecule, e.g., DNA molecule. For example, a DNA encoding a guide RNA can be operably linked to promoter control sequence for expression of the guide RNA in a microorganism of interest. A RNA coding sequence can be operably linked to a promoter sequence that is recognized by an RNA polymerase. Plasmid vectors that can be used to express guide RNA include, but are not limited to, px330 vectors and px333 vectors. In some cases, a plasmid vector (e.g., px333 vector) can comprise two guide RNA-encoding DNA sequences.

A DNA sequence encoding a guide RNA can also be part of a vector. Further, a vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. A DNA molecule encoding a guide RNA can also be linear. A DNA molecule encoding a guide RNA can also be circular.

When DNA sequences encoding an RNA-guided endonuclease and a guide RNA are introduced into a cell, each DNA sequence can be part of a separate molecule (e.g., one vector containing an RNA-guided endonuclease coding sequence and a second vector containing a guide RNA coding sequence) or both can be part of a same molecule (e.g., one vector containing coding (and regulatory) sequence for both an RNA-guided endonuclease and a guide RNA).

### Donor DNA

As used herein, the term “donor DNA” and its grammatical equivalents can refer to a polynucleotide that provides a template for “repair” during the insertion of one or more nucleotides during genome editing. For example, the Cas9 enzyme can provide specific double stranded DNA breaks using a guide RNA. Should one or more nucleotides be desired to be inserted into this double stranded break, the donor DNA can be used. The donor DNA can be inserted into the double stranded break site. Further, a single strand of DNA can be provided at the break site and a microorganism's repair mechanisms can be used to complete the full insertion of a double stranded DNA.

### Timing of Transformation

The timing of the expression of the specific components used in genome editing can be important in its efficacy. For example, for some microorganisms, transformation of plasmids expressing a Cas protein, guide RNA, and/or donor DNA can be simultaneously introduced into the microorganism and effectively be used to insert or delete one or more nucleotides.

However, for certain microorganisms that are capable of converting a C₁ carbon into a product, e.g., methanotrophs, the order in which a Cas protein, guide RNA, and/or donor DNA are transformed into the microorganism makes a significant difference in the effectiveness of genome editing as well as survival of the microorganism. For example, the transformation of Cas proteins prior to the transformation of the guide RNA and/or donor DNA results in an increased amount of cell death and decreased editing efficiency. To increase editing efficiency and to reduce unwanted cell death, the microorganisms can be transformed with a guide RNA and/or donor DNA prior to the transformation of a Cas protein.

In some cases, the guide RNA, donor DNA, and/or Cas enzyme, are found on separate plasmids/vectors. In some cases, the guide RNA and donor DNA are on a single plasmid/vector, while the Cas enzyme is expressed from a separate plasmid/vector. In some cases, the guide RNA is expressed on a single plasmid/vector, while the donor DNA and Cas enzyme are expressed on a separate plasmid/vector. In some cases, the donor DNA is expressed on a single plasmid/vector, while the guide RNA and Cas enzyme are expressed on a separate plasmid/vector.

In some cases, the guide RNA and Cas enzyme can be controlled by different promoters. For example, in some cases, the guide RNA can be controlled by a constitutively expressed promoter while the expression of the Cas enzyme is controlled by an inducible promoter. In some cases, should a donor DNA be required, it can be expressed under the control of either a constitutively expressed promoter or an inducible promoter. This, in some cases, can allow for transformation of a microorganism with guide RNA and Cas enzyme (and optionally donor DNA) at the same time.

### Site-Specific Insertion

Insertion of the one or more nucleotides (e.g., genes) can be site-specific. For example, one or more nucleotides (e.g., genes) can be inserted adjacent to a promoter.

Modification of a targeted locus of a microorganism can be produced by introducing DNA into microorganisms, where the DNA has homology to the target locus. DNA can include a marker gene, allowing for selection of cells comprising the integrated construct. Homologous DNA in a target vector can recombine with DNA at a target locus. A marker gene can be flanked on both sides by homologous DNA sequences, a 3′ recombination arm, and a 5′ recombination arm.

A variety of enzymes can catalyze insertion of foreign DNA into a microorganism genome. For example, site-specific recombinases can be clustered into two protein families with distinct biochemical properties, namely tyrosine recombinases (in which DNA is covalently attached to a tyrosine residue) and serine recombinases (where covalent attachment occurs at a serine residue). In some cases, recombinases can comprise Cre, fC31 integrase (a serine recombinase derived from Streptomyces phage fC31), or bacteriophage derived site-specific recombinases (including Flp, lambda integrase, bacteriophage HK022 recombinase, bacteriophage R4 integrase and phage TP901-1 integrase).

The CRISPR/Cas system can be used to perform site specific insertion. For example, a nick on an insertion site in the genome can be made by CRISPR/Cas to facilitate the insertion of a transgene at the insertion site.

The techniques which can be used to allow a DNA or RNA construct entry into a host microorganism in the methods described herein include, but are not limited to, calcium phosphate/DNA coprecipitation, microinjection of DNA into a nucleus, electroporation, bacterial protoplast fusion with intact cells, transfection, lipofection, infection, particle bombardment, sperm mediated gene transfer, or any other technique.

Certain aspects disclosed herein can utilize vectors (including the ones described above). Any plasmids and vectors can be used as long as they are replicable and viable in a selected host microorganism. Vectors known in the art and those commercially available (and variants or derivatives thereof) can be engineered to include one or more recombination sites for use in the methods herein. Vectors that can be used include, but are not limited to, expression vectors such as pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), pXT1, pSG5, pPbac, pMbac, p.MClneo, and pOG44 (Stratagene, Inc.), and pYES2, pAC360, pBlueBa-cHis A, B, and C, pVL1392, pBlueBac111, pCDM8, pcDNA1, pZeoSV, pcDNA3, pREP4, pCEP4, and pEBVHis (Invitrogen, Corp.), and variants or derivatives thereof.

These vectors can be used to express a gene or portion of a gene of interest. A gene or a portion of a gene can be inserted by using known methods, such as restriction enzyme-based techniques.

### IV. Methods of Altering Nucleic Acids

The nucleic acids contained within the microorganism disclosed throughout can be altered in specific ways. Depending on the type of modification desired, guide RNAs can be made and targeted to specific sequences. For example, the nucleic acids described throughout can be within the microorganism described herein. Then if specific modifications are desired, the modifications can be made within the microorganism without going through an entire process of genetically engineering the microorganism from the beginning.

In some cases, wild-type, unmodified microorganisms can be altered by the methods described. For example, wild-type methylotrophs, such as methanotrophs, e.g. Methylococcus capsulatus, can be genetically altered. In some cases, previously genetically modified microorganisms can be altered by the methods described. For example, the genetically modified microorganism described herein, such as those that produce 2,3-BDO, 1,4-BDO, isobutanol and/or isobutyraldehyde, can be further genetically modified using the methods described throughout. The nucleic acids within the microorganism (both heterologous or native) can be introduced with point mutations, addition of one or more nucleic acids, and/or deletion of one or more nucleic acids.

### General Methods

Generally described throughout are methods of genetic engineering. In one example, described herein is a method of genetic engineering comprising: (a) contacting a microorganism that is capable of converting a C₁ carbon to a multicarbon product with a polynucleotide encoding for a Cas enzyme and a guide ribonucleic acid (gRNA); and (b) growing the microorganism until a genetic modification occurs.

In some cases, the microorganism is a microorganism as described throughout, such as a methylotroph. For example, the methylotroph can be a methanotroph, such as from the genera Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, or Methyloacidophilum. In some cases, the methanotroph can be from the genus Methylococcus, such as a Methylococcus capsulatus.

In some cases, the C₁ carbon can be any C₁ carbon disclosed throughout. For example, in some cases the C₁ carbon is carbon monoxide (CO), carbon dioxide (CO₂), methane (CH₄), or any combination thereof. In some cases, the C₁ carbon is CH₄.

The Cas enzymes that can be used for any of the methods described throughout include but are not limited to Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof. In some cases, the Cas enzyme is a Cas9 enzyme.

In some cases, the gRNA can be at least partially or fully homologous to any one of the genes or promoters described throughout. In some instances, the gRNA is at least partially homologous or fully homologous to a promoter, intron, or coding sequence of an rpoB gene or a gene within the 2,3-butanediol (2,3-BDO), 1,4-butanediol (1,4-BDO), isobutyraldehyde, or isobutanol pathway.

In some cases, the term “at least partially homologous” can refer to having at least two or more nucleotides identical in a sequence. In most cases, the term at least partially homologous refers to a polynucleotide that is identical in at least a 10 nucleotide stretch. Thus, at least 10 or more nucleotides from the gRNA can bind to the polynucleotide that is being pinpointed and/or altered.

In some cases, the gRNA is directed to a promoter, intron, or coding sequence of gene within the 2,3-BDO pathway. The gene within the 2,3-BDO can be an acetoin reductase, alpha-acetolactate decarboxylase, and/or acetolactate synthase gene.

In other cases, the gRNA is directed to a promoter, intron, or coding sequence of a gene within the 1,4-BDO pathway. The gene can be within the 1,4-BDO pathway can be a pyruvate dehydrogenase (aceEF), citrate synthase (gltA), aconitate hydratase 1 (acnA), isocitrate dehydrogenase (icdA), citrate synthase (gltA), succinyl-CoA synthetase (SucC), CoA-dependent succinate semialdehyde dehydrogenase (SucD), 4-hyrobutyrate dehydrogenase (4hbD), 4-hydroxybutyryl-CoA transferase (Cat2), aldehyde dehydrogenase (Ald), alcohol dehydrogenase (Adh), and/or α-ketoglutarate decarboxylase (kgd) gene.

In some cases, the gRNA is directed to a promoter, intron, or coding sequence of a gene within the isobutyraldehyde pathway. The gene within the isobutyraldehyde pathway can be an acetolactate synthase (AlsS), ketol-acid reductoisomerase (IlvC), dihydroxy-acid dehydratase (IlvD), and/or 2-keto acid decarboxylase (KDC) gene.

In other cases, the gRNA is directed to a gene within the isobutanol pathway. The gene within the isobutanol pathway can be an AlsS, IlvC, IlvD, KDC, and/or ADH gene.

In some cases, the gRNA is transformed prior to a polynucleotide encoding for a Cas enzyme.

In some cases, the microorganism is also contacted with a donor polynucleotide. In some cases, the donor polynucleotide is contacted with the microorganism prior to being contacted with a polynucleotide encoding for a Cas enzyme. In some cases, the microorganism is contacted concurrently with a donor polynucleotide and a guide RNA. In some cases, the donor polynucleotide and guide RNA are on a single plasmid.

If a donor polynucleotide is used, the donor polynucleotide can be less than 10,000 bases. For example, the donor polynucleotide can be less than 5,000 bases. In some cases, the donor polynucleotide is less than 4,000 bases. In some cases, the donor polynucleotide is less than 3,000 bases. In some cases, the donor polynucleotide is less than 2,000 bases. In some cases, the donor polynucleotide is less than 1,000 bases. In some cases, the donor polynucleotide is less than 950 bases. In some cases, the donor polynucleotide is less than 900 bases. In some cases, the donor polynucleotide is less than 850 bases. In some cases, the donor polynucleotide is less than 800 bases. In some cases, the donor polynucleotide is less than 750 bases. In some cases, the donor polynucleotide is less than 700 bases. In some cases, the donor polynucleotide is less than 650 bases. In some cases, the donor polynucleotide is less than 600 bases. In some cases, the donor polynucleotide is less than 550 bases. In some cases, the donor polynucleotide is less than 500 bases. In some cases, the donor polynucleotide is less than 450 bases. In some cases, the donor polynucleotide is less than 400 bases. In some cases, the donor polynucleotide is less than 350 bases. In some cases, the donor polynucleotide is less than 300 bases. In some cases, the donor polynucleotide is less than 250 bases. In some cases, the donor polynucleotide is less than 200 bases. In some cases, the donor polynucleotide is less than 150 bases. In some cases, the donor polynucleotide is less than 100 bases. In some cases, the donor polynucleotide is less than 95 bases. In some cases, the donor polynucleotide is less than 90 bases. In some cases, the donor polynucleotide is less than 85 bases. In some cases, the donor polynucleotide is less than 80 bases. In some cases, the donor polynucleotide is less than 75 bases. In some cases, the donor polynucleotide is less than 70 bases. In some cases, the donor polynucleotide is less than 65 bases. In some cases, the donor polynucleotide is less than 60 bases. In some cases, the donor polynucleotide is less than 55 bases. In some cases, the donor polynucleotide is less than 50 bases. In some cases, the donor polynucleotide is less than 45 bases. In some cases, the donor polynucleotide is less than 40 bases. In some cases, the donor polynucleotide is less than 35 bases. In some cases, the donor polynucleotide is less than 30 bases. In some cases, the donor polynucleotide is less than 25 bases. In some cases, the donor polynucleotide is less than 20 bases. In some cases, the donor polynucleotide is less than 15 bases. In some cases, the donor polynucleotide is less than 10 bases. In some cases, the donor polynucleotide is less than 5 bases.

If a donor polynucleotide is used, the donor polynucleotide can be from 10,000 bases to 1 base. For example, the donor polynucleotide can be from 5,000 to 5 bases. In some cases, the donor polynucleotide can be from 2,500 to 10 bases. In some cases, the donor polynucleotide can be from 2,000 to 15 bases. In some cases, the donor polynucleotide can be from 1,500 to 25 bases. In some cases, the donor polynucleotide can be from 1,000 to 100 bases. In some cases, the donor polynucleotide can be from 750 to 125 bases. In some cases, the donor polynucleotide can be from 500 to 250 bases. In some cases, the donor polynucleotide can be from 1,000 bases to 1 base. In some cases, the donor polynucleotide can be from 900 to 5 bases. In some cases, the donor polynucleotide can be from 750 to 10 bases. In some cases, the donor polynucleotide can be from 650 to 5 bases. In some cases, the donor polynucleotide can be from 700 to 10 bases. In some cases, the donor polynucleotide can be from 600 to 10 bases. In some cases, the donor polynucleotide can be from 500 to 5 bases.

In some cases, the polynucleotide encoding for a Cas enzyme is within a plasmid.

In some cases, the plasmids used do not comprise a strong promoter. For example, the plasmid can comprise a mutated promoter. In some cases, the mutation can lead to a decrease in activity. In some cases, the promoter is a pMxaF promoter.

In some cases, the microorganism that is being used does not efficiently take up nucleic compared to an E. coli bacteria. Transformation efficiency can refer to the number of viable transformants obtained based on a predetermined amount of a compound to be transformed, which is often measured as colony forming units (CFU) per μg compound used. For example, transfection/transformation efficiency of highly competent E. coli cells can reach approximately 2×10¹⁰-4×10¹⁰ cfu/μg of nucleic acid used for the transformations. In some cases, the microorganisms used throughout have very low transformation efficiency. In some cases, the transformation efficiency of the microorganisms used herein is lower than 2×10¹⁰ cfu/μg. For example, the transformation efficiency of the microorganism used herein can be 0 cfu/μg. In some cases, the transformation efficiency can be 0 to 1×10² cfu/μg; 1×10² cfu/μg to 1×10³ cfu/μg; 1×10³ cfu/μg to 1×10⁴ cfu/μg; 1×10⁴ cfu/μg to 1×10⁵ cfu/μg; 1×10⁵ cfu/μg to 1×10⁶ cfu/μg; 1×10⁶ cfu/μg to 1×10⁷ cfu/μg; 1×10⁷ cfu/μg to 1×10⁸ cfu/μg; 1×10⁸ cfu/μg to 1×10⁹ cfu/μg; 1×10⁹ cfu/μg to 1×10¹⁰ cfu/μg; or 1×10¹⁰ cfu/μg to 1.9999×10¹⁰ cfu/μg. In some cases, the transformation efficiency can be 0 cfu/μg to less than 1 cfu/μg; 1 cfu/μg to 5 cfu/μg; 5 cfu/μg to 10 cfu/μg; 10 cfu/μg to 20 cfu/μg; 20 cfu/μg to 30 cfu/μg; 30 cfu/μg to 40 cfu/μg; 40 cfu/μg to 50 cfu/μg; 50 cfu/μg to 100 cfu/μg; 100 cfu/μg to 150 cfu/μg; 150 cfu/μg to 200 cfu/μg; 200 cfu/μg to 250 cfu/μg; 250 cfu/μg to 500 cfu/μg; 500 cfu/μg to 1000 cfu/μg; 1000 cfu/μg to 1500 cfu/μg; 1500 cfu/μg to 2000 cfu/μg; or 2000 cfu/μg to 5000 cfu/μg.

In some cases, the microorganism is made electroporation competent prior to transformation. In some cases, some chemical is made to make the microorganism electroporation competent prior to transformation. In some cases, a microorganism can be made to take up nucleic acids more efficiently compared to a non-modified microorganism.

### Point Mutations

Described herein is a method of replacing a single nucleotide within the genome of a microorganism comprising: (a) contacting the microorganism with a polynucleotide encoding for i) a Cas enzyme and ii) a gRNA; and (b) growing the microorganism until a single nucleotide is replaced within the genome of the microorganism.

As described above, the microorganism used can be a microorganism that is capable of converting a C₁ carbon to a multicarbon product, for example, a methylotroph or any other microorganism described throughout the disclosure.

Further, as described throughout, the C₁ carbon can be carbon monoxide (CO), carbon dioxide (CO₂), methane (CH₄), or any combination thereof.

Additionally, as described through, the Cas enzyme can be any described throughout, including but not limited to Cas9.

As described throughout, the gRNA can target particular pathway genes. For example, as described throughout, the gRNA can be at least partially complementary to a polynucleotide that is within a promoter, intron, or coding sequence of an rpoB gene or a gene within the 2,3-butanediol (2,3-BDO), 1,4-butanediol (1,4-BDO), isobutyraldehyde, or isobutanol pathway.

In some cases, the gRNA is transformed prior to a polynucleotide encoding for a Cas enzyme.

In some cases, the polynucleotide encoding for a Cas enzyme is within a plasmid.

In some cases, the plasmids used do not comprise a strong promoter. For example, the plasmid can comprise a mutated promoter. In some cases, the mutation can lead to a decrease in activity. In some cases, the promoter is a pMxaF promoter.

In some cases, when the replacement method is used, the replacement results in a different nucleotide. For example, should a nucleotide within a specific genetic sequence be desired, this method can change the desired nucleotide, e.g., from A to T, C, or G; from T to A, C, or G; from C to A, T, or G; or from G to A, T, or C.

In some cases, the replacement occurs at a single nucleotide within a promoter, intron, or coding sequence of an rpoB gene or a gene within the 2,3-butanediol (2,3-BDO), 1,4-butanediol (1,4-BDO), isobutyraldehyde, or isobutanol pathway. The replacement can in some cases result in a change of expression of one or more genes. The replacement in some cases can also result in a change of activity of one or more enzymes.

### Adding Nucleotides

Described herein is a method of adding one or more nucleotides to the genome of a microorganism comprising: (a) contacting the microorganism with a polynucleotide encoding for i) a Cas enzyme and ii) a gRNA; and (b) growing the microorganism until one or more nucleotides is added to the genome of the microorganism.

As described above, the microorganism used can be a microorganism that is capable of converting a C₁ carbon to a multi-carbon product, for example, a methylotroph or any other microorganism described throughout the disclosure.

Further, as described throughout, the C₁ carbon can be carbon monoxide (CO), carbon dioxide (CO₂), methane (CH₄), or any combination thereof.

Additionally, as described through, the Cas enzyme can be any described throughout, including but not limited to Cas9.

As described throughout, the gRNA can target particular pathway genes to insert one or more nucleotides. For example, as described throughout, the gRNA can be at least partially complementary to a polynucleotide that is within a promoter, intron, or coding sequence of an rpoB gene or a gene within the 2,3-butanediol (2,3-BDO), 1,4-butanediol (1,4-BDO), isobutyraldehyde, or isobutanol pathway.

In some cases, the gRNA is transformed prior to a polynucleotide encoding for a Cas enzyme.

In some cases, the polynucleotide encoding for a Cas enzyme is within a plasmid.

In some cases, the plasmids used do not comprise a strong promoter. For example, the plasmid can comprise a mutated promoter. In some cases, the mutation can lead to a decrease in activity. In some cases, the promoter is a pMxaF promoter.

In some cases, the microorganism is also contacted with a donor polynucleotide. In some cases, the donor polynucleotide is contacted with the microorganism prior to being contacted with a polynucleotide encoding for a Cas enzyme. In some cases, the microorganism is contacted concurrently with a donor polynucleotide and a guide RNA. In some cases, the donor polynucleotide and guide RNA are on a single plasmid.

If a donor polynucleotide is used, the donor polynucleotide can be less than 10,000 bases. For example, the donor polynucleotide can be less than 5,000 bases. In some cases, the donor polynucleotide is less than 4,000 bases. In some cases, the donor polynucleotide is less than 3,000 bases. In some cases, the donor polynucleotide is less than 2,000 bases. In some cases, the donor polynucleotide is less than 1,000 bases. In some cases, the donor polynucleotide is less than 950 bases. In some cases, the donor polynucleotide is less than 900 bases. In some cases, the donor polynucleotide is less than 850 bases. In some cases, the donor polynucleotide is less than 800 bases. In some cases, the donor polynucleotide is less than 750 bases. In some cases, the donor polynucleotide is less than 700 bases. In some cases, the donor polynucleotide is less than 650 bases. In some cases, the donor polynucleotide is less than 600 bases. In some cases, the donor polynucleotide is less than 550 bases. In some cases, the donor polynucleotide is less than 500 bases. In some cases, the donor polynucleotide is less than 450 bases. In some cases, the donor polynucleotide is less than 400 bases. In some cases, the donor polynucleotide is less than 350 bases. In some cases, the donor polynucleotide is less than 300 bases. In some cases, the donor polynucleotide is less than 250 bases. In some cases, the donor polynucleotide is less than 200 bases. In some cases, the donor polynucleotide is less than 150 bases. In some cases, the donor polynucleotide is less than 100 bases. In some cases, the donor polynucleotide is less than 95 bases. In some cases, the donor polynucleotide is less than 90 bases. In some cases, the donor polynucleotide is less than 85 bases. In some cases, the donor polynucleotide is less than 80 bases. In some cases, the donor polynucleotide is less than 75 bases. In some cases, the donor polynucleotide is less than 70 bases. In some cases, the donor polynucleotide is less than 65 bases. In some cases, the donor polynucleotide is less than 60 bases. In some cases, the donor polynucleotide is less than 55 bases. In some cases, the donor polynucleotide is less than 50 bases. In some cases, the donor polynucleotide is less than 45 bases. In some cases, the donor polynucleotide is less than 40 bases. In some cases, the donor polynucleotide is less than 35 bases. In some cases, the donor polynucleotide is less than 30 bases. In some cases, the donor polynucleotide is less than 25 bases. In some cases, the donor polynucleotide is less than 20 bases. In some cases, the donor polynucleotide is less than 15 bases. In some cases, the donor polynucleotide is less than 10 bases. In some cases, the donor polynucleotide is less than 5 bases.

If a donor polynucleotide is used, the donor polynucleotide can be from 10,000 bases to 1 base. For example, the donor polynucleotide can be from 5,000 to 5 bases. In some cases, the donor polynucleotide can be from 2,500 to 10 bases. In some cases, the donor polynucleotide can be from 2,000 to 15 bases. In some cases, the donor polynucleotide can be from 1,500 to 25 bases. In some cases, the donor polynucleotide can be from 1,000 to 100 bases. In some cases, the donor polynucleotide can be from 750 to 125 bases. In some cases, the donor polynucleotide can be from 500 to 250 bases. In some cases, the donor polynucleotide can be from 1,000 bases to 1 base. In some cases, the donor polynucleotide can be from 900 to 5 bases. In some cases, the donor polynucleotide can be from 750 to 10 bases. In some cases, the donor polynucleotide can be from 650 to 5 bases. In some cases, the donor polynucleotide can be from 700 to 10 bases. In some cases, the donor polynucleotide can be from 600 to 10 bases. In some cases, the donor polynucleotide can be from 500 to 5 bases.

The method described herein can result in a polynucleotide where two or more nucleotides are added. In some cases, the number of nucleotides added can be up to 10 kb. In some cases, however, the efficiency of gene editing efficiency can be increased by inserting less than 1000 base pairs. In some cases, the efficiency of gene editing can be significantly increased by inserting 500 nucleotides or less. In some cases, the efficiency of gene editing can be even more significantly increased by inserting 100 nucleotides or less.

The amount of nucleotides that can be inserted using the techniques described herein can potentially be endless. In some cases, the number of nucleotides inserted can be from 1 to 5, 4 to 10, 9 to 15, 14 to 20, 19 to 25, 24 to 30, 29 to 35, 34 to 40, 39 to 45, 44 to 50, 49 to 55, 54 to 60, 59 to 65, 64 to 70, 69 to 75, 74 to 80, 79 to 85, 84 to 90, 89 to 95, or 94 to 100. In some cases, the number of nucleotides that can be inserted can be under 5000 kb, for example, from 99 to 500, 499 to 1000, 999 to 1500, 1499 to 2000, 1999 to 2500, 2499 to 3000, 2999 to 3500, 3499 to 4000, 3999 to 4500, or 4499 to 4999.

As described throughout, the gRNA can target particular pathway genes. For example, as described throughout, the gRNA can be at least partially complementary to a polynucleotide that is within a promoter, intron, or coding sequence of an rpoB gene or a gene within the 2,3-butanediol (2,3-BDO), 1,4-butanediol (1,4-BDO), isobutyraldehyde, or isobutanol pathway. Targeting these sequences can add additional nucleotides within the promoter, intron, or coding sequence. This addition can affect the expression of their respective genes. This addition can also affect the activity of the gene product, e.g., an enzyme of the pathway.

### Deleting Nucleotides

Described herein is a method of removing one or more nucleotides from the genome of a genetically modified microorganism comprising: (a) contacting the microorganism with a polynucleotide encoding for i) a Cas enzyme and ii) a gRNA; and (b) growing the microorganism until one or more nucleotides within the genome of the microorganism is removed.

As described above, the microorganism used can be a microorganism that is capable of converting a C₁ carbon to a multicarbon product, for example, a methylotroph or any other microorganism described throughout the disclosure.

Further, as described throughout, the C₁ carbon can be carbon monoxide (CO), carbon dioxide (CO₂), methane (CH₄), or any combination thereof.

Additionally, the Cas enzyme can be any described throughout, including but not limited to Cas9.

In some cases, the polynucleotide encoding for a Cas enzyme is within a plasmid.

In some cases, the plasmids used do not comprise a strong promoter. For example, the plasmid can comprise a mutated promoter. In some cases, the mutation can lead to a decrease in activity. In some cases, the promoter is a pMxaF promoter.

In some cases, the gRNA is transformed prior to a polynucleotide encoding for a Cas enzyme.

As described throughout, the gRNA can target particular pathway genes to delete one or more nucleotides. For example, as described throughout, the gRNA can be at least partially complementary to a polynucleotide that is within a promoter, intron, or coding sequence of an rpoB gene or a gene within the 2,3-butanediol (2,3-BDO), 1,4-butanediol (1,4-BDO), isobutyraldehyde, or isobutanol pathway.

The method described herein can result in a polynucleotide where one or more nucleotides are deleted. The amount of nucleotides that can be deleted using the techniques described herein can potentially be endless. In some cases, the number of nucleotides deleted can be from 1 to 5, 4 to 10, 9 to 15, 14 to 20, 19 to 25, 24 to 30, 29 to 35, 34 to 40, 39 to 45, 44 to 50, 49 to 55, 54 to 60, 59 to 65, 64 to 70, 69 to 75, 74 to 80, 79 to 85, 84 to 90, 89 to 95, or 94 to 100. In some cases, the number of nucleotides that can be deleted can be under 5000 kb, for example, from 99 to 500, 499 to 1000, 999 to 1500, 1499 to 2000, 1999 to 2500, 2499 to 3000, 2999 to 3500, 3499 to 4000, 3999 to 4500, or 4499 to 4999. In some cases, the number of nucleotides that are deleted can be up to 10 kb or more.

As described throughout, the gRNA can target particular pathway genes. For example, as described throughout, the gRNA can be at least partially complementary to a polynucleotide that is within a promoter, intron, or coding sequence of an rpoB gene or a gene within the 2,3-butanediol (2,3-BDO), 1,4-butanediol (1,4-BDO), isobutyraldehyde, or isobutanol pathway. Targeting these sequences can delete nucleotides within the promoter, intron, or coding sequence. This deletion can affect the expression of their respective genes. This deletion can also affect the activity of the gene product, e.g., an enzyme of the pathway.

### IV. Methods of Inhibiting Gene Expression

Modified Cas enzyme

The Cas enzymes can be genetically altered (by the methods described throughout or any other method) so that it is catalytically inactive. For example, one or more nucleotides encoding an amino acid sequence that is a part of the catalytic domain of the Cas enzyme can be altered. In other words, one or more nucleotides that encode for the catalytic domain of the Cas enzyme can be deleted, added, or substituted. The resulting sequence can encode for a Cas enzyme that is catalytically inhibited and/or inactive.

The catalytically inactive enzyme can be used to inhibit expression of one or more genes. For example, a specific gRNA can be used to target the promoter, intron, and/or coding sequence of a particular gene. The specific gRNA and inactive Cas enzyme can be expressed within a microorganism. Once this happens, gene expression can be reduced or inhibited.

The binding of the catalytically inactive Cas enzyme can result in steric hindrance of the transcription mechanism. For example, the inactive Cas enzyme that is bound to the gRNA can interrupt transcript initiation or elongation by RNA polymerase.

In some cases, the binding of this blocking complex can be permanent. In some cases the binding of this blocking complex can be temporary. Further, the inactive Cas enzyme can be expressed within a microorganism and be under the control of an inducible or repressible promoter. Additionally, in some cases the gRNA can be expressed within a vector and also be controlled by an inducible and/or repressible promoter. This way, the induction or repression of the desired gene can be specifically controlled at any time by the addition or removal of the inducing/repressing agent.

### Targeting Specific Genes

The expression of any number of genes can be inhibited by the methods described throughout. For example, an rpoB gene or any of the genes described within the 2,3-BDO, 1,4-BDO, isobutanol, and/or isobutyraldehyde pathways can be targeted by the gRNA and thus by the inactive Cas enzyme. Any of the gRNA described throughout can be used herein. The gRNA can be substantially similar to the genes described throughout.

### Inhibiting Gene Expression

Described herein is a method of inhibiting the expression of a gene within a microorganism comprising contacting the microorganism with a polynucleotide encoding for i) a modified Cas enzyme and ii) a gRNA, where the modified Cas enzyme does not cleave nucleic acids. Also described herein is a method of inhibiting the expression of a gene within a microorganism comprising contacting the microorganism with i) a gRNA and ii) a polynucleotide encoding for a modified Cas enzyme, where the modified Cas enzyme does not cleave nucleic acids.

In some cases, the microorganism used can be a microorganism that is capable of converting a C₁ carbon to a multicarbon product. For example, the microorganism can be a microorganism as described throughout, such as a methylotroph. For example, the methylotroph can be a methanotroph, such as from the genera Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, or Methyloacidophilum. In some cases, the methanotroph can be from the genus Methylococcus, such as a Methylococcus capsulatus.

In some cases, the C₁ carbon can be any C₁ carbon disclosed throughout. For example, in some cases the C₁ carbon is carbon monoxide (CO), carbon dioxide (CO₂), methane (CH₄), or any combination thereof. In some cases, the C₁ carbon is CH₄.

The modified Cas enzymes that can be used for any of the methods described throughout include but are not limited to Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, or homologues thereof. In some cases, the Cas enzyme is a modified Cas9 enzyme. As described above, the modification can be a modified that renders the Cas enzyme partially inactive. The partial inactivity can mean that the Cas enzyme has the ability to bind to its targeted sequence, but does not have the ability to cleave the nucleic acids. In some cases, the polynucleotide encoding for a Cas enzyme or a modified Cas enzyme is within a plasmid.

In some cases, the gRNA can be at least partially or fully homologous to any one of the genes or promoters described throughout. In some instances, the gRNA is at least partially homologous or fully homologous to a promoter, intron, or coding sequence of an rpoB gene or a gene within the 2,3-butanediol (2,3-BDO), 1,4-butanediol (1,4-BDO), isobutyraldehyde, or isobutanol pathway.

In some cases, the gRNA is directed to a promoter, intron, or coding sequence of gene within the 2,3-BDO pathway. The gene within the 2,3-BDO can be an acetoin reductase, alpha-acetolactate decarboxylase, and/or acetolactate synthase gene.

In other cases, the gRNA is directed to a promoter, intron, or coding sequence of a gene within the 1,4-BDO pathway. The gene can be within the 1,4-BDO pathway can be a pyruvate dehydrogenase (aceEF), citrate synthase (gltA), aconitate hydratase 1 (acnA), isocitrate dehydrogenase (icdA), citrate synthase (gltA), succinyl-CoA synthetase (SucC), CoA-dependent succinate semialdehyde dehydrogenase (SucD), 4-hyrobutyrate dehydrogenase (4hbD), 4-hydroxybutyryl-CoA transferase (Cat2), aldehyde dehydrogenase (Ald), alcohol dehydrogenase (Adh), and/or α-ketoglutarate decarboxylase (kgd) gene.

In some cases, the gRNA is directed to a promoter, intron, or coding sequence of a gene within the isobutyraldehyde pathway. The gene within the isobutyraldehyde pathway can be an acetolactate synthase (AlsS); ketol-acid reductoisomerase (IlvC); dihydroxy-acid dehydratase (IlvD); and/or 2-keto acid decarboxylase (KDC) gene.

In other cases, the gRNA is directed to a gene within the isobutanol pathway. The gene within the isobutanol pathway can be an AlsS, IlvC, IlvD, KDC, and/or ADH gene.

In some cases, the plasmids used do not comprise a strong promoter. For example, the plasmid can comprise a mutated promoter. In some cases, the mutation can lead to a decrease in activity. In some cases, the promoter is a pMxaF promoter.

In some cases, the gRNA is transformed prior to a polynucleotide encoding for a Cas enzyme.

In some cases, the microorganism that is being used does not efficiently take up nucleic acid compared to an E. coli bacteria. Transformation efficiency can refer to the number of viable transformants obtained based on a predetermined amount of a compound to be transformed, which is often measured as colony forming units (CFU) per μg compound used. For example, transfection/transformation efficiency of highly competent E. coli cells can reach approximately 2×10¹⁰-4×10¹⁰ cfu/μg of nucleic acid used for the transformations. In some cases, the microorganisms used throughout have very low transformations efficiency. In some cases, the transformation efficiency of the microorganisms used herein is lower than 2×10¹⁰ cfu/μg. For example, the transformation efficiency of the microorganism used herein can be 0 cfu/μg. In some cases, the transformation efficiency can be 0 to 1×10² cfu/μg; 1×10² cfu/μg to 1×10³ cfu/μg; 1×10³ cfu/μg to 1×10⁴ cfu/μg; 1×10⁴ cfu/μg to 1×10⁵ cfu/μg; 1×10⁵ cfu/μg to 1×10⁶ cfu/μg; 1×10⁶ cfu/μg to 1×10⁷ cfu/μg; 1×10⁷ cfu/μg to 1×10⁸ cfu/μg: 1×10⁸ cfu/μg to 1×10⁹ cfu/μg; 1×10⁹ cfu/μg to 1×10¹⁰ cfu/μg; or 1×10¹⁰ cfu/μg to 1.9999×10¹⁰ cfu/μg. In some cases, the transformation efficiency can be 0 cfu/μg to less than 1 cfu/μg; 1 cfu/μg to 5 cfu/μg; 5 cfu/μg to 10 cfu/μg; 10 cfu/μg to 20 cfu/μg; 20 cfu/μg to 30 cfu/μg; 30 cfu/μg to 40 cfu/μg; 40 cfu/μg to 50 cfu/μg; 50 cfu/μg to 100 cfu/μg; 100 cfu/μg to 150 cfu/μg; 150 cfu/μg to 200 cfu/μg; 200 cfu/μg to 250 cfu/μg; 250 cfu/μg to 500 cfu/μg; 500 cfu/μg to 1000 cfu/μg; 1000 cfu/μg to 1500 cfu/μg; 1500 cfu/μg to 2000 cfu/μg; or 2000 cfu/μg to 5000 cfu/μg.

In some cases, the microorganism is made electroporation competent prior to transformation. In some cases, some chemical is made to make the microorganism electroporation competent prior to transformation.

In some cases, the inhibition of gene expression is greater than 10%, 20%, 30%, 40%, 50%, 60%, 70%, 80%, or 90%, compared to a wild-type microorganism of the same species. In some cases, the inhibition of gene expression is greater than 5%, 15%, 25%, 35%, 45%, 55%, 65%, 75%, 85%, or 95% compared to a wild-type microorganism of the same species. In some cases, the inhibition of gene expression is greater than 10% compared to a wild-type microorganism of the same species. In some cases, the inhibition of gene expression is greater than 50% compared to a wild-type microorganism of the same species. In some cases, the inhibition of gene expression is 100% compared to a wild-type microorganism of the same species.

### Examples

### Example 1: Single Plasmid

The Streptococcus pyogenes Cas9 gene was codon optimized for expression in Methylococcus capsulatus. The codon optimized Cas9 gene was expressed using a pMxaF promoter. A single guide RNA (gRNA) targeting multiple sites along a gene of interest were expressed driven by a Pₘₘₒ₂ or J23115 promoter. Double stranded DNA having a 1.5 kb homologous region were created. Two gRNA constructs were tested initially.

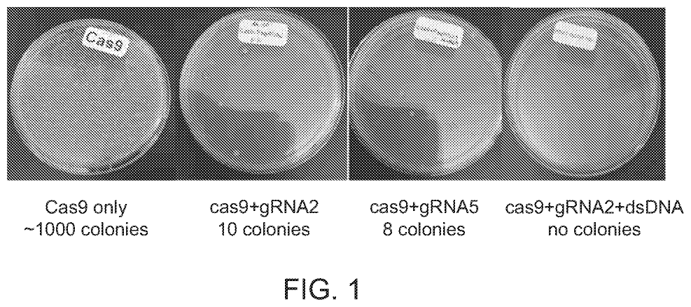

Initial tests proved to be ineffective at gene editing. As seen in FIG. 1, no colonies were seen when Cas9, gRNA, and double stranded donor DNA were transfected into a Methylococcus capsulatus. Later tests showed that pMxaF driving the expression of Cas9 is toxic to Methylococcus capsulatus.

Four (4) different promoters were used to drive Cas9 expression: pMxaF_Cas9; pMxaF*_Cas9; pBAD_Cas9; PJ23115_gRNA. pMxaF*_Cas9 was found to be the most efficient and least toxic.

### Example 2: Two Plasmid System

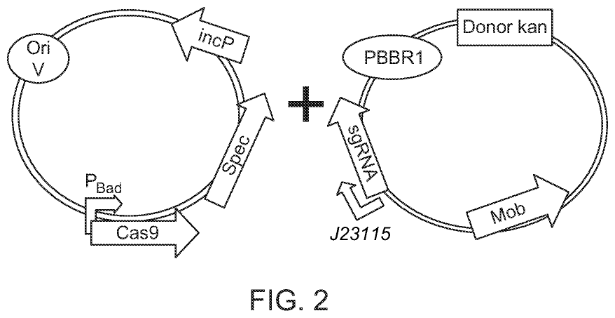

In order to mitigate the toxic effects of Cas9, a two plasmid system was designed. See FIG. 2. In one plasmid, the Cas9 was driven by either pBAD or a mutant pMxaF (a “weak” promoter), whereas the sgRNA was driven by a J23115 promoter.

It was found that pBAD or mutant pMxaF produces enough Cas9 expression to cleave the dsDNA without much off-target effects. However, it was still found that in the presence of gRNA, very few colonies were observed. Of those few colonies, no correct gene editing was observed.

### Example 3: Rifamycin Testing

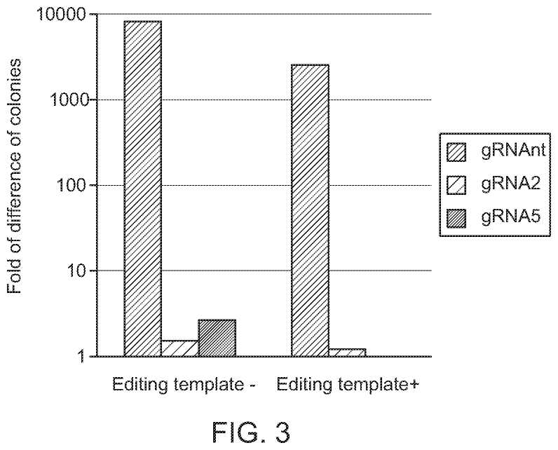

In order to quickly and efficiently troubleshoot the issues with genome editing within Methylococcus capsulatus, gene editing targeting the rpoB was used. In Methylococcus capsulatus the rpoB gene confers resistance to the antibiotic rifamycin. Therefore, in the presence of rifamycin, Methylococcus capsulatus having an active rpoB gene will form many colonies, while the Methylococcus capsulatus that were edited will be killed. A 1 kb long rpoB dsDNA was designed and used as the editing template in this experiment. As shown in FIG. 3, the gRNA targeting rpoB was effective and resulted in four orders of magnitude drop of colony forming units (CFU). However, verification revealed that the efficiency of gene editing was only approximately 0.5 to 1%.

Even though the use of a mutant pMxaF gave low background, editing efficiency was still low. In order to improve the efficiency of genome editing, the number of unedited clones needed to be reduced. Therefore, additional testing focused on “killing” efficiency.

### Example 4: Promoters and Transformation Order

It was found that promoter strength of Cas9/gRNA affects the activity of CRISPR-Cas9 system. A stronger promoter that drives gRNA expression works better than a weaker promoter. On the other hand, Cas9 expression by a strong promoter is lethal to the microorganism.

The best “killing” rate was observed when the gRNA was present first then Cas9 encoding plasmid was subsequently transformed. However, even though the kill rate went up, increased editing efficiency did not improve.

A new approach was taken. The gRNA and donor DNA, contained on the same plasmid, were first transformed into a Methylococcus capsulatus. After this plasmid containing both the gRNA and donor DNA was inserted, a plasmid expression Cas9 was then transformed. Editing efficiency was achieved at about 70%, using rpoB as a target.

Other systems were tested, including a Red promoter driving Cas9 expression. However, this promoter did not result in high editing efficiency. Additionally, Cas9 was first transformed and then gRNA and donor DNA was later transformed. This procedure resulted in very low editing. It was found that high rates of transformation and recombination was required for advance genome editing.

### Example 5: Cas9 Expression in Methanotrophs

The Streptococcus pyogenes Cas9 gene was codon optimized for expression in Methylococcus capsulatus. The codon optimized Cas9 gene was expressed using a variant pMxaF promoter referred to as pMxaF*. The pMxaF*-Cas9 DNA cassette was then cloned into an OriV-based plasmid. The final construct was named pSL95. The pMxaF* and Cas9 sequences used are displayed in Table 1.

TABLE 1

| SEQ ID NO. | NAME | SEQUENCE |
|---|---|---|
| 78 | pMxaF* | GAGGTTCAGGCGAAACCGCAGACTCAAGGGCGCTTGCTCCCGGGAA |
| | | AGATCGTATTAGTTTGCCTCGATCGGCGGTCCTTGTGACAGGGAGAT |
| | | ATTCCCGACGGATCCGGGGCATTCGAGCGGAACCGCCCGCCGTGGG |
| | | AGTTTTTCCAGCGAGCATTCGAGAGTTTTTCAAGGCGGCTTCGAGGG |
| | | GTTATTCCGTAACGCCGCCGACATGATCTGTCCCGGAATCTCCGCCG |
| | | CTGTTCGTAGAGCGCCGATGCAGGGTCGGCATCAATCATTCTTGGAG |
| | | GAGACAC |
| 79 | Cas9 | ATGGACAAGAAGTATTCGATCGGCCTGGACATCGGCACCAACAGCG |
| | | TCGGCTGGGCGGTCATCACGGATGAGTACAAGGTGCCGTCGAAGAA |
| | | GTTCAAGGTGCTGGGCAATACCGACCGCCATAGCATCAAGAAGAAT |
| | | CTCATCGGCGCACTGCTGTTCGACTCCGGCGAAACCGCCGAAGCGAC |
| | | CCGCCTCAAGCGCACGGCCCGGCGGCGCTATACGCGCCGGAAGAAC |
| | | CGCATCTGCTACCTCCAGGAAATCTTCTCCAACGAGATGGCCAAGGTG |
| | | GATGACTCCTTCTTCCATCGCCTGGAAGAATCCTTCCTGGTCGAAGAA |
| | | GATAAGAAACATGAGCGCCACCCCATCTTCGGCAATATCGTGGACGA |
| | | GGTGGCGTATCACGAGAAATACCCGACCATCTATCACCTGCGGAAAA |
| | | AGCTGGTGGACTCGACGGACAAAGCCGACCTGCGCCTCATCTATCTGG |
| | | CCCTGGCCCACATGATCAAGTTCCGGGGCCATTTCCTGATCGAAGGCG |
| | | ACCTGAACCCCGATAACAGCGACGTGGACAAGCTCTTCATCCAGCTCG |
| | | TCCAGACCTATAACCAGCTGTTCGAGGAGAACCCCATCAACGCCTCGG |
| | | GCGTGGACGCCAAGGCCATCCTGAGCGCACGGCTCTCCAAGTCGCGCC |
| | | GCCTGGAAAACCTGATCGCGCAGCTGCCGGGCGAAAAGAAAAACGGC |
| | | CTGTTCGGCAACCTGATCGCCCTGTCCCTCGGCCTCACCCCGAACTTCA |
| | | AGTCCAACTTCGACCTGGCCGAGGACGCGAAGCTCCAGCTGTCGAAAG |
| | | ACACCTACGATGACGACCTGGACAACCTCCTGGCGCAGATCGGCGACC |
| | | AGTACGCCGACCTCTTCCTCGCGGCCAAGAATCTGTCGGACGCCATCCT |
| | | GCTGTCGGATATCCTGCGGGTGAATACGGAGATCACGAAGGCCCCCCT |
| | | CTCGGCCTCGATGATCAAGCGCTACGACGAGCACCATCAGGACCTGAC |
| | | GCTGCTCAAGGCCCTCGTCCGGCAGCAGCTGCCGGAGAAGTATAAAGA |
| | | GATCTTCTTCGACCAGTCCAAGAACGGCTACGCGGGCTACATCGACGG |
| | | CGGCGCGTCGCAGGAGGAGTTCTATAAATTCATCAAGCCGATCCTGGA |
| | | GAAAATGGACGGCACCGAAGAACTCCTCGTCAAGCTGAACCGGGAGGA |
| | | TCTGCTCCGCAAGCAGCGCACCTTCGACAATGGCTCCATCCCGCACCAG |
| | | ATCCATCTCGGCGAGCTGCACGCCATCCTGCGCCGCCAGGAGGACTTCT |
| | | ACCCCTTCCTCAAAGACAACCGGGAGAAAATCGAGAAGATCCTGACGTT |
| | | CCGCATCCCCTACTACGTGGGCCCCCTCGCCCGCGGCAACTCGCGGTTCG |
| | | CGTGGATGACCCGGAAGAGCGAGGAGACGATCACCCCGTGGAATTTCGA |
| | | GGAGGTCGTCGATAAAGGCGCGTCGGCGCAGTCGTTCATCGAGCGCATG |
| | | ACCAACTTCGATAAAAATCTGCCGAACGAAAAAGTCCTGCCCAAGCATA |
| | | GCCTGCTGTACGAGTACTTCACGGTCTACAACGAGCTGACGAAAGTGAA |
| | | ATATGTCACGGAGGGCATGCGCAAACCGGCCTTCCTGTCCGGCGAGCAG |
| | | AAAAAGGCCATCGTGGATCTGCTGTTCAAGACGAACCGGAAGGTCACCG |
| | | TGAAACAGCTGAAGGAAGATTACTTCAAGAAAATCGAGTGCTTCGATTC |
| | | CGTCGAAATCAGCGGCGTGGAGGACCGCTTCAATGCCTCGCTGGGCACC |
| | | TATCACGATCTCCTCAAGATCATCAAGGACAAGGACTTCCTGGACAACG |
| | | AAGAGAACGAGGACATCCTGGAAGACATCGTCCTCACCCTGACCCTGTT |
| | | CGAGGACCGCGAAATGATCGAAGAGCGCCTGAAGACCTACGCCCACCTG |
| | | TTCGACGACAAGGTCATGAAGCAGCTCAAGCGCCGCCGGTACACCGGCT |
| | | GGGGCCGCCTGTCCCGGAAGCTGATCAACGGCATCCGCGATAAGCAGAG |
| | | CGGCAAGACGATCCTGGACTTCCTCAAGAGCGACGGCTTCGCCAATCGGA |
| | | ATTTCATGCAGCTCATCCACGACGATAGCCTGACCTTCAAAGAGGATATC |
| | | CAGAAGGCGCAGGTGTCCGGCCAGGGCGACAGCCTGCACGAACATATCG |
| | | CCAACCTGGCGGGCTCCCCCGCGATCAAGAAAGGCATCCTCCAGACGGTC |
| | | AAAGTCGTGGACGAGCTGGTCAAGGTGATGGGCCGCCACAAACCGGAGA |
| | | ATATCGTCATCGAGATGGCACGCGAGAACCAGACCACGCAGAAGGGCCA |
| | | GAAGAACAGCCGGGAACGCATGAAACGGATCGAAGAGGGCATCAAGGA |
| | | ACTGGGCTCGCAGATCCTGAAGGAGCACCCCGTCGAAAACACGCAGCTC |
| | | CAGAACGAGAAGCTGTATCTGTACTATCTCCAGAACGGCCGGGACATGTA |
| | | TGTCGATCAGGAACTGGATATCAACCGCCTCTCCGATTACGATGTGGACC |
| | | ACATCGTGCCGCAGAGCTTCCTGAAAGACGACTCGATCGACAACAAGGTC |
| | | CTGACCCGGTCGGACAAGAACCGCGGCAAGTCGGATAACGTGCCGTCGG |
| | | AAGAAGTCGTGAAAAAGATGAAGAACTACTGGCGGCAGCTCCTGAACGC |
| | | GAAGCTCATCACGCAGCGCAAGTTCGACAATCTGACCAAGGCCGAGCGC |
| | | GGCGGCCTCTCGGAACTCGACAAGGCGGGCTTCATCAAACGGCAGCTCG |
| | | TCGAGACGCGCCAGATCACCAAACACGTGGCCCAGATCCTGGACAGCCG |
| | | GATGAACACCAAATACGACGAAAACGACAAGCTGATCCGCGAAGTCAAG |
| | | GTCATCACGCTGAAGAGCAAGCTGGTGTCGGATTTCCGCAAGGATTTCCA |
| | | GTTCTACAAGGTGCGCGAGATCAACAATTACCATCACGCGCACGATGCCT |
| | | ATCTCAATGCGGTCGTGGGCACCGCCCTGATCAAAAAGTACCCGAAACTG |
| | | GAGTCCGAGTTCGTCTACGGCGACTATAAGGTCTATGATGTCCGCAAGAT |
| | | GATCGCCAAATCGGAGCAGGAGATCGGCAAGGCGACCGCGAAATATTTC |
| | | TTCTACTCGAACATCATGAATTTCTTCAAGACCGAGATCACGCTGGCGAA |
| | | CGGCGAAATCCGCAAGCGGCCCCTGATCGAAACCAATGGCGAGACCGGC |
| | | GAGATCGTGTGGGACAAAGGCCGGGATTTCGCCACCGTCCGCAAGGTCCT |
| | | CTCGATGCCGCAGGTGAACATCGTCAAGAAGACGGAGGTCCAGACCGGC |
| | | GGCTTCAGCAAAGAAAGCATCCTCCCCAAGCGGAATAGCGACAAACTGA |
| | | TCGCCCGGAAGAAGGACTGGGACCCGAAGAAGTATGGCGGCTTCGATAG |
| | | CCCCACCGTCGCCTATTCCGTCCTGGTGGTGGCGAAGGTGGAGAAAGGCA |
| | | AAAGCAAGAAACTGAAGAGCGTGAAGGAGCTGCTGGGCATCACCATCAT |
| | | GGAACGCAGCAGCTTCGAGAAGAACCCGATCGACTTCCTGGAAGCCAAA |
| | | GGCTATAAGGAAGTGAAGAAGGACCTCATCATCAAACTCCCGAAGTATT |
| | | CGCTGTTCGAGCTGGAAAATGGCCGCAAACGGATGCTCGCCTCCGCGGG |
| | | CGAACTCCAGAAGGGCAACGAACTGGCGCTGCCGTCCAAATACGTCAAC |
| | | TTCCTCTATCTGGCCAGCCATTACGAAAAGCTGAAGGGCTCGCCCGAAGA |
| | | TAACGAGCAGAAACAGCTGTTCGTCGAGCAGCACAAGCACTACCTCGAC |
| | | GAGATCATCGAGCAGATCAGCGAGTTCTCCAAGCGGGTGATCCTCGCGG |
| | | ACGCCAACCTGGACAAGGTGCTGTCGGCGTACAACAAACATCGGGATAA |
| | | GCCGATCCGCGAGCAGGCCGAAAATATCATCCACCTGTTCACCCTGACGA |
| | | ACCTCGGCGCCCCCGCCGCCTTCAAGTATTTCGATACCACCATCGACCGGA |
| | | AGCGCTATACCTCCACCAAAGAGGTCCTGGATGCCACCCTCATCCACCAG |
| | | TCCATCACGGGCCTGTACGAGACCCGCATCGACCTGTCGCAGCTGGGCGG |
| | | CGACTAA |

### Example 6: Synthetic gRNA Expression

Synthetic gRNA were made which contained a 20 bp target region and a 83 bp Cas9 handle and terminator region. This synthetic gRNA was made to be driven by a constitutively expressed J23111 promoter. This J23111 promoter-gRNA sequence was cloned into a pBBR1-based plasmid (pSL90). Other J series promoters such as J23115 were also tested resulting in high editing efficiency (>50%). Additionally, a donor sequence was cloned in the same pBBR1-based plasmid containing the gRNA. The J23111 and Cas9 sequences used are shown in Table 2.

TABLE 2

| SEQ ID NO. | NAME | SEQUENCE |
|---|---|---|
| 80 | J23111 | TTGACGGCTAGCTCAGTCCTAGGTATAGTGCTAGC |
| 81 | Cas9 | GTTTTAGAGCTAGAAATAGCAAGTTAAAATAAGGCTAGT |
| | handle | CCGTTATCAACTTGAAAAAGTGGCACCGAGTCGGTGCTT |
| | and | TTTTT |
| | terminator | |
| | region | |

### Example 7: Preparation for Genome Editing

A two-step two-plasmid system was employed in order to achieve high editing efficiency via CRISPR-Cas9 genome editing in Methylococcus capsulatus.

A “base” strain was created by transforming a pBBR1_gRNA_donor plasmid into Methylococcus capsulatus through electroporation. The base strains were frozen as a stock for future editing.

To perform gene editing, the base strain was pre-cultured by thawing out frozen stock vial of the base strain. The pre-cultures were grown to saturation (Optical Density (OD) at 600 nm of approximately 1-1.5). Once saturation was reached, a 1:100 dilution was made. The diluted cells were allowed to grow to an appropriate cell density (OD of 0.4-0.8). The cells were then prepared for electroporation.

The cells were washed three times in a solution comprising 2.5% sucrose and concentrated. The concentrated cells were then re-suspend in the solution comprising 2.5% sucrose to achieve an OD between 40-90. 300 ng of genetic materials to be electroporated, for example pSL95 (from example 1), were used for electroporation per 50 ul electrocompetent cells. The cells were cultured in IM5 media supplemented with 2.5% sucrose for at least 4 hours up to overnight. Then cells were plated on agar plates containing spectinomycin and kanamycin. The plates were placed in incubators containing 95% methane and 5% CO₂ until the appearance of colonies.

Editing efficiency was measured by confirming the proper nucleotide sequence using PCR and sequencing. First, a pair of primers were designed so that they anneal to the outside of donor region. PCR reactions were performed on several colonies (e.g., 8-12 colonies (colony PCR)), using the aforementioned primers for the amplification reaction. DNA sequencing of the amplified DNA products was used to determine the number of isolates that display correct editing (editing efficiency).

### Example 8: Incorporating Point Mutations Using AGE

Point mutations were incorporating using AGE within the ppdK locus.

Two gRNAs were designed to knock out ppdK function through introducing stop codons and frame shift mutations. The gRNA and donor sequences are listed in Table 3A.

TABLE 3A

| | | Targeting | | | Editing |
|---|---|---|---|---|---|
| Plasmid | gRNA1 | Sequence | Full sgRNA sequence | Donor sequence | efficiency |
| pSL148 | gRNA1 | GCTCGAAG | TTGACGGCTAGCTCAGTCC | GTTCGACGCCGAATTCGAAGCC | 83% |
| | | CCCATTAC | TAGGTATAGTGCTAGCGCT | ATCAAGCACCAGGCCGGGGTCG | |
| | | CACG (SEQ | CGAAGCCCATTACCACGGT | CCGCCGACATCGGCCTGAGCGC | |
| | | ID NO. 82) | TTTAGAGCTAGAAATAGCA | CGTCCATCTCGCCGACATCGGCG | |
| | | | AGTTAAAATAAGGCTAGTC | AACGTTTCCTCGCCGTCGTACGC | |
| | | | CGTTATCAACTTGAAAAAG | CGCCATACCGGCAAGCCTTTCCC | |
| | | | TGGCACCGAGTCGGTGCTT | CGAGGACGTCTACGAGCAGCTC | |
| | | | TTTTT (SEQ ID NO. 83) | GAGATCGCGATCCGGGCGGTAT | |
| | | | | TCGACTCCTGGATGGGAAACGC | |
| | | | | (SEQ ID NO. 84) | |
| pSL150 | gRNA2 | TCAACCAC | TTGACGGCTAGCTCAGTCC | GTGACCGAGCCTATCTGGAATTG | 100% |
| | | GATACACT | TAGGTATAGTGCTAGCTCA | CTCAAGGCCCTGGCCCAGTTGCG | |
| | | CCAG (SEQ | ACCACGATACACTCCAGGT | CCTGCTGGAACGGCTGAATGCTC | |
| | | ID NO. 85) | TTTAGAGCTAGAAATAGCA | GAAAAATGGGCCGGAACCGGCT | |
| | | | AGTTAAAATAAGGCTAGTC | CTGATCCTGCAGTGACAAGCGG | |
| | | | CGTTATCAACTTGAAAAAG | CGAACGCGACAGTTCGAGGAGA | |
| | | | TGGCACCGAGTCGGTGCTT | CCATCATGACGATGAAAAAGCG | |
| | | | TTTTT (SEQ ID NO. 86) | TGTCTACGCCTTCTCCGAAGGCG | |
| | | | | ACGGCAAGAACAAACGCCT | |
| | | | | (SEQ ID NO. 87) | |

Additional targets were chosen for point mutations. For example, the genes rpoB, MCA2598, and MDH were targeted for point mutation editing. Editing efficiency for these genes ranged from 88% to 96%. The gRNA and donor sequences are listed in Table 3B.

TABLE 3B

| | | | Full | | |
|---|---|---|---|---|---|
| | Gene | Targeting | sgRNA | | Editing |
| Plasmid | Target | Sequence | sequence | Donor sequence | efficiency |
| pSL128 | rpoB | GGACCAGA | TTGACGGC | TACCATGCGCATCGATCTGACCGAGACGCAGCTGGATGCCCTG | 96% |
| | | ACAATCCG | TAGCTCAG | GTGGAAATCTACCGGATGATGCGGCCGGGCGAACCGCCGACC | |
| | | TTGT (SEQ | TCCTAGGT | AAGGAGGCCGCCCAGACCCTGTTCGAAAATCTGTTTTTCTCGG | |
| | | ID NO. 88) | ATAGTGCT | CCGAACGCTATGATCTGTCGGCTGTCGGCCGGATGAAGTTCAA | |
| | | | AGCGGACC | CCGGCGCCTGGGGCGGACCGATCCTACCGGCCCCGGCGTGCT | |
| | | | AGAACAAT | GGAAAACGATGACATCATCGCGGTGCTGAAGGAACTGATCAA | |
| | | | CCGTTGTG | CATCCGTAACGGGGGGGGCACGGTCGACGACATCGACCATCT | |
| | | | TTTTAGAG | GGGTAACCGCCGCGTCCGGTCCGTGGGGGAGATGGTGGAGA | |
| | | | CTAGAAAT | ATCAGTTCAGGCTCGGGCTGGTCCGGGTCGAACGGGCCGTGA | |
| | | | AGCAAGTT | AGGAGCGTCTGTCGCTGCCCGACGCCGATGGTCTGATGCCACA | |
| | | | AAAATAAG | GGAGATCATCAACGCCAAACCGGTGGCCGCTTCCATCAAGGA | |
| | | | GCTAGTCC | ATTCTTCGGTTCGAGCCAGCTTTCGCGGTTCATGGACCAGAAT | |
| | | | GTTATCAA | AACCCATTATCTGAGGTCACTCACAAGCGCCGTGTCTCGGCTCT | |
| | | | CTTGAAAA | GGGGCCGGGGGGGCTGGCGCGTGAGCGCGCCGGCTTCGAAG | |
| | | | AGTGGCAC | TGCGCGACGTGCATACCACCCACTACGGCCGTGTCTGTCCGAT | |
| | | | CGAGTCGG | CGAAACGCCCGAAGGTCCGAACATCGGCCTGATCAACTCGCTC | |
| | | | TGCTTTTTT | GCGGTCTACTCGCGCACCAACGAATACGGCTTTCTGGAGACGC | |
| | | | T (SEQ ID | CGTATCGAAAGGTGATCGACGGCCGGGTGACCGATCAGATCG | |
| | | | NO. 89) | AGTACCTGTCGGCTATCGAGGAGGGCCAGTACTACATTGCTCA | |
| | | | | GGCCAGCGCCTCGGTCGATGAAAACGGCATGCTCAAGGATGA | |
| | | | | ACTGGTGTCGTGTCGCCACAAGGATGAATTCACCCTGGCGTCG | |
| | | | | CGGGAAAACATCAACTACATGGATGTGTCGTCCAAACAGATCG | |
| | | | | TGTCGGTCGCGGCCTCGCTGATCCCCTTCCTCGAACACGATGA | |
| | | | | TGCCAACCGCGCCCTGATGGGCTCGAACATGCAGCGGCAGGC | |
| | | | | CGTCCCGACGCTGCGTACGGAGAA (SEQ ID NO. 90) | |
| gap | MCA25 | CGTCGTGC | TTGACGGC | CCGGGACGGAACGCTTGCCAGGCCATCTGGATGTGGCGGGGA | 88% |
| gRNA5 | 98 | ATGAGTGC | TAGCTCAG | CCGGCGCCGTCGACGAACACGATCTCGAGGTCGTCGGCCACC | |
| | | ACCG | TCCTAGGT | GCCAGATAGAGCATGTTGCGAATGGGGACGAACAGTGGGTCG | |
| | | (SEQ ID | ATAGTGCT | GGGCGCCGGCGGCCGAGCAGTACCCGGCTGAGGTTGTAGGTT | |
| | | NO. 91) | AGCCGTCG | GCGGCGCCGAGCAGGCGTGCCTCGCGTCGGAGTTCGCGCGAA | |
| | | | TGCATGAG | CGATGGAAGGTTTCCTGCATGTGGATGGCGTCGCTTGCTGGGT | |
| | | | TGCACCGG | CCCGGTTTAAGCTATCAGCCTTGGTGCGCGAAGGGCAAGAACT | |
| | | | TTTTAGAG | GGACGCTCGGCGCGGCGCTTTGCTATATTGGCCACGCTTTGTA | |
| | | | CTAGAAAT | TTGACAGATTCCCCGGAGAATGCCCGATTCTCCCTCGCCGAAG | |
| | | | AGCAAGTT | GCCCGCAGGCGTCCGGCCGGGACGGCCTCGACTGCTCTCCCCA | |
| | | | AAAATAAG | CCCCACTCCGCCGGTCGCCAAGGGTGCGAGGGCTCTCGCGCAT | |
| | | | GCTAGTCC | GACCCCATCCTCACTTTATTTCAGCATTTCTGGAGCAGGGCAAT | |
| | | | GTTATCAA | GACGATTAAGATTGCAATCAATGGATATGGGCGCATCGGCCG | |
| | | | CTTGAAAA | CAACATCCTGCGGGCGATTTACGAAACCGGGCGCAAGGATGT | |
| | | | AGTGGCAC | GGAGATCGTCGCCATCAATGACCTGGGGGATGCCCAGATCAA | |
| | | | CGAGTCGG | CGCCCATCTCACCCGCCATGACACCGTGCACGGGCCGTTCCGG | |
| | | | TGCTTTTTT | GGGACCGTGGAGGTCGGCGAGGGCGAAATCATCGTCAACGG | |
| | | | T (SEQ ID | CGACCGCATCAGGGTTTTTTCCGAGAAGGATCCTTCCAAGCTG | |
| | | | NO. 92) | CCCTGGGGGGCTTTGGGCGTGGACGTCGTGCATGAGTGATAG | |
| | | | | CACCTGTTCCGCACCAAGGCCAAATGCCAGCCGCATCTCGATG | |
| | | | | CCGGCGCCAAGAAGGTGATCATTTCGGCCCCGGCCGACAAGA | |
| | | | | ACGAGTGCGACGCGACCATCGTCTACGGGGTCAATGAGCATA | |
| | | | | CGCTGAAAGCCGCCCACACCGTCATCTCGAATGCATCCTGCAC | |
| | | | | CACCAACTGCCTGGCGCCGCTGGTCAAGCCGCTGCTGGGAAA | |
| | | | | AATCGGGATCGTGTCCGGCCTCATGACCACCGTGCATTCCTAC | |
| | | | | ACCAACGACCAGGTGCTCACCGACGTTTATCACAAGGATCTGT | |
| | | | | ACCGGGCACGGGCGGCGGCGCTGAACATGATCCCGACCAAGA | |
| | | | | CCGGCGCGGCGCAGGCCGTGGGGCTGGTGCTGCCGGAGCTG | |
| | | | | GACGGCAAACTGTCCGGTTTCGCCATCCGGGTGCCGACCGCCA | |
| | | | | ATGTATCGGTCGTGGACCTGACCTTCATCGCGGCCCGGGAAAC | |
| | | | | CGACAAGGACGAGATCAACGCCATCCTCAAGGC (SEQ ID NO. | |
| | | | | 93) | |
| plK11 | MDH | CCCATCAC | TTGACGGC | AAGATCGATGACACCGTCAACTGGGTGAAAAAGGTCGATCTG | 88% |
| | | CTATCAGC | TAGCTCAG | AAGACCGGCCTGCCGATCCGCGATCCGGAGTACAGCACCCGC | |
| | | ACAA | TCCTAGGT | ATGGACCACAATGCCAAAGGCATCTGTCCCTCGGCCATGGGCT | |
| | | (SEQ ID | ATAGTGCT | ATCACAACCAGGGCATCGAGTCCTACGATCCGGACAAGAAGCT | |
| | | NO. 94) | AGCCCCAT | GTTCTTCATGGGCGTGAACCACATCTGCATGGACTGGGAGCCG | |
| | | | CACCTATC | TTCATGCTGCCCTACCGCGCCGGCCAGTTCTTTGTGGGGGCGA | |
| | | | AGCACAAG | CCCTCAACATGTATCCGGGACCCAAGGGGATGCTGGGTCAGG | |
| | | | TTTTAGAG | TCAAGGCGATGAACGCGGTCACCGGCAAGATGGAATGGGAA | |
| | | | CTAGAAAT | GTGCCGGAGAAGTTTGCGGTCTGGGGTGGCACCTTGGCGACC | |
| | | | AGCAAGTT | GCCGGCGACCTCGTGTTCTACGGTACCCTCGACGGCTTCATCA | |
| | | | AAAATAAG | AGGCCCGCGACACCCGTACCGGCGAGCTGAAGTGGCAGTTCC | |
| | | | GCTAGTCC | AGTTGCCCTCCGGCGTGATCGGCCATCCCATCACGTACCAACA | |
| | | | GTTATCAA | TAACGGCAAGCAATACATTGCCATCTACTCCGGCGTCGGCGGC | |
| | | | CTTGAAAA | TGGCCAGGAGTAGGGCTGGTATTCGACCTGAAGGACCCGACC | |
| | | | AGTGGCAC | GCAGGTCTGGGAGCTGTGGGTGCGTTCAGGGAACTGGCGCAT | |
| | | | CGAGTCGG | TACACCCAGATGGGTGGATCGGTGTTCGTGTTCTCGCTTTGAG | |
| | | | TGCTTTTTT | TCGAAGGGGTGGAGGCGCTCCTGGGGGAGCGCCCCTATCCCA | |
| | | | T (SEQ ID | TGCTGTCGAAAGGATGAATCATGCGAATGAACCGTATTGCAGC | |
| | | | NO. 95) | CGCGGGGTTGGCCGCCTCCCTCGCGGTCGTGGGATGCGTGCA | |
| | | | | GGCAGCGACGAGCGTCGAACCGCTCAAGGTCTGCTCCGCGGA | |
| | | | | AAACGAGATGCCGTATTCGGACAAGGCCGGAGAGGGTTTCGA | |
| | | | | AAATAAGTTGGCTGAGCTCCTTGGAAAGGGATTGGGACGGCC | |
| | | | | AGTCGAGAACGTGTGGTGGACCGATGCCCGCTATTTCGTCCGG | |
| | | | | GATTATCTGGACAGGGGTTTGTGCGATGTGGTCATCGGCGTCG | |
| | | | | ATACCGGCGACCCGCGGATGCTCACCAGCAGTCCTTATTACCG | |
| | | | | GTCCGGCTACGTATTCGTCTACCGCAAGGACACGGGACTGAGC | |
| | | | | ATCCAAGATTGGAACAGCGCGGCACTGAAGACCGTGAAGCGG | |
| | | | | ATC (SEQ ID NO. 96) | |

### Example 9: Deletion of Portions of Genes

Portions of genes were deleted using AGE within specific genes such as the ppdK locus.

gRNA2 was designed to knock out ppdK function through gene deletion. Table 4A contains the gRNA and donor sequences used for creating the ppdK deletions.

TABLE 4A

| | | Targeting | | | Editing |
|---|---|---|---|---|---|
| Plasmid | gRNA1 | Sequence | Full sgRNA sequence | Donor sequence | efficiency |
| pSL178 | gRNA2 | TCAACCAC | TTGACGGCTAGCTCAGTCC | GTGTTCCCTGGAACACGCCATCA | 72% |
| | | GATACACT | TAGGTATAGTGCTAGCTCA | ATTTCGATCTCGTGCTCAACACC | |
| | | CCAG (SEQ | ACCACGATACACTCCAGGT | GACCATCTGCCAGCCGGTAACG | |
| | | ID NO. 97) | TTTAGAGCTAGAAATAGCA | CTCTGCCGACCGTACTCATGGCG | |
| | | | AGTTAAAATAAGGCTAGTC | GTACGGCAGTTCGGCTTCGAAAT | |
| | | | CGTTATCAACTTGAAAAAG | CTTCGATCTCGGTCAGCGGGAA | |
| | | | TGGCACCGAGTCGGTGCTT | GCCTCGTGAGCCCGGCGGTGGG | |
| | | | TTTTT (SEQ ID NO. 98) | ACAGACCGCATCCCGTCTGCTAA | |
| | | | | CGGTCGAGATCGTCGACGTCTG | |
| | | | | CCGGGAGATATTTTCCGGGCGTT | |
| | | | | GTAGCCGGGTGGTCGCGCCAGC | |
| | | | | GGCAGACGGTGAGGTCGGCGTT | |
| | | | | CTGCCCCGTCATACGCCGTTCCT | |
| | | | | GACCCGGCTCCGGCCCGGCGAG | |
| | | | | ATAAGGC (SEQ ID NO. 99) | |

Additional targets were chosen for deletions within the ppdK gene. The deletion size of this second locus was 200 base pairs. Editing efficiency for the second locus was 88%. The gRNA and donor sequences are listed in Table 4B.

TABLE 4B

| | | | Full | | |
|---|---|---|---|---|---|
| | Gene | Targeting | sgRNA | | Editing |
| Plasmid | target | Sequence | sequence | Donor sequence | efficiency |
| pSL179 | ppdK | TCAACCAC | TTGACGGC | GTGTTCCCTGGAACACGCCATCAATTTCGATCTCGTGCTCAACACC | 88% |
| | | GATACACT | TAGCTCAG | GACCATCTGCCAGCCGGTAACGCTCTGCCGACCGTACTCATGGCGG | |
| | | CCAG (SEQ | TCCTAGGT | TACGGCAGTTCGGCTTCGAAATCTTCGATCTCGGTCAGCGGGAAGC | |
| | | ID NO. | ATAGTGCT | CTCGTGAGCCCGGCGGTGGGACAGACCGCATCCCGTCTGCTAACG | |
| | | 100) | AGCTCAAC | GTCGAGATCGTCGACGTCTGCCGGGAGATATTTTCCGGGCGTTGT | |
| | | | CACGATAC | AGCCGGGTGGTCGCGCCAGCGGCAGACGGTGAGGTCGGCGTTCT | |
| | | | ACTCCAGG | GCCCCGTCATACGCCGTTCCTGACCCGGCTCCGGCCCGGCGAGATA | |
| | | | TTTTAGAG | AGGCTCAGGACCGAGGCAGGCGAAGACCAGTATTTCTACCTCTCC | |
| | | | CTAGAAAT | GGGGGCTACATGGAGGTGCAGCGCTGGGAGGTCAGCATCCTGGC | |
| | | | AGCAAGTT | CGACCAGGTGCTCCGCTCCCAAGAGATCGACCGGGAAGCGGCCCT | |
| | | | AAAATAAG | GGCGGCCAAGCGCAACGCAGAGCGGATGCTCCGCGAGAACCGGA | |
| | | | GCTAGTCC | TTCCCGGCGAGCGTGACCGAGCCTATCTGGAATTGCTCAAGGCCCT | |
| | | | GTTATCAA | GGCCCAGTTGCGCCTGCTGGAACGGCTGAATGCTCGAAAAATGGG | |
| | | | CTTGAAAA | CCGGAACCGGCTCTGATCCTGCAGTGACAAGCGGCGAACGCGACA | |
| | | | AGTGGCAC | GTTCGAGGAGACCATCATGACGATGAAAAAGCGTGTCTACGCCTT | |
| | | | CGAGTCGG | CTCCGAAGGCGACGGCAAGAACAAACGCCTGCTCGGCGGCAAGG | |
| | | | TGCTTTTTT | GCGCCAACCTCTGCGAAATGACGCAGATCGGGCTCAACGTGCCGC | |
| | | | T (SEQ ID | CGGGTTTCGTTATTACCACGGAAGCCTGCCTCGAATACCTGGCAGA | |
| | | | NO. 101) | CAAGAAGCTGCCGGCCGGCTTGATGGACGAAGTCCGGGAGCACAT | |
| | | | | GGCCCGGCTCGAACGGGCTACCGGCAAGCGCTTCGGCGATCCCGC | |
| | | | | CAATCCACTCTTGGTTTCGGTGCGTTCCGGTTCGGCCCTGTCCATGC | |
| | | | | CGGGCATGATGGATACCATTCTCAACCTCGGCCTCAACCACGATAC | |
| | | | | ACTCTTTCCTCGCCGTCGTACGCCGCCATACCGGCAAGCCTTTCCCC | |
| | | | | GAGGACGTCTACGAGCAGCTCGAGATCGCGATCCGGGCGGTATTC | |
| | | | | GACTCCTGGATGGGAAAGCGCGCGGTGGATTACCGCCGCGAATTC | |
| | | | | CACATCACGCCCGACCAGGCCAACGGCACGGCGGTGAACGTGGTG | |
| | | | | ACCATGGTGTTCGGCAACATGGGCGACGACTCCGCCACCGGTGTC | |
| | | | | GGCTTCACCCGCAATCCGGGTACCGGTGAGAACGAGATGTTCGGC | |
| | | | | GAGTATCTGGTCAACGCCCAGGGTGAGGATGTGGTAGCCGGAATC | |
| | | | | CGCACGCCCAAGCCCGTGCACGAGATGGCAACCGAAATGCCGGCG | |
| | | | | CTTTACGCCCAACTGGTGGAACTGCGCGACAAGCTCGAAGCCCATT | |
| | | | | ACCACGAGGTGCAGGACTTCGAGTACACCATCGAGAAGGGGGTCT | |
| | | | | TGTACTGTCTGCAGACGCGCAACGGCAAGATGAACGCCCAGGCGA | |
| | | | | TGGTGCGCACCTCGGTCGAGATGTGCCGGGAAGGACTGATCACGC | |
| | | | | GGGATCAGGCCCTCTTGCGGGTCAACCCCGCCCATCTGGAACAGTT | |
| | | | | ACTCCATCCCTGCCTCGACACCTCGCACAACCCCACGCCGCTGGCG | |
| | | | | CAGGGGCTGCCTGCCTCGCCCGGCGCCGCCAGCGGCCGTTGCGTG | |
| | | | | TTCGATGCGGATCAGGCCGAACTGTTGGGACGGGCCGGTGAAAA | |
| | | | | GGTCATCCTGGTGCGTGAGGAGACCAAGCCGGAAGACATCCACGG | |
| | | | | CTTCTTCGCGGCCCAGGGAATCCTCACCAGTCGCGGCGGCAAGAC | |
| | | | | CTCGCATGCCGCCGTGGTCGCCCGCGGCATGGGCAAGGCCTGCGT | |
| | | | | GGCCGGGGCCGAAGGCATCAGGGTGGACAGCCGGGCGCGGCTG | |
| | | | | GCAACGGTGGGAGAGGTCACGTTGCACGAAGGTGACATCATCACC | |
| | | | | ATCGACGGCAGCACCGGCCGTGTCTATCTCGGCGCGATCCCGACG | |
| | | | | ATCGCGCCGACCTTCTCCGAACACCTCAGGACACTGCTGTC (SEQ | |
| | | | | ID NO. 102) | |

### Example 10: Insertion of Nucleotides

Nucleotides were inserted using AGE within the ppdK locus.

gRNA2 was designed to integrate gamma protein gene (417 bp) at ppdK locus. Table 5A indicates the gRNA and donor sequences used for creating the gamma protein gene integration.

TABLE 5A

| | | Targeting | | | Editing |
|---|---|---|---|---|---|
| Plasmid | gRNA1 | Sequence | Full sgRNA sequence | Donor sequence | efficiency |
| pSL162 | gRNA2 | TCAACCAC | TTGACGGCTAGCTCAGTCC | GTGACCGAGCCTATCTGGAATTG | 11% |
| | | GATACACT | TAGGTATAGTGCTAGCTCA | CTCAAGGCCCTGGCCCAGTTGCG | |
| | | CCAG (SEQ | ACCACGATACACTCCAGGT | CCTGCTGGAACGGCTGAATGCTC | |
| | | ID NO. | TTTAGAGCTAGAAATAGCA | GAAAAATGGGCCGGAACCGGCT | |
| | | 103) | AGTTAAAATAAGGCTAGTC | CTGATCCTGCAGTGACAAGCGG | |
| | | | CGTTATCAACTTGAAAAAG | CGAACGCGACAGTTCGAGGAGA | |
| | | | TGGCACCGAGTCGGTGCTT | CCATCATGACGATGAAAAAGCG | |
| | | | TTTTT (SEQ ID NO. | TGTCTACGCCTTCTCCGAAGGCG | |
| | | | 104) | ACGGCAAGAACAAACGCCT | |
| | | | | (SEQ ID NO. 105) | |

Additional targets were chosen for insertions within specific genes. The size of the fragments were relatively small, i.e., 34 and 39 bps. Editing efficiency for the second locus was 83% for both of these genes. The gRNA and donor sequences are listed in Table 5B.

TABLE 5B

| | | Targeting | | | |
|---|---|---|---|---|---|
| | Gene | Sequence | Full | | Editing |
| Plasmid | target | sequence | sgRNA | Donor sequence | efficiency |
| pSL154 | ppdK | TCAACCAC | TTGACGGC | GTGACCGAGCCTATCTGGAATTGCTCAAGGCCCTGGCCCAGTTGCG | 83% |
| | (attB | GATACACT | TAGCTCAG | CCTGCTGGAACGGCTGAATGCTCGAAAAATGGGCCGGAACCGGCT | |
| | site) | CCAG (SEQ | TCCTAGGT | CTGATCCTGCAGTGACAAGCGGCGAACGCGACAGTTCGAGGAGAC | |
| | | ID NO. | ATAGTGCT | CATCATGACGATGAAAAAGCGTGTCTACGCCTTCTCCGAAGGCGAC | |
| | | 106) | AGCTCAAC | GGCAAGAACAAACGCCTGCTCGGCGGCAAGGGCGCCAACCTCTGC | |
| | | | CACGATAC | GAAATGACGCAGATCGGGCTCAACGTGCCGCCGGGTTTCGTTATT | |
| | | | ACTCCAGG | ACCACGGAAGCCTGCCTCGAATACCTGGCAGACAAGAAGCTGCCG | |
| | | | TTTTAGAG | GCCGGCTTGATGGACGAAGTCCGGGAGCACATGGCCCGGCTCGAA | |
| | | | CTAGAAAT | CGGGCTACCGGCAAGCGCTTCGGCGATCCCGCCAATCCACTCTTGG | |
| | | | AGCAAGTT | TTTCGGTGCGTTCCGGTTCGGCCCTGTCCATGCCGGGCATGATGGA | |
| | | | AAAATAAG | TACCATTCTCAACCTCGGCCTCAACCACGATACACTCTAAGTGCCAG | |
| | | | GCTAGTCC | GGCGTGCCCTTGGGCTCCCCGGGCGCGGGGTTGATCCGGCAGACC | |
| | | | GTTATCAA | GGCAACGAGCGCTTCGGTCACGATGCCTACCGGCGGTTCATCCAG | |
| | | | CTTGAAAA | TTGTTCGGCAAGGTTGCCCTCGGTGTTCCCGACGAGCTGTTCGACG | |
| | | | AGTGGCAC | CCGAATTCGAAGCCATCAAGCACCAGGCCGGGGTCGCCGCCGACA | |
| | | | CGAGTCGG | TCGGCCTGAGCGCCGTCCATCTCGCCGACATCGGCGAACGTTTCCT | |
| | | | TGCTTTTTT | CGCCGTCGTACGCCGCCATACCGGCAAGCCTTTCCCCGAGGACGTC | |
| | | | T (SEQ ID | TACGAGCAGCTCGAGATCGCGATCCGGGCGGTATTCGACTCCTGG | |
| | | | NO. 107) | ATGGGAAAGCGCGCGGTGGATTACCGCCGCGAATTCCACATCACG | |
| | | | | CCCGACCAGGCCAACGGCACGGCGGTGAACGTGGTGACCATGGT | |
| | | | | GTTCGGCAACATGGGCGACGACTCCGCCACCGGTGTCGGCTTCAC | |
| | | | | CCGCAATCCGGGTACCGGTGAGAACGAGATGTTCGGCGAGTATCT | |
| | | | | GGTCAACGCCCAGGGTGAGGATGTGGTAGCCGGAATCCGCACGCC | |
| | | | | CAAGCCCGTGCACGAGATGGCAACCGAAATGC (SEQ ID NO. 108) | |
| pSL175 | aacC | TACTACGG | TTGACGGC | GCCAGGACAGAAATGCCTCGACTTCGCTGCTGCCCAAGGTTGCCG | 83% |
| | (attP) | AGCAAGTT | TAGCTCAG | GGTGACGCACACCGTGGAAACGGATGAAGGCACGAACCCAGTTG | |
| | site) | CCCG (SEQ | TCCTAGGT | ACATAAGCCTGTTCGGTTCGTAAACTGTAATGCAAGTAGCGTATGC | |
| | | ID NO. | ATAGTGCT | GCTCACGCAACTGGTCCAGAACCTTGACCGAACGCAGCGGTGGTA | |
| | | 109) | AGCTACTA | ACGGCGCAGTGGCGGTTTTCATGGCTTGTTATGACTGTTTTTTTGTA | |
| | | | CGGAGCAA | CAGTCTATGCCTCGGGCATCCAAGCAGCAAGCGCGTTACGCCGTG | |
| | | | GTTCCCGG | GGTCGATGTTTGATGTTATGGAGCAGCAACGATGTTACGCAGCAG | |
| | | | TTTTAGAG | CAACGATGTTACGCAGCAGGGCAGTCGCCCTAAAACAAAGTTAGG | |
| | | | CTAGAAAT | TGGCTCAAGTATGGGCATCATTCGCACATGTAGGCTCGGCCCTGAC | |
| | | | AGCAAGTT | CAAGTCAAATCCATGCGGGCTGCTCTTGATCTMCGGTCGTGAGT | |
| | | | AAAATAAG | TCGGAGACGTAGCCACCTACTCCCAACATCAGCCGGACTCCGATTA | |
| | | | GCTAGTCC | GCTCCCCCAACTGAGAGAACTCAAAGGTTACCCCAGTTGGGGCGG | |
| | | | GTTATCAA | GTAACTTGCTCCGTAGTAAGACATTCATCGCGCTTGCTGCCTTCGA | |
| | | | CTTGAAAA | CCAAGAAGCGGTTGTTGGCGCTCTCGCGGCTTACGTTCTGCCCAGG | |
| | | | AGTGGCAC | TTTGAGCAGCCGCGTAGTGAGATCTATATCTATGATCTCGCAGTCT | |
| | | | CGAGTCGG | CCGGCGAGCACCGGAGGCAGGGCATTGCCACCGCGCTCATCAATC | |
| | | | TGCTTTTTT | TCCTCAAGCATGAGGCCAACGCGCTTGGTGCTTATGTGATCTACGT | |
| | | | T (SEQ ID | GCAAGCAGATTACGGTGACGATCCCGCAGTGGCTCTCTATACAAA | |
| | | | NO. 110) | GTTGGGCATACGGGAAGAAGTGATGCACTTTGATATCGACCCAAG | |
| | | | | TACCGCCACCTAACAATTCGTTCAAGCCGAGATCGGCTTCATAACTT | |
| | | | | CGTATAGCATACATTATACGAAGTTATTGGCAGAGCATTACGCTGA | |
| | | | | CTTGACCAGAGGCTGCATTTCCACCGCTGATTGCGATTCGGAAGGT | |
| | | | | GCAGGCCGGAGGGTCCGGACCGCCGCTCCACCCGTTGTTTTC(SEQ | |
| | | | | ID NO. 111) | |

Larger insertional targets were chosen. Targets with DNA insertion size of 684 bp and 1083 bp were chosen. Editing efficiency was lower for larger insertional size fragments, i.e., under 100% efficiency. The gRNA and donor sequences are listed in Table 5C.

TABLE 5C

| | | | Full | | |
|---|---|---|---|---|---|
| | Gene | Targeting | sgRNA | | Editing |
| Plasmid | target | Sequence | sequence | Donor sequence | efficiency |
| pSL163 | ppdK | TCAACCAC | TTGACGGC | GTGACCGAGCCTATCTGGAATTGCTCAAGGCCCTGGCCCAGTTGCG | 6% |
| | (mcherry) | GATACACT | TAGCTCAG | CCTGCTGGAACGGCTGAATGCTCGAAAAATGGGCCGGAACCGGCT | |
| | | CCAG (SEQ | TCCTAGGT | CTGATCCTGCAGTGACAAGCGGCGAACGCGACAGTTCGAGGAGAC | |
| | | ID NO. | ATAGTGCT | CATCATGACGATGAAAAAGCGTGTCTACGCCTTCTCCGAAGGCGAC | |
| | | 112) | AGCTCAAC | GGCAAGAACAAACGCCTGCTCGGCGGCAAGGGCGCCAACCTCTGC | |
| | | | CACGATAC | GAAATGACGCAGATCGGGCTCAACGTGCCGCCGGGTTTCGTTATT | |
| | | | ACTCCAGG | ACCACGGAAGCCTGCCTCGAATACCTGGCAGACAAGAAGCTGCCG | |
| | | | TTTTAGAG | GCCGGCTTGATGGACGAAGTCCGGGAGCACATGGCCCGGCTCGAA | |
| | | | CTAGAAAT | CGGGCTACCGGCAAGCGCTTCGGCGATCCCGCCAATCCACTCTTGG | |
| | | | AGCAAGTT | TTTCGGTGCGTTCCGGTTCGGCCCTGTCCATGCCGGGCATGATGGA | |
| | | | AAAATAAG | TACCATTCTCAACCTCGGCCTCAACCACGATACACTCTAATTGACGG | |
| | | | GCTAGTCC | CTAGCTCAGTCCTAGGTATAGTGCTAGCAAAAAGCTCAACGAGAG | |
| | | | GTTATCAA | GAAGTTCCATGGCCATCATCAAGGAGTTCATGCGCTTCAAGGTGCA | |
| | | | CTTGAAAA | CATGGAGGGCTCCGTGAACGGCCACGAGTTCGAGATCGAGGGCG | |
| | | | AGTGGCAC | AGGGCGAGGGCCGCCCCTACGAGGGCACCCAGACCGCCAAGCTG | |
| | | | CGAGTCGG | AAGGTGACCAAGGGTGGCCCCCTGCCCTTCGCCTGGGACATCCTGT | |
| | | | TGCTTTTTT | CCCCTCAGTTCATGTACGGCTCCAAGGCCTACGTGAAGCACCCCGC | |
| | | | T (SEQ ID | CGACATCCCCGACTACTTGAAGCTGTCCTTCCCCGAGGGCTTCAAG | |
| | | | NO. 113) | TGGGAGCGCGTGATGAACTTCGAGGACGGCGGCGTGGTGACCGT | |
| | | | | GACCCAGGACTCCTCCCTCCAGGACGGCGAGTTCATCTACAAGGTG | |
| | | | | AAGCTGCGCGGCACCAACTTCCCCTCCGACGGCCCCGTAATGCAGA | |
| | | | | AGAAGACCATGGGCTGGGAGGCCTCCTCCGAGCGGATGTACCCCG | |
| | | | | AGGACGGCGCCCTGAAGGGCGAGATCAAGCAGAGGCTGAAGCTG | |
| | | | | AAGGACGGCGGCCACTACGACGCTGAGGTCAAGACCACCTACAAG | |
| | | | | GCCAAGAAGCCCGTGCAGCTGCCCGGCGCCTACAACGTCAACATC | |
| | | | | AAGTTGGACATCACCTCCCACAACGAGGACTACACCATCGTGGAAC | |
| | | | | AGTACGAACGCGCCGAGGGCCGCCACTCCACCGGCGGCATGGAC | |
| | | | | GAGCTGTACAAGTAAGGGTTGATCCGGCAGACCGGCAACGAGCG | |
| | | | | CTTCGGTCACGATGCCTACCGGCGGTTCATCCAGTTGTTCGGCAAG | |
| | | | | GTTGCCCTCGGTGTTCCCGACGAGCTGTTCGACGCCGAATTCGAAG | |
| | | | | CCATCAAGCACCAGGCCGGGGTCGCCGCCGACATCGGCCTGAGCG | |
| | | | | CCGTCCATCTCGCCGACATCGGCGAACGTTTCCTCGCCGTCGTACG | |
| | | | | CCGCCATACCGGCAAGCCTTTCCCCGAGGACGTCTACGAGCAGCTC | |
| | | | | GAGATCGCGATCCGGGCGGTATTCGACTCCTGGATGGGAAAGCGC | |
| | | | | GCGGTGGATTACCGCCGCGAATTCCACATCACGCCCGACCAGGCC | |
| | | | | AACGGCACGGCGGTGAACGTGGTGACCATGGTGTTCGGCAACATG | |
| | | | | GGCGACGACTCCGCCACCGGTGTCGGCTTCACCCGCAATCCGGGT | |
| | | | | ACCGGTGAGAACGAGATGTTCGGCGAGTATCTGGTCAACGCCCAG | |
| | | | | GGTGAGGATGTGGTAGCCGGAATCCGCACGCCCAAGCCCGTGCAC | |
| | | | | GAGATGGCAACCGAAATGC (SEQ ID NO. 114) | |
| pSL165 | ppdK | TCAACCAC | TTGACGGC | GTGACCGAGCCTATCTGGAATTGCTCAAGGCCCTGGCCCAGTTGCG | 5% |
| | (adh) | GATACACT | TAGCTCAG | CCTGCTGGAACGGCTGAATGCTCGAAAAATGGGCCGGAACCGGCT | |
| | | CCAG (SEQ | TCCTAGGT | CTGATCCTGCAGTGACAAGCGGCGAACGCGACAGTTCGAGGAGAC | |
| | | ID NO. | ATAGTGCT | CATCATGACGATGAAAAAGCGTGTCTACGCCTTCTCCGAAGGCGAC | |
| | | 115) | AGCTCAAC | GGCAAGAACAAACGCCTGCTCGGCGGCAAGGGCGCCAACCTCTGC | |
| | | | CACGATAC | GAAATGACGCAGATCGGGCTCAACGTGCCGCCGGGTTTCGTTATT | |
| | | | ACTCCAGG | ACCACGGAAGCCTGCCTCGAATACCTGGCAGACAAGAAGCTGCCG | |
| | | | TTTTAGAG | GCCGGCTTGATGGACGAAGTCCGGGAGCACATGGCCCGGCTCGAA | |
| | | | CTAGAAAT | CGGGCTACCGGCAAGCGCTTCGGCGATCCCGCCAATCCACTCTTGG | |
| | | | AGCAAGTT | TTTCGGTGCGTTCCGGTTCGGCCCTGTCCATGCCGGGCATGATGGA | |
| | | | AAAATAAG | TACCATTCTCAACCTCGGCCTCAACCACGATACACTCTAATTGACGG | |
| | | | GCTAGTCC | CTAGCTCAGTCCTAGGTATAGTGCTAGCAAAAAGCTCAACGAGAG | |
| | | | GTTATCAA | GAAGTTCCATGAGCTATCCCGAGAAGTTCGAGGGGATCGCCATCC | |
| | | | CTTGAAAA | AGAGCCACGAGGACTGGAAGAACCCGAAAAAGACCAAGTATGAT | |
| | | | AGTGGCAC | CCGAAGCCCTTCTACGATCACGACATCGACATCAAGATCGAGGCCT | |
| | | | CGAGTCGG | GCGGCGTCTGCGGCAGCGATATCCATTGTGCGGCTGGCCACTGGG | |
| | | | TGCTTTTTT | GCAACATGAAGATGCCGTTGGTCGTCGGCCACGAGATCGTGGGCA | |
| | | | T (SEQ ID | AGGTCGTGAAGTTAGGCCCGAAAAGCAACAGCGGCTTGAAGGTG | |
| | | | NO. 116) | GGCCAGCGCGTGGGTGTGGGTGCGCAGGTCTTCAGCTGTCTGGAG | |
| | | | | TGCGACCGTTGCAAGAACGACAACGAACCGTACTGCACCAAGTTC | |
| | | | | GTCACCACCTACTCGCAGCCCTACGAGGACGGCTACGTCTCGCAGG | |
| | | | | GCGGTTACGCCAACTATGTCCGAGTCCACGAACACTTCGTGGTGCC | |
| | | | | CATCCCGGAAAATATCCCCAGCCATCTGGCGGCTCCCCTGCTGTGC | |
| | | | | GGTGGCTTGACCGTCTACAGCCCCCTCGTCCGCAATGGCTGCGGTC | |
| | | | | CCGGCAAGAAGGTGGGTATCGTGGGCCTCGGCGGTATAGGCTCTA | |
| | | | | TGGGCACGCTGATCTCGAAAGCGATGGGCGCAGAAACGTACGTGA | |
| | | | | TCTCGCGTTCCTCGCGCAAGCGCGAGGATGCGATGAAGATGGGTG | |
| | | | | CGGACCACTACATCGCCACGCTGGAGGAGGGTGACTGGGGTGAG | |
| | | | | AAGTACTTCGACACGTTCGACCTCATCGTGGTGTGCGCGAGTTCCC | |
| | | | | TGACGGACATCGACTTCAATATCATGCCCAAGGCGATGAAGGTCG | |
| | | | | GAGGGCGCATCGTCTCCATCTCGATCCCGGAGCAGCACGAAATGC | |
| | | | | TGTCGCTGAAGCCCTACGGCCTGAAAGCCGTCTCCATTAGCTACTC | |
| | | | | GGCGCTCGGTAGTATCAAGGAGCTCAACCAGCTGTTGAAGTTGGT | |
| | | | | TTCCGAAAAGGACATCAAGATCTGGGTGGAAACGCTCCCGGTGGG | |
| | | | | CGAAGCCGGTGTGCACGAGGCCTTTGAGCGGATGGAGAAGGGGG | |
| | | | | ATGTCCGTTATCGGTTTACACTCGTCGGCTACGATAAAGAGTTCTC | |
| | | | | GGATTAAGGGTTGATCCGGCAGACCGGCAACGAGCGCTTCGGTCA | |
| | | | | CGATGCCTACCGGCGGTTCATCCAGTTGTTCGGCAAGGTTGCCCTC | |
| | | | | GGTGTTCCCGACGAGCTGTTCGACGCCGAATTCGAAGCCATCAAG | |
| | | | | CACCAGGCCGGGGTCGCCGCCGACATCGGCCTGAGCGCCGTCCAT | |
| | | | | CTCGCCGACATCGGCGAACGTTTCCTCGCCGTCGTACGCCGCCATA | |
| | | | | CCGGCAAGCCTTTCCCCGAGGACGTCTACGAGCAGCTCGAGATCG | |
| | | | | CGATCCGGGCGGTATTCGACTCCTGGATGGGAAAGCGCGCGGTG | |
| | | | | GATTACCGCCGCGAATTCCACATCACGCCCGACCAGGCCAACGGC | |
| | | | | ACGGCGGTGAACGTGGTGACCATGGTGTTCGGCAACATGGGCGAC | |
| | | | | GACTCCGCCACCGGTGTCGGCTTCACCCGCAATCCGGGTACCGGTG | |
| | | | | AGAACGAGATGTTCGGCGAGTATCTGGTCAACGCCCAGGGTGAGG | |
| | | | | ATGTGGTAGCCGGAATCCGCACGCCCAAGCCCGTGCACGAGATGG | |
| | | | | CAACCGAAATGC (SEQ ID NO. 117) | |

### Example 11: Inhibition of Gene Expression Using AGEi-dCas9

Catalytically inactive Cas9 protein (dCas9) can form a complex with sgRNA and can bind to a target region to create a steric block interrupting transcript initiation or elongation by RNA polymerase. This can, if done correctly and in certain organisms, result in repression of a target gene, which is referred to as AGEi.

AGEi was performed in Methylococcus capsulatus using a two-plasmid based lacZ reporter assay (from Invitrogen lacZ assay kit): 1) OriV origin based plasmid carries pBAD_dCas9 and gRNA. 2) pBBR1 based plasmids carries Ptrc_lacZ. Five gRNA were designed to target different regions of lacZ. One gRNA that targets the promoter region of the reporter repressed lacZ expression consistently over a 72 hour time point. The relevant information related to the sequences is presented in Table 6A, Table 6B, and Table 6C below.

TABLE 6A

| SEQ ID | | |
|---|---|---|
| NO. | NAME | SEQUENCE |
| 118 | dCas9 | ATGGACAAGAAGTATTCGATCGGCCTGGCCATCGGCACCAACAGCGTCGGCTGGGCGGTCATCACGGATGAGTAC |
| | | AAGGTGCCGTCGAAGAAGTTCAAGGTGCTGGGCAATACCGACCGCCATAGCATCAAGAAGAATCTCATCGGCGCA |
| | | CTGCTGTTCGACTCCGGCGAAACCGCCGAAGCGACCCGCCTCAAGCGCACGGCCCGGCGGCGCTATACGCGCCGG |
| | | AAGAACCGCATCTGCTACCTCCAGGAAATCTTCTCCAACGAGATGGCCAAGGTGGATGACTCCTTCTTCCATCGCCT |
| | | GGAAGAATCCTTCCTGGTCGAAGAAGATAAGAAACATGAGCGCCACCCCATCTTCGGCAATATCGTGGACGAGGT |
| | | GGCGTATCACGAGAAATACCCGACCATCTATCACCTGCGGAAAAAGCTGGTGGACTCGACGGACAAAGCCGACCT |
| | | GCGCCTCATCTATCTGGCCCTGGCCCACATGATCAAGTTCCGGGGCCATTTCCTGATCGAAGGCGACCTGAACCCC |
| | | GATAACAGCGACGTGGACAAGCTCTTCATCCAGCTCGTCCAGACCTATAACCAGCTGTTCGAGGAGAACCCCATCA |
| | | ACGCCTCGGGCGTGGACGCCAAGGCCATCCTGAGCGCACGGCTCTCCAAGTCGCGCCGCCTGGAAAACCTGATCG |
| | | CGCAGCTGCCGGGCGAAAAGAAAAACGGCCTGTTCGGCAACCTGATCGCCCTGTCCCTCGGCCTCACCCCGAACTT |
| | | CAAGTCCAACTTCGACCTGGCCGAGGACGCGAAGCTCCAGCTGTCGAAAGACACCTACGATGACGACCTGGACAA |
| | | CCTCCTGGCGCAGATCGGCGACCAGTACGCCGACCTCTTCCTCGCGGCCAAGAATCTGTCGGACGCCATCCTGCTG |
| | | TCGGATATCCTGCGGGTGAATACGGAGATCACGAAGGCCCCCCTCTCGGCCTCGATGATCAAGCGCTACGACGAG |
| | | CACCATCAGGACCTGACGCTGCTCAAGGCCCTCGTCCGGCAGCAGCTGCCGGAGAAGTATAAAGAGATCTTCTTCG |
| | | ACCAGTCCAAGAACGGCTACGCGGGCTACATCGACGGCGGCGCGTCGCAGGAGGAGTTCTATAAATTCATCAAGC |
| | | CGATCCTGGAGAAAATGGACGGCACCGAAGAACTCCTCGTCAAGCTGAACCGGGAGGATCTGCTCCGCAAGCAGC |
| | | GCACCTTCGACAATGGCTCCATCCCGCACCAGATCCATCTCGGCGAGCTGCACGCCATCCTGCGCCGCCAGGAGGA |
| | | CTTCTACCCCTTCCTCAAAGACAACCGGGAGAAAATCGAGAAGATCCTGACGTTCCGCATCCCCTACTACGTGGGCC |
| | | CCCTCGCCCGCGGCAACTCGCGGTTCGCGTGGATGACCCGGAAGAGCGAGGAGACGATCACCCCGTGGAATTTCG |
| | | AGGAGGTCGTCGATAAAGGCGCGTCGGCGCAGTCGTTCATCGAGCGCATGACCAACTTCGATAAAAATCTGCCGA |
| | | ACGAAAAAGTCCTGCCCAAGCATAGCCTGCTGTACGAGTACTTCACGGTCTACAACGAGCTGACGAAAGTGAAATA |
| | | TGTCACGGAGGGCATGCGCAAACCGGCCTTCCTGTCCGGCGAGCAGAAAAAGGCCATCGTGGATCTGCTGTTCAA |
| | | GACGAACCGGAAGGTCACCGTGAAACAGCTGAAGGAAGATTACTTCAAGAAAATCGAGTGCTTCGATTCCGTCGA |
| | | AATCAGCGGCGTGGAGGACCGCTTCAATGCCTCGCTGGGCACCTATCACGATCTCCTCAAGATCATCAAGGACAAG |
| | | GACTTCCTGGACAACGAAGAGAACGAGGACATCCTGGAAGACATCGTCCTCACCCTGACCCTGTTCGAGGACCGC |
| | | GAAATGATCGAAGAGCGCCTGAAGACCTACGCCCACCTGTTCGACGACAAGGTCATGAAGCAGCTCAAGCGCCGC |
| | | CGGTACACCGGCTGGGGCCGCCTGTCCCGGAAGCTGATCAACGGCATCCGCGATAAGCAGAGCGGCAAGACGATC |
| | | CTGGACTTCCTCAAGAGCGACGGCTTCGCCAATCGGAATTTCATGCAGCTCATCCACGACGATAGCCTGACCTTCAA |
| | | AGAGGATATCCAGAAGGCGCAGGTGTCCGGCCAGGGCGACAGCCTGCACGAACATATCGCCAACCTGGCGGGCT |
| | | CCCCCGCGATCAAGAAAGGCATCCTCCAGACGGTCAAAGTCGTGGACGAGCTGGTCAAGGTGATGGGCCGCCACA |
| | | AACCGGAGAATATCGTCATCGAGATGGCACGCGAGAACCAGACCACGCAGAAGGGCCAGAAGAACAGCCGGGAA |
| | | CGCATGAAACGGATCGAAGAGGGCATCAAGGAACTGGGCTCGCAGATCCTGAAGGAGCACCCCGTCGAAAACAC |
| | | GCAGCTCCAGAACGAGAAGCTGTATCTGTACTATCTCCAGAACGGCCGGGACATGTATGTCGATCAGGAACTGGAT |
| | | ATCAACCGCCTCTCCGATTACGATGTGGACGCCATCGTGCCGCAGAGCTTCCTGAAAGACGACTCGATCGACAACA |
| | | AGGTCCTGACCCGGTCGGACAAGAACCGCGGCAAGTCGGATAACGTGCCGTCGGAAGAAGTCGTGAAAAAGATG |
| | | AAGAACTACTGGCGGCAGCTCCTGAACGCGAAGCTCATCACGCAGCGCAAGTTCGACAATCTGACCAAGGCCGAG |
| | | CGCGGCGGCCTCTCGGAACTCGACAAGGCGGGCTTCATCAAACGGCAGCTCGTCGAGACGCGCCAGATCACCAAA |
| | | CACGTGGCCCAGATCCTGGACAGCCGGATGAACACCAAATACGACGAAAACGACAAGCTGATCCGCGAAGTCAAG |
| | | GTCATCACGCTGAAGAGCAAGCTGGTGTCGGATTTCCGCAAGGATTTCCAGTTCTACAAGGTGCGCGAGATCAACA |
| | | ATTACCATCACGCGCACGATGCCTATCTCAATGCGGTCGTGGGCACCGCCCTGATCAAAAAGTACCCGAAACTGGA |
| | | GTCCGAGTTCGTCTACGGCGACTATAAGGTCTATGATGTCCGCAAGATGATCGCCAAATCGGAGCAGGAGATCGG |
| | | CAAGGCGACCGCGAAATATTTCTTCTACTCGAACATCATGAATTTCTTCAAGACCGAGATCACGCTGGCGAACGGC |
| | | GAAATCCGCAAGCGGCCCCTGATCGAAACCAATGGCGAGACCGGCGAGATCGTGTGGGACAAAGGCCGGGATTT |
| | | CGCCACCGTCCGCAAGGTCCTCTCGATGCCGCAGGTGAACATCGTCAAGAAGACGGAGGTCCAGACCGGCGGCTT |
| | | CAGCAAAGAAAGCATCCTCCCCAAGCGGAATAGCGACAAACTGATCGCCCGGAAGAAGGACTGGGACCCGAAGA |
| | | AGTATGGCGGCTTCGATAGCCCCACCGTCGCCTATTCCGTCCTGGTGGTGGCGAAGGTGGAGAAAGGCAAAAGCA |
| | | AGAAACTGAAGAGCGTGAAGGAGCTGCTGGGCATCACCATCATGGAACGCAGCAGCTTCGAGAAGAACCCGATC |
| | | GACTTCCTGGAAGCCAAAGGCTATAAGGAAGTGAAGAAGGACCTCATCATCAAACTCCCGAAGTATTCGCTGTTCG |
| | | AGCTGGAAAATGGCCGCAAACGGATGCTCGCCTCCGCGGGCGAACTCCAGAAGGGCAACGAACTGGCGCTGCCG |
| | | TCCAAATACGTCAACTTCCTCTATCTGGCCAGCCATTACGAAAAGCTGAAGGGCTCGCCCGAAGATAACGAGCAGA |
| | | AACAGCTGTTCGTCGAGCAGCACAAGCACTACCTCGACGAGATCATCGAGCAGATCAGCGAGTTCTCCAAGCGGG |
| | | TGATCCTCGCGGACGCCAACCTGGACAAGGTGCTGTCGGCGTACAACAAACATCGGGATAAGCCGATCCGCGAGC |
| | | AGGCCGAAAATATCATCCACCTGTTCACCCTGACGAACCTCGGCGCCCCCGCCGCCTTCAAGTATTTCGATACCACC |
| | | ATCGACCGGAAGCGCTATACCTCCACCAAAGAGGTCCTGGATGCCACCCTCATCCACCAGTCCATCACGGGCCTGT |
| | | ACGAGACCCGCATCGACCTGTCGCAGCTGGGCGGCGACTAA (SEQ ID NO. 118) |

TABLE 6B

| | gRNA | Targeting | |
|---|---|---|---|
| Plasmid | name | Sequence | Full sgRNA sequence |
| oriv_ | gRNA3 | ATAATGTG | TTTATAGCTAGCTCAGCCCTTGGTACAATGCTAGCATAATGTGTGGAATTGTGAGGTTTTAGAGCTAG |
| dCas9_ | | TGGAATTG | AAATAGCAAGTTAAAATAAGGCTAGTCCGTTATCAACTTGAAAAAGTGGCACCGAGTCGGTGCTTTT |
| gRNA3 | | TGAG (SEQ | TTT (SEQ ID NO. 120) |
| | | ID NO. 119) | |

TABLE 6C

| Plasmid | Promoter | LacZ sequence |
|---|---|---|
| Ptrc_ | TTGACAAT | ATGACCATGATTACGGATTCACTGGCCGTCGTTTTACAACGTCGTGACTGGGAAAACCCTGGCGTTACCCAACTT |
| laxZ | TAATCATCC | AATCGCCTTGCAGCACATCCCCCTTTCGCCAGCTGGCGTAATAGCGAAGAGGCCCGCACCGATCGCCCTTCCCAA |
| | GGCTCGTA | CAGTTGCGCAGCCTGAATGGCGAATGGCGCTTTGCCTGGTTTCCGGCACCAGAAGCGGTGCCGGAAAGCTGGCT |
| | TAATGTGT | GGAGTGCGATCTTCCTGAGGCCGATACTGTCGTCGTCCCCTCAAACTGGCAGATGCACGGTTACGATGCGCCCAT |
| | GGAATTGT | CTACACCAACGTGACCTATCCCATTACGGTCAATCCGCCGTTTGTTCCCACGGAGAATCCGACGGGTTGTTACTC |
| | GAGCGGAT | GCTCACATTTAATGTTGATGAAAGCTGGCTACAGGAAGGCCAGACGCGAATTATTTTTGATGGCGTTAACTCGGC |
| | AACAATTA | GTTTCATCTGTGGTGCAACGGGCGCTGGGTCGGTTACGGCCAGGACAGTCGTTTGCCGTCTGAATTTGACCTGA |
| | ACGAGAGG | GCGCATTTTTACGCGCCGGAGAAAACCGCCTCGCGGTGATGGTGCTGCGCTGGAGTGACGGCAGTTATCTGGAA |
| | AAGTTCC | GATCAGGATATGTGGCGGATGAGCGGCATTTTCCGTGACGTCTCGTTGCTGCATAAACCGACTACACAAATCAG |
| | (SEQ ID | CGATTTCCATGTTGCCACTCGCTTTAATGATGATTTCAGCCGCGCTGTACTGGAGGCTGAAGTTCAGATGTGCGG |
| | NO. 121) | CGAGTTGCGTGACTACCTACGGGTAACAGTTTCTTTATGGCAGGGTGAAACGCAGGTCGCCAGCGGCACCGCGC |
| | | CTTTCGGCGGTGAAATTATCGATGAGCGTGGTGGTTATGCCGATCGCGTCACACTACGTCTGAACGTCGAAAAC |
| | | CCGAAACTGTGGAGCGCCGAAATCCCGAATCTCTATCGTGCGGTGGTTGAACTGCACACCGCCGACGGCACGCT |
| | | GATTGAAGCAGAAGCCTGCGATGTCGGTTTCCGCGAGGTGCGGATTGAAAATGGTCTGCTGCTGCTGAACGGC |
| | | AAGCCGTTGCTGATTCGAGGCGTTAACCGTCACGAGCATCATCCTCTGCATGGTCAGGTCATGGATGAGCAGAC |
| | | GATGGTGCAGGATATCCTGCTGATGAAGCAGAACAACTTTAACGCCGTGCGCTGTTCGCATTATCCGAACCATCC |
| | | GCTGTGGTACACGCTGTGCGACCGCTACGGCCTGTATGTGGTGGATGAAGCCAATATTGAAACCCACGGCATGG |
| | | TGCCAATGAATCGTCTGACCGATGATCCGCGCTGGCTACCGGCGATGAGCGAACGCGTAACGCGAATGGTGCA |
| | | GCGCGATCGTAATCACCCGAGTGTGATCATCTGGTCGCTGGGGAATGAATCAGGCCACGGCGCTAATCACGACG |
| | | CGCTGTATCGCTGGATCAAATCTGTCGATCCTTCCCGCCCGGTGCAGTATGAAGGCGGCGGAGCCGACACCACG |
| | | GCCACCGATATTATTTGCCCGATGTACGCCCGCGTGGATGAAGACCAGCCCTTCCCGGCTGTGCCGAAATGGTCC |
| | | ATCAAAAAATGGCTTTCGCTACCTGGAGAGACGCGCCCGCTGATCCTTTGCGAATACGCCCACGCGATGGGTAA |
| | | CAGTCTTGGCGGTTTCGCTAAATACTGGCAGGCGTTTCGTCAGTATCCCCGTTTACAGGGCGGCTTCGTCTGGGA |
| | | CTGGGTGGATCAGTCGCTGATTAAATATGATGAAAACGGCAACCCGTGGTCGGCTTACGGCGGTGATTTTGGCG |
| | | ATACGCCGAACGATCGCCAGTTCTGTATGAACGGTCTGGTCTTTGCCGACCGCACGCCGCATCCAGCGCTGACG |
| | | GAAGCAAAACACCAGCAGCAGTTTTTCCAGTTCCGTTTATCCGGGCAAACCATCGAAGTGACCAGCGAATACCT |
| | | GTTCCGTCATAGCGATAACGAGCTCCTGCACTGGATGGTGGCGCTGGATGGTAAGCCGCTGGCAAGCGGTGAA |
| | | GTGCCTCTGGATGTCGCTCCACAAGGTAAACAGTTGATTGAACTGCCTGAACTACCGCAGCCGGAGAGCGCCGG |
| | | GCAACTCTGGCTCACAGTACGCGTAGTGCAACCGAACGCGACCGCATGGTCAGAAGCCGGGCACATCAGCGCCT |
| | | GGCAGCAGTGGCGTCTGGCGGAAAACCTCAGTGTGACGCTCCCCGCCGCGTCCCACGCCATCCCGCATCTGACC |
| | | ACCAGCGAAATGGATTTTTGCATCGAGCTGGGTAATAAGCGTTGGCAATTTAACCGCCAGTCAGGCTTTCTTTCA |
| | | CAGATGTGGATTGGCGATAAAAAACAACTGCTGACGCCGCTGCGCGATCAGTTCACCCGTGCACCGCTGGATAA |
| | | CGACATTGGCGTAAGTGAAGCGACCCGCATTGACCCTAACGCCTGGGTCGAACGCTGGAAGGCGGCGGGCCAT |
| | | TACCAGGCCGAAGCAGCGTTGTTGCAGTGCACGGCAGATACACTTGCTGATGCGGTGCTGATTACGACCGCTCA |
| | | CGCGTGGCAGCATCAGGGGAAAACCTTATTTATCAGCCGGAAAACCTACCGGATTGATGGTAGTGGTCAAATGG |
| | | CGATTACCGTTGATGTTGAAGTGGCGAGCGATACACCGCATCCGGCGCGGATTGGCCTGAACTGCCAGCTGGCG |
| | | CAGGTAGCAGAGCGGGTAAACTGGCTCGGATTAGGGCCGCAAGAAAACTATCCCGACCGCCTTACTGCCGCCTG |
| | | TTTTGACCGCTGGGATCTGCCATTGTCAGACATGTATACCCCGTACGTCTTCCCGAGCGAAAACGGTCTGCGCTG |
| | | CGGGACGCGCGAATTGAATTATGGCCCACACCAGTGGCGCGGCGACTTCCAGTTCAACATCAGCCGCTACAGTC |
| | | AACAGCAACTGATGGAAACCAGCCATCGCCATCTGCTGCACGCGGAAGAAGGCACATGGCTGAATATCGACGG |
| | | TTTCCATATGGGGATTGGTGGCGACGACTCCTGGAGCCCGTCAGTATCGGCGGAATTCCAGCTGAGCGCCGGTC |
| | | GCTACCATTACCAGTTGGTCTGGTGTCAAAAATAA (SEQ ID NO. 122) |

1. A method of genetic engineering comprising contacting a microorganism capable of converting a C1 carbon to a multi-carbon product with: (a) a polynucleotide encoding a CRISPR-associated protein; and (b) a polynucleotide encoding a guide ribonucleic acid (gRNA).

2. The method of claim 1, wherein the microorganism is a methylotroph.

3. The method of claim 2, wherein the methylotroph is a methanotroph.

4. The method of claim 3, wherein the methanotroph is from the genera Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, or Methyloacidophilum.

5. The method of claim 3, wherein the methanotroph is from the genus Methylococcus.

6. The method of claim 3, wherein the methanotroph is a Methylococcus capsulatus.

7. The method of claim 1, wherein the C1 carbon is carbon monoxide (CO), carbon dioxide (CO₂), or methane (CH₄).

8. The method of claim 1, wherein the C1 carbon is CH₄.

9. The method of claim 1, wherein the CRISPR-associated protein is Cas1, Cas1B, Cas2, Cas3, Cas4, Cas5, Cas5d, Cas5t, Cas5h, Cas5a, Cas6, Cas7, Cas8, Cas9, Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, or DinG, or a homologue or modified version of any of the foregoing.

10. The method of claim 1, wherein the CRISPR-associated protein is Cas9.

11. The method of claim 1, wherein the polynucleotide encoding a gRNA is at least partially homologous to a promoter, intron, or coding sequence of: (a) a gene encoding an RNA polymerase beta-subunit (rpoB); or (b) a gene within the 2,3-butanediol (2,3-BDO), 1,4-butanediol (1,4-BDO), isobutyraldehyde, or isobutanol pathway.

12-22. (canceled)

23. The method of claim 1, wherein the microorganism is first transformed with the polynucleotide encoding the gRNA and then transformed with the polynucleotide encoding the CRISPR-associated protein.

24. The method of claim 1, further comprising contacting the microorganism with a donor polynucleotide.

25-27. (canceled)

28. The method of claim 24, wherein the donor polynucleotide comprises fewer than 1000 bases.

29-149. (canceled)

150. The method of claim 1, wherein the polynucleotide encoding the CRISPR-associated protein is operably linker to a weak promoter.

151. The method of claim 150, wherein the weak promoter is a pBAD, J23110, lacO, J23116, J23106, J23105, J23108, J23107, J23115, J23114, or a mutant pMxaF promoter.

152. The method of claim 150, wherein the promoter is a mutant pMxaF promoter.

153. The method of claim 152, wherein the promoter comprises the sequence of SEQ ID NO: 78.

154. The genetically-modified microorganism of claim 1, wherein the polynucleotide encoding the gRNA is operably linked to a strong promoter.

155. The genetically-modified microorganism of claim 154, wherein the strong promoter is pMxaF, J2311, J12100, or J23102.


SEQUENCE LISTING (126 sequences)

SEQ ID NO: 1 - DNA, 1713 bp, Bacillus subtilis
  source 1..1713: mol_type = genomic DNA
atgaccaaggccaccaaggaacagaaaagcctggtcaagaaccgcggtgctgaactggttgtggactgcctcgtggaacagggcgtgacccatgtcttcggcatcccgggcgccaagatcgacgccgtcttcgacgccctgcaggataaaggtccggaaatcatcgtggcacgccatgagcagaacgcagccttcatggcccaggccgtcggtcggctgacgggtaagcccggcgtggtgctggtcacctccggtccgggagcctcgaacctggccacgggactgctcaccgccaacaccgaaggcgacccggtggtcgccctggccggtaatgtcatccgggcggatcgcctgaagcgcacccatcagtccctggataacgcggccctgttccagccaatcaccaaatatagtgtcgaagtgcaggatgtgaagaacatcccggaagccgtcaccaatgcgttccgaatcgcgtccgccggccaagcaggggcagcattcgtgagcttcccccaggacgtggtcaatgaagtgaccaacaccaaaaacgtcagagccgtagccgccccgaagctgggccctgcagcagatgacgccatctccgctgccatcgcgaagatccagaccgcaaagctgccggtcgtgctggtcggaatgaagggcggacgcccggaggccatcaaggccgtgcgtaaactgctgaagaaggtgcagctaccgttcgtggaaacctaccaggccgccggcaccctgagtcgggacttggaagaccagtatttcggccgtatcggcctgttccgcaaccagccgggcgacctgctcctggaacaagccgatgtggtgctgaccatcggctacgacccgatcgaatatgacccgaagttctggaacatcaatggcgaccgcacgatcatccatctggacgaaatcatcgccgacatcgaccatgcctatcagccggacctggaactgatcggcgacatcccgagcaccatcaaccacatcgaacacgatgccgtgaaggtggaatttgccgaacgcgaacagaagatcctgtcggacctgaagcagtatatgcatgagggcgaacaggtgcctgccgactggaagtcggacagagcccatccgctggaaatcgtgaaggaactgcgtaacgccgtcgacgaccatgtcaccgtcacctgcgatatcggcagccatgccatttggatgagccgctacttccggagctatgaaccgctgaccctgatgatctccaacggtatgcagaccctcggcgtcgccctcccgtgggccatcggcgcaagtctggtgaagccgggcgaaaaagtggtcagcgtgtccggcgacggcggcttcctgttctccgctatggaactggaaaccgcggtccgcctgaaggccccgatcgtgcatatcgtgtggaacgacagcacctacgacatggtcgccttccagcagctgaaaaagtacaaccgcaccagcgccgtggacttcggcaatatcgacatcgtgaagtatgccgaatccttcggagccaccggactgcgcgtggaatccccggaccagctggcggacgttctgcgtcagggcatgaatgccgaaggtcccgtgattatcgatgtgcccgtcgactacagcgacaacatcaacctggcctcggacaaattgccgaaggagttcggcgaactgatgaaaacaaaagcactataa

SEQ ID NO: 2 - DNA, 1719 bp, Bacillus licheniformis
  source 1..1719: mol_type = genomic DNA
atgaataacgtcgcggccaagaacgaaaccctgaccgtccggggcgccgaactcgtggtggatagcctgatccagcagggcgtgacccatgtcttcggcatcccgggcgccaaaatcgacgcggtcttcgacgtgctgaaggataagggccccgaactgatcgtctgccgccatgagcagaacgcggccttcatggccgccgccgtcggccgcctgacgggcaagccgggcgtctgcctggtcacctccggcccgggcgcctcgaatctcgcgaccggcctggtcaccgcgaacacggaaggcgacccggtggtcgccctggcgggcgccgtgaagcgggcggatcggctgaagaagacgcaccagtcgatggataacgccgccctgttccagcccatcacgaagtacagcgcggaggtggaagacgcgaacaacatcccggaggccgtgacgaacgccttccgcgccgcggcgtccggccaggccggcgcggccttcctcagcttcccccaggatgtcaccgccggcccggccaccgccaagccggtcaaaaccatgcccgccccgaagctgggcgccgcgagcgatgaacagatctccgccgcgatcgcgaagatccacaacgcgaatctgccggtggtcctcgtgggcatgaagggcggccggccggaagccatcgaagccgtgcgccgcctgctccgcaaggtcaagctcccgttcgtggaaacctaccaggcggccggcacgctgtcgcacgatctggaggatcagtacttcggccggatcggcctgttccggaaccagccgggcgacatgctcctggaaaaggccgacgtggtcctgaccgtgggctacgacccgatcgagtacgatccggtgttctggaatggcaaaggcgaacgctcggtcatccacctcgacgaaatccaggccgatatcgatcacgactaccagcccgagatcgaactcatcggcgacatcgcggaaaccctcaatcacatcgagcatgactcgctgccggtgtccatcgacgaatccttcgcgcccgtgctcgactatctcaagaaggcgctcgaagaacagtcggagcccccgaaggaaacgaagaccgatctggtccacccgctccagatcgtgcgcgacctgcgcgagctgctctccgatgacatcaccgtcacctgcgacatcggcagccacgccatctggatgtcccgctatttccgcacctatcgcccgcatggcctcctgatctccaacggcatgcagacgctgggcgtcgccctgccgtgggcgatcgccgcgaccctggtgaacccgggccagaaggtggtgtcggtcagcggcgatggcggcttcctcttctccgcgatggaactcgaaaccgccgtccgcctcaaggcgccgatcgtgcacatcgtgtggaacgactccacgtacgacatggtcgcgttccagcaggaaatgaagtacaagcgcacctccggcgtcgatttcggcggcatcgacatcgtcaagtatgcggaatccttcggcgccaaaggcctccgcgtgaatagccccgatgaactggccgaggtcctgaaggccggcctcgacgcggagggcccggtggtcatcgacatccccgtcgactactcggataacatccacctggccgaccagcgcttcccgaagaagttcgaggagcacttcaacaaggaagcgtcgaagcagtcctga

SEQ ID NO: 3 - DNA, 1476 bp, Escherichia coli
  source 1..1476: mol_type = genomic DNA
atggctaactacttcaacaccctgaacttgcgtcagcagctggcccagctgggtaagtgccggttcatgggccgtgatgagttcgccgatggcgccagctacctgcagggcaaaaaggtggtgatcgtgggctgcggagcccagggcctgaaccagggcctgaatatgcgcgatagcggcctggacatctcctatgctctgcgcaaggaagcgatcgcggaaaagcgggcatcctggcgcaaggccaccgaaaacggtttcaaagtgggcacctacgaagaactgatcccgcaggccgatttggtcataaacctgaccccggacaagcagcattccgatgtggttcgcaccgtccagccgctgatgaaggacggggcagccctgggttactcccacggcttcaacatcgtggaagtcggcgaacagatccgcaaggacatcaccgtcgtcatggtcgcaccgaagtgtccgggcaccgaagtccgggaagaatataagcgcggattcggcgtaccgaccctgatcgccgtccatcccgaaaacgacccgaagggcgaaggcatggccatcgccaaggcctgggctgccgccaccggaggccatcgcgctggcgtgctggaaagctcgttcgtcgccgaagtgaagagcgacctgatgggcgaacagaccatcctgtgcggcatgctgcaggccggtagcctgctgtgtttcgacaagctggtcgaagaaggcaccgaccctgcgtatgccgaaaagctgatccagttcggctgggaaaccatcaccgaagcgctgaaacagggcggtatcaccctgatgatggaccgcctgtcgaaccctgccaagttacgtgcctatgccctgagcgaacagctgaaggaaatcatggcgcctctgttccagaaacatatggacgatatcatcagcggcgagttcagctccggcatgatggcggactgggcgaatgacgacaagaagctgctgacctggcgggaagaaaccggcaagacggccttcgaaaccgcaccgcagtacgaaggcaaaatcggcgaacaggagtacttcgacaaaggcgtcctgatgatcgcgatggtcaaggcgggagtcgaactggccttcgaaacaatggtcgatagcggcatcatcgaggaatccgcctactacgaaagcctgcatgaactcccgctgatcgccaataccatagcccgcaagcggctgtacgaaatgaacgtcgtgatctccgacactgccgaatacggcaattatctcttctcctatgcctgcgtgccgctcctgaagcccttcatggccgaactgcagccaggcgacctggggaaggcgatccccgaaggcgctgtcgacaacggccagctgcgcgatgtcaacgaagccattcgctcccatgccatcgagcaggtcggcaagaagctgcgtggctatatgacggacatgaagcgcatcgcagtagccggataa

SEQ ID NO: 4 - DNA, 1851 bp, Escherichia coli
  source 1..1851: mol_type = genomic DNA
atgccgaagtatcggtcagccaccactacacatggccgcaacatggcaggcgctcgtgccctgtggcgtgctaccggcatgaccgatgccgacttcggcaagccgatcatcgccgtggtcaactctttcacccagttcgtcccagggcacgttcatctgcgcgacctgggcaagctggtggccgaacagatcgaggccgcaggtggcgtcgcgaaagagttcaacaccatcgccgtcgacgatggcatcgctatgggccacggtggcatgctgtatagcctgccgtcccgcgaactgatcgccgatagcgtcgaatatatggtgaacgcccattgcgccgatgctatggtgtgcatcagcaactgcgacaagatcacaccggggatgctgatggccagcctgcggctgaacatcccggtgatcttcgtgagcggtggcccgatggaagccggcaagaccaagctgtcggatcagatcatcaagctggatctggtcgacgccatgatccaaggtgccgatccgaaggtgagcgactcccagtccgatcaggtggaacggagcgcctgcccgacttgcggctcatgcagcggcatgttcaccgccaactccatgaattgcctgacggaagccctgggcctgtcccagcccggtaacgggagcctgttggcgacccatgccgaccgcaagcagctgttcctgaatgccggcaagcgcatcgtggaactgaccaagcgctattatgaacagaacgacgaatccgccctgccccgtaatatcgcctcaaaagccgccttcgaaaacgccatgaccctggacatcgctatgggtggcagcaccaacaccgtgctgcacctgctggctgccgctcaggaagccgagatcgacttcaccatgtccgacatcgacaagctgagtcggaaggtgccgcagctgtgcaaggtggcaccgtccacccagaagtatcatatggaagacgtgcatcgcgcaggcggtgtgatcggcatcctgggcgaactggatcgcgctggcctgctgaatcgcgatgtgaagaacgtcctgggcctgaccctgccgcagaccctggaacagtacgacgttatgctgacccaggatgatgccgtcaagaatatgttccgcgcaggccctgccggcattcgcaccacccaagccttcagccaggactgccggtgggataccctggatgacgatcgcgccaatggctgcatccgtagcctggaacatgcctattccaaggatggcggtctggccgttctgtatggcaacttcgcggaaaacggctgcatcgtcaagaccgctggcgtggatgattcgatcctgaagttcaccggaccggccaaagtctacgaaagccaggacgatgccgtcgaagccatcctgggaggcaaggtcgtcgccggagatgtcgtggtgatccgctatgaaggcccgaaaggcggtccgggcatgcaggagatgctgtatccgacctccttcttaaagagcatgggcttgggcaaagcgtgtgcgctcatcaccgatggccgcttcagtggcggcaccagcggcctgtccatcggccatgtctcgccggaagccgccagtggcggcagcatcggcctcatcgaagacggcgatctgatcgccatcgatatcccgaatcgtggcatccagctgcaggtgtccgacgccgaactagccgcacggagggaagcgcaggatgcccgaggcgacaaggcctggaccccgaagaaccgtgaacgtcaggtgtccttcgcgcttcgcgcctatgccagcctggccaccagcgccgataagggtgccgtgcgcgacaagagcaaactaggaggataa

SEQ ID NO: 5 - DNA, 1689 bp, Methylococcus capsulatus
  source 1..1689: mol_type = genomic DNA
atgaccgacaagcacccccgtccccattcgtcccaggtcgtcgacggcatggagcgggccccgagccgcgcgatgctgcacgccgtcggcttcgccgatgccgacttcgccaaaccgcagatcggcatcgcttccacctgggcgatggtgacgccgtgcaacatgcacatcaacaagctcgccgaggacgcagcacgcggcgtcgacggcggcggcggcaaggcagtgatcttcaacaccatcaccatttccgacggcatctcgatgggcaccgaaggaatgaaatactccctcgtgtcgcgggaagtcatcgccgactcgatcgaaaccgtggtggcctgtcagggttatgacggcgtggtcgccatcggcggctgcgacaagaacatgcccggctgcctgatcgccctcgcccgcctcaaccgtccggcggtgttcgtctatggcggcaccatcctgccgggctgccacgacggcaagaagctggacgtggtgtcggtgttcgaagcggtcggcgcccgcgccaaccaccgcatcgacgatgccgaactgcacgccatcgaatccaatgccatccccggtccgggctcctgcggtggcatgtataccgcgaacacgatggcctccgccatcgaggcattagggatgagcctgccgggcagttcggcccaggtggccatttcccgcgccaaggaactggattgcgagcgggccggcgcccaggtcctcaagctcctggacctggggctcaaaccccgcgacatcatgaccaagaaggcgttcgagaacgccatcacggtggtgatcgccctgggcggctccaccaacgccgtgctgcacctcctggccatggccaacgcctgcggcgtcgacctgaagctcgacgatttcacccgcatcgggcgcaaagtgccgatgctggcggatctgaaacccagcggcagatactcgatggccgaactggtggaaatcggcggcatccagccgctgatgaagaccttgctggacgcgggactcctgcacggcgactgcatgaccgtaaccggcaagaccctggaagaaaacctggccgacgcgcccgactacccggccggacaagacatgatccggtcgctggacaaccccatcaaaaaggacagccatctggtgatcctcaagggcaacctggcgccggaaggcgcggtcgccaagatcaccggcaaggaaggactgagcttcaccggcaccgcccgcgtattcgactgcgaggaagcggcgctcacggccatcctcgacggcacgatcgtgaaaggcgacgtaatcgtcatccgctatgaaggccccaagggcggccccggcatgcgcgagatgctctcgccgacctcggcggtcatgggcaagggattgggcaaggaggtcgccctcatcaccgacggccgcttttccggcggcacccacggcttcgtggtcggccacatcacgccggaagcctacaccggcggccccctggcgatcgtccgggacggcgataccatcaccatcgacgccgaaacccgcgaattgagcctgcacgtcaccgacgatgaaatcggccggcgcctggcgcagtggactcaaccggcgccgcgctacaccaagggcgtgctggccaaatacgccaggttggtgagcccggcctcggaaggcgccgtcaccgacgacggcctctga

SEQ ID NO: 6 - DNA, 1647 bp, Carnobacterium divergens
  source 1..1647: mol_type = genomic DNA
atgtataccgtgggcgactatctcctggagcggctctcggaactgggcatcaaagagatcttcggcgtgccgggcgactacaacctgaagttcctggatcacatcgtggagcatccgaacctgaagtggatcggcaacgcgaatgaactcaacgcggcgtatgccgccgacggctacgcccgcacgaagggcgtctccgcgctggtgaccaccttcggcgtcggcgagctctccgccatcaacggcatcgccggctcgtatgccgagaaagtcccggtcatccagatcgtgggcagccccacgatggcggtgcagaatgcccataagctggtgcatcataccctgggcgatggcaaattcgaccacttcgagaacatgcatgagtccgtcaccgaagccatcggcagcctcaccaaggagaacgcggtgaccgagatcgatcgcgtgctgcgggccgccgtgctcaaacggcgcccggtgtatctgaacctcccgatcgacgtggccgaaatggtcgtcgaaaaaccgtcgggccccctgctgcccaagcaggcgagcctgagcgcccgcgaggtcgaactcgtgcatgagctggagaaggccctgcagcaggcgaagaacccggtggtcctggcgggcaacgagctggcgtcgttccacctcgaaacgtacctcgccgacttcatccacaagttcaacctccccatcacgaccctccccttcggcaagggcgtcttcaacgaggaagacgagcattatctgggcgtctatgcgggctcgccgaccgaagaaggcctgcggaagcgcgtcgatacggcggacctggtcgtggcgctgggcgcgaagctgacggactccgccacctccggcttctcgtacgacttctccgaaaaacagctcttcagcctggcgtccgacgaagtcatcgtcaaagaggaacacctcgaaggcatccatctgccggccgtcatgaaggcgctgacgagcatcgactaccagggctaccagggcgacatccagccgatggcccggctgaagagcatcaaacccaccaaccaggtgctgacccagcgccacttctgggaggccatcgaaggcttcctggaaaagggcgacaccgccgtcgcggagcagggcacgagcttcttcggcctctcgaccgtgccgctgaagagcgaaatgtcgttcatcggccagccgctgtggggctccatcggctatacgttcccggcgatgctgggcagccagctcgccaacccgtccagccggcacctcctgttcatcggcgacggcagcctgcagctgacgatccaggagctcggcatggccctccgcgaaaaactcaccccgatcgtgttcgtcatcaacaataacggctatacggtcgaacgggaaatccacggcccgaatgaaatctataacgacatcccgatgtgggactaccagaaactcccgctcgtcttcggcggctccgagcagtcggtcatcacctataaagtgacgaccgaactggaactggcgaacgcgctcaaggcggcccggctggacaacaaccgcctgcagtggatcgaagtggtgatggaccagaccgatgcgccggagctcctcatgaagctgggcaagatcttcgcgaagcagaatagctga

SEQ ID NO: 7 - DNA, 1650 bp, Methylococcus capsulatus
  source 1..1650: mol_type = genomic DNA
atgggcacggttgagcctggcgctatcggacaacatctgctcgcctgcctttaccaggcgggcgtcgggcacatcttcggcgttcccggcgattacgtgctgggcttctatgatctgatggccaaaggtcccgtccggcatatcgggaccacgcgggaggacaccgccgccttcgccgccgacggctatgcccgctgccggggcatgggcgccctggcggtgacttacggggtcggtgcgctcaacaccgtcaacgccgtcgccggcgcctatgcggaatcctcgccggtggtggtcatcagcggtgcgccgggggtgcgcgagcaaagggaagacccgttgatccaccaccgcttcgggccgttccggttccagcgcgagatattcgaacggatcacctgcgccgccgtggtgctggacgatccggtgatcgccttccggcaggtggagcgtgcgctcgcagccgcccgtcagcactgcaagccggtgtacatcgagattcccgccgaccgggtgatggcgccgggatatccgattccacaggaaaccccggaaacgccttccagcgacgattcggccctggcggaggcggtcgccgaggccgcggagctcctgggccgtgcggtgtcgccggtgatccttgcaggcgtcgagttgcaccggcgagggctccaggacgccctcgtcggcctcgtcgagcaggcccgcctgccggtggcggcgaccttgaccggcaagtcggtgttcgccgagcgccatcccgcctatctgggggtgtacgagggtgcgatgagcacggaaaacgcccgctacatggtcgagcagtccgacctcctgctgatgctcggggtcacgctgaacgatgtcgacacgggcatctacacggcgcgtctcgatccgcagcgcatcgtccgcgcagcccagaacgaggtcgtgattcgccatcaccgctatccccgcgtcctgctcgcggacttcgtcacggccctggcgcggtccgtcaaggcccggggcgaggcgtttccgatgccggcggggccggaaccgtgggactttcccgcgccggaccggccgatgacgatcgcccggctggtggagcggctcgaccgcgccctgacctccgacatgatcgtagtgtgcgatgtcggcgactgcctgttcgcagccaccgacctgcgcgtgcacgagcgcagcgaatttctggcgtccgccttctatacctcgatggggttcgcggtgcccgccgccctcggggcccagatcgcccgtccggaccaccgggcgctgatcctggtcggcgacggtgccttccagatgaccggaacggagctgtcgacccatgcccgtctcggcctggcgcccatcgtggtggtgctcgacaatcgcggttacagcaccgagcgcttcatcctcgacggagccttcaacgacatcgccgactggcgcttccaccggctgggcgaggtgttcggccccctacagggctacgacgcgcccgacgaagcggcgttcgaaaacgcgctcagcgaagcgctggtcaaccgaaacatgccgagcctcatcaacgtccgtctttcccccggcgatgcctcgatagccatgaagcgtctcgccgggcatctgcagtgccgggtcaagggcgagggctaa

SEQ ID NO: 8 - DNA, 1647 bp, Methylocaldum szegediense
  source 1..1647: mol_type = genomic DNA
atgggcaccgccaaggcggatagcatcggccagtacctcctgaaacgcctgtacgaggccggcgtgaaacatatcttcggcgtgcccggcgattacgtgctgggcttctacgatctgatggcgaagagcccgatccagcacgtcggcacgacccgcgaggacaccgcggccttcgccgccgacggctacgcccgctgtcgcggcctcggcgccctggcggtcacctatggcgtcggcgccctgaacaccgtgaatgccgtcgcgggcgcctatgccgaatcgagcccggtgatcgtcatcagcggcgccccgggcgtgcgcgaacagaaggaagacccgatgatccatcatcggttcggcccgttcaccttccagcgggaaatcttcgaccggatcacctgcgccgcggtcacgctggacgatcccatcatcgccttccgccagatcgaccgggtgatcgcggcggcccgccactcgtgcaaacccgtgtatatcgaactgccccgcgacctggtgatggccgaaggccatccggtcccgacggagcccccggaagagcccgcctccgatgaggcggccctgagcgaagcggtggccgaaaccgcggaactgatgtccaagtcggtgagccccaccgtcctggcgggcgtcgaactgcaccggcgcggcctgcaggacgccctggtggaactggtggaacgcgcccggctgccggtggccgccaccctcaccggcaagagcgtgatcgccgaacgccaccccgcctatctgggcgtctacgaaggcgccatgtcctcggaaaacgcccgctacatggtggaacagtccgatctcctgctcatgctgggcgtgacgctgaacgacatcgacaccggcgtctataccgcccgcctcgacccgcatcgcatcgtgcgggcggcccagaacgaggtggtgatccgctaccatcgctatccgcgggtcaccctgtcggacttcgtcctgagcctggcccgcaccgtgaaagcgaagcatgaaaccttccccgccccggtcacgacccccgaagccacggagttccccatgcccgagcgcccgatgacgatcgcccgcctcatcgaacgcctggaccgcgccctgacccccgacatgatcgtcgtgagcgatgtgggcgattgcctgttcgcggccatcgatctccgggtctacgagcggagcgagttcctctcgtcggccttctacaccacgatgggcttcgccgtcccggccgccctgggcgcccagatcgcgcggccggaccaccgggccctgatcctggtgggcgatggcgcgttccagatgacgggcaccgagctcagcacgcacatccgcttcggcctcgcgcccatcgtggtcgtgttcaacaactgtggctattcgaccgaacggtacatcctggacggcccgttcaacgacatctcgtgctggaacttcgaccggctgggcgaactgttcggcccgctgaacggctatgacgcgccggacgaggagagcttcgagaaggccctcgtcgaggccctggccaaccatgccacgcccagcatcatcaacgtccacatcagccgcgacgacagctccagcgcgatgcggcggctggccgaagtcctgaagagccgcgtccggggctga

SEQ ID NO: 9 - DNA, 1653 bp, Methylosarcina lacus
  source 1..1653: mol_type = genomic DNA
atgaacaccgccaagttcgacaccatcggccagtacctgctcaagcggctgtacgacgcgggcgtgaaacacatcttcggcgtgccgggcgattatatcctgggcttctatgacctgatggtgaacagcccggtccagcacatcggcaccacgcgcgaggacaccgcggccttcgcggcggacgcctacgcccgctgcctgggcctcggcgcgatggcggtcacgtacggcgtgggcgccctgaataccgtgaatgccgtcgccggcgcgtatgccgaaagctcgccggtgatcgtcatctccggcgcccccggcatccgcgagcagcgggaggacccgctgatccatcatcggttcggcccgttcaccttccagcgggagatcttcgagcgcatcacctgcgcgaccgaagtgctcaacgacccggtgatcgccttccgccagatcgatcgcgcgatcgccaccgcccggcggctgtgcaagccggtgtacatcgagatcccgcgcgatctggtgatggcggaaggctaccccatgccggacgaggccctcgaacccctggagagcgatgaaaccgccctgagcgaggcgctggcggaaacgatggaactgatggcgaaaagcgtctccccgatgatcatcgccggcgtcgaactgcatcggcggggcctgcagagcgccctcgtcaacctggtggaacgcgcccatctcccggtggtcgccaccctgtcgggcaaaagcgtcatggcggaacggcacccggcctacctgggcatctacgaaggcgcgatgtcctcggaaaatgcccggtacatggtcgaacagagcgacctcctcctcatgctgggcgtcaccctcaacgacatcgataccggcatctatacggccaagctcgacccgcatcatatgatccgggcggcccagaacgaggtggtgatctcctcgcatcgctacccgcgggtcaccctctcggacttcctgacggcgctggtgggcctggtcaagacccggagcgaaggcttcagctcgccgccggccgcctacgaggccagcgccttcccggaaccgaagcggccgatcaccacggcgcggatgatcggccggctgaaccaggcgctgtcgccggaaatgatcgtggtgtgcgacgtgggcgactgcctcttcgccgccatcgatctgcaggtgcacgagcagtccgagttcctcgccagctgctactatgccacgatgggcttcgccgtgcccgcggcgctcggcgcccagatcgcgcggcccgatcaccggctgctcgtgctggtgggcgacggcgccttccagatgacgggcaccgaactgagcacccatgcctatctgggcctcaacccgatcgtcgtggtgttcaacaactccggctacggcaccgaacgcggcatcctggaaggccccttcaacgacatcagcagctggcggttcgaccgcctcggcgaagtgttcggcccgctgaagggctatgacgccgccaccgaggaggccttcgaagcggcgctcatcaactccctcaacaaccggacgatgccgagcatcatcaacgtccatctctccgccgacgacgcctcctccgccatgaagcgcctcgccgagcacctgaagagccgcgtgaagggcggctcgtga

SEQ ID NO: 10 - DNA, 1653 bp, Methylomonas denitrificans
  source 1..1653: mol_type = genomic DNA
atgtcgaccgcgaagttcgacaccatcggccagtatctgctgaagcggctgtatcaggccggcgtcaaggatatcttcggcgtccccggcgactacgtgctgggcttctatgacctgatgatcaagtcgcaggtgcgccatatcggcaccacgcgggaggacagcgcggccttcgcggccgacggctacgcccggtgtgtgggcatgggcgccctggcggtgacgtacggcgtcggcgccctgaacacggtgaacgccatcgccggcgcgtacgccgaatcgtcccccgtcgtcctcatcagcggcgccccgggcgtgtccgagcagaaggacgatccgctgatccatcatcgcttcggccccttcacgttccagcgcgaaatcttcgaacggatcagctgtgcctccgtcgtcctgaatgacccggtcatcgcgttccggcagatcgaccatgccatcgaggccgcccggcgcttctgcaagccggtgtatatcgaactgccgcgggacctcgtgatggcggagggctaccccatgcccaccgagacggtcgaaaagttcacctccgatgaggccgcgctgtccgaagcgatcgcggaaaccatgaccctgctcagcaaggcggtcagcccgatgatcgtggcgggcgtggagctgcatcggcgcggcctgcagggcgccctggccgacttcgtggaacggacctgtctgcccgtggtggccaccctgacgggcaagtcggtgatgtcggagcgccatcccgcctacctgggcatctacgaaggcgccatgtcctcggaagcggtgcgcgatcgggtggagaagagcgatctgctcctgatgctgggcgtgaccctcaacgaaatcgacaccggcatctacacggccaaactgaacagccatagcacgatccgcgccgcgctgaacgaggtcgtgatctccgcccaccgctatcccggcatcgcgctggaagatttcctgggcgccctggcctcgtcggtgtccctcagctcgcgcgaggtggtcagcagcccgaaacccccggagtcgatcgccttcccggaaccggaccggccgatcacgacggcccgcctggtcgaacggctcaactccgccctcagcaatgacatgatcgtcgtgtgtgacgtgggcgactgcctgttcgcggccatcgacctccgcgtgcatgagcagagcgagttcctggcctccgccttctacaccacgatgggcttcgcggtgcccgcggccctcggcgcccagatcgcccgccccgaccggcgggcgctgatcctggtcggcgacggcgcgttccagatgaccggcaccgaactgtccacgcacgcgcggctgggcctgaacccgatcgtcgtcgtgttcaataatggcggctactcgaccgaacgctgcatcctggaaggcccgttcaatgatatcaacccctggcgcttcgaccgcctgggcgagctgttcggccccctggccggctatgaggccgccacggaagccgagttcgaggaagccctgctgaacgcgctggacaaccacggcatgccgtccatcatcaatgtgcatctcgccgccgacgatagctccgaggcgatgaaacgcctggccgagcacctgcagagcaaaatcaagcgggacgcctga

SEQ ID NO: 11 - DNA, 1662 bp, Methylomonas methanica
  source 1..1662: mol_type = genomic DNA
atgaacacggtcaaactggaaacgatgggccagtacctgctgaaccgcctctatgaggcgggcgtgaaacatgtcttcggcgtcccgggcgactatgtcctgggcttctacgacctgatggagaagtcccccatccagcatatcggcaccacccgcgaagataccgcggccttcgccgccgacggctacgcccgctgccgcggcctgggcgccctggccgtcacgtacggcgtcggcgccctcaacacggtgaatgcggtggcgggcgcctacgcggagtcgtcgcccgtgatcgtcatcagcggcgccccgggcgtctgtgagcagcgcgatgacccgctgatccaccaccgcttcggccccttcaccttccagcgcgagatcttcgaacggatcacctgcgccaccgcggtgctgaacgacccggtgatcgcgttccggcagatcgatcacgccatcgcctccgcccgccattattgcaagccggtctatatcgaaatcccgcgggacctcgtgtccgtggaaggctacccgatgcccgccatcgccgcgatggagccgtcgggctccgacaagtccgccctcagcgaggccgtggcggaaaccatgtcgctgctggaaaagtccgtgtccccgatggtcatcgccggcatcgagctccaccgccgcggcctgcagaaccgcctcctcgaactgatcgaacgcgcccgcctgcccgtgacggccaccctcaccggcaaaagcgtgatcgcggagcggcaccccgcgtacctgggcatctatgagggcgcgatgtccagcgaacacgcccgctatatggtcgagcagtccgacctcctcctcatgctgggcgtgaccctgaacgaggtcgacacgggcatctacaccgcgaagctggacccccagcataccatccgggcggcgctgaacgaggtggtgatctcggcccaccgctatccgaatatcgccctggccgactatctgaacgccctggtggacgcggtgaagccgtcggaggcgggcttctccgccaaaccgggcaaacccgtcgcgcgggcgttcccggagcccgaccgcccgatctcgatcaaccgcctgatcgaacgcatcaatcaggcgctggaaccggagacgatcgtggtgtgcgacgtgggcgactgtctcttcgccgccatcgatctggaggtccacgagcagtcggagttcctggcctcgggcttctataccacgatgggcttcgccgtgcccgccgccctgggcgcccaggtcgcccggcccggccaccgcgccctgatcctggtgggcgacggcgcgttccagatgaccggcacggaactctccacccaggcccggctgggcctggactcgatcgtgatcgtcttcaacaattcgggctattccacggagcggtgcatcctggagggcccgttcaacgatatcgcccgctggcgcttcgaccgcctcggcgaagtcttcggcccgctgcagggcttcgacgcggcgaccgaggagtccttcgagtcggcgctgatccaggccctgaacaaccgcagcatgcccagcatcatcaacgtccacctcgcgagcgacgacacgagcagcgcgatgcggcgcctggcggaacacctgaagtcgaaggtgcagggcgaacgccccgcctga

SEQ ID NO: 12 - DNA, 1659 bp, Methylohalobius crimeensis
  source 1..1659: mol_type = genomic DNA
atggccacgcgcaactcgacgacctcgatcggcgagtatctcctccagcgcctgcacgaggccggcgcccaccacatcttcggcgtcccgggcgattatatcctgaaattctatgaacagatctcgcagggcccggtccgccacatcggcaccacgcgcgaagacaccgcggcgttcgcggcggacggctatgcccggtgccagggcatcggcgcgatggccatcacctacggcgtgggcgccctcaacgtggtgaatgccgtcgcgggcgcgtacgccgaaagctcgccggtggtcgtgatctcgggcgcccccggcgtgtgggaacagcgcgaggacccgctgctgcaccaccgcttcggcccctacacgttccagcgggagatcttcgaccggatcacctgtgccaccacggtcctggacgatccgatcacggcgttccgccagatcgatcgcacgatcgccgcggcgcagcgcgaacataagccggtctacatcgaactgccgcgcgatcgggtcacggtggccggcgtgccgctcccggccgtcgcggaggcgacgccccaggaaacgtccgacgccgccaccctggacgaggccgtggccgaaacgctggccctcctcgcccaggccaagagccccgtgctgatcgcgggcgtggaggtccaccgctgcggcctccaggatgcgctcgtcgacctggtcgtccgcgcgggcctgccggtcgcggccaccctgacgggcaaatccgtcgtgggcgaacgccacccggcgtatatcggcgtctacgagggcgccgcgtcgtccgaacacacccgccagatggtggaacgcgcggacgtgctcatcatgctcggcgtgacgctgaacgacgtggacaccggcgtctatacggcgaacctggacccccatcgcctggtccgcgcgtcccagggcgaggtgaacatccgctaccatcggtatccgcgcgtccagctgcaggacttcatcggcgccctcgcccgccaggtcagcccccgccgcgaggccctgccgtcccagcccttcgtcgatagcggccccgccttccccgtcccgggccaggcgatgaccacggcgcggctcatcgcccgcctgaactgcgccctcacccccgaaatgatcgtggtgtccgatgtgggcgattgcctgttcgccgccatcgaactccgggtctgcgaacggagcgagttcctggcctccgcctactatacgacgatgggcttcgcggtgccggccgccctgggcgcccaggtcgcgcggccggatcggcgggccctgatcctggtgggcgacggcgccttccagatgacgggcaccgagctgagcacccacgcccgcctgaacctggcgccgatcatcatcgtcttcaacaacgccggctacagcacggagcgcaatatcctggagggcccgttcaatgacatcgcggcctggcggttcgaccgcctcggcgaagtgttcggccccctgcatggctacgacgccaagacggaagatgcgttcgagacggcgctcgcccgcgccctggccgagacgggctgcccgtcgctcatcaatgtgcatctgtccccggacgacgcctcgccggccatgcgccgcctgacggagcggctgtcccatcgcgtgggcaatcagtga

SEQ ID NO: 13 - DNA, 1635 bp, Methylobacter marinus
  source 1..1635: mol_type = genomic DNA
atgagcagcaacccgtcgatcggccactacctgctcacgcggctgtacgaatccggcgtgcaccacatcttcggcgtcccgggcgactacatcctccgcttctaccagcagctgagcgagagcccggtgcagcatatcggcaccacgcgcgaagacacggccgccttcgcgaccgatgcgtatgcccgctgtcggggcctgggcgcgatggccgtgacctacggcgtcggcgccctcaatgtggtcaacgccgtggcgggcgcccatgccgagtcgagccccatcgtggtcatctccggcgcccccggcatcaaggaacgccgggaacaccccctgctgcatcaccgcttcggcccgttccgcctgcagcgcgagatcttcgaacggatcacctgcgccgtcgcggtgctggatgacccctataccgccttccggcagatcgaccgggtcctggcggcggcgcgggagcactgcaagcccgtctacatcgagctgccgcgcgatcgcgtggataccgagggctatccgatcccgtcggagtccctgccggcccccgcctccgacgccgcgagcctcaacgaagccgtggaggaagccctgcagctcctcgatgaggccgcgtcgccggtgctggtcgccggcgtggaactgcaccgccggggcctccaggatcagctcctctcgctggtggataaaacccatctgccggtcgccgccacgctcaccggcaagtccgtcctgggcgagcggcacccgtgttatctgggcatctacgagggcgcgatgggctcccccctggcgcgggaccgcgtcgagcaggccgatttcctgctgatgctgggcgtcacgctgaacgatgtggacctcggcatcttcaccgcccgcctcgacgccaatcgcatcatccgggcctcccaggacgaagtcatcatccaccaccaccgctatccgcaggtcctcctgcgggacttcgtgtccatgctgaacgaacggatgaccccgcgcccccagacgggcccggccgtcgccgcgaagccggccgccttcgatttcccggtcaaaggccagccgatgaagatcatccggctgatcgcccggctgaatcggttcctcaccccggacatggtcgtggtcagcgacgtgggcgattgcctgttcagcgcgatcgacctgcgggtgcacgaaaacagcgagttcctcgcctcggcctactacaccagcatgggcttcgcggtcccggccgcgctgggcgcccagatcgcccgccccacccggcgcacgctggtcctcgtgggcgacggcgcgttccagatgaccggcaccgagctctcgaccatcgcgcacctcggcctgaacccgatcgtgatcgtcttcaataacaagggctactcgaccgaacgctatatcctggatggcccgttcaatgatatcccggcctggcagttcgagcgcctgggcgaactcttcggcccgctgaccggctatgccgccagcaccgaggacgagttcgaagattgtctgaaccaggccctggcccagcggtcgagcccctccctcatcaacgtccacctctccccggacgatccgagcgccgccatgcggggcctcgccgaacatctgggcaagcgcgtctga

SEQ ID NO: 14 - DNA, 1650 bp, Methylobacter luteus
  source 1..1650: mol_type = genomic DNA
atggaggtgcatctgatgtcctccaacccgtccatcggccattatctgctcgcccgcctgtacgagtccggcgtgcatcacatcttcggcgtccccggcgactacatcctgcgcttctaccagcagctcagcgagtccccgatccagcacatcggcaccacgcgcgaggacaccgccgccttcgcgaccgacgcctacgcccgctgccggggcctgggcgcgatggccgtgacctacggcgtgggcgccctgaacgtcgtgaacggcgtggccggcgcgtacgcggaatcctcgccggtcgtggtgatctccggcgccccgggcatcaaggaacgccatgaacatccgctgctgcatcatcgcttcggcccgttccgcctgcagcatgaaatcttcgaacgcatcacctgcgcgacggcggtcctggacgatccctacatggcgttccggcagatcgaccgggtgctggccgccgcccgcgagcactgcaagcccgtgtatatcgagctgccgcgcgaccgggtggatgtcgaaggctatccgatgccgagcgagagcatgccggccccggcctccgacgcggaaagcctgaacgaagcggtcgaagaaacgctgcagctgctgggcaaggccgcgtcgcccgtgctgatcgcgggcgtcgaactgcaccgccgcggcctccaggacaaactgctctccctggtcgacaagacccatctcccggtggccgcctcgctgaccggcaaatcggtcctgggcgaacgccacccgtgctatctgggcatctatgaaggcgcgatgggctcgtccctggcccgggactccgtggagcagtcggacttcctcctgatgctgggcgtcaccatgaacgacatcgatctgggcatcttcaccgcgaagctggacgcgaaccgcatcatccgcgccacgcaggatgaggtcatcatccaccatcatcgctatccccacgtcctgctgcgggacttcgtcaccgtcctgaatgagcgcatcacgccccgcccgggcatccgcccggccgtcgcggcggagccggccgccttcgacttcccggtcaaggaccagcccatgaaaatcctccggctgatcgaacggctcaaccgcttcctgacccccgacatggccgtcgtctccgatgtcggcgattgcctcttcgcggcgatcgacctccgggtgcacgagaactcggagttcctggcgtcggcgtattataccagcatgggcttcgccgtgcccgcggcgctcggcgcccagatcgcgaacccgacccgccgcacgctggtgctggtcggcgacggcgccttccagatgaccggcaccgaactgtccaccatcgcccggttcggcctgaacccgatcgtgatcgtcttcaataactgtggctattcgaccgagcggtacatcctggatggccccttcaacgatatcgcctgctggcagttcgaacggctgagcgaggtgttcggcccgctctcgggctattcggcccggacggaggatgagttcgaaaactgcctgacccaggccttcgcccagcagtcctcgccgtccctcatcaacgtccacctccccccggatgacccgagcgcggccatgcgcggcctcgcggaacacctcggcaagcgcgtctga

SEQ ID NO: 15 - DNA, 1629 bp, Lamprocystis purpurea
  source 1..1629: mol_type = genomic DNA
atgggcgactacctcctgctccgcctgaaggaagcgggcgtcgaccactgcttcggcgtccccggcgactacgtcctgcggttctacgatcggctctgccggtcggacatccggcacatcggcaccacccgcgaggacaccgcggccttcgcggccgacggctacgcccggtcccgcggcctgggcgccctggccgtgacgtatggcgtgggcgccctcaacgtggtcaacgcggtcgccggcgccaacgcggagtcctccccggtcgtcgtgatctccggcgccccgggcgtcgcggaacagcgcgacgatccccagctgcaccatcggttcggcccgttccggttccagcgcgagatcttcgaacgcatcacctgcgcgtgtgccgtgctcgacgacccgtacaccgccctgcgcgagatcgaccgcacgctcgacgcggcgcggcggtacagccggcccgtgtatatcgaactcccgcgcgaccgggtcgacacgcccgccttccccatcccccacgagccggaagaggaagccggcagcgacccggaggccctggccgaggcggtcgccgaaaccctggcgctcgtcggccgcgcccaggcccccgtcatcctggccggcgtggagctgcaccgccgcggcctgcaggacctcctggccggcttcgtgctgaaggcccacctcccggtggccgcgacgctgacgggcaagtcggtggtcgccgagcgccagccgggctacctgggcgtgtacgagggcgcgatgggcccggaaggcgcccgccgcgtcgtggaagaagccgacctgctgctcctgctgggcgtgaccccgaacgacatcgacctgggcatcaacaccgcccggctggaccccgcccggaccgtccgggccggccaggaagaaatctgggtccatcggcaccgctacccgcatgtgcacctgcgggatttcctggccgcgctgacggatgccgtggtgccccaccccggccccctgccggatgtccccggcccggtcggcgccccggacttcccccagccgggccagcccatgacgatggcccgcatgatggcccgcctgaacgacttcctgacccccgacatgcaggtggtggccgactccggcgactgtctgttcgcgtccgtcgacctgcgcgtccatgcccgcagcgagttcctggcctcggcctattacacgacgatgggcttcgccgtcccggccgcgctcggcgcccaggtcgccaaccccggccgccgcccgctcgtgctggtgggcgacggcgccttccagatgaccggcacggagctgtcgaccgccgcccgcctgggcctcgatccgatcgtcatcatcggcaataatcgcggctacaccaccgaacgcttcatcctggaaggcccgttcaacgacatcgccgactggcggttccatcgcctgggcgaactgttcggcccgctgcggggcttctcggcccccacggaagacgccttcgacgccgccctgggcgccgccctggccttccgggacggcccctccgtcatcgaggtggcgctgcgcccggacgactgctccgcggccctgacccggctctccgaacgcctgcgcgatgtggtgcagcagagcgcctga

SEQ ID NO: 16 - DNA, 1656 bp, Andreprevotia chitinilytica
  source 1..1656: mol_type = genomic DNA
atgcacatgcgggagacggacatggatacgatgggcggctacctgctgcaggccctgcatcgcgagggcgtccggcatgtgttcggcgtgccgggcgactacatcctgcgctggtatcagctgctctcgcagagcaacctgaagcacgtcggcacctcgcgcgaagactgcgccgcgttcgccgccgacggctacgcgcggtgccacggcctgggcgccctggccgtcacctacggcgtcggcgccctgaacgtggtgaacgcggtggccggcgcgaacgcggagtccagccccgtggtggtcatcagcggcgccccgggcgtcgcggaacagcgccagaacccgctgctccaccaccgcttcggcccgttctgcttccagcgcgaaatcttcgaacgcatgacgtgctacgcggcggcgctcgacgatcccctcctggcgcggcgccagatcgaccgcgccctggagctggcccagctccatcacaagccggtctacctcgaactgccgcgggatctcgtggatgcggaactgccgccggccctctcgccgcccacctcctcggcgcccatctcggattgggacgcgctcgaagaagcggtcgcggaaaccctcagcctcctggcgaaggcgaaaagcgccgcggtcctcgccggctcggagctgcaccgctaccagctgcaggacgagctgacgcagctcgtggaacggggcgccctccccgtggccgccaccctgaccggcaagtccgtgatcgccgagcgccacccggcctacatgggcatctacgaaggcgcgatgggcggcgcccgcacgcgggaactgatcgagcgggcggacgtgctgctgctgctgggcgccaccctcaatgatgtggatctgggcatcttcaccgccaagctggatgtccagcacatggtgcaggcgaccgccgatggcgtccagatccaccaccaccgctacaccggcgtccccctcggcgactatgtgcgggccctgacggcgggcatcgaacgctcgggccgctccctcccggtggtggaaccccccctggcggccatcggcttcccgatcacctcgcagcccatgaccgtggcccgcctgatcggccgcctcaatgataccctgccgcaggacatgatcgtcgtctgtgacaccggcgactgcctcttcgcctccctggagctccgcgtccatgcccgcaccgccttcctggcctcggcgttctacacgacgatgggcttcgccgtgcccgcctcgctgggcgcccagctcggctcgggccggcgccccctggtgctggtgggcgatggcgccttccagatgacgggcaccgagctggccaccgccgcgtggaagggcctgaaccccatcgtcatcgtcttcaacaacgccggctactccaccgagcgcttcatcctggacggccccttcaacgacatcccgtcgtggcagttccatcgcctgggcgaactgttcggcccgctggccggcttcgatgtccacgacgaagagtcgttcgactccgcctggcgctcggccctcgcccagaccgatcgcccgtcgctgctgaacgtgcatctggcccccgacgacccctcgcccgcgatgcggcggctgggcgaacatctgggcaagcgggtgcgggcgggctga

SEQ ID NO: 17 - DNA, 1647 bp, Lactococcus lactis
  source 1..1647: mol_type = genomic DNA
atgtacaccgtaggagactacctacttgacagactccatgagctgggcatagaggaaatcttcggagtcccgggtgattacaacctgcagttcctggaccagatcatcagccacaaggacatgaagtgggtgggcaacgcgaacgaactgaacgcgagctatatggcggacggttatgcccgcaccaagaaggcagccgcgttcctcaccactttcggcgtgggcgaactcagcgccgtgaacggcttggcaggcagctatgcggaaaacctgccggtggtggaaatcgtcggctccccgacctcgaaggtgcagaacgagggcaagttcgtgcatcataccctggcggacggggacttcaagcatttcatgaagatgcacgaacccgtcaccgctgcccgcacgctgctgaccgcggaaaacgcgaccgtggagatcgaccgggtcctctccgccctgctgaaggaacggaagcccgtgtacatcaatctacccgtcgacgtagcagccgccaaggccgaaaagccctcgctcccgctgaagaaggagaactcgacgagcaacacgagtgaccaggaaatcctgaacaagatccaggagtcgttaaagaacgcgaagaagccgatcgtcatcaccggccatgagatcatcagcttcggccttgagaagaccgtgacacagttcatctccaagaccaagctgcccatcaccaccctcaacttcggcaagtccagcgtggacgaagccctgccgagcttcctgggcatctacaacggcaccctgtcggaacccaacctgaaggagttcgtcgaaagcgcggacttcatcctgatgctgggcgtgaagctgaccgactcctcgacgggagccttcacccatcacctgaacgaaaacaagatgatctccctcaacatcgatgaaggcaagatcttcaacgagcgcatccaaaacttcgacttcgaaagcctgatctcctcgctgctggacctgtccgagatcgagtacaagggcaagtacatcgacaagaagcaggaagacttcgtgccgtcgaacgcgctgctgtcgcaggaccgcctgtggcaggcggtcgaaaacctgacgcagtcaaacgaaaccatcgtcgccgaacagggaacctcgttcttcggtgcctcaagtatcttcctgaagtccaagtcccacttcatcggccagcccctgtggggctcgatcggctataccttcccggcagcgctaggctcccagatcgcggacaaggaatcgcggcacctgctcttcatcggcgacggctccctgcagctgaccgtccaggagctgggcctcgccatccgggaaaagatcaacccgatctgcttcatcattaacaacgacggctacaccgtggagcgcgagattcatggcccgaaccagagctacaacgacatccccatgtggaactactctaagctgccggaatcgtttggcgccacggaggaccgggtggtcagcaagatcgtccggacggagaacgagttcgtctcggtgatgaaggaagcgcaggcggaccccaaccggatgtattggatcgagctcatcttggccaaggagggcgctccgaaggtcctgaagaagatgggcaagctcttcgccgaacagaacaagtcgtaa

SEQ ID NO: 18 - DNA, 1713 bp, Lactococcus lactis
  source 1..1713: mol_type = genomic DNA
atgggctcgtcccatcatcatcaccaccattccagcggcctggtcccgcgcggcagccacatggcctcgatgtacaccgtgggcgattacctgctggaccggctccacgaactgggcatcgaagagatcttcggcgtcccgggcgactataacctccagttcctggatcagatcatctcccgcgaggacatgaaatggatcggcaatgcgaacgagctgaacgcctcctatatggccgacggctatgcccgcaccaaaaaagccgccgccttcctgaccacgttcggcgtgggcgaactgtcggccatcaacggcctggccggctcctacgccgagaacctgcccgtggtggaaatcgtgggctcgccgacctcgaaggtccagaacgacggcaagttcgtgcaccacacgctcgcggacggcgacttcaagcacttcatgaagatgcacgagccggtcaccgccgcccgcaccctgctgacggccgagaacgcgacctatgaaatcgaccgggtgctctcccagctcctgaaggagcggaagccggtgtacatcaacctccccgtcgatgtggccgccgccaaggccgagaaacccgcgctgagcctggagaaggagtcctccacgaccaacaccaccgaacaggtgatcctcagcaagatcgaagaatccctcaagaacgcccagaaacccgtggtgatcgcgggccatgaggtgatctcgttcggcctggagaaaaccgtcacccagttcgtgagcgaaaccaagctccccatcaccaccctcaacttcggcaagtcggccgtggacgagtccctgccgtccttcctgggcatctataatggcaaactgtcggaaatcagcctgaaaaatttcgtcgaaagcgccgacttcatcctcatgctgggcgtgaagctgaccgactcctcgaccggcgccttcacccaccacctcgatgaaaacaagatgatctccctgaacatcgacgaaggcatcatcttcaacaaggtggtcgaagacttcgacttccgggccgtcgtgtcgagcctgtccgaactgaaaggcatcgaatatgaaggccagtacatcgacaagcagtatgaagagttcatcccgtcgtcggcgccgctcagccaggatcgcctctggcaggccgtggaaagcctgacccagtccaacgaaacgatcgtcgccgaacagggcacctcgttcttcggcgccagcacgatcttcctgaagtccaactcccggttcatcggccagccgctgtggggctcgatcggctacacgttcccggccgcgctgggcagccagatcgccgacaaggaatcccgccacctcctgttcatcggcgacggctccctccagctgaccgtgcaggagctgggcctgtcgatccgcgaaaagctgaatccgatctgcttcatcatcaacaacgacggctacacggtcgaacgggagatccacggcccgacgcagtcctacaacgacatccccatgtggaactacagcaaactcccggaaaccttcggcgccaccgaagaccgcgtcgtctcgaagatcgtgcggaccgaaaacgagttcgtgtccgtgatgaaagaggcgcaggcggatgtgaaccggatgtactggatcgaactggtcctggagaaagaggacgccccgaagctcctgaagaagatgggcaagctcttcgccgagcagaacaaatga

SEQ ID NO: 19 - DNA, 1650 bp, Streptococcus didelphis
  source 1..1650: mol_type = genomic DNA
atgtataccgtcggcgattatctgctggatcggctcaaggaaatcggcatcgaccatatcttcggcgtcccgggcgactacaacctccagttcctggatcagatcaccgcccgcgacgacctcaaatgggtgggcaacgccaacgaactcaacgcgagctacatgtccgacggctatgcccgcaccaagaaggccgccgccttcgtcaccaccttcggcgtgggcgaactgtcggcgatcaacggcctggccggctcgttcgcggaaaacgtccccgtgatcgagatcgtcggctcgccgacgaccaaggtgcaggaagccggcaaactggtgcaccatacgctcggcgacggcaacttcaatcacttccaggagatgcacaaatcggtcaccgtggcccaggtcaaagtcagcgccgaacatgcccagaccgacatcgaccaggtgctcctgtccctgctgaaggagcggaagccggtgtatatcaacctgcccatcgatgtggcccagatgcccgcccagaagccggaatcggccctcctggtcgagaaggtcatctccgagcaggacaagatcatcctgcaggcgatcgagaaaggcctgaagaccgccaagcagcccctcatcatggtcggccatgaagtcgccagcttcggcctggaagccaccatcaacaacttcatcaaaaagaagaagtaccccgtgacctcgctcagcctgggcaaaggcatcgtcaacgagtcgccggaaaccttcctgggcatctactccggcgccctgtccccgcaggccctgaaagactacgtcgatcaggcggacttcatcctcaccctcggcgtcaagctgaccgactccgtgacgggcggcttcagccagggcttcgatgcgaagcaggtgctgagcctggccgccaaccaggcgtcgctcttcggcgaaaactaccagggctatcacttctccgacgtgatccgcgaaatcgaaaatctggacatccccagctattcgggctcctatatcgccaagaccaaggtcgccgatttcgaagcggagaagggccaggtcctgtcccagaagcgcttctggcaggcgatggaatcgttcgtccaggccggcgacaccatcttcgccgagcagggcacctcgtacttcggcgcgagccagctcaacctgaaagagaacgtggcctaccagggccagccgctctggggcagcatcggctataccttcccggccgtcttcggcagccagctcgcgaaccccgattcgcggcatatcctgttcgtcggcgacggcagcctgcagctgaccgtccaggacatcggcctggcgctgcgcgagcagctgaacacgatcgtgttcgtcatcaacaacgacggctataccgtggagcgcaagatccacggcccggaagaggtctataatgacatcccgcagtggcagtacagccagctccccgcctccttcggcggcaacgactcgcaggtcctggcccggaaagtgagcaccgaagaggagctggtcgaaatcctggaaaaggcgcgggccgacgtgtcccggatgtattggatcgagctcatgctccccaaaatggacgcgccggagtatctggaaaagctgggcaagctgttcgcccagcagaataaagcctga

SEQ ID NO: 20 - DNA, 1647 bp, Enterococcus caccae
  source 1..1647: mol_type = genomic DNA
atgtacaccgtcgccgattacctgctggatcgcctgaaggagctgggcatcgatgagctgttcggcgtgccgggcgactacaacctgcagttcctcgaccacatcaccgcccgccaggacctggagtggatcggcaacgccaacgaactgaacgccgcgtatatggccgacggctacgcccggaccaagggcatctcggcgttcgtgaccaccttcggcgtgggcgagctgtccgccatcaatggcctcgccggcagcttcgcggagaacgtcccggtcgtcgagatcgtgggctccccgaccaccacggtccagaacgacaagaagctggtgcatcacacgctgggcgacggcaatttcctgcatttcgagaagatgcatgaagaggtcaccgccgccatcgcgcacctcaccgccgaaaacgcgctgaccgaaatcgaccgggtgctcatcatcgccatgatcgagaagcgcccggtctatatcaacctcccgatcgacatcgccgagttcaaagccaccccgccgctcagccccctgagccggtcggccgaaaagctgacggatgtcgaaatcgccatcctcgacaaggtggaaaaggcgctgtcgcaggcgaagaaccccgtcgtgatcgccggccacgaaatcctcagctaccatatcgaacatcagctggacgagttcatccagaagttcaacctcccgatcaccacgctgcccctgggcaaacgcgcgttcaatgaggaagacccgcactatctcggcacctacagcggctccaccaccgaggagccgctgaagacccgcgtggataccgcggatctggtcctgctgctgggcgcgaagctgacggactcggccacctcgggcttctccttcggcttcaccgaccagcagatcatcagcatcggctccacggaggtcctcttctacggcgaaaccttcaaagccgtgcagctcgaccgcttcgtctcggcgctcaccaccctgagcttctcccggtacgaagatgaaatccagccggtgacccggatcagcaaccaggcgatcaaggacgagaagctgtcgcagaagcagttctgggagatggtcgagacgttcctgatcccgggcgacaccgtgatcggcgagcagggcacctcgttcttcggcctgaccaacgtcgccctgaagcggaatatgcacttcatcggccagccgctgtggggcagcatcggctatacgttcccgtcggccctcggctcgcagatcgccaacaaggaaagccggcatctgctgttcatcggcgatggctccctccagctgaccgtgcaggaactgggcaccgccctgcgcgagaagctgacgccgatcgtcttcgtcatcaacaacaatggctataccgtggaacgggagatccacggcgccaccgagcagtacaacgacatccccatgtgggattatcagaacctgccgctcgtgttcggcggcaacaaccagaccgtcgccacctacaaggtcaccaccgcgatcgaactggatgaggtcatgaaaaccgcccgcaaggacaccaagcgcctgcagtggatcgaagtggtcatggcgcaggatgacgcgccggaactgctcaagaaactcgccaaaatcttcgccaaacagaacagctga

SEQ ID NO: 21 - DNA, 1647 bp, Enterococcus haemoperoxidus
  source 1..1647: mol_type = genomic DNA
atgtacaccatcagcgactacctcctggaccggctgaaggaactcggcatcgacgaagtcttcggcgtcccgggcgactacaacctccagttcctcgatcatatcaccgcccgcgaagacctgaaatggatcggcaacgcgaatgaactcaatgccgcctacatggcggacggctatgcccgcaccaagggcatcagcgccttcgtgaccaccttcggcgtcggcgaactgagcgcggtgaacggcctggccggctcgtacgccgagaacgtgccggtggtcgagatcatcggcagccccaccaccaccgtgcagaacaacaagaaactggtccaccacacgctgggcgatggcgacttcctgcggttcgaaaagatgcacgaggaagtcaccgccgcgatcgcccatctgaccatcgagaatgccacgagcgaaatcgaccgcgtcctgacgatcgccatgaccgagaagcgccccgtgtatatcaacctgccgatcgatatcgcggaaaccaagacgaacaagccgaacaaaccgctgcagaagatgaccgagcggctcacggaagccgaagcgaccatcctgtcgaaggtcgagaaggccctccagcaggcggagaacccggtcatcatcgccggccatgagatcctgtcctaccacatcgagcaccagctgaatgagttcatccagaagttcaatctccccatcaccacgctgccgctgggcaagggcgccttcgacgaggaagactcgcactacatgggcacgtattccggctcccccacggaagagcccctgaagagccgcgtggataacgccgatctggtcctgctgctcggcgccaagctgaccgattccgcgacctccggcttctcgttcggcttcaccgacaagcagatcatcagcatcggcgcgaccgaagtcctgttctacggcgagaaacacgaggccatccagctcgatcgcttcgtgtccgccctgtccacgctctccttctcccgcttcaccggcgatctgctcccggtgaaacggatcagcaaggtcgagttcaaggacgagcagctcacccagaagcgcttctggaagatggtcgaaacgttcctgctccagggcgacaccgtcgtgggcgaacagggcaccagcttcttcggcctgaccaatgtgcccctgaagaaggatatgcacttcatcggccagccgctctggggcagcatcggctataccttccccagcaccctgggctcgcagatcgccaacaaggactcccgccacctgctgttcatcggcgatggctcgctgcagctgaccgtgcaggagctcggcacggccatccgggagaagctcacgccgatcgtcttcgtcatcaacaacaacggctacaccgtggaacgcgaaatccacggcgccaccgagcagtacaacgacatcccgatgtgggactaccagaacctccccctggtcttcggcggcacgagccagaccgtggcgacgtataaagccacgaccgaagcggagctggccgaggtcatgaagtccgcccggaaggatacggagcggctgcagtggatcgaggtggtgatggaccaggaagacgcgcccctgctgctgcagaagctggccaagatcttcgccaagcagaactcgtga

SEQ ID NO: 22 - DNA, 1647 bp, Enterococcus moraviensis
  source 1..1647: mol_type = genomic DNA
atgtacacggtggcggactatctgctggaccggctgaaagagctgggcatcgacgaagtgttcggcgtgcccggcgactacaacctgcagttcctcgaccacatcacggcccggaaggacctcgaatggatcggcaatgccaacgaactgaacgccgcctacatggccgacggctacgcccgcaccaagggcatctccgcgctggtcaccaccttcggcgtcggcgaactgtcggccatcaatggcctggccggctcgtacgcggaaagcatcccggtgatcgaaatcgtgggctccccgacgacgacggtgcagcagaacaagaagctcgtgcatcatacgctgggcgacggcgatttcctgcggttcgagcgcatccacgaggaggtgtcggcggccatcgcgcacctgtccaccgagaacgccccctccgagatcgaccgcgtgctgacggtggccatgaccgaaaagcgcccggtctatatcaacctcccgatcgatatcgcggagatgaaagcgtcggcccccaccacgcccctgaaccacaccacggatcagctgacgaccgtcgagacggccatcctcaccaaggtcgaggatgcgctgaagcagtccaagaatcccgtcgtcatcgccggccacgagatcctgagctaccacatcgaaaatcagctggaacagttcatccagaagttcaacctgccgatcaccgtgctcccgttcggcaagggcgccttcaacgaagaggacgcgcattacctgggcacctatacgggcagcacgaccgacgagtccatgaagaatcgcgtcgaccatgcggacctggtcctgctgctcggcgccaagctcaccgactcggccacctcgggcttcagcttcggcttcacggagaagcagatgatctcgatcggctcgaccgaagtgctgttctatggcgagaagcaggagacggtgcagctcgaccgcttcgtgagcgccctgtcgaccctgtccttctcccgcttcaccgacgagatgccgagcgtgaaacgcctggccaccccgaaggtgcgcgatgagaagctgacccagaagcagttctggcagatggtcgagagcttcctgctccagggcgacaccgtcgtgggcgagcagggcacgagcttcttcggcctgacgaatgtgcccctgaaaaaggacatgcacttcatcggccagccgctgtggggcagcatcggctatacgttccccagcgccctgggcagccagatcgccaacaaagagtcccgccacctgctgttcatcggcgacggctcgctccagctgacggtccaggagctgggcaccgcgatccgcgaaaagctgacccccatcgtgttcgtcatcaacaacaacggctataccgtggaacgcgagatccacggcgcgaccgagcagtacaacgacatccccatgtgggactaccagaaactgccgttcgtgttcggcggcaccgatcagacggtggccacctataaggtgtccaccgaaatcgaactcgataacgcgatgacccgggcccggacggacgtggaccgcctccagtggatcgaagtcgtgatggaccagaacgacgccccggtcctgctgaagaagctcgccaagatcttcgcgaaacagaactcctga

SEQ ID NO: 23 - DNA, 1647 bp, Carnobacterium maltaromaticum
  source 1..1647: mol_type = genomic DNA
atgtataccgtgggcaactacctgctggaccgcctcaccgaactgggcatccgggatatcttcggcgtcccgggcgattataacctcaagttcctggaccatgtcatgacccataaggaactgaattggatcggcaacgccaacgagctgaatgcggcctatgccgccgacggctacgcgcggaccaagggcatcgcggccctggtcaccaccttcggcgtgggcgaactgagcgcggccaatggcaccgcgggctcctatgccgaaaaggtgcccgtggtgcagatcgtgggcacgcccacgacggcggtgcagaactcccacaaactggtgcaccataccctgggcgacggccgcttcgatcacttcgaaaagatgcagaccgagatcaatggcgccatcgcgcatctgaccgcggacaacgccctggcggagatcgatcgcgtgctgcggatcgccgtgaccgaacggtgcccggtctatatcaacctggccatcgatgtcgcggaggtggtggccgaaaaaccgctgaagcccctgatggaggaatcgaagaaagtcgaggaggagacggccctcgtcctcaacaagatcgaaaaggcgctccaggactccaaaaacccggtggtcctgatcggcaacgagatcgccagcttccatctggaatcggcgctggccgatttcgtcaagaagttcaacctcccggtcacggtgctgcccttcggcaagggcggcttcgacgaggaggatgcgcacttcatcggcgtctataccggcgccccgaccgccgaaagcatcaaggagcgggtggaaaaggccgacctcatcctcatcatcggcgcgaagctgaccgatagcgccaccgcgggcttctcctacgacttcgaggaccgccaggtcatcagcgtcggcagcgacgaagtgtccttctatggcgagatcatgaaacccgtggcgttcgcccagttcgtgaacggcctgaactccctgaattacctgggctacaccggcgaaatcaagcaggtggagcgggtggcggacatcgaggcgaaggcgtcgaatctcacccagaacaacttctggaagttcgtggaaaagtacctgtcgaacggcgacaccctggtggccgagcagggcaccagcttcttcggcgcctcgctcgtgccgctgaaatcgaagatgaagttcatcggccagccgctgtggggcagcatcggctatacgttccccgccatgctgggcagccagatcgcgaatcccgcgagccggcatctgctcttcatcggcgacggctccctgcagctgaccatccaggagctcggcatgaccttccgggagaaactgaccccgatcgtgttcgtcatcaacaacgatggctacaccgtcgagcgggaaatccacggcccgaacgagctctacaacgatatcccgatgtgggattatcagaacctcccgtacgtgttcggcggcaacaagggcaacgtcgccacctataaggtcaccaccgaggaagaactggtggccgccatgtcccaggcccggcaggacaccacccggctgcagtggatcgaggtcgtgatgggcaaacaggattcgccggacctcctggtccagctgggcaaggtgttcgccaagcagaacagctga

SEQ ID NO: 24 - DNA, 1647 bp, Brochothrix thermosphacta
  source 1..1647: mol_type = genomic DNA
atgtatacgatcggcgactacctcctcgaccggctgaacgagctgggcgtggaagacatcttcggcgtgccgggcgactacaacctgacgttcctcgatcatatcaccgcgcatccgcagctctcctgggtcggcaatgccaacgaactcaacgccgcctacgccgccgatggctatgcgcggacgaaaggcttcgccgcgctggtgacgaccttcggcgtcggcgagctgtcggccatcaacggcctcgccggctccttcgccgaacgggtgccggtcatcgagatcgtcggcagcccggtcagcaccgtgcagaccgacaagaagctggtgcaccacaccctgggcgacggcgacttcctgcacttcgagaagatgcatgacgccgtcacggtggcctcggcgcacctcacgatccagaacgccaccagcgaaatcgaccgggtcctgacgaccgccctctcgctgcggcgccccggctatatcaacctgcccatcgacgtggcggcggccccggccgaaaaagcccagaaaaagctccagctgaaagtgaccagcccgatcgacagcacgctgctggaaaagatccagacggccttctcctcggccaagcagcccgtcttcatcacgggccatgaaatccagtcctaccacctggaggataccgtggcgaagatcgccgcgcacacgaccgtgccggtcgcggccctctcgctgggcaagagctccatcgatgaaacccatccccagttcgtcggcatctactccggcgccctgacggcggagcccctgaaaacctacgtcgataacgccgatctggtgatcctgctcggcgcccagctgaccgacacggccacctcgggcttcagccagtccttctcggccagcaaaatcatcgccatccacccggaaacgaccaccgtgttcggccaggattacccgtcgaacgatttcaaggaactgatcgaggccctcacgaccatcgattaccgcatggaaacctccgcggccctgaagacgatgccgtccaccaaggagttcatcgccaccgacacgctgctcacccagaatcgcttctgggaagccatcgaaaccaacttcaagcagaacgataccatcgtggcggaacagggcacgtcgttcttcggcatcaccaatacccagttcaagaaagatatgcgcctgatcggccagcccctgtggggctcgatcggctatacgttccccgccgccctgggctcgcagctggccgcccgctccaagcggcatctgctcttcatcggcgatggctcgctgcagctgaccatccaggaactgggcatggcgctccgcgccaaactcacccccctgatcttcgtcatcaacaacaacggctacaccgtggaacgcgagatccacggcccgaacgaacggtataatgacatcccgacctgggactatgcgcagctgccgaccgtgttcggcggcacggaccagaacgtggccacgtataaggtgacgaccgaaaccgaactggcggaggcgctcgtcaccgccaaggcggacaccacccgcctgcagtggatcgaggtggtgatggaccagacggacgccccggaactgctgaaagagatgggccgcatcttcgccaagcagaacacccattga

SEQ ID NO: 25 - DNA, 1647 bp, Carnobacterium gallinarum
  source 1..1647: mol_type = genomic DNA
atgtacacggtcgccgattacctgctcgatcggctgaaggaactgggcatcaatgacatcttcggcgtcccgggcgattataacctcaagttcctggaccacatcacggcccgcgacgacctgaaatggatcggcaacgcgaacgagctgaacgcggcgtatatggccgatggctacgcccgcaccaaaggcatggccgcgctggtcacgaccttcggcgtcggcgaactctcggccatgaacggcatcggcggctcgttcgccgagaaagtcccggtcatcgagatcgtgggctcccccaccaccgccgtccagaatgcccagaaactggtccatcatacgctgggcgacggccgcttcaaccatttcgagaagatgcatgaggcgatcaccgtgggcatcggctcgctgaccaaggagaatgccatcaccgaaatcgatcgcatcctgggcctggcgtccgagaagcggcagcccggctacctgaacctgccgatcgatgtggccgaaatggaagtggagaaaccgaacaagccgctcttcgataccaaggtcatggaaatcaaaatggaacaggagctcatcaagagcatcgagaaagtgctcaactcggtcaagcatccggtcatcatcgccggcaacgagatcgccagcttccatctggaggccaagctggccgagttcatcgagaagttcaatctccccgtcaccacgctgccgttcggcaagggcgtcttcaacgaagaagataagcattatctgggcgtgtatacgggcacccccacgacggaacccctgaagtcctacgtcgaccaggcggatctggtcctcctgctgggcgccaagctgaccgactccgccaccagcggcttcagccagggcttcaccgagaagcagatgatctcgctggcctcggacgaggtcatcttccagggcgagcacctcgccggcatccagctccccaccgtcctggatgagctgctgatgatcaactatccgggctaccacggcgagatccagccgatgtcgcggctggcggaagtgaagtcgtcctccagcctcgtcacccaggcgtacttctgggaggccgtcgagtcgtacctggaagaaggcgatacgctcgtcgccgaacagggcaccagcttcttcggcgcctccaccgtccccatgaagaagggcatgagcttcatcggccagccgctgtggggctccatcggctacacgttcccggccatgctcggctcgcagatcgccaagaagggctcgcgccatctgctgttcatcggcgacggctcgctccagctgaccgtccaggaactgggcatgacgctccgggaaaagctggcgccgatcgtgttcatcatcaacaataacggctacaccgtggaacgggaaatccacggccccgaagaaatctataacgacatcccgatgtgggactaccagaagctcccgtccgtcttcggcggcaccgccgaaaacgtggtgacctataaggtccagaccgaggcggagctggccaccgccatgcgcaaggcccgcctggactcgaagcggctgcagtggatcgaagtggtgatgaaccagaaggacgccccggacctcctggtgcagatgggcaagatcttcgccaagcagaatagctga

SEQ ID NO: 26 - DNA, 1647 bp, Carnobacterium divergens; identical to SEQ ID NO: 6
  source 1..1647: mol_type = genomic DNA

SEQ ID NO: 27 - DNA, 1104 bp, Helicobacter bizzozeronii
  source 1..1104: mol_type = genomic DNA
atgcagacgacgatcggccagtatctcctggaccggctgaagtcctacggcgtgcagcatctcttcggcgtgcccggcgactataacctggccttcctcgacctgatcgaagacgatccgcacatccagtgggtcggcaactgcaacgaactgaatgcgtcctacgccgccgacggctacgcgcggctcaagagcatgggcgccctcctgacgaccttcggcgtcggcgagctgagcgccatcaacggcatcgccggctcgtacgcggaatccgtgccggtcgtgaagatcgtcggcatgccctcccgcggcgtggtccattcccgcaagctggtgcaccacaccctgggcgacggcgagttcctcaagttctacaacatgtatgccgaagtgagcgtcgcccagacgatcctcaacaaacagaacgcccagagcgaaatcgaccgcgtcctgggcgaatgcttcctgcataaaaagccggtctacatcggcctcccggtggacgtgacgcacatcccgatcgaaacgtacgccccctcccccctggtggccaagagcgacccgaaaatcctcaacgccttcctgaaggacgcccaggagctgctgtcgaagagcaaatcccaggtggtcatggcggattatgaagtgaaccgctaccagttcaaccaggagctgacgcgcttcatcgaagccgtgaacctgcccatcgtgtcgctggcgatgggcaagggcgtcttcgatgaaacgcacccgaacttcatcggcgtgtataacggcatcctctcggacgcccgggtgagctcgctgatgaagcacgccgactgcgcgatcctggtgggcgtgaagctgacggactcgctgacggccggcttccactatatccgcgaacatcacctgtccatccagatccaccccttctactcccagatcggcgaaaagacgtacgacgatatcctcatgcaggacgtgctgaaagcgctcgcccagctgaagttccaggcctcgttcccgaaggagacgcaccccaaaacgccgcacctgaacggcaagctgacccaggacaagttcttcaagatcgactcgcgcatcctgacccccccgtga

SEQ ID NO: 28 - DNA, 822 bp, Staphylococcus aureus
  source 1..822: mol_type = genomic DNA
atgaaacagcgcatcggcgcctacctgatcgatgccatccaccgcgccggcgtggacaagatcttcggcgtcccgggcgatttcaacctcgccttcctggacgatatcatcagcaacccgaacgtggattgggtcggcaacaccaacgagctgaacgcctcgtatgcggccgatggctatgcccgcctcaacggcctggcggccctggtcaccaccttcggcgtgggcgaactgtcggcggtgaatggcatcgcgggcagctatgccgagcgcatcccggtgatcgccatcaccggcgcccccacccgcgccgtcgagcaggccggcaagtatgtgcatcatagcctgggcgaaggcacgttcgatgactaccggaagatgttcgcccatatcaccgtggcccagggctacatcacgcccgagaatgcgacgaccgaaatcccccgcctcatcaacacggccatcgccgagcgccgcccggtgcatctccacctgcccatcgatgtggcgatctcggagatcgagatccccaccccgttcgaggtgacggcggcgaaagacacggacgcctcgacctatatcgagctgctggccagcaaactgcaccagagcaagcagcccatcatcatcacgggccatgagatcaactccttccatctgcaccaggaactggaagatttcgtcaatcagacccagatccccgtggcgcagctctcgctgggcaaaggcgccttcaacgaggaaaacccgtactatatgggcatctacgatggcaagctgcccaaaatcaagtatgcgatcatgtggaccacggcgatctga

SEQ ID NO: 29 - DNA, 1665 bp, Fictibacillus macauensis
  source 1..1665: mol_type = genomic DNA
atgaacaaccattataccgtcggcacctatctgctgcatcgcctgtccgagctgggcgtccgccatatgttcggcgtgcccggcgactataatctgacgttcctggacgatgtcatcgacttcgaaggcatggaatggatcggcaactgtaacgagctcaacgccgcctacgcggccgacggctatgcccgcatcaacggcatggccgccctggtgaccaccttcggcgtcggcgagctgtcggccatcaacggcatcgccggctcgtacgccgaaaaagtgccggtcgtcaaaatcacgggcatgcccaccaccaacgtgatgaaccagaatctgtacgtccatcacacgctgggcgacggcaacttccagcacttcggcaacatgttccaggaggtcaccgccgcgcagacgatgctgacccaggaaaacgcggcgcaggagatcgatcgcgtgctgctcgcctgctggcacgaaaagcgcccggtgcacatcaacctcccgatcgatgtctacaacaagccggtcaacccccccgagcattcgctcctggaacggggcatctcgtcgaacgcgaccgcgctcgaacagatgctgaccaccgtgatcccgacgatcaaggaggccacctcgcccgtgatcctggcggattatgaggtgtatcgctaccaggcccaggaagccctgatgctgctggcggaaaagaccggcttcccggtggccaccctgagcatgggcaagggcgtgttcaacgaaacccatccccagttcatcggcgtgtacaacggcgacctgtcgtccgactacgtgaagaatatggtcgaccatgccgactgtatcctctccatcggcgtcaagctgaccgacagcatcacgggcggcttcagccatgagttcaccgaggagcaggtcatcgacatctccccgtatagcgtgagcaaaaaagccctcaaatgggcgcccatcacgatgctggatgcgctgggcgccatcacggatgccctggagcagaagccgacccccgccaccaccgcgcggctcgccgcctactcgaacgagagctccttcaccgcgacgaacacgacgctgacccaggagcgcttcttcgaccaggtgtcccacttcctccaggagggcgacgtgatcctggcggaacagggcaccagcttcttcggcgcggccacgatgccgctcccgaagggcgccacgttcatcggccagccgctgtggggcagcatcggctacaccctgccggccctgctgggcagccagctggccgacgaatcccgccgcaatctcctgctcatcggcgatggctcgttccagctcaccgcccaggagctgtcgacgatgctgcgccagcggatcgcgccgatcatcttcctcatcaacaacgacggctacaccgtggaacgggcgatccacggcgagaatcaggtgtataacgacatccagatgtgggactattcgaagctgccggcggtcttcggcgcggcggacgccagcgtcacctacaaggtccggaccgaagaggagctggaggcggccctgcatagcgcccagaactcgtcccagctggtcttcatcgaagtgatgatggagaagaatgacacccccgaactgctgacggccctgagcaagcgcttcgcgaatcagaacaactga

SEQ ID NO: 30 - DNA, 1083 bp, Saccharomyces cerevisiae
  source 1..1083: mol_type = genomic DNA
atgagctatcccgagaagttcgaggggatcgccatccagagccacgaggactggaagaacccgaaaaagaccaagtatgatccgaagcccttctacgatcacgacatcgacatcaagatcgaggcctgcggcgtctgcggcagcgatatccattgtgcggctggccactggggcaacatgaagatgccgttggtcgtcggccacgagatcgtgggcaaggtcgtgaagttaggcccgaaaagcaacagcggcttgaaggtgggccagcgcgtgggtgtgggtgcgcaggtcttcagctgtctggagtgcgaccgttgcaagaacgacaacgaaccgtactgcaccaagttcgtcaccacctactcgcagccctacgaggacggctacgtctcgcagggcggttacgccaactatgtccgagtccacgaacacttcgtggtgcccatcccggaaaatatccccagccatctggcggctcccctgctgtgcggtggcttgaccgtctacagccccctcgtccgcaatggctgcggtcccggcaagaaggtgggtatcgtgggcctcggcggtataggctctatgggcacgctgatctcgaaagcgatgggcgcagaaacgtacgtgatctcgcgttcctcgcgcaagcgcgaggatgcgatgaagatgggtgcggaccactacatcgccacgctggaggagggtgactggggtgagaagtacttcgacacgttcgacctcatcgtggtgtgcgcgagttccctgacggacatcgacttcaatatcatgcccaaggcgatgaaggtcggagggcgcatcgtctccatctcgatcccggagcagcacgaaatgctgtcgctgaagccctacggcctgaaagccgtctccattagctactcggcgctcggtagtatcaaggagctcaaccagctgttgaagttggtttccgaaaaggacatcaagatctgggtggaaacgctcccggtgggcgaagccggtgtgcacgaggcctttgagcggatggagaagggggatgtccgttatcggtttacactcgtcggctacgataaagagttctcggattaa

SEQ ID NO: 31 - DNA, 1164 bp, Escherichia coli
  source 1..1164: mol_type = genomic DNA
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 32 - DNA, 1149 bp, Escherichia coli
  source 1..1149: mol_type = genomic DNA
atggcgaatcggatgatcctcaatgaaacggcctggttcggccgcggcgcggtcggcgccctcaccgatgaggtcaagcggcggggctaccagaaggccctgatcgtcacggataaaaccctggtgcagtgcggcgtcgtcgccaaggtgaccgacaagatggatgcggccggcctggcctgggcgatctacgacggcgtggtgcccaaccccaccatcaccgtggtgaaggaaggcctgggcgtgttccagaactcgggcgcggattatctcatcgcgatcggcggcggcagcccccaggacacctgcaaggccatcggcatcatctcgaacaaccccgagttcgcggacgtgcgctccctggagggcctgtcgccgacgaacaagccctccgtcccgatcctcgccatcccgacgacggccggcaccgcggccgaggtgaccatcaattacgtcatcaccgacgaggaaaagcggcgcaagttcgtgtgtgtggacccccatgacatcccccaggtcgccttcatcgacgccgacatgatggatggcatgccccccgccctcaaggccgcgacgggcgtggacgcgctgacgcatgccatcgaaggctacatcacccggggcgcctgggccctgacggatgccctgcatatcaaggccatcgaaatcatcgccggcgccctgcgcggctccgtggccggcgacaaggatgcgggcgaggagatggcgctgggccagtacgtggccggcatgggcttctccaatgtgggcctgggcctggtgcatggcatggcccatccgctcggcgccttctacaacacgccgcatggcgtcgcgaacgcgatcctcctgccgcatgtcatgcgctacaatgcggacttcacgggcgagaaataccgcgatatcgcccgggtcatgggcgtgaaggtcgagggcatgtcgctggaagaggcgcggaacgccgcggtcgaagccgtcttcgccctgaaccgggatgtgggcatcccgccgcacctgcgcgatgtcggcgtccgcaaggaagacatccccgcgctggcgcaggccgccctggacgatgtgtgcaccggcggcaacccccgcgaggcgacgctggaagacatcgtcgaactctaccataccgcgtggtga

SEQ ID NO: 33 - DNA, 1170 bp, Clostridium acetobutylicum
  source 1..1170: mol_type = genomic DNA
atgctctcgttcgactacagcatccccaccaaggtcttcttcggcaaaggcaagatcgacgtgatcggcgaagagatcaaaaagtacggctcccgcgtgctgatcgtctacggcggcggctcgatcaaacgcaacggcatctatgaccgggccacggcgatcctgaaggaaaacaacatcgccttctacgagctgtccggcgtggagcccaacccgcggatcaccacggtcaagaagggcatcgaaatctgtcgcgaaaacaacgtcgacctggtgctggccatcggcggcggcagcgcgatcgattgctccaaggtgatcgccgccggcgtctattatgacggcgacacctgggacatggtcaaagaccccagcaagatcaccaaagtgctgccgatcgcctccatcctcaccctgagcgcgacgggcagcgaaatggatcagatcgccgtgatctcgaacatggagacgaacgaaaagctcggcgtgggccacgacgatatgcggccgaagttctcggtcctcgatccgacgtataccttcacggtgccgaagaaccagaccgccgccggcacggcggacatcatgtcgcataccttcgaatcgtatttcagcggcgtcgaaggcgcgtatgtccaggacggcatcgcggaagccatcctccgcacctgcatcaagtatggcaagatcgcgatggaaaagaccgacgactacgaggcccgcgcgaatctgatgtgggcctcgtccctggccatcaatggcctgctgagcctcggcaaggatcggaaatggtcgtgccacccgatggagcacgagctgagcgcctattacgacatcacccacggcgtgggcctggccatcctgacccccaactggatggaatatatcctgaacgacgacacgctgcataaattcgtgtcgtacggcatcaacgtctggggcatcgataagaacaaggacaactacgagatcgcccgcgaagccatcaaaaatacgcgggagtacttcaacagcctgggcatcccgtcgaagctgcgcgaggtcggcatcggcaaagataaactggagctgatggccaagcaggcggtgcgcaactcgggcggcacgatcggcagcctccgccccatcaacgcggaggatgtgctggagatcttcaagaagagctattga

SEQ ID NO: 34 - DNA, 1173 bp, Clostridium acetobutylicum
  source 1..1173: mol_type = genomic DNA
atggtcgatttcgagtattcgatcccgacgcggatcttcttcggcaaggacaaaatcaacgtcctgggccgcgaactcaagaaatacggcagcaaagtgctgatcgtctacggcggcggctcgatcaagcggaacggcatctacgataaggccgtgtcgatcctggaaaagaatagcatcaagttctatgaactggcgggcgtcgaaccgaacccccgcgtgaccaccgtcgagaagggcgtcaagatctgccgggaaaacggcgtggaagtcgtgctggcgatcggcggcggctccgcgatcgactgcgccaaggtgatcgcggcggcctgcgagtacgacggcaatccctgggacatcgtcctggacggctccaagatcaagcgcgtcctcccgatcgccagcatcctgaccatcgccgcgacgggctcggaaatggacacctgggccgtcatcaacaatatggataccaacgaaaagctcatcgcggcccacccggacatggccccgaagttctcgatcctcgatcccacctacacctacaccgtcccgacgaaccagaccgcggccggcaccgccgatatcatgtcccatatcttcgaggtgtatttctccaacaccaagacggcgtacctccaggaccgcatggcggaggcgctcctccggacctgcatcaagtacggcggcatcgccctggagaagccggacgactatgaggcccgcgccaacctcatgtgggcgtcctccctggcgatcaatggcctgctgacgtacggcaaagacacgaactggtccgtgcatctcatggagcacgagctgtcggcctattatgatatcacccacggcgtgggcctcgcgatcctcacgcccaactggatggaatacatcctcaacaatgatacggtgtacaagttcgtcgagtacggcgtcaatgtctggggcatcgataaggaaaaaaatcactatgacatcgcgcatcaggcgatccagaagacgcgcgactacttcgtcaacgtgctcggcctgccctcccgcctccgcgacgtgggcatcgaagaggaaaagctggatatcatggccaaggagagcgtgaagctgaccggcggcaccatcggcaacctgcgccccgtgaacgcctccgaggtcctccagatcttcaaaaagtcggtgtga

SEQ ID NO: 35 - DNA, 1011 bp, Escherichia coli
  source 1..1011: mol_type = genomic DNA
atgaaggccgcggtcgtgaccaaggaccatcacgtcgatgtcacgtacaagacgctgcgctccctgaagcatggcgaagcgctgctgaagatggagtgctgtggcgtctgccacacggacctgcatgtgaaaaacggcgacttcggcgacaagaccggcgtcatcctcggccacgaaggcatcggcgtcgtggccgaggtgggccccggcgtcacgtccctcaagccgggcgatcgggcctcggtggcgtggttctatgagggctgcggccactgcgaatattgcaactcgggcaacgaaaccctgtgtcggtcggtgaaaaatgcgggctactccgtcgacggcggcatggcggaagaatgtatcgtggtggccgactacgccgtgaaggtcccggatggcctggacagcgccgccgcctcgtcgatcacctgcgccggcgtcaccacctataaggcggtgaaactgagcaaaatccgcccgggccagtggatcgccatctacggcctgggcggcctgggcaacctggccctgcagtacgccaagaatgtcttcaacgcgaaggtcatcgccatcgatgtcaatgatgaacagctgaagctggccacggagatgggcgcggacctggcgatcaacagccacaccgaagacgcggccaagatcgtccaggagaagaccggcggcgcccatgccgccgtggtgaccgccgtggccaaagccgccttcaattccgccgtcgacgccgtccgggccggcggccgggtcgtcgcggtgggcctgccgccggagtcgatgtccctcgacatcccgcgcctggtgctggatggcatcgaggtggtgggctccctggtcggcacccgccaggacctgaccgaagccttccagttcgccgccgaaggcaaggtcgtgcccaaggtcgccctgcggcccctcgccgacatcaacaccatcttcacggagatggaggaaggcaagatccggggccgcatggtcatcgatttccgccactga

SEQ ID NO: 36 - DNA, 1020 bp, Escherichia coli
  source 1..1020: mol_type = genomic DNA
atgtccatgatcaaaagctacgccgcgaaagaggcgggcggcgagctggaggtgtatgagtatgacccgggcgagctgcggccccaggacgtggaagtgcaggtcgactactgcggcatctgccattcggacctctcgatgatcgataacgagtggggcttcagccagtaccccctggtggccggccacgaggtgatcggccgcgtggtcgccctgggctcggccgcgcaggataaaggcctgcaggtcggccagcgcgtcggcatcggctggacggcccgcagctgcggccattgcgatgcctgcatcagcggcaatcagatcaattgcgaacagggcgcggtcccgaccatcatgaaccggggcggcttcgccgaaaagctgcgggccgattggcagtgggtgatcccgctgccggagaacatcgatatcgagtcggccggccccctgctgtgcggcggcatcaccgtcttcaagccgctcctgatgcatcatatcacggcgaccagccgggtcggcgtgatcggcatcggcggcctcggccacatcgcgatcaaactgctgcacgcgatgggctgcgaggtcaccgcgttctcctcgaaccccgccaaggagcaggaagtgctggcgatgggcgccgataaagtcgtgaactcgcgcgacccccaggccctcaaagccctggccggccagttcgatctcatcatcaacacggtgaacgtgtcgctggactggcagccctacttcgaagccctgacctatggcggcaacttccataccgtcggcgccgtgctgaccccgctgtccgtcccggccttcaccctgatcgccggcgaccgcagcgtgtccggcagcgccaccggcacgccgtatgagctgcgcaagctgatgcgcttcgccgcccgcagcaaggtcgccccgaccaccgagctgttccccatgtccaagatcaatgacgcgatccagcatgtccgggacggcaaggcccgctatcgcgtcgtcctcaaggcggacttctga

SEQ ID NO: 37 - DNA, 1149 bp, Escherichia coli; identical to SEQ ID NO: 32
  source 1..1149: mol_type = genomic DNA

SEQ ID NO: 38 - DNA, 1020 bp, Escherichia coli
  source 1..1020: mol_type = genomic DNA
atgtccatgatcaaaagctatgccgcgaaggaagccggcggcgagctggaggtctacgagtacgaccccggcgaactccgcccgcaggacgtggaggtgcaggtggattactgcggcatctgccacagcgacctgtcgatgatcgacaacgagtggggcttcagccagtacccgctggtggccggccatgaagtgatcggccgcgtcgtcgcgctgggctccgccgcccaggataaaggcctgcaggtcggccagcgcgtgggcatcggctggaccgcccggtcgtgcggccactgcgacgcctgcatctcgggcaaccagatcaattgcgagcagggcgccgtccccaccatcatgaaccgcggcggcttcgcggagaagctccgcgcggactggcagtgggtgatcccgctgccggaaaatatcgatatcgaatccgccggccccctgctgtgcggcggcatcaccgtcttcaagccgctcctgatgcatcatatcaccgccacctcccgcgtcggcgtcatcggcatcggcggcctcggccacatcgccatcaaactcctgcatgcgatgggctgtgaagtgaccgccttcagcagcaaccccgcgaaagagcaggaagtgctcgcgatgggcgcggacaaggtcgtgaacagccgcgatccccaggccctcaaagcgctggccggccagttcgatctcatcatcaacaccgtcaacgtctcgctcgactggcagccgtacttcgaagcgctgacgtacggcggcaacttccacaccgtgggcgcggtcctgacgcccctgtcggtgccggcgttcaccctgatcgccggcgatcggagcgtgtcgggctccgccaccggcaccccgtatgagctgcggaagctgatgcggttcgcggcccgcagcaaggtcgccccgacgaccgagctgttccccatgagcaagatcaacgacgccatccagcatgtgcgcgatggcaaagcccggtatcgcgtggtcctgaaagcggatttctga

SEQ ID NO: 39 - DNA, 1164 bp, Escherichia coli
  source 1..1164: mol_type = genomic DNA
atgaacaatttcaacctccacaccccgacccgcatcctcttcggcaagggcgccatcgccggcctgcgcgagcagatcccgcacgacgcccgcgtcctcatcacctatggcggcggctccgtcaaaaagaccggcgtgctcgatcaggtcctggacgccctgaagggcatggacgtgctggagttcggcggcatcgagccgaacccggcctacgagacgctgatgaatgcggtgaagctggtgcgcgagcagaaggtcacgttcctgctcgcggtcggcggcggctcggtgctggacggcaccaagttcatcgccgccgcggcgaattatcccgagaacatcgatccctggcacatcctgcagacgggcggcaaggagatcaagtcggccatcccgatgggctgcgtcctgaccctccccgccaccggctcggagagcaacgccggcgccgtgatctcgcgcaaaaccaccggcgacaaacaggcgttccactccgcccatgtgcagccggtcttcgcggtgctggaccccgtctacacgtacaccctcccgccgcggcaggtcgccaacggcgtggtcgatgccttcgtgcatacggtggagcagtacgtcaccaagccggtggatgccaagatccaggaccgcttcgcggagggcatcctgctgacgctgatcgaggacggcccgaaagccctcaaggaaccggaaaactacgatgtgcgggcgaacgtcatgtgggccgcgacccaggccctgaacggcctgatcggcgccggcgtgccccaggattgggcgacgcacatgctgggccacgaactcaccgcgatgcacggcctcgaccacgcccagacgctcgccatcgtcctgccggccctgtggaatgagaagcgggacaccaagcgggcgaagctcctgcagtatgccgaacgggtgtggaacatcaccgaaggctcggacgatgaacgcatcgatgccgccatcgcggccacgcggaacttcttcgagcagctgggcgtcccgacccatctctccgactacggcctggatggctcctccatccccgcgctgctgaaaaaactggaagaacacggcatgacccagctgggcgaaaaccacgacatcaccctggatgtctcgcgccgcatctacgaagccgcccggtga

SEQ ID NO: 40 - DNA, 1011 bp, Geobacillus stearothermophilus
  source 1..1011: mol_type = genomic DNA
atgcgggcggccgtggtgaccaaggaccacaaggtcagcatcgaagataagaaactgcgggccctgaaacccggcgaggcgctggtgcagaccgaatattgtggcgtgtgtcatacggatctccatgtcaaaaacgccgatttcggcgatgtgaccggcgtgacgctcggccatgagggcatcggcaaggtgatcgaagtcgccgaagacgtggaaagcctcaagatcggcgatcgcgtgtccatcgcctggatgttcgagtcgtgtggccgctgcgagtattgcacgaccggccgggaaaccctgtgtcggagcgtcaagaatgccggctacaccgtggacggcgcgatggccgaacaggtcatcgtgacggccgactatgcggtcaaggtcccggaaaagctggacccggccgcggcgtcgtcgatcacctgcgcgggcgtcaccacctataaggccgtcaaggtgagcaatgtcaaaccgggccagtggctgggcgtcttcggcatcggcggcctgggcaacctggccctgcagtacgcgaagaatgtcatgggcgccaaaatcgtggccttcgatatcaacgatgacaagctggcgttcgccaaagaactcggcgcggatgcgatcatcaactcgaaggacgtggacccggtggccgaggtgatgaaactgacggacaacaagggcctggacgcgacggtcgtcaccagcgtcgcgaagacccccttcaatcaggcggtcgacgtggtcaaggcgggcgcccgcgtggtggccgtgggcctgccggtcgacaaaatgaacctggatatcccgcgcctcgtgctggacggcatcgaggtggtgggcagcctggtcggcacccgccaggacctgcgggaggccttcgagttcgcggccgagaataaagtgacgcccaaggtccagctccggaagctcgaagaaatcaacgatatcttcgaggagatggaaaacggcacgatcaccggccggatggtcatcaagttctga

SEQ ID NO: 41 - DNA, 1020 bp, Geobacillus stearothermophilus
  source 1..1020: mol_type = genomic DNA
atgaaggcggccgtcgtgaacgagttcaagaaggcgctggaaatcaaggaggtcgagcggcccaaactcgaagagggcgaggtcctggtgaagatcgaggcctgcggcgtgtgccataccgacctgcacgccgcccacggcgactggccgatcaagccgaaactgcccctgatcccgggccacgagggcgtgggcatcgtcgtggaagtggcgaagggcgtgaaaagcatcaaagtgggcgatcgcgtcggcatcccgtggctgtacagcgcgtgcggcgagtgcgagtactgcctgacgggccaggaaacgctctgcccgcatcagctgaatggcggctattccgtggacggcggctatgccgagtattgcaaagccccggccgactatgtcgccaagatcccggacaatctggaccccgtcgaggtcgcccccatcctgtgcgccggcgtgaccacctataaggcgctgaaagtctcgggcgcccggccgggcgagtgggtcgcgatctacggcatcggcggcctgggccacatcgccctgcagtacgccaaggcgatgggcctgaacgtggtcgcggtcgacatctccgacgagaaatcgaagctggcgaaagatctcggcgcggacatcgccatcaatggcctgaaggaagacccggtcaaggcgatccatgaccaggtcggcggcgtccatgccgccatctccgtcgcggtgaataagaaagccttcgagcaggcctatcagtccgtcaagcgcggcggcaccctggtcgtggtgggcctcccgaatgcggacctgccgatccccatcttcgatacggtgctcaacggcgtgtcggtgaagggcagcatcgtcggcacccgcaaggacatgcaggaagccctggatttcgccgcgcggggcaaggtccgccccatcgtggaaacggccgagctggaggaaatcaacgaagtgttcgagcgcatggaaaaaggcaaaatcaacggccgcatcgtcctgaagctgaaggaggattga

SEQ ID NO: 42 - DNA, 1041 bp, Geobacillus themoglucosidas
  source 1..1041: mol_type = genomic DNA
atgaaagccctgacctatctgggcccgggcaaaaaagaactgatggaaaaaccgaagccgaagatcgaaaaagagacggatgccatcgtcaagatgatcaaaaccaccatctgcggcaccgacctccatatcctgtcgggcgacgtgcccaccgtggaagagggccgcatcctgggccacgagggcgtcggcatcatcgaggaagtgggctccgccgtcaagaacttcaagaaaggcgaccgggtgctgatctcgtgcatcaccagctgtggcaagtgcgagaattgcaagaagggcctgtacgcccactgcgaggacggcggctggatcctgggccatctgatcgacggcacccaggccgagtacgtgcgcatcccccatgcggacaacagcctgtacccgatccccgagggcgtcgacgaggaagccctggtcatgctgtcggatatcctgcccaccggcttcgagatcggcgtgctgaacggcaaggtccagcccggccagaccgtggcgatcatcggcgccggcccggtgggcatggccgcgctgctgaccgcccagttctacagcccggccgagatcatcatggtggacctggacgataaccgcctcgaagtggcgaagaagttcggcgcgacccaggtcgtcaacagcgcggatggcaaggcggtggagaagatcatggaactcaccggcggcaagggcgtggacgtggcgatggaagccgtcggcatcccggccaccttcgatatctgccaggagatcgtgaagccgggcggctacatcgccaacatcggcgtgcatggcaagtccgtggagttccatatcgaaaaactgtggatccgcaacatcaccctgacgacgggcctggtgaacaccacgagcacgcccatgctgctgaagacggtgcagtcgaagaagctcaagcccgaacagctcatcacccaccgcttcgccttcagcgatatcatgaaagcgtacgaagtcttcggcaacgccgccaaggagaaggcgctgaaagtcatcatcagcaattcgtga

SEQ ID NO: 43 - DNA, 1023 bp, Geobacillus themoglucosidas
  source 1..1023: mol_type = genomic DNA
atgaaagccgcggtggtcaatgagttcaagcagaaactcgaaatcaaggaagtcgaaaagccgaagctcaactacggcgaagtgctggtgaaaatcgaggcctgcggcgtctgccacaccgacctccatgcggcccacggcgactggcccgtcaagccgaagctgcccctgatcccgggccatgagggcgtgggcatcgtcgtggaagtcgcggagggcgtcaagagcgtcaaggtcggcgaccgggtgggcatcccctggctgtattccgcctgcggcgaatgcgaatactgcctgagcggccaggaaaccctgtgcccccaccagctgaacggcggctatagcgcggatggcggctatgccgagtactgtaaagcccccgccaactacgtggcgaagatcccggaacatctggaccccgtggaagtggcgcccatcctctgcgcgggcgtgaccacctataaagccctcaaggtgtccaacgccaaacccggcgagtgggtcgccatctacggcatcggcggcctcggccatatcgcgctgcagtacgccaaggcgatgggcctcaacgtcatcgccgtggatatcagcgacgagaaaatcgaactggcgaaacagctcggcgcggacatcgcgatcaacggcctgaaagaagatccggtggaagccatccagcagaacgtcggcggcgcccacgccgcgatcagcgtcgccgtgaccaagaaggcgttcgaacaggcctatcagagcgtccggcggggcggctgcctggtcgtggtcggcctgcccaacgaggacctgcccatcccgatcttcaacaccgtcctgaatggcatcaccgtcaagggctccatcgtgggcacgcggaaggatatgcaggaagcgctggatttcgcggcgcggggcaaggtgcggccgatcgtcgagacggccccgctggagaagatcaatgaggtcttcgaacgcatggagaagggcaagatcaatggccgcgtcgtgctcaccatcggcgtcaaccgctga

SEQ ID NO: 44 - DNA, 1023 bp, Lactococcus lactis
  source 1..1023: mol_type = genomic DNA
atgaaggcggccgtcgtgcggcacaacccggacggctatgccgatctggtggagaaggagctccgcgcgatcaagccgaacgaggccctcctggacatggagtactgcggcgtgtgccacaccgacctgcacgtcgccgccggcgatttcggcaacaaagccggcaccgtcctgggccatgagggcatcggcatcgtgaaggaaatcggcaccgatgtgtcgtccctccaggtgggcgaccgggtcagcgtcgcctggttcttcgaaggctgcggccactgcgagtactgcgtgtccggcaacgagacgttctgccgggaagtcaagagcgcgggctacagcgtcgatggcggcatggccgaggaagccatcgtcgtcgccgactacgccgtgaaggtcccggatggcctcgatcccatcgaggccagcagcatcacctgcgcgggcgtgaccacctacaaggccatcaaggtgtccggcgtcaaaccgggcgattggcaggtgatcttcggcgccggcggcctgggcaacctcgcgatccagtacgccaaaaacgtcttcggcgcgaaggtcatcgcggtggacatcaaccaggacaagctgaacctcgcgaagaagatcggcgccgatgtcaccatcaactcgggcgatgtcaacccggtcgacgagatcaagaaaatcaccggcggcctgggcgcccagtcggccatcgtgtgcgcggtcgcccgcatcgcgttcgaacaggcggtggccagcctcaaaccgatgggcaaaatggtggcggtggccgtcccgaacaccgagatgaccctgagcgtgccgaccgtggtcttcgacggcgtcgaggtcgcgggctcgctcgtcggcacccggctggacctcgccgaggccttccagttcggcgcggagggcaaagtcaagccgatcgtcgcgacccggaagctggaggagatcaatgacatcatcgatgagatgaaggccggcaagatcgaaggccggatggtcatcgatttcaccaagtga

SEQ ID NO: 45 - DNA, 1023 bp, Lactococcus lactis
  source 1..1023: mol_type = genomic DNA
atgaaggcggcggtcgtgcgccataacccggacggctacgccgacctggtggaaaaggagctgcgggccatcaagccgaacgaggcgctgctcgacatggagtactgcggcgtctgccatacggacctccacgtcgccgccggcgactacggcaacaaggcgggcacggtgctgggccatgagggcatcggcatcgtgaaggaaatcggcaccgacgtgtcctccctgcaggtcggcgaccgggtcagcgtcgcctggttcttcgagggctgtggccactgcgagtattgtgtcagcggcaatgaaacgttctgtcgcgaagtcaaaaacgccggctactcggtcgatggcggcatggcggaagaagccatcgtggtcgcggactatgccgtgaaggtgccggacggcctggaccccatcgaagcgtcctcgatcacctgcgcgggcgtcacgacctacaaggcgatcaaagtgtcgggcgtcaagccgggcgactggcaggtgatcttcggcgcgggcggcctcggcaacctcgccatccagtacgccaagaacgtcttcggcgccaaagtgatcgccgtcgacatcaatcaggacaaactgaatctggcgaaaaagatcggcgccgatgtcatcatcaacagcggcgatgtgaacccggtggacgagatcaaaaagatcacgggcggcctcggcgcccagagcgcgatcgtgtgcgccgtggcccgcatcgccttcgaacaggccgtcgcgtccctgaagccgatgggcaagatggtcgccgtcgccctcccgaacacggaaatgacgctgtccgtcccgaccgtggtcttcgacggcgtggaagtggccggctcgctggtcggcacccggctggacctcgccgaggccttccagttcggcgcggaaggcaaggtcaagccgatcgtggccacgcgcaagctcgaagagatcaatgatatcatcgatgagatgaaggcgggcaagatcgaaggccgcatggtcatcgatttcaccaagtga

SEQ ID NO: 46 - DNA, 1206 bp, Oenococcus oeni
  source 1..1206: mol_type = genomic DNA
atgcgcggcagccatcatcaccatcaccacggcagcgccgaacgggcctacgatttcctgatgccctccgtcaacttcttcggcccgggcgtgatctccaagatcggcgaacgggcgaaaatgctcggcatgaagaagccggtgatcgtcaccgataagttcctggagaatctgaaaaatggcgccgtggcccagaccctggccagcctcaagaagagcggcgtcgattacgtcgtgtataacggcgtggagcccaaccccaaaatccacaacatcaaggaggtgaaaaccctgtacgaaaaggaagacgccgacagcatcatcaccgtgggcggcggctcggcccacgatacgggcaagggcgccggcatcatcatgacgaacggcgatgacatcaccaagctggccggcatcgaaaccctgaagaatcccctcccccccctgatcgccgtgaataccaccgcgggcaccggctcggaactcacgcggcacgccgtcatcacgaacgaggaaacccatctgaagttcgtcgtggtgtcctggcgcaacatcccgctggtcagcttcaatgaccccaccctgatgctggacatccccaagggcctcaccgcggccacgggcatggacgccttcgtccaggcggtcgaaccgtacgtgagcgtggatcacaatcccatcaccgactcccagtgtatccaggccatcaagctgatcgaatcgtcgctgcgggaggccgtggcgaacggccataacctgcaggcccgcaccaaaatggtggaagccgaaatgctcgcgggcatggcgttcaataacgccaacctgggctacgtccacgcgatggcccatcagctgggcggccagtacgacgccccgcatggcgtctgctgcgccctgctcctgccgtatgcggaggagtacaacctgatcgccgacccggaacgcttcgcggaactggcccgcatcatgggcgagaacaccgacggcctctcgacccgcgacgcggccgaactgtccatcaaggcgatgaaacagctgtcggaggacgtgggcatcccgcactcgatcaaggacatcggcgccaagccggaggacttcgacctgatggccgaaaatgcgctgaaggacggcaatgccttctccaacccgcgcaaaggcaccaaggaggatatcgtcaagatcttccaggaggcctatgacgccaaatga

SEQ ID NO: 47 - DNA, 1002 bp, Pectobacterium atrosepticum
  source 1..1002: mol_type = genomic DNA
atgctgaatttcaccctccataccccgacgaagatcctgttcggcgagggccagatcgcggagctgggcaaggagatcccggccgacgcccgcatcctcatcacctacggcggcggctcggtcaaacacaacggcgtgctggatcaggtgtaccgggcgctggaaggccggaacgtgcgggagttctccggcatcgagcccaacccgacctacgaaacgctcatgaaggccgtggaggtggtccgggcggaaaagatcgatttcctgctcgccgtgggcggcggcagcgtcgtcgacggcaccaagttcatcgccgcggcggccgactaccaggccgcgcaggacccgtggcacatcctccagaccggcggcgccgaaatcgaccggggcgtggccctcgccgcggtgctgaccctgcccgccacgggcagcgaatccaataacggcgccgtcatcacccgcaaaagcaccaatgacaagctcgcgttccggtccccgcatacgcagcccctcttcgccgtcctcgacccggtggtcacgtacaccctgccggcccggcagatcgcgaatggcgtcgtcgacgccttcgtccacaccgtcgagcagtacctgacctactccgtcgacgcgaaagtccaggatcgcttcgccgagggcctgctgctcaccctggtcgaagagggcccccgggccctggccgaacccgaaaactacaaagtgcgggcgaatgtcatgtggagcgccacgatggcgctgaacggcctcatcggcgccggcgtcccccaggattggtcgacccacatgctgggccacgaactcacggccctgcacggcctcgaccacgcgcagacgctggccatcgtcctgccggccatgctggcggcccggaaatcccagaaacgggataaactcctgcagtacgccgagcgcgtctggaacctccgcgacggctcggaagatcagcggatcgacggcgccatcgccgccacgcgcgatttctga

SEQ ID NO: 48 - DNA, 1056 bp, Psychrobacter cryohalolentis
  source 1..1056: mol_type = genomic DNA
atggccaataccaaggcctacgccgccacggcgccggattcgggcctggccccgtacgcgatcgaccggcgggaactgcgggccgatgacgtggcgatcgaaatcgactactgtggcgtgtgccatagcgatctccataccgtggaaaacgactggggcggctcgaagtacccggtgatcccgggccatgagatcgtcggccgggtgacggcggtgggccccgaggtcagccatttcaaggccggcgacctcgtgggcgtgggctgcatggtggattcgtgtcgctcgtgcagcgcctgcgacagcggcctcgaacagtattgcatcgagggcagcacgatgacctacggcagcctggaccgccacgatggctccgtcacccacggcggctacagcgaacgcatcgtcgtctcggaacggttcgtcgtgcgggtgcccgaaaaactggacccggcctcggccgccccgatcctgtgcgccggcatcacgacgtacagcccgctgaagcacttcaaggtgggcaagggccataaagtcggcgtcctgggcatgggcggcctgggccatatgggcgtgaagttcgccaaggccctgggcgccgaggtgacgatcttcacccggtccgaggcgaaagtggccgaagcgaagaaacagggcgccgaccatgtcatcatctcgaccgataaggagcagatgaaggccgccgccgacagcttcgatttcctcctcgacaccatcccggtggcgcacgacctgaatccgtatctgaagtgtctgaaatacgatggcacccacatcctcgtgggcctgctgacccccatcgaaccggcgctgcaggccggcctcctggtcaccaagcggcgcgtcgtggcgggcagcctgatcggcggcatgcccgagacgcaggaagtcctggacttctgcgccgaacatgacatcacctgtgatatcgagatgctcgacatccgcaacatcaacgaagcgtacgtccgcatgaaaaagggcgatgtcaagtaccgcttcgtgatcgatatgaagacgctgaaggaaggctga

SEQ ID NO: 49 - DNA, 1713 bp, Bacillus subtilis; identical to SEQ ID NO: 1
  source 1..1713: mol_type = genomic DNA

SEQ ID NO: 50 - DNA, 1671 bp, Clostridium autoethanogenum
  source 1..1671: mol_type = genomic DNA
atgaatcgggatatcaagaaagaggtgcagctcaacacggcccagatgctggtcaagtgtctggaagccgagggcgtcaagtatatcttcggcatcccgggcgaggagaatctcgaaatcatgaacgccatctcggattccacgatcgagttcatcaccacccgccatgaacagggcgcggccttcatggccgacgtgtacggccggctgaccggcaaggcgggcgtgtgtctgagcaccctcggccccggcgcgaccaacctggtcaccggcgtggccgacgccgactccgacggcgcccccgtggtcgcgatcaccggccaggtgggcacggagcggatgcacatcacctcccatcagttcctcgacctctgcaagatgttcgagccgatcaccaagcggagcaagcagatcgtccgcccggacacggtgtcggagatcatccgcctggtgttcaagtacgccgaaagcgaaaagcccggcgcctgtcatatcgacctgccggtcaacatcgccaagatgcccgtcggcgccctggagaagccgctggagaaaaaaatcccgccgaaggaacacgcggacctgtccaccatcgaggaagcggcgtccgagatcttcaaggccaaaaaccccatcatcctggccggcagcggcgccatccgcggcaacagcagcaaggcggtcaccgagttcgccaccaagctgaagatccccgtcatcaacacgatgatggccaagggcatcatcccgatggacaacaagtatagcatgtggaccatcggcatcccccagaaggactatgtgaacaagatcatcgaagaggccgacctggtcatcaccatcggctacgacatcgtggaatatgccccgtcgaaatggaacatcaacggcgacatcaagatcgtccatatcgacgcccgcccctcgcacatcaacaaactctaccagcccatcgtggaggtggtcggcgacatcagcgacgcgctgtataacatcctgcgccgcaccagctcgaaagacgagccggtcaaggcgctggagatcaagtcggaaatgctggcggagcacgagtcctacgcgaacgacaatgcgttcccgatgaagccgcagcgcatcctcaacgatgtgcgcaaagtcatgggcccgcacgacatcgtgatctccgatgtgggcgcccataaaatgtggatcgcccgccactataactgctacgagccgaatacctgcatcatctcgaacggcttcgccacgatgggcatcggcgtcccgggcgcgatcgccgccaaactcatcaacccggataagaaggtcctggccatcgtcggcgacggcggcttcatgatgaataaccaggaactggagacggcgctgcgcatcaaaacgcccatcgtggtcctcatcttcaacgactccaattacggcctcatcaagtggaagcaggaggagcattatggcaaatcgtgctatgtggacttcaccaacccggacttcgtgaagctggccgagagcatgtacgccaaaggctatcgcgtggagaaagccgaggatctgatcccgaccctcgaagaggccttcaagcagaatgtcccggcggtcatcgactgccaggtggactatggcgagaatatcaagctcaccaagcacctcaaggaggtctatgaaaacatgtga

SEQ ID NO: 51 - DNA, 1719 bp, Bacillus licheniformis; identical to SEQ ID NO: 2
  source 1..1719: mol_type = genomic DNA

SEQ ID NO: 52 - DNA, 720 bp, Clostridium autoethanogenum
  source 1..720: mol_type = genomic DNA
atggatgatgaggtgaaagtcccgaaccacatctaccagatgtcgaccatcaatgccctggtcagcggcctctacgacggctgtgtgtcgctctcgaagctcctgaaaaagggcaatttcggcatcggcacgttcaagggcctggatggcgagctgaccctcctgaacggcacgttctatcgcaccaaaccggatggctccgtgtacgtgtgcagcaagaacgtgagcgtccccttcgcggtcgtcaccgagctggagaactacaatacctataacatccagaatcgcacctcctatgaggacatccgcaaggagctggactcgttcatcgagtcgaagaacatcttctatgccttctatatggaaggcaaattcaactacgtcaaaacccgcaccgtcgtgaagcagaacatgccgtacaagccgatggccgaggtggtcaaagaccagccgatgttcgaatacaacggcgtcgatggctacgtcgtcggcttccggtgcccggattatgtggaaggcctcaatgtgcccggctaccatttccacttcatcaacaaggacaaaaagttcggcggccacatctccgagttctcgatcgagaacgccaaagtctacgtccagaactgctcctgtttccgcatggagctcccgaagaatgagagcttctacaacatggaggtccaggaccgcaacgacgaaatcacgtccgtggagaaatga

SEQ ID NO: 53 - DNA, 780 bp, Klebsiella pneumoniae
  source 1..780: mol_type = genomic DNA
atgaaccactcggccgaatgcacctgcgaagagagcctctgcgaaaccctccgggccttctcggcccagcacccggagagcgtcctgtaccagacgagcctgatgtcggcgctgctgtcgggcgtgtatgaaggctcgacgaccatcgccgacctgctgaagcatggcgacttcggcctgggcaccttcaatgaactggacggcgagctcatcgccttcagctcgcaggtgtatcagctccgggccgatggctccgcccggaaggcccagcccgaacagaagaccccgttcgccgtgatgacctggttccagccgcagtatcggaagaccttcgaccaccccgtgagccgccagcagctccacgaggtgatcgaccagcagatcccgagcgacaacctcttctgcgccctgcgcatcgacggccatttccgccacgcgcatacccgcaccgtcccgcggcagaccccgccctaccgcgccatgaccgatgtcctggatgaccagccggtcttccggttcaaccagcgcgagggcgtcctggtcggcttccgcaccccgcagcacatgcagggcatcaacgtcgcgggctatcatgaacacttcatcaccgatgatcgcaagggcggcggccacctcctcgactaccagctggaccacggcgtcctgaccttcggcgaaatccataagctgatgatcgacctccccgccgacagcgccttcctgcaggcgaatctgcatccggacaacctcgatgccgccatccgctccgtcgagtcgtga

SEQ ID NO: 54 - DNA, 1074 bp, Clostridium autoethanogenum
  source 1..1074: mol_type = genomic DNA
atgaaggccgtcctgtggtacgacaaaaaggatgtccgcgtggaagaaatcgaggaaccgaaggtgaaagaaaacgccgtgaagatcaaagtcaagtggtgcggcatctgcggctcggacctgcatgagtatctcggcggcccgatcttcatcccggtcggcaccccccacccgctgtcgaagagcaccgcgcccgtcgtgctgggccacgagttctcgggcgaagtggtggagatcggcagcaaagtgaccaagttcaaggcgggcgaccgcgtcatcgtggaaccgatcgtcgcctgcggcaaatgcccggcctgcctggaaggcaagtacaatctgtgcgaggcgctgggcttccacggcctgtgcggcagcggcggcggcttcgccgagtacacggtgttcccggaagatttcgtgcacaagatccccgacacgatggattatgaacaggccgcgctggtggagccgatggcggtcgcgctgcactccctgcgggtgggcaacttcaccacgggcaacaccgccctggtcctgggcgcgggcccgatcggcctggccaccatccagtgcctcaaagcgtcgggcgcccggatcgtcatcgtcttccagcgcaaatcggtgcggcaggaatacgccaagaagttcggcgcggacgtggtcctcgacccgaatgaggtggacgtgatcgaggaaatcaaaaagctgaccggcggcgtgggcgtggacacgagcttcgaaaccaccggcgccaacgtcggcatcaacaccgcgatccaggcgctgaaatatgagggcaccgccgtcatcacctccgtctgggagaagaacgccgagatcaatccgaacgacctggtcttcaccgaaaagaaggtcgtcggcaccctcgcgtaccggcacgagttcccgtcgaccatcgccctgatgaacgacggccgcatcaagaccgatggctatatcaccaagcggatcgccctggaagacatcgtcaaggagggcttcgaaaccctgaccggcccggagaagaaaaagcacgtcaaaatcatcgtcacgcccgataaaagcctcctgtga

SEQ ID NO: 55 - DNA, 1053 bp, Bacillus subtilis
  source 1..1053: mol_type = genomic DNA
atgaaagccctgctgtggcataaccagcgcgacgtgcgggtggaagaggtcccggagcccgccgtccgcagcggcgcggtgaaaatcaaagtgaaatggtgcggcatctgtggcaccgacctgcatgaatatctggccggccccatcttcatcccgacggaggaacatccgctgacgcacgtcaaggccccggtcatcctcggccatgagttcagcggcgaggtggtggagatcggcgaaggcgtcaccaatcacaaagtcggcgatcgcgtggtcgtcgaaccgatctactcgtgcggcaagtgtgaggcgtgcaagcacggccactataatgtctgcgagcagctggtgttccacggcctgggcggcgacggcggcggcttctcggagtacaccgtggtgccggcggatatggtccaccacatcccggatgaaatgacctacgagcagggcgccctggtcgagccggccgccgtggcggtgcacgcggtgcgccagagcaaactcaaggagggcgaagccgtggccgtcttcggctgcggcccgatcggcctgctggtcatccaggcggccaaagcggcgggcgcgacccccgtcatcgcggtcgagctgtcgaaggaacgccaggagctcgccaagctggcgggcgcggattatgtcctgaaccccgccgaacaggacgtggtggcggaaatccggaacctgaccaacggcctgggcgtcaacgtctccttcgaggtcaccggcgtggaagtcgtcctgcggcaggcgatcgaatcgacctcgttcgagggccagacggtcatcgtgtcggtctgggagaaggacgccaccatcacgcccaataatctggtcctgaaagagaaggaagtggtcggcatcctcggctaccggcatatcttcccgtccgtcatcaagctgatctcgtcgggccagatccaggccgagaaactcatcaccaagaagatcacggtggaccaggtggtcgaagaaggcttcgaagcgctggtcaaggataagaagcaggtgaagatcctcgtgtcgccgaagtga

SEQ ID NO: 56 - DNA, 1053 bp, Paenibacillus polymyxa
  source 1..1053: mol_type = genomic DNA
atgcaggcgctgcgctggcacggcatcaaggacctgcggctggagaacatcgagcagcccgccgccctcccgggcaaggtgaagatcaaggtggaatggtgcggcatctgcggcagcgacctgcatgaatatgtcgccggcccgatcttcatccccgaaaacgcgcagcatccgctcacgggcgagaagtcgcccatcgtgatgggccatgagttctccggccagttcttcgacttcggcgaaggcgtgacgaaaatccaggtgggcgaccgcgaagtggtggagccggtcttcgcgtgtggcgaatgcgatgcgtgccggcagggcaaatataacctgtgcgataagatgggcttcctgggcctggccggcggcggcggcggcttctcggaatatgtcgccgcggatgagcatatggtgcacaaaatccccgagtccgtgtccttcgaacagggcgccctggtcgagccgtccgccgtcgccctctacgcggtccgccagatccagctgaaggtcgatgacaaggcggtggtcttcggcgccggccccatcggcctgctcgtcatcgaagcgctgaacgccagcggcgcgagcgaaatctatgcggaagagctcagcgaagagcgcaccgccaaagccgaagacctgggcgccatcgtgctcgaccccaacacgtacgatgtcgtcgaggaactccataagcgcacgaatggcggcgtctacgtcccctatgaggtcacggaagtcccgcccgtgctgacccaggccatcgagtccgccaagatctccggcgaaatcatgatcgtcatcatcttcgaaaaggaggccctcatcaagccgaacaacatcgtcatgaatgaacggaacctgacgggcctgatctgctacgacgatgtgttcccggccctgatctccctcatggagaatggctacttccccgccgacaagctggtcatcaaacggatcaagctggtggatgtcatcgaagcggccttcgagtcgctcctgatcgaggagtaccaggtgaccatcctcgtgtcgccgcacgcctga

SEQ ID NO: 57 - DNA, 3696 bp, Mycobacterium bovis
  source 1..3696: mol_type = genomic DNA
atggccaatatctcgtccccgttcggccagaacgagtggctcgtggaggccatgtatcggaagttccgggacgacccgtccagcgtcgacccgtcctggcacgagttcctcgtggactactcccccgagccgaccagccagccggcggccgagccgacccgggtcacgtcgccgctcgtcgccgaacgggccgccgccgccgccccccaggcgccccccaagccggccgacacggccgccgccggcaacggcgtggtggcggccctggccgccaagaccgccgtgccgcccccggccgaaggcgacgaagtcgccgtcctccggggcgcggcggcggcggtcgtgaagaacatgagcgcgtccctggaggtgccgaccgccacgtcggtccgggccgtccccgcgaaactcctcatcgacaaccgcatcgtcatcaataaccagctgaagcggacgcggggcggcaagatctcgttcacgcacctcctcggctacgcgctcgtgcaggccgtcaagaagttcccgaacatgaaccggcactacacggaagtggacggcaaacccacggccgtcacccccgcccatacgaacctcggcctggccatcgacctgcagggcaaggacggcaagcggtcgctggtggtcgccggcatcaagcgctgcgagacgatgcgcttcgcgcagttcgtcacggcctatgaagacatcgtccgccgggcccgggacggcaagctcaccacggaagatttcgcgggcgtcaccatctcgctcaccaatcccggcacgatcggcaccgtccatagcgtgccgcgcctgatgcccggccagggcgcgatcatcggcgtcggcgcgatggaataccccgccgagttccagggcgcgtccgaggagcggatcgcggaactgggcatcggcaagctcatcaccctcaccagcacgtatgaccaccgcatcatccagggcgccgagtcgggcgacttcctccggacgatccatgagctgctgctgagcgacggcttctgggatgaggtgttccgggaactgtccatcccgtatctgccggtccggtggagcacggacaaccccgatagcatcgtggacaaaaacgcccgcgtgatgaacctcatcgccgcctaccgcaaccgcggccacctgatggccgacaccgatccgctgcggctcgacaaagcccgcttccggagccatcccgacctggaagtcctgacccacggcctcaccctgtgggatctggatcgggtcttcaaggtcgacggcttcgccggcgcccagtacaagaagctgcgcgatgtgctcggcctgctgcgggacgcctattgccgccacatcggcgtggagtacgcgcacatcctcgacccggaacagaaagaatggctggaacagcgcgtcgaaaccaagcatgtgaagcccacggtggcgcagcagaagtacatcctgtcgaagctgaatgcggccgaagccttcgaaaccttcctgcagaccaagtacgtcggccagaagcgcttctccctggaaggcgccgagtccgtcatcccgatgatggatgccgcgatcgaccagtgcgccgaacacggcctggacgaagtcgtgatcggcatgccccatcgcggccggctcaacgtcctcgcgaacatcgtcggcaagccgtactcgcagatcttcacggagttcgaaggcaacctgaacccctcccaggcccacggcagcggcgacgtgaaatatcacctgggcgccaccggcctgtatctgcagatgttcggcgacaacgacatccaggtcagcctgaccgccaacccctcgcatctggaagccgtcgaccccgtcctggaaggcctggtccgcgcgaagcaggacctcctggaccacggcagcatcgatagcgacggccagcgggccttctccgtggtgcccctcatgctgcacggcgacgccgccttcgccggccagggcgtggtcgccgagacgctcaacctggcgaacctgcccggctaccgcgtcggcggcacgatccacatcatcgtcaacaatcagatcggcttcaccaccgcccccgagtactcccgcagctcggaatactgcaccgatgtggcgaaaatgatcggcgccccgatcttccatgtcaatggcgatgatccggaagcgtgcgtgtgggtggcccgcctggcggtggatttccgccagcggttcaagaaggatgtggtgatcgacatgctgtgctaccgccgccggggccataatgaaggcgacgatccctccatgaccaacccctacatgtacgatgtcgtggacacgaagcgcggcgcccgcaagtcctataccgaggccctcatcggccggggcgatatcagcatgaaagaagccgaggatgcgctccgcgactatcagggccagctggaacgggtgttcaacgaggtgcgggaactcgaaaaacacggcgtccagccgtccgagagcgtggagtccgatcagatgatcccggcgggcctcgccacggcggtcgataagtccctgctggcccggatcggcgatgccttcctggccctccccaatggcttcacggcgcacccgcgggtgcagccggtgctcgaaaaacgccgggaaatggcctacgaaggcaagatcgactgggccttcggcgagctgctggcgctgggctccctggtggccgaaggcaaactggtgcgcctgtccggccaggactcgcgccgcggcaccttctcccagcgccactcggtcctgatcgatcgccataccggcgaggagttcaccccgctccagctcctggccacgaactccgacggctcgcccaccggcggcaagttcctggtgtatgacagccccctgagcgagtatgcggcggtcggcttcgagtacggctacaccgtcggcaatcccgacgccgtcgtcctgtgggaagcgcagttcggcgacttcgtcaatggcgcccagtcgatcatcgacgagttcatctcgtcgggcgaggccaaatggggccagctctcgaacgtcgtgctgctgctcccgcacggccacgaaggccagggcccggatcacacctccgcccggatcgaacggttcctgcagctctgggccgagggctccatgacgatcgccatgcccagcaccccgtccaactatttccacctcctgcggcgccatgcgctcgacggcatccagcggcccctgatcgtcttcacgccgaagagcatgctgcgccataaggccgccgtctcggagatcaaggacttcaccgagatcaaattccggtcggtgctggaagaaccgacctatgaagatggcatcggcgaccgcaataaggtgtcgcgcatcctgctgacctcgggcaagctgtactacgagctggccgcccgcaaggccaaagacaaccggaacgacctggccatcgtccggctggagcagctggcgcccctcccgcgccggcgcctgcgcgaaaccctggatcgctatgagaacgtcaaggagttcttctgggtgcaggaggaacccgcgaatcagggcgcctggccccgcttcggcctggagctgccggagctgctccccgacaagctggccggcatcaagcggatcagccgccgcgcgatgtcggccccgagctcgggctccagcaaggtccatgcggtcgagcagcaggagatcctggatgaagcgttcggctga

SEQ ID NO: 58 - DNA, 3696 bp, Mycobacterium tuberculosis
  source 1..3696: mol_type = genomic DNA
atggccaacatcagcagccccttcggccagaatgaatggctcgtggaggagatgtaccggaagttccgggatgatcccagctcggtcgatccgtcgtggcacgagttcctggtcgactatagccccgaacccacctcccagccggcggccgagcccacgcgggtgacctccccgctggtcgcggaacgcgccgcggcggcggccccccaggccccgccgaaacccgccgatacggccgccgcgggcaacggcgtcgtcgcggccctggccgcgaagaccgccgtgcccccccccgccgagggcgacgaggtggcggtgctccggggcgccgccgcggccgtggtcaagaacatgagcgcctccctcgaagtgccgaccgccaccagcgtccgggcggtgccggccaagctgctgatcgataatcggatcgtcatcaacaatcagctgaaacggacccgcggcggcaagatcagcttcacccatctgctcggctatgcgctggtccaggccgtgaagaagttccccaacatgaatcgccactataccgaagtcgacggcaaacccaccgccgtgaccccggcgcacacgaacctcggcctggcgatcgacctgcagggcaaggacggcaagcggagcctcgtggtggccggcatcaaacggtgcgagacgatgcggttcgcgcagttcgtgacggcctatgaggatatcgtgcgccgcgcccgggacggcaaactgaccaccgaagacttcgccggcgtcacgatctcgctgaccaacccgggcaccatcggcacggtccattcggtcccgcgcctcatgccgggccagggcgccatcatcggcgtgggcgcgatggaatatcccgccgagttccagggcgcctccgaggagcggatcgccgagctgggcatcggcaaactcatcacgctgacctccacgtatgaccaccgcatcatccagggcgcggaaagcggcgacttcctccggacgatccatgagctgctgctctccgacggcttctgggatgaagtgttccgcgagctgtcgatcccctatctcccggtgcgctggagcaccgacaacccggactcgatcgtggacaaaaacgcccgcgtcatgaatctgatcgccgcgtaccgcaatcggggccacctcatggcggacacggaccccctgcggctcgataaggcccggttccgctcccaccccgacctcgaagtcctgacccacggcctgaccctgtgggacctggatcgcgtcttcaaggtcgatggcttcgccggcgcccagtataagaagctgcgcgatgtgctgggcctgctccgcgatgcctattgccggcatatcggcgtggagtacgcccacatcctggaccccgagcagaaagaatggctggaacagcgcgtcgaaacgaagcacgtcaagcccaccgtcgcgcagcagaaatacatcctcagcaaactcaacgccgccgaagcgttcgaaacgttcctccagacgaagtacgtgggccagaaacgcttcagcctggaaggcgcggagagcgtcatccccatgatggacgccgccatcgaccagtgcgccgaacacggcctggatgaagtggtcatcggcatgccgcaccgcggccggctcaacgtcctggccaatatcgtgggcaagccctacagccagatcttcaccgagttcgaaggcaacctgaacccgtcgcaggcgcacggctcgggcgatgtcaagtaccatctcggcgccacgggcctgtatctgcagatgttcggcgacaacgacatccaggtgtccctgacggcgaaccccagccacctggaggcggtcgatcccgtcctggaaggcctggtgcgggccaagcaggatctcctcgaccacggctcgatcgactccgatggccagcgggcgttcagcgtggtgcccctcatgctgcatggcgacgcggcgttcgcgggccagggcgtggtggcggaaaccctcaacctcgcgaacctcccgggctatcgggtgggcggcaccatccatatcatcgtgaacaaccagatcggcttcaccacggcgccggaatattcccggagctcggaatattgcacggacgtggccaagatgatcggcgccccgatcttccacgtcaatggcgatgacccggaggcctgtgtgtgggtcgcccggctggccgtcgatttccgccagcgcttcaaaaaagatgtggtcatcgacatgctctgctaccgccgccggggccataatgagggcgacgacccctccatgacgaacccgtacgtctacgacgtggtcgacaccaagcgcggcgcccgcaagtcctatacggaggcgctcatcggccgcggcgacatctcgatgaaggaagcggaagacgcgctccgcgactaccagggccagctggagcgcgtcttcaacgaagtgcgggaactcgaaaagcatggcgtgcagccctccgaatcggtggagagcgatcagatgatcccggccggcctggccaccgccgtcgataaaagcctgctcgcccgcatcggcgacgccttcctggccctgccgaacggcttcacggcccatccccgcgtccagccggtgctggagaaacgccgcgaaatggcctacgagggcaagatcgattgggccttcggcgagctgctggcgctcggcagcctggtggccgaaggcaagctcgtgcggctctccggccaggactcgcggcgcggcacgttctcgcagcgccattccgtgctgatcgaccggcacaccggcgaagagttcacccccctccagctgctggccaccaattcggacggcagcccgacgggcggcaagttcctggtctatgactccccgctgagcgagtatgccgccgtcggcttcgagtatggctacaccgtcggcaatccggacgcggtcgtgctgtgggaagcgcagttcggcgacttcgtgaacggcgcccagtcgatcatcgatgagttcatctcgtcgggcgaagccaagtggggccagctcagcaacgtggtgctcctgctcccgcacggccacgagggccagggcccggatcatacgagcgcccgcatcgagcggttcctgcagctgtgggccgagggctccatgacgatcgcgatgccctcgacgccgtccaactacttccacctgctgcgccggcacgcgctggacggcatccagcgcccgctgatcgtcttcacccccaagagcatgctccggcacaaagccgccgtgtccgagatcaaggacttcacggagatcaaattccggagcgtgctggaggagcccacgtacgaggacggcatcggcgaccgcaacaaggtgtcccgcatcctcctgacgagcggcaagctgtactacgagctcgccgcccgcaaggccaaggataaccggaacgacctggccatcgtgcgcctcgaacagctggccccgctgccgcgccgccggctgcgggaaaccctggaccgctacgagaacgtcaaagagttcttctgggtccaggaagaaccggccaaccagggcgcgtggccgcgcttcggcctggaactgcccgagctcctgccggacaagctggccggcatcaagcgcatctcgcggcgggcgatgagcgccccgtcgagcggctcctccaaggtgcatgccgtggagcagcaggaaatcctggacgaggcgttcggctga

SEQ ID NO: 59 - DNA, 3738 bp, Corynebacterium terpenotabidum
  source 1..3738: mol_type = genomic DNA
atgaactcgaccaacttcggcagcaatggctggctggtcgaccagatgcaccagcagttcaaggaagatccgcagtcggtcgacaaggaatggcgcgacttcttcaccgccggctcggcgtccggcccggatgccccggaaacgaccgccccgacgaccaccgccgccacgaccacgaccgcgaccacgagcgcggcccccgccaccgcgcagaccagcggcgtggccgtcccgtccaccgcgggcaccacccaccgggcctcggccgccgcgaccgtggcctcgaacgtggccctgagcgcgaacccgatccccgaacgggtcgcccccccggccccgaaggacgcggccgagccgctgaccccgggcgaggccgccctgaaaggcgcccagcgggcgatcgccaaaaacatggacgccagcctggatatccccaccgcgacgacggtccgcgacatgccggccaaactcatgttcgaaaaccgggccatgatcaacaaccatctgcgcagccagggccggggcaagatctcgttcacccacatcctgggctgggcgatggtcaacgccgtgaaagcccaccccaccatgaataacaattacaaggtcatcgacggcaagccgtcggtcgtgacccccgaacatatcaatctcggcctcgccatcgatctggtcagcaaaaacggctcgcggaacctggtcgtggccgccatccgcgcgtgcgaaaccttcgacttcgagggcttcgtggacgcctacgaggacatcgtggtccgcgcccggaagggcaaactgacgatggacgacttcagcggcgtcaccatccagctgaccaacccgggcggcatcggcacccgccactccgtcccgcgcctgacgcacggccagggcgcgatcatcggcgtgggcgcgatggactacccggcggagttcgccggcgcctcggaagaccgcctcgcggacctgggcgtgggcaaactggtgaccatcacgagcacgtatgaccaccggatcatccagggcgccgagtcgggcgagttcctgcgcgacatgagccgccagctcatcgacgatggcttctgggacggcatctacgccagcctgaaagtcccgtatgcgcccgtgcggtggtcgcaggacgtgcccaacaccggcgtcgacaagagcacccgcgtcatgcagctcatcgaggcgtaccgctcccgcggccacctgatcgccgacatcgacccgctgcattggacccagcccggcctgccggtgccggaccatagcgacctggacatcgaatcccacggcctgaccctgtgggacttcgaccggcgcttccacgtcggcggcttcgccggccgcgaatcgatgaccctgcgggaagtgctggccacgctgcggaaggcgtatacgctgaaggtcggctccgaatatacccatatcctcgacaaggacgaacgcctctggctccaggagcacatcgaggccggccagcagaagctcagcaaccccgaacagaagtatctgctgcagacgctgaactccgccgaggcgttcgaaaacttcctgcagaccaaatatatcggccagaagcgcttcagcctggaaggcgccgaagccctgatcccgctgctcgatgccgccgcggatcaggccgccgaacagggcctcgaagaggtggtgatcggcatgccgcatcgcggccggctgaatgtcctcgcgaacatcgtcggcaaaccgtacagcaccatcttcggcgagttcgagggcaacatcgagccggccgccgcgggcggctccggcgatgtcaaataccatctgggcgccgagggcgtgtatacgcagatgttcggcgacaacgacatcaaggtgacgctgaccgccaacccctcccacctggaagcggtcaatcccgtcatggagggcctcgcgcgggcgcatcaggatatctcgccccgggcggaggatcggccgatcatgccgatcctgatgcacggcgacgccgccttcaccggcctgggcatcgtcccggaaacgatcaacatggcccagctgcggggctactccgtgggcggcacggtgcatgtggtggtcaacaaccagatcggcttcaccacgacgccggatcgcggccggtccacgcactacgcgacggacatcgcgaagggcttcgattgccccgtcttccacgtcaacggcgacgatccggaggccgtcgtctgggtcgcccggctcgccgtggaataccggcgccgcttcggcaaagacgtgttcatcgacctggtgtgttatcggcgccgcggccataatgaagccgatgaccccagcatgacccagcccgaactgtacagcatcatcgagagccggccgacggtccggagcctgtatcatgacacgctggtcggccggggcgacatcacggcggaagacgcccagcgggccgccgacgacttccacggccagctggaaagcgtcttcaaccaggtcaaggaaggcgtgaagggcgtgaccccggcggcccagaccggcatcgccggcggccaggacctgtccaccggcctcgacaccagcatcaccgcggatgtgatcgccgagatcggcgattcctataccgtggatgcccccgaagacttcaatgtccaccagcgcgtgaagcccgtggtcaaacggcggcaccagatgtcgcgccagggcaagatcgattgggcgttcggcgaactcctcgcgttcggctcgctggcccgggaaggccggctggtccggctcgccggcgaggactcgcagcggggcaccttcacccagcggcacgccatcctgttcgacagcaccaccaacaagcccttcagccccctggaaatggtcgcgcggaactccggcaacggcggctcgttccgggccttcaattccccgctgacggaatatgcgggcatgggcttcgagtatggctactcggtgggcaacctggatgccgtggtggcctgggaggcgcagttcggcgacttcgcggacggcgcccagacgatcatcgacgagtacatctcctccggcgaggccaaatggggccagctgtcgagcgtgatcctgctgctcccgcacggctacgagggccagggcccggaccactcctccgcccgcatcgaacgctacctgcagatggccgccgaaggctcgatgaccatcgcgcagccgtcgacgcccgccaaccacttccacctgctccgccgccacgccctgggcaccatgcggcgcccgctggtcgtcttcaccccgaagtccatgctgcgcaacaaggcggccgtctcctcggtcgaagacttcacggaagtcaccaagttccggagcgtcctggatgacccccggttcgcggatggcaccgccgaccggagcggcgtcaagacggtgctgatgtgttccggcaagatctactatgatctggagaagaagcgggcggaagacggccgcgacgatatcgccatcgtccgcgtcgaaatgctgcatcccatcccgcataaccgcatccgcgaaacggtcgtggagggctaccccggcgcggaggtgcgctgggtgcaggacgagcccgccaaccagggcgcctggccgttcctggccctcaacctccccgagcgcatccccggcttcaccatgaagcgcgtcagccgccgggcccagagcagcaccgccacgggcgtggccaaggtccaccatctggagcaggagacgctgctgaccgaagccttcgccacctga

SEQ ID NO: 60 - DNA, 3777 bp, Rhodococcus jostii
  source 1..3777: mol_type = genomic DNA
atgagctcgtcctcgacctcccagttcggccagaaccagtggctcgtggatgaaatgtaccagcgcttccaggatgatccgtcgtccgtggacgcctcctggcatgagttcctgacggactatagccccgacgccgccgccaaggcgggcgcggcgaacggccacggcaccaacggcaccaccacggcggcgccggcggcggcgccgtccgccaaagccgcgacccccccggtccccgagagcgagacggccccgaaaccccagaccaagacggcgaacggcgcggcgccgaaagccgcgcccaacggcgcggcgccgaaggccgcggcccccaagaccgaggcccccaagaaggccgcccccgcgaaggaaacggccgcgacggacgcgaaagcctcggccccggcccccgccgtggaggaatccaaggtgctgcggggcgccgccgccgccgtcgccaagaatatgtccgcgtcgctcgccatcccgaccgccacctccgtccgggccatcccggcgaagctgatgttcgacaaccgcatcgtcatcaacaaccacctggcgcggacccggggcggcaagatctcgttcacccatctgctgggctacgcgatcgtgcaggccgtgaaagcgttccccaacatgaaccgccacttcgcggagatcgatggcaagcccaatgcggtgacccccgcccataccaatctgggcctcgcgatcgatctgccgggcaaagatggcagccgctcgctggtcgtcgcggccatcaagaacaccgatacgcacaatttcacccagttctatagcgcctacgaggatatcgtgcgccgggcgcgggacggcaaactgaccgccgaagacttcagcggcgtcacgatctcgctgaccaacccgggcggcatcggcaccgtccattccgtcccgcgcctgatgaacggccagggcgccatcatcggcgcgggcgcgatggagtacccggccgagttccagggcgcctcggacgagcggctggccgaaatcggcgtcggcaagctgatgaccctcacgagcacctacgatcatcggatcatccagggcgccgaatccggcgacttcctccggacgatccataatctgctcatcagcgacgagttctacgacgaaatcttccatgccctgcatatcccctatgaacccgtccggtggcgcaaagacgtgcccgaaggcgccgtggataagaacacccgggtcctggagctcatcgccgcctaccggaaccgcggccacctgatggcggacaccgacccgctgcagttcgtgaaggacaagttccggtcgcacccggacctcgacgtgcgcacccatgatctgacgctctgggacctggatcgggagttcaaagtgggcggcttccacggccaggagaagatgaagctgcgcgatgtcctgtcggtgctgcgcgatgcgtattgtcgccatgtcggcgtggagtatacccacatcctggagccggagcagcagcagtggctgcaggatcgcgtcgaggcccaccacgtcaaaccgacggtcgcccagcagaagtatatcctgtccaaactgaacgccgccgaggccttcgagacgttcctgcagacgaagtatgtcggccagaaacgcttctcgctggagggcgcggagagcgtgatccccatgatggacgccgtcatcgaccaggccgccgagcatcagctggacgaggtggtgatcggcatgccgcatcggggccgcctgaatgtcctggcgaacatcgtgggcaagccgtattccaagatcttcaccgagttcgagggcaacatgaacccggcggcggcccacggcagcggcgacgtgaagtatcacctgggcgccgaaggcacgtatatccagatgttcggcgataacgacatcaccgtctcgctcaccgcgaacccgagccatctggaagccgtggacccggtgctggagggcctggtccgggccaagcaggacatcctcgacaagggcgaagacggcttcacggtgctccccctcatgctccacggcgacgcggccttcgccggccagggcgtggtcgccgagacgctcaacctcgccctgctgcgcggctaccggaccggcggcaccgtgcacatcgtggtgaacaaccaggtcggcttcacgaccgcccccgagtatagccgctccagcgagtactgcacggatgtggcgaagatgatcggcgcccccatcttccacgtcaacggcgatgaccccgaggcgtgcgtgtgggtggcccagctggcggtggacttccgcgagaagttccagaaggacgtggtcatcgacatgatctgttatcggcggcggggccataatgagggcgacgatccgagcatgacccagcccgcgatgtacgacgtgatcgacacgaaacgctccgtccgcaaatcctataccgagtcgctgatcggccgcggcgacatctcgctgaaggaagcggaggacgccctgcgcgactatcagggccagctggaacgggtcttcaatgaggtgcgcgagctggagaagtacacgcccgaaccgagcgagagcgtcgagctggaccaggtcctgcccacgaagctgaaaacctccgtcgatgaatcggtgctggagcgcatcggcgatgccttcgtgaacgtcccggagggcttcacggtccatccgcgggtgaagccggtgatcgaaaagcgccgcgagatgagccgcgaaggcaagatcgactgggccttcgccgagctgctggccttcggctccctggtggatcagggcaagatggtgcgcctctccggccaggacagcaagcgcggcacgttcacccagcgccactcggtcctcatcgaccgcaaaaccggcgccgaatatacccccctccagaacctgggcagcgaaaacccgggcaagttcctggtctacgattccgcgctgagcgagttcgcggcggtcggcttcgagtacggctatagcgtcggcaaccccgacgcgctggtgctgtgggaagcccagttcggcgatttcgtgaatggcgcccagagcatcatcgacgagttcatctcgtcgggcgaagccaaatggggccagctgagcgacgtggtcctgctgctgccccacggccacgaaggccagggcccggaccatacgtccggccgcatcgaacggttcctccagctgtgtgccgaaggctccatgaccgtggcggtccccagcacgccggcctcgtacttccacctgctgcgccggcatagcctcgacggcatccgccggccgctcgtcgtgttcacccccaaaagcatgctgcgcaacaaagccgccgtgagcgacgtggaagacttcacgaccggcaagttccggagcgtcttcgaggagcccacctacgagacgggcgatgccgagcgggacaaggtgcgccgggtcctcctggtgtcgggcaagctgtactgggagctgctggccaagaagcagaaggataaccgcgaggacatcgccatcgtgcgcatcgaacagctgtaccccgtcccctcgcggcggctccgcgagacgctggaccgctaccccaacgccaccgagttccgctgggtgcaggaggagcccgccaaccagggcgcctggcccttcttcggcctcgcgctgcccgaactgctgcccgacaaactgtcgggcatcaagcgcatcagccgccggtccatgtcggccccgtcctccggctcgtccaaggtgcacgcggtcgaacagcaggaaatcatcgacgaggccttcggctga

SEQ ID NO: 61 - DNA, 1116 bp, Porphyromonas gingivalis
  source 1..1116: mol_type = genomic DNA
atgcagctgttcaaactcaaaagcgtgacgcaccacttcgatacgttcgcggagttcgccaaggagttctgcctcggcgagcgggacctggtcatcaccaatgagttcatctacgagccctatatgaaggcgtgccagctcccgtgccacttcgtcatgcaggaaaagtacggccagggcgaaccgtcggatgagatgatgaacaacatcctggcggatatccggaacatccagttcgaccgggtcatcggcatcggcggcggcaccgtcatcgacatctccaaactgttcgtcctgaaaggcctcaacgacgtgctggacgccttcgaccgcaaaatcccgctcatcaaggagaaggagctcatcatcgtgcccacgacctgcggcaccggctcggaagtgaccaacatctcgatcgccgagatcaagtcgcgccacaccaagatgggcctggcggatgacgcgatcgtggccgaccatgccatcatcatccccgagctgctgaagagcctgccgttccacttctatgcgtgctccgccatcgatgccctgatccacgccatcgagagctacgtctcgccgaaggcctccccgtacagccggctgttcagcgaggcggcctgggacatcatcctggaagtcttcaagaagatcgccgagcacggccccgaatatcggttcgaaaagctcggcgagatgatcatggccagcaattacgccggcatcgccttcggcaacgccggcgtcggcgccgtccacgccctgtcgtacccgctgggcggcaattatcatgtgccgcatggcgaagcgaattaccagttcttcaccgaggtgttcaaggtctatcagaagaaaaacccgttcggctacatcgtcgagctgaactggaagctgtcgaagatcctgaactgccagccggagtatgtgtatcccaagctggatgaactcctgggctgcctgctgaccaaaaagccgctgcatgaatatggcatgaaggacgaagaggtgcggggcttcgccgaatcggtgctgaagacccagcagcgcctgctcgccaacaactacgtggagctgaccgtcgacgaaatcgaaggcatctaccgccggctgtactga

SEQ ID NO: 62 - DNA, 1116 bp, Clostridium kluyveri
  source 1..1116: mol_type = genomic DNA
atgaagctcctgaagctggccccggatgtgtacaaattcgataccgccgaggagttcatgaagtatttcaaggtcggcaagggcgacttcatcctgacgaacgagttcctctataaacccttcctggaaaagttcaatgatggcgccgacgccgtgttccaggaaaagtacggcctgggcgagccgtccgacgaaatgatcaacaacatcatcaaggatatcggcgacaaacagtacaatcggatcatcgcggtgggcggcggcagcgtcatcgatatcgccaaaatcctgtcgctgaaatacacggacgattccctggacctgttcgaaggcaaggtcccgctggtgaagaataaagagctcatcatcgtcccgaccacctgcggcaccggctcggaagtgacgaatgtctcggtcgccgaactgaagcggcgccatacgaagaaaggcatcgcctccgacgaactctatgcgacgtacgcggtcctggtgcccgagttcatcaaaggcctgccctataagttcttcgtgacgagctccgtcgacgccctcatccatgcgaccgaggcctacgtgtcgccgaacgccaacccctacaccgacatgttctcggtcaaggcgatggaactgatcctgaacggctacatgcagatggtggagaagggcaacgattatcgcgtggaaatcatcgaggatttcgtgatcggctccaactacgcgggcatcgcgttcggcaacgcgggcgtcggcgcggtccatgccctcagctaccccatcggcggcaactaccatgtcccgcatggcgaagccaactacctcttcttcacggagatcttcaagacctactacgagaagaacccgaatggcaagatcaaggacgtgaataagctcctggcgggcatcctcaagtgcgatgagtccgaggcgtacgattcgctctcgcagctgctggacaaactgctctcgcgcaagccgctgcgcgagtacggcatgaaggaggaagagatcgaaaccttcgccgacagcgtgatcgaaggccagcagcggctgctggtcaacaactacgagcccttcagccgcgaggatatcgtcaacacctacaaaaagctctactga

SEQ ID NO: 63 - DNA, 1296 bp, Porphyromonas gingivalis
  source 1..1296: mol_type = genomic DNA
atgaaggacgtgctggccgagtacgcctcgcgcatcgtctcggcggaagaagcggtcaagcacatcaagaacggcgagcgcgtcgcgctctcgcatgccgccggcgtcccgcagtcctgcgtcgatgccctggtccagcaggccgatctcttccagaatgtcgaaatctaccatatgctctgcctgggcgagggcaagtacatggcccccgagatggcgccgcatttccgccatatcacgaatttcgtcggcggcaactcgcggaaagccgtggaggagaaccgggccgacttcatccccgtgttcttctacgaggtccccagcatgatccgcaaagatatcctccacatcgacgtggcgatcgtgcagctgagcatgccggatgagaacggctactgcagcttcggcgtgagctgtgactacagcaagccggccgccgaatccgcccacctcgtcatcggcgagatcaaccggcagatgccgtacgtccacggcgacaacctgatccacatcagcaagctggactacatcgtcatggccgactatccgatctatagcctggcgaaacccaagatcggcgaggtcgaggaggccatcggccggaactgcgccgagctgatcgaagacggcgccaccctccagctgggcatcggcgccatcccggacgccgccctgctcttcctgaaagacaagaaagacctgggcatccacaccgaaatgttcagcgacggcgtcgtcgaactggtccggagcggcgtcatcaccggcaagaaaaaaaccctgcacccgggcaaaatggtcgcgacgttcctgatgggctccgaagatgtctatcatttcatcgacaagaaccccgacgtggagctctacccggtggactatgtgaacgacccgcgcgtcatcgcccagaacgacaacatggtcagcatcaactcctgtatcgagatcgacctcatgggccaggtcgtgagcgaatgtatcggctccaagcagttctcgggcacgggcggccaggtggactacgtccgcggcgcggcgtggtcgaagaacggcaagtcgatcatggccatcccctccaccgccaagaacggcaccgcctcgcgcatcgtgccgatcatcgcggagggcgccgcggtgacgaccctgcggaacgaagtggattacgtggtcacggagtatggcatcgcccagctgaagggcaagtcgctccgccagcgggcggaagcgctgatcgcgatcgcgcacccggacttccgggaggagctgaccaaacacctgcggaaacggttcggctga

SEQ ID NO: 64 - DNA, 1296 bp, Porphyromonas gingivalis
  source 1..1296: mol_type = genomic DNA
atgcagtggcaggaactctaccggcagcgcgtgtgctccgcggatgaagccgtcgtggatagcctgaagcccggcaccaaggtggtcttcggccatgccgccgccgcgcccgtgcggttctcgcaggccatgtatcgccagcgcgaaaagctcgaaaacatcaccgtgttccatatgctctatttcggcgatgccccccacctggcccccgaaatgcgctcccatgtccacccgacgctcaacttcctggaaggcaatagccgccccgcgtcgcgggatcggcgcgtggatttcatcccctgccacttccacgaagtgcccgagctgttccgccagggcttcttccccctcgacgtggcggtggtgcaggtgtcgaccccgaacgaggagggctattgtagcttcggcgtctcgtgtgactatacgaaggccgcggccgaatgtgccccggtcgtggtggccgaggtgaacaagcagatgccgttcatcggcggcgagaacctgatccatatctccaagctgacccatatcatcgaagtggatgagccgatcgccgaggtgctgccccccgcgatcagcgacctggagctgcgcatcggccagaactgcgcgtccctcatcaaggacggcgatacgctccagctgggcatcggcggcatcccggacgcggtgctgcgggccctggaaggccataaagacctcggcatccacacggagatgttcaccgacggcgtgatgcggatgatccggaagggcatcatcaacggcaagaaaaagaccctgcacccggaaaaagtggtgaccagcctgatcttcggcagcaaagaactgtacgacttcgtcaacaacaacccggtgatcgagtgctatccggtcgattatatcaacaatccggatgtcatcggcaagaacgaccgcatggtgagcatcaactcctgcctggaaatggacctgatgggccaggccgcgagcgaaagcatcggctacgaacagttctccggctcgggcggccaggtggatttcctgcggggcgcgaagcggtccaaaggcggcatcagcatcatggccttcccctccacggccaagaagggcacggagtcccgcatcgtgcccatcctgaaggagggcgcgtgcgtcacgaccggccgcaatgaggtggattacgtcgtcaccgagtacggcgtcgcccgcctccggggcgccaccctccggcagcgggccgaggccctgacggccatcgcccatcccgacttccgccccgccctggaggaagagatccgccggcgcttcgagtga

SEQ ID NO: 65 - DNA, 1317 bp, Clostridium aminobutyricum
  source 1..1317: mol_type = genomic DNA
atggattggaaaaagatctacgaagatcgcacctgcaccgccgatgaagccgtcaagtccatcaagtccggcgaccgcgtcctgttcgcccactgtgtcgccgaacccccggtgctggtcgaagcgatggtggccaacgccgcggcgtacaagaacgtcaccgtgagccatatggtgaccctgggcaagggcgaatatagcaagcccgagtacaaggagaacttcacgttcgagggctggttcacctccccgtcgacgcggggctcgatcgcggagggccacggccagttcgtgcccgtgttcttccatgaggtgccgtccctgatccgcaaggatatcttccacgtcgacgtgttcatggtcatggtgtccccgcccgatcataacggcttctgctgtgtcggcgtcagcagcgactacacgatgcaggccatcaagtcggccaagatcgtgctggcggaagtcaacgaccaggtgcccgtggtgtatggcgatacgttcgtgcatgtctcgaacatcgataagttcgtggaaacctcgcatccgctcccggaaatcggcctgccgaaaatcggcgaggtggaagcggccatcggcaaacactgtgcctccctgatcgaagacggctcgacgctgcagctgggcatcggcgccatccccgacgccgtgctcagccagctgaaagacaagaagcacctgggcatccactcggagatgatctccgacggcgtcgtggacctgtatgaggcgggcgtgatcgactgctcccagaagtcgatcgacaagggcaaaatggcgatcaccttcctgatgggcaccaagcgcctgtacgacttcgcggcgaacaatccgaaggtggagctgaaaacggtggactacatcaaccatccgtcggtggtggcccagtgctccaagatggtgtgcatcaacgcctgcctgcaggtggatttcatgggccagatcgtgagcgacagcatcggcaccaagcgcttctcgggcgtgggcggccaggtcgacttcgtgcggggcgcctccatgagcatcgacggcaagggcaaggcgatcatcgccatgccgtcggtcgccaagaagaaggacggcagcatgatctcgaaaatcgtccccttcatcgatcacggcgccgccgtgaccacctcgcggaacgacgccgattatgtggtcacggagtacggcatcgccgagatgaaaggcaagtccctccaggaccgggcccgggcgctcatcaacatcgcgcacccggatttcaaggatgagctgaaggcggagttcgagaagcggttcaacgcggcgttctga

SEQ ID NO: 66 - DNA, 2577 bp, Clostridium aminobutyricum
  source 1..2577: mol_type = genomic DNA
atgaaggtgaccaaccagaaagaactgaagcagaaactgaacgaactgcgggaggcgcagaaaaagttcgccacctatacccaggagcaggtggacaaaatcttcaagcagtgtgcgatcgccgccgccaaagaacggatcaacctggcgaaactcgccgtcgaagaaaccggcatcggcctcgtggaggacaagatcatcaaaaatcacttcgccgccgaatatatctataacaaatacaagaacgagaagacctgcggcatcatcgaccatgacgactcgctgggcatcaccaaagtcgccgagcccatcggcatcgtggccgcgatcgtgcccaccaccaacccgacgagcaccgccatcttcaagtcgctgatctccctgaagacgcgcaatgcgatcttcttcagcccccatccgcgggccaaaaagtcgacgatcgcggcggccaaactgatcctggacgccgcggtcaaggccggcgccccgaaaaatatcatcggctggatcgacgaaccgtccatcgagctgtcccaggacctcatgtccgaagccgacatcatcctggcgaccggcggcccgagcatggtgaaggccgcctattccagcggcaaaccggccatcggcgtcggcgccggcaacacccccgcgatcatcgacgagtccgccgacatcgacatggccgtctcctcgatcatcctgagcaagacctacgataacggcgtcatctgcgcgtcggaacagagcatcctggtcatgaactccatctatgaaaaggtcaaggaagagttcgtgaagcgcggctcgtatatcctgaaccagaatgaaatcgcgaagatcaaggagacgatgttcaagaacggcgccatcaacgccgacatcgtcggcaagagcgcctatatcatcgcgaagatggccggcatcgaagtgccgcagaccaccaagatcctgatcggcgaagtccagtccgtcgaaaagtccgaactgttctcccacgaaaagctgagcccggtgctcgccatgtacaaagtcaaagacttcgacgaggccctcaaaaaggcgcagcggctcatcgaactgggcggcagcggccatacctcgtcgctgtacatcgatagccagaacaacaaggacaaagtcaaagagttcggcctggcgatgaagacgagccgcaccttcatcaacatgccgagcagccagggcgcgtccggcgacctgtacaacttcgccatcgccccgagcttcacgctcggctgcggcacctggggcggcaattcggtgtcgcagaacgtggaaccgaaacatctgctgaacatcaaatccgtcgcggagcgccgcgaaaatatgctctggttcaaggtcccgcagaagatctatttcaaatatggctgcctgcgcttcgccctcaaagaactgaaagacatgaataagaaacgcgccttcatcgtcaccgataaagatctgttcaagctgggctacgtgaacaaaatcacgaaggtcctggatgagatcgatatcaagtacagcatcttcaccgacatcaagtccgatcccaccatcgactccgtcaagaagggcgccaaagagatgctcaacttcgaaccggacaccatcatcagcatcggcggcggctcgccgatggacgcggccaaggtcatgcatctgctctatgaatatccggaggccgagatcgaaaacctggccatcaacttcatggacatccggaaacgcatctgcaacttcccgaagctcggcacgaaagccatcagcgtggccatccccacgaccgcgggcaccggctcggaagccacgccgttcgcggtcatcacgaatgacgaaaccggcatgaaataccccctgacctcctacgagctcacccccaatatggccatcatcgacacggagctcatgctcaacatgccgcggaagctcaccgccgccaccggcatcgatgccctggtgcacgccatcgaagcctacgtgagcgtgatggcgaccgactataccgatgagctggccctccgggccatcaagatgatcttcaagtatctgccgcgggcctacaaaaacggcacgaatgacatcgaggcccgcgaaaagatggcgcatgcgagcaacatcgcgggcatggccttcgcgaacgccttcctgggcgtgtgccatagcatggcgcacaaactgggcgcgatgcatcacgtcccgcatggcatcgcctgtgccgtcctgatcgaggaggtcatcaagtacaacgccacggattgtccgaccaaacagaccgccttcccgcagtacaagtccccgaacgcgaagcggaaatacgcggagatcgcggagtacctgaatctgaaaggcaccagcgacaccgaaaaagtcacggccctcatcgaagccatctcgaaactgaaaatcgatctgagcatcccccagaacatctccgcggccggcatcaataagaaggatttctataacacgctggacaagatgagcgagctggcgttcgatgaccagtgcacgaccgccaacccccggtatccgctgatctccgaactcaaggacatctacatcaagtccttctga

SEQ ID NO: 67 - DNA, 2676 bp, Escherichia coli
  source 1..2676: mol_type = genomic DNA
atggcggtgacgaacgtcgccgaactgaacgccctcgtcgaacgggtgaaaaaggcgcagcgggagtacgcctcgttcacgcaggagcaggtggataaaatcttccgcgccgccgccctggccgcggccgacgcccgcatcccgctcgccaaaatggcggtggccgagtccggcatgggcatcgtggaggacaaggtcatcaagaaccatttcgcctccgagtacatctacaacgcctacaaggacgagaagacctgcggcgtcctgtcggaggacgataccttcggcaccatcacgatcgccgagcccatcggcatcatctgcggcatcgtcccgaccaccaatcccacctccaccgccatcttcaaaagcctcatctccctgaaaacccgcaatgcgatcatcttcagcccccatccccgcgccaaagacgccacgaacaaggcggccgacatcgtcctccaggccgccatcgcggccggcgcccccaaagacctgatcggctggatcgaccagccgtcggtggagctgtccaacgccctcatgcatcacccggacatcaatctcatcctggccacgggcggcccgggcatggtgaaggccgcctactccagcggcaagccggcgatcggcgtgggcgcgggcaacacgcccgtggtcatcgacgaaacggccgatatcaaacgcgccgtcgcctccgtgctcatgagcaagaccttcgacaacggcgtgatctgcgcgtcggaacagtcggtcgtggtcgtcgactccgtgtacgacgcggtgcgcgagcgcttcgccacgcatggcggctacctgctgcagggcaaggagctgaaagccgtgcaggacgtgatcctcaaaaacggcgccctgaacgccgccatcgtcggccagcccgcgtataagatcgcggagctggccggcttctcggtcccggagaacacgaagatcctgatcggcgaggtcacggtcgtcgacgagagcgaacccttcgcccacgagaagctgtccccgaccctggcgatgtaccgggcgaaagacttcgaagacgcggtcgagaaggccgagaaactggtggcgatgggcggcatcggccatacgagctgtctgtacaccgaccaggacaatcagcccgcccgcgtgtcctacttcggccagaaaatgaagaccgcccgcatcctcatcaatacgcccgcgagccagggcggcatcggcgatctctacaatttcaagctcgcgccgtcgctgaccctcggctgcggctcctggggcggcaactccatctccgagaacgtgggcccgaaacacctcatcaacaagaagaccgtggccaagcgggcggagaacatgctgtggcataagctgcccaagtcgatctacttccgccggggctcgctgcccatcgcgctggacgaagtcatcacggacggccacaagcgggcgctgatcgtgacggaccgcttcctgttcaataacggctacgccgaccagatcacctccgtcctgaaggccgcgggcgtcgaaacggaagtcttcttcgaagtcgaagccgacccgaccctctccatcgtccggaagggcgccgagctggccaattccttcaagccggatgtcatcatcgcgctgggcggcggctcgccgatggacgccgccaaaatcatgtgggtgatgtacgagcaccccgaaacgcacttcgaagaactggcgctgcgcttcatggatatccgcaagcggatctacaaattcccgaaaatgggcgtgaaagcgaagatgatcgcggtgacgaccaccagcggcacgggctccgaggtgacgccgttcgcggtcgtcaccgatgatgccaccggccagaaataccccctggccgactacgccctgacccccgacatggccatcgtggacgccaatctcgtgatggacatgccgaagagcctctgcgccttcggcggcctggatgccgtgacgcatgcgatggaggcgtatgtcagcgtgctggcgtccgagttctcggacggccaggcgctgcaggccctgaagctgctgaaggagtacctgccggcctcctaccacgagggctccaaaaacccggtggcccgggagcgggtccactcggcggccaccatcgcgggcatcgccttcgcgaacgccttcctcggcgtgtgccattcgatggcccacaagctgggctcccagttccatatcccccacggcctggcgaacgcgctgctgatctgtaacgtcatccggtacaacgcgaacgataacccgaccaaacagaccgccttctcccagtatgaccgcccccaggcccggcgccgctatgcggaaatcgccgaccatctcggcctgtcggccccgggcgaccgcaccgccgccaagatcgagaagctgctggcctggctcgaaacgctgaaggccgaactgggcatcccgaagtcgatccgcgaagccggcgtccaggaagcggacttcctggcgaatgtggataaactgagcgaggacgccttcgatgaccagtgcacgggcgccaacccgcgctacccgctcatcagcgagctgaaacagatcctgctcgacacctattatggccgcgattacgtcgagggcgaaaccgccgcgaagaaggaggccgccccggccaaagccgaaaagaaggccaagaaaagcgcctga

SEQ ID NO: 68 - DNA, 1164 bp, Escherichia coli; identical to SEQ ID NO: 31
  source 1..1164: mol_type = genomic DNA

SEQ ID NO: 69 - DNA, 1149 bp, Escherichia coli; identical to SEQ ID NO: 32
  source 1..1149: mol_type = genomic DNA

SEQ ID NO: 70 - DNA, 1407 bp, Clostridium beijerinckii
  source 1..1407: mol_type = genomic DNA
atgaacaaggacacgctgatccccaccacgaaagacctcaaggtgaagaccaacggcgagaacatcaacctgaaaaactacaaggacaacagcagctgcttcggcgtgttcgaaaacgtcgaaaacgccatcagctcggcggtgcatgcccagaaaatcctcagcctgcactatacgaaggaacagcgcgaaaagatcatcaccgaaatccgcaaggccgcgctccagaacaaagaggtgctggccacgatgatcctggaggaaacccatatgggccgctacgaagataaaatcctgaagcacgagctggtggcgaaatacacgcccggcaccgaagacctgacgaccaccgcctggagcggcgataacggcctgacggtggtggagatgtcgccgtatggcgtgatcggcgcgatcaccccgtccaccaacccgaccgaaacggtcatctgtaactcgatcggcatgatcgcggccggcaacgcggtcgtgttcaatggccacccgtgcgccaagaagtgcgtcgccttcgccgtcgagatgatcaacaaggccatcatctcctgcggcggcccggagaacctcgtgacgacgatcaaaaacccgacgatggagtccctggatgccatcatcaagcacccgtccatcaagctgctctgcggcaccggcggcccgggcatggtcaaaacgctgctgaactccggcaaaaaagccatcggcgcgggcgcgggcaacccccccgtcatcgtggatgacaccgccgacatcgagaaagccggccggtcgatcatcgaaggctgctccttcgacaataacctcccgtgcatcgccgagaaagaggtcttcgtcttcgagaacgtcgcggacgatctgatctccaacatgctgaagaacaacgccgtcatcatcaacgaagaccaggtgtccaagctgatcgacctcgtgctgcagaagaacaatgaaacccaggagtacttcatcaacaagaagtgggtcggcaaggacgcgaagctgttcctggacgaaatcgatgtggagtccccgagcaatgtgaagtgcatcatctgcgaggtcaacgcgaaccaccccttcgtcatgaccgagctcatgatgccgatcctgccgatcgtgcgggtgaaggatatcgacgaagccatcaagtacgcgaaaatcgcggaacagaatcggaagcattcggcctatatctacagcaagaacatcgataacctcaaccgcttcgaacgggagatcgacaccaccatcttcgtcaagaacgccaaatccttcgccggcgtgggctatgaggcggaaggcttcaccaccttcacgatcgccggctccaccggcgagggcatcacctccgcccggaacttcacccgccagcgccgctgcgtgctggcgggctga

SEQ ID NO: 71 - DNA, 1083 bp, Saccharomyces cerevisiae; identical to SEQ ID NO: 30
  source 1..1083: mol_type = genomic DNA

SEQ ID NO: 72 - DNA, 1023 bp, Acinetobacter baylyi
  source 1..1023: mol_type = genomic DNA
atgacgaccaacgtcatccatgcctatgcggccatgcaggccggcgaggcgctggtgccgtatagcttcgacgcgggcgagctccagccgcatcaggtcgaggtgaaggtggaatactgcggcctgtgtcattccgatgtgtcggtcctgaataacgaatggcactcctccgtctatccggtggtcgccggccacgaggtcatcggcacgatcacccagctgggcagcgaagccaagggcctgaagatcggccagcgcgtcggcatcggctggacggccgagagctgccaggcctgcgatcagtgcatctccggccagcaggtgctgtgcaccggcgagaacaccgccaccatcatcggccatgccggcggcttcgcggataaagtgcgggccggctggcagtgggtgatcccgctcccggatgagctcgatcccacgagcgccggcccgctgctgtgtggcggcatcaccgtgttcgacccgatcctgaagcaccagatccaggccatccaccatgtcgccgtgatcggcatcggcggcctgggccacatggcgatcaagctgctcaaggcctggggctgtgaaatcaccgccttcagcagcaatcccaacaagaccgacgaactgaaggcgatgggcgccgaccatgtggtcaactcccgcgatgacgcggaaatcaagagccagcagggcaagttcgacctgctgctgtcgacggtgaatgtccccctcaactggaacgcctacctgaataccctggccccgaacggcacgttccacttcctgggcgtcgtcatggagccgatcccggtgccggtgggcgccctcctgggcggcgcgaaaagcctgaccgcctcgcccacgggcagccccgccgccctccgcaagctgctggagttcgccgcccgcaagaacatcgcgccccagatcgaaatgtatcccatgagcgagctgaacgaagccatcgagcgcctgcatagcggccaggcccgctatcggatcgtgctcaaagcggacttctga

SEQ ID NO: 73 - DNA, 1020 bp, Escherichia coli; identical to SEQ ID NO: 36
  source 1..1020: mol_type = genomic DNA

SEQ ID NO: 74 - DNA, 1167 bp, Escherichia coli
  source 1..1167: mol_type = genomic DNA
atgaacctgcatgaatatcaggccaagcagctgttcgcgcggtatggcctcccggcgccggtcggctacgcctgtacgaccccgcgggaagcggaggaggccgcctccaagatcggcgccggcccgtgggtggtcaaatgccaggtgcatgcgggcggccggggcaaggcgggcggcgtgaaggtcgtcaactccaaggaggacatccgcgccttcgccgagaactggctgggcaagcggctggtgacctatcagacggacgccaatggccagcccgtcaatcagatcctggtcgaggcggccacggacatcgcgaaagaactgtacctcggcgccgtcgtggaccggagcagccggcgggtggtgttcatggcgtccaccgagggcggcgtggaaatcgaaaaagtggccgaggaaaccccgcacctgatccataaagtcgcgctggacccgctgaccggccccatgccgtatcagggccgggaactcgcgttcaagctcggcctggagggcaagctggtgcagcagttcacgaaaatcttcatgggcctggcgaccatcttcctggagcgcgacctggccctgatcgaaatcaacccgctggtcatcacgaagcagggcgacctgatctgcctggacggcaagctcggcgccgacggcaacgccctgttccgccagccggacctgcgggaaatgcgcgatcagtcgcaggaggacccccgggaggcccaggcggcccagtgggagctgaattatgtggcgctcgatggcaatatcggctgcatggtcaatggcgcgggcctggcgatgggcacgatggacatcgtgaagctgcatggcggcgagcccgccaacttcctggacgtgggcggcggcgcgaccaaagagcgggtgacggaagcgttcaagatcatcctgagcgacgataaagtcaaggccgtgctggtcaacatcttcggcggcatcgtccgctgcgacctgatcgccgacggcatcatcggcgcggtggcggaggtcggcgtcaatgtgccggtcgtggtccgcctggagggcaacaacgccgaactgggcgccaagaagctggccgattccggcctgaacatcatcgcggcgaagggcctgaccgatgccgcgcagcaggtcgtggccgccgtcgagggcaagtga

SEQ ID NO: 75 - DNA, 870 bp, Escherichia coli
  source 1..870: mol_type = genomic DNA
atgtccatcctcatcgacaagaacaccaaggtgatctgccagggcttcaccggctcccagggcaccttccacagcgagcaggcgatcgcctacggcaccaagatggtgggcggcgtcacccccggcaagggcggcaccacccatctcggcctcccggtgttcaatacggtgcgggaagccgtggcggccaccggcgcgaccgccagcgtcatctatgtgcccgcgccgttctgcaaggactccatcctggaagccatcgacgccggcatcaagctcatcatcaccatcaccgagggcatccccaccctcgacatgctgacggtgaaagtcaagctggacgaggcgggcgtccggatgatcggccccaactgcccgggcgtcatcacccccggcgagtgcaaaatcggcatccagccgggccacatccacaaaccgggcaaggtgggcatcgtctcgcgctccggcaccctcacctatgaagccgtcaagcagaccaccgactatggcttcggccagtcgacctgcgtcggcatcggcggcgaccccatcccgggctcgaacttcatcgacatcctggagatgttcgagaaagacccccagaccgaggccatcgtgatgatcggcgagatcggcggctcggcggaggaggaggcggcggcgtacatcaaagagcatgtgaccaagccggtcgtgggctatatcgcgggcgtgacggcgcccaagggcaagcgcatgggccatgcgggcgccatcatcgcgggcggcaaaggcaccgcggatgagaagttcgccgccctggaggccgccggcgtcaagaccgtccgcagcctcgccgatatcggcgaagccctgaagaccgtgctcaagtga

SEQ ID NO: 76 - DNA, 1362 bp, Clostridium kluyveri
  source 1..1362: mol_type = genomic DNA
atgtcgaatgaagtctcgatcaaggaactgatcgaaaaggcgaaggtggcccagaagaaactggaagcctactcgcaggagcaggtggatgtcctggtcaaggccctgggcaaggtcgtgtatgataacgcggagatgttcgccaaagaagcggtggaagaaacggaaatgggcgtgtacgaggacaaggtggcgaagtgccacctgaaatccggcgccatctggaaccatatcaaggacaagaaaaccgtgggcatcatcaaggaagagccggagcgggccctggtctacgtcgccaagcccaagggcgtggtggccgccaccacgcccatcaccaaccccgtcgtcacgccgatgtgtaatgcgatggccgcgatcaagggccgcaatacgatcatcgtggccccgcaccccaaagcgaaaaaggtgtccgcccacaccgtcgagctgatgaacgccgagctgaagaagctgggcgccccggagaacatcatccagatcgtggaagcgccctcgcgggaggcggcgaaggaactcatggagagcgccgacgtggtcatcgccaccggcggcgcgggccgggtgaaggccgcctactcgtccggccgccccgcgtacggcgtcggcccgggcaactcgcaggtcatcgtcgacaagggctatgactacaacaaggccgcgcaggacatcatcaccggccgcaagtatgacaacggcatcatctgctcctccgagcagagcgtgatcgcgcccgccgaagactatgacaaggtgatcgccgccttcgtcgagaatggcgcgttctacgtggaggatgaagaaaccgtggaaaagttccgctccaccctgttcaaagacggcaagatcaattccaagatcatcggcaagagcgtccagatcatcgccgacctggcgggcgtcaaggtccccgagggcacgaaggtcatcgtgctgaagggcaagggcgcgggcgagaaggatgtcctgtgcaaggaaaagatgtgcccggtgctggtggccctgaagtacgacaccttcgaagaggccgtcgaaatcgcgatggccaactatatgtatgaaggcgccggccacaccgccggcatccattcggacaacgacgagaacatccgctacgcgggcaccgtgctccccatcagccgcctcgtggtgaaccagccggccaccaccgccggcggcagcttcaataacggcttcaaccccaccaccaccctgggctgcggcagctggggccgcaacagcatctcggaaaacctcacgtacgaacatctcatcaacgtctcccgcatcggctatttcaacaaggaagcgaaggtgccctcctatgaagagatctggggctga

SEQ ID NO: 77 - DNA, 1356 bp, Porphyromonas gingivalis
  source 1..1356: mol_type = genomic DNA
atggagatcaaggaaatggtgtccctggcccgcaaggcccagaaggagtaccaggccacccataatcaggaagccgtggacaacatctgccgcgccgcggcgaaagtcatctacgaaaatgccgccatcctcgcccgcgaggcggtcgatgaaaccggcatgggcgtgtacgaacacaaggtggccaagaatcagggcaaaagcaagggcgtgtggtacaacctgcataacaagaaaagcatcggcatcctgaacatcgacgagcgcaccggcatgatcgagatcgccaagcccatcggcgtcgtcggcgcggtcacgccgaccaccaatcccatcgtcaccccgatgtccaacatcatcttcgcgctgaaaacctgcaacgcgatcatcatcgccccccacccgcgcagcaaaaaatgctcggcccacgccgtgcggctcatcaaggaagccatcgcgccgttcaacgtgccggagggcatggtgcagatcatcgaagagccgtcgatcgagaagacccaggaactgatgggcgccgtcgacgtggtggtggccaccggcggcatgggcatggtgaagtcggcgtatagctcgggcaagccgagcttcggcgtgggcgccggcaacgtgcaggtcatcgtcgattcgaacatcgatttcgaagccgccgcggaaaagatcatcacgggccgcgcgttcgataacggcatcatctgctccggcgaacagagcatcatctacaacgaggcggataaggaggccgtcttcacggccttccggaaccacggcgcgtacttctgcgatgaagccgaaggcgatcgcgcccgggcggcgatcttcgaaaacggcgccatcgccaaagacgtggtgggccagagcgtggcgttcatcgccaagaaagcgaacatcaatatccccgaaggcacgcgcatcctggtggtggaagcccgcggcgtcggcgcggaagatgtgatctgcaaggaaaagatgtgccccgtgatgtgcgccctgtcctacaaacacttcgaagagggcgtcgagatcgcccgcaccaacctcgcgaacgagggcaacggccacacctgcgccatccattcgaataatcaggcccacatcatcctggccggcagcgagctcaccgtcagccgcatcgtggtcaacgcgccctccgccaccaccgccggcggccatatccagaacggcctggccgtgacgaacaccctgggctgcggctcgtggggcaacaacagcatctcggaaaacttcacctataagcatctgctcaatatcagccgcatcgccccgctgaacagcagcatccatatcccggacgacaaagagatctgggaactctga

SEQ ID NO: 78 - DNA, 287 bp, synthetic construct
  misc_feature 1..287: note = Variant of mxaF promoter
  source 1..287: mol_type = other DNA
gaggttcaggcgaaaccgcagactcaagggcgcttgctcccgggaaagatcgtattagtttgcctcgatcggcggtccttgtgacagggagatattcccgacggatccggggcattcgagcggaaccgcccgccgtgggagtttttccagcgagcattcgagagtttttcaaggcggcttcgaggggttattccgtaacgccgccgacatgatctgtcccggaatctccgccgctgttcgtagagcgccgatgcagggtcggcatcaatcattcttggaggagacac

SEQ ID NO: 79 - DNA, 4107 bp, synthetic construct
  misc_feature 1..4107: note = Cas9 sequence used in Expression Vector
  source 1..4107: mol_type = other DNA
atggacaagaagtattcgatcggcctggacatcggcaccaacagcgtcggctgggcggtcatcacggatgagtacaaggtgccgtcgaagaagttcaaggtgctgggcaataccgaccgccatagcatcaagaagaatctcatcggcgcactgctgttcgactccggcgaaaccgccgaagcgacccgcctcaagcgcacggcccggcggcgctatacgcgccggaagaaccgcatctgctacctccaggaaatcttctccaacgagatggccaaggtggatgactccttcttccatcgcctggaagaatccttcctggtcgaagaagataagaaacatgagcgccaccccatcttcggcaatatcgtggacgaggtggcgtatcacgagaaatacccgaccatctatcacctgcggaaaaagctggtggactcgacggacaaagccgacctgcgcctcatctatctggccctggcccacatgatcaagttccggggccatttcctgatcgaaggcgacctgaaccccgataacagcgacgtggacaagctcttcatccagctcgtccagacctataaccagctgttcgaggagaaccccatcaacgcctcgggcgtggacgccaaggccatcctgagcgcacggctctccaagtcgcgccgcctggaaaacctgatcgcgcagctgccgggcgaaaagaaaaacggcctgttcggcaacctgatcgccctgtccctcggcctcaccccgaacttcaagtccaacttcgacctggccgaggacgcgaagctccagctgtcgaaagacacctacgatgacgacctggacaacctcctggcgcagatcggcgaccagtacgccgacctcttcctcgcggccaagaatctgtcggacgccatcctgctgtcggatatcctgcgggtgaatacggagatcacgaaggcccccctctcggcctcgatgatcaagcgctacgacgagcaccatcaggacctgacgctgctcaaggccctcgtccggcagcagctgccggagaagtataaagagatcttcttcgaccagtccaagaacggctacgcgggctacatcgacggcggcgcgtcgcaggaggagttctataaattcatcaagccgatcctggagaaaatggacggcaccgaagaactcctcgtcaagctgaaccgggaggatctgctccgcaagcagcgcaccttcgacaatggctccatcccgcaccagatccatctcggcgagctgcacgccatcctgcgccgccaggaggacttctaccccttcctcaaagacaaccgggagaaaatcgagaagatcctgacgttccgcatcccctactacgtgggccccctcgcccgcggcaactcgcggttcgcgtggatgacccggaagagcgaggagacgatcaccccgtggaatttcgaggaggtcgtcgataaaggcgcgtcggcgcagtcgttcatcgagcgcatgaccaacttcgataaaaatctgccgaacgaaaaagtcctgcccaagcatagcctgctgtacgagtacttcacggtctacaacgagctgacgaaagtgaaatatgtcacggagggcatgcgcaaaccggccttcctgtccggcgagcagaaaaaggccatcgtggatctgctgttcaagacgaaccggaaggtcaccgtgaaacagctgaaggaagattacttcaagaaaatcgagtgcttcgattccgtcgaaatcagcggcgtggaggaccgcttcaatgcctcgctgggcacctatcacgatctcctcaagatcatcaaggacaaggacttcctggacaacgaagagaacgaggacatcctggaagacatcgtcctcaccctgaccctgttcgaggaccgcgaaatgatcgaagagcgcctgaagacctacgcccacctgttcgacgacaaggtcatgaagcagctcaagcgccgccggtacaccggctggggccgcctgtcccggaagctgatcaacggcatccgcgataagcagagcggcaagacgatcctggacttcctcaagagcgacggcttcgccaatcggaatttcatgcagctcatccacgacgatagcctgaccttcaaagaggatatccagaaggcgcaggtgtccggccagggcgacagcctgcacgaacatatcgccaacctggcgggctcccccgcgatcaagaaaggcatcctccagacggtcaaagtcgtggacgagctggtcaaggtgatgggccgccacaaaccggagaatatcgtcatcgagatggcacgcgagaaccagaccacgcagaagggccagaagaacagccgggaacgcatgaaacggatcgaagagggcatcaaggaactgggctcgcagatcctgaaggagcaccccgtcgaaaacacgcagctccagaacgagaagctgtatctgtactatctccagaacggccgggacatgtatgtcgatcaggaactggatatcaaccgcctctccgattacgatgtggaccacatcgtgccgcagagcttcctgaaagacgactcgatcgacaacaaggtcctgacccggtcggacaagaaccgcggcaagtcggataacgtgccgtcggaagaagtcgtgaaaaagatgaagaactactggcggcagctcctgaacgcgaagctcatcacgcagcgcaagttcgacaatctgaccaaggccgagcgcggcggcctctcggaactcgacaaggcgggcttcatcaaacggcagctcgtcgagacgcgccagatcaccaaacacgtggcccagatcctggacagccggatgaacaccaaatacgacgaaaacgacaagctgatccgcgaagtcaaggtcatcacgctgaagagcaagctggtgtcggatttccgcaaggatttccagttctacaaggtgcgcgagatcaacaattaccatcacgcgcacgatgcctatctcaatgcggtcgtgggcaccgccctgatcaaaaagtacccgaaactggagtccgagttcgtctacggcgactataaggtctatgatgtccgcaagatgatcgccaaatcggagcaggagatcggcaaggcgaccgcgaaatatttcttctactcgaacatcatgaatttcttcaagaccgagatcacgctggcgaacggcgaaatccgcaagcggcccctgatcgaaaccaatggcgagaccggcgagatcgtgtgggacaaaggccgggatttcgccaccgtccgcaaggtcctctcgatgccgcaggtgaacatcgtcaagaagacggaggtccagaccggcggcttcagcaaagaaagcatcctccccaagcggaatagcgacaaactgatcgcccggaagaaggactgggacccgaagaagtatggcggcttcgatagccccaccgtcgcctattccgtcctggtggtggcgaaggtggagaaaggcaaaagcaagaaactgaagagcgtgaaggagctgctgggcatcaccatcatggaacgcagcagcttcgagaagaacccgatcgacttcctggaagccaaaggctataaggaagtgaagaaggacctcatcatcaaactcccgaagtattcgctgttcgagctggaaaatggccgcaaacggatgctcgcctccgcgggcgaactccagaagggcaacgaactggcgctgccgtccaaatacgtcaacttcctctatctggccagccattacgaaaagctgaagggctcgcccgaagataacgagcagaaacagctgttcgtcgagcagcacaagcactacctcgacgagatcatcgagcagatcagcgagttctccaagcgggtgatcctcgcggacgccaacctggacaaggtgctgtcggcgtacaacaaacatcgggataagccgatccgcgagcaggccgaaaatatcatccacctgttcaccctgacgaacctcggcgcccccgccgccttcaagtatttcgataccaccatcgaccggaagcgctatacctccaccaaagaggtcctggatgccaccctcatccaccagtccatcacgggcctgtacgagacccgcatcgacctgtcgcagctgggcggcgactaa

SEQ ID NO: 80 - DNA, 35 bp, synthetic construct
  misc_feature 1..35: note = J23111 Promoter-gRNA sequence
  source 1..35: mol_type = other DNA
ttgacggctagctcagtcctaggtatagtgctagc

SEQ ID NO: 81 - DNA, 83 bp, synthetic construct
  misc_feature 1..83: note = Cas9 handle and terminator region
  source 1..83: mol_type = other DNA
gttttagagctagaaatagcaagttaaaataaggctagtccgttatcaacttgaaaaagtggcaccgagtcggtgcttttttt

SEQ ID NO: 82 - DNA, 20 bp, synthetic construct
  misc_feature 1..20: note = pSL148 - Targeting Sequence
  source 1..20: mol_type = other DNA
gctcgaagcccattaccacg

SEQ ID NO: 83 - DNA, 138 bp, synthetic construct
  misc_feature 1..138: note = pSL148 - Full sgRNA sequence
  source 1..138: mol_type = other DNA
ttgacggctagctcagtcctaggtatagtgctagcgctcgaagcccattaccacggttttagagctagaaatagcaagttaaaataaggctagtccgttatcaacttgaaaaagtggcaccgagtcggtgcttttttt

SEQ ID NO: 84 - DNA, 201 bp, synthetic construct
  misc_feature 1..201: note = pSL148 - Donor Sequence
  source 1..201: mol_type = other DNA
gttcgacgccgaattcgaagccatcaagcaccaggccggggtcgccgccgacatcggcctgagcgccgtccatctcgccgacatcggcgaacgtttcctcgccgtcgtacgccgccataccggcaagcctttccccgaggacgtctacgagcagctcgagatcgcgatccgggcggtattcgactcctggatgggaaacgc

SEQ ID NO: 85 - DNA, 20 bp, synthetic construct
  misc_feature 1..20: note = pSL150 - Targeting Sequence
  source 1..20: mol_type = other DNA
tcaaccacgatacactccag

SEQ ID NO: 86 - DNA, 138 bp, synthetic construct
  misc_feature 1..138: note = pSL150 - Full sgRNA sequence
  source 1..138: mol_type = other DNA
ttgacggctagctcagtcctaggtatagtgctagctcaaccacgatacactccaggttttagagctagaaatagcaagttaaaataaggctagtccgttatcaacttgaaaaagtggcaccgagtcggtgcttttttt

SEQ ID NO: 87 - DNA, 199 bp, synthetic construct
  misc_feature 1..199: note = pSL150 - Donor Sequence
  source 1..199: mol_type = other DNA
gtgaccgagcctatctggaattgctcaaggccctggcccagttgcgcctgctggaacggctgaatgctcgaaaaatgggccggaaccggctctgatcctgcagtgacaagcggcgaacgcgacagttcgaggagaccatcatgacgatgaaaaagcgtgtctacgccttctccgaaggcgacggcaagaacaaacgcct

SEQ ID NO: 88 - DNA, 20 bp, synthetic construct
  misc_feature 1..20: note = pSL128 - Targeting Sequence
  source 1..20: mol_type = other DNA
ggaccagaacaatccgttgt

SEQ ID NO: 89 - DNA, 138 bp, synthetic construct
  misc_feature 1..138: note = pSL128 - Full sgRNA sequence
  source 1..138: mol_type = other DNA
ttgacggctagctcagtcctaggtatagtgctagcggaccagaacaatccgttgtgttttagagctagaaatagcaagttaaaataaggctagtccgttatcaacttgaaaaagtggcaccgagtcggtgcttttttt

SEQ ID NO: 90 - DNA, 1044 bp, synthetic construct
  misc_feature 1..1044: note = pSL128 - Donor Sequence
  source 1..1044: mol_type = other DNA
taccatgcgcatcgatctgaccgagacgcagctggatgccctggtggaaatctaccggatgatgcggccgggcgaaccgccgaccaaggaggccgcccagaccctgttcgaaaatctgtttttctcggccgaacgctatgatctgtcggctgtcggccggatgaagttcaaccggcgcctggggcggaccgatcctaccggccccggcgtgctggaaaacgatgacatcatcgcggtgctgaaggaactgatcaacatccgtaacggggggggcacggtcgacgacatcgaccatctgggtaaccgccgcgtccggtccgtgggggagatggtggagaatcagttcaggctcgggctggtccgggtcgaacgggccgtgaaggagcgtctgtcgctgcccgacgccgatggtctgatgccacaggagatcatcaacgccaaaccggtggccgcttccatcaaggaattcttcggttcgagccagctttcgcggttcatggaccagaataacccattatctgaggtcactcacaagcgccgtgtctcggctctggggccgggggggctggcgcgtgagcgcgccggcttcgaagtgcgcgacgtgcataccacccactacggccgtgtctgtccgatcgaaacgcccgaaggtccgaacatcggcctgatcaactcgctcgcggtctactcgcgcaccaacgaatacggctttctggagacgccgtatcgaaaggtgatcgacggccgggtgaccgatcagatcgagtacctgtcggctatcgaggagggccagtactacattgctcaggccagcgcctcggtcgatgaaaacggcatgctcaaggatgaactggtgtcgtgtcgccacaaggatgaattcaccctggcgtcgcgggaaaacatcaactacatggatgtgtcgtccaaacagatcgtgtcggtcgcggcctcgctgatccccttcctcgaacacgatgatgccaaccgcgccctgatgggctcgaacatgcagcggcaggccgtcccgacgctgcgtacggagaa

SEQ ID NO: 91 - DNA, 20 bp, synthetic construct
  misc_feature 1..20: note = gap gRNA5 - Targeting Sequence
  source 1..20: mol_type = other DNA
cgtcgtgcatgagtgcaccg

SEQ ID NO: 92 - DNA, 138 bp, synthetic construct
  misc_feature 1..138: note = gap gRNA5 - Full sgRNA sequence
  source 1..138: mol_type = other DNA
ttgacggctagctcagtcctaggtatagtgctagccgtcgtgcatgagtgcaccggttttagagctagaaatagcaagttaaaataaggctagtccgttatcaacttgaaaaagtggcaccgagtcggtgcttttttt

SEQ ID NO: 93 - DNA, 1307 bp, synthetic construct
  misc_feature 1..1307: note = gap gRNA5 - Donor Sequence
  source 1..1307: mol_type = other DNA
ccgggacggaacgcttgccaggccatctggatgtggcggggaccggcgccgtcgacgaacacgatctcgaggtcgtcggccaccgccagatagagcatgttgcgaatggggacgaacagtgggtcggggcgccggcggccgagcagtacccggctgaggttgtaggttgcggcgccgagcaggcgtgcctcgcgtcggagttcgcgcgaacgatggaaggtttcctgcatgtggatggcgtcgcttgctgggtcccggtttaagctatcagccttggtgcgcgaagggcaagaactggacgctcggcgcggcgctttgctatattggccacgctttgtattgacagattccccggagaatgcccgattctccctcgccgaaggcccgcaggcgtccggccgggacggcctcgactgctctccccaccccactccgccggtcgccaagggtgcgagggctctcgcgcatgaccccatcctcactttatttcagcatttctggagcagggcaatgacgattaagattgcaatcaatggatatgggcgcatcggccgcaacatcctgcgggcgatttacgaaaccgggcgcaaggatgtggagatcgtcgccatcaatgacctgggggatgcccagatcaacgcccatctcacccgccatgacaccgtgcacgggccgttccgggggaccgtggaggtcggcgagggcgaaatcatcgtcaacggcgaccgcatcagggttttttccgagaaggatccttccaagctgccctggggggctttgggcgtggacgtcgtgcatgagtgatagcacctgttccgcaccaaggccaaatgccagccgcatctcgatgccggcgccaagaaggtgatcatttcggccccggccgacaagaacgagtgcgacgcgaccatcgtctacggggtcaatgagcatacgctgaaagccgcccacaccgtcatctcgaatgcatcctgcaccaccaactgcctggcgccgctggtcaagccgctgctgggaaaaatcgggatcgtgtccggcctcatgaccaccgtgcattcctacaccaacgaccaggtgctcaccgacgtttatcacaaggatctgtaccgggcacgggcggcggcgctgaacatgatcccgaccaagaccggcgcggcgcaggccgtggggctggtgctgccggagctggacggcaaactgtccggtttcgccatccgggtgccgaccgccaatgtatcggtcgtggacctgaccttcatcgcggcccgggaaaccgacaaggacgagatcaacgccatcctcaaggc

SEQ ID NO: 94 - DNA, 20 bp, synthetic construct
  misc_feature 1..20: note = pIK11 - Targeting Sequence
  source 1..20: mol_type = other DNA
cccatcacctatcagcacaa

SEQ ID NO: 95 - DNA, 138 bp, synthetic construct
  misc_feature 1..138: note = pIK11 - Full sgRNA sequence
  source 1..138: mol_type = other DNA
ttgacggctagctcagtcctaggtatagtgctagccccatcacctatcagcacaagttttagagctagaaatagcaagttaaaataaggctagtccgttatcaacttgaaaaagtggcaccgagtcggtgcttttttt

SEQ ID NO: 96 - DNA, 1149 bp, synthetic construct
  misc_feature 1..1149: note = pIK11 - Donor Sequence
  source 1..1149: mol_type = other DNA
aagatcgatgacaccgtcaactgggtgaaaaaggtcgatctgaagaccggcctgccgatccgcgatccggagtacagcacccgcatggaccacaatgccaaaggcatctgtccctcggccatgggctatcacaaccagggcatcgagtcctacgatccggacaagaagctgttcttcatgggcgtgaaccacatctgcatggactgggagccgttcatgctgccctaccgcgccggccagttctttgtgggggcgaccctcaacatgtatccgggacccaaggggatgctgggtcaggtcaaggcgatgaacgcggtcaccggcaagatggaatgggaagtgccggagaagtttgcggtctggggtggcaccttggcgaccgccggcgacctcgtgttctacggtaccctcgacggcttcatcaaggcccgcgacacccgtaccggcgagctgaagtggcagttccagttgccctccggcgtgatcggccatcccatcacgtaccaacataacggcaagcaatacattgccatctactccggcgtcggcggctggccaggagtagggctggtattcgacctgaaggacccgaccgcaggtctgggagctgtgggtgcgttcagggaactggcgcattacacccagatgggtggatcggtgttcgtgttctcgctttgagtcgaaggggtggaggcgctcctgggggagcgcccctatcccatgctgtcgaaaggatgaatcatgcgaatgaaccgtattgcagccgcggggttggccgcctccctcgcggtcgtgggatgcgtgcaggcagcgacgagcgtcgaaccgctcaaggtctgctccgcggaaaacgagatgccgtattcggacaaggccggagagggtttcgaaaataagttggctgagctccttggaaagggattgggacggccagtcgagaacgtgtggtggaccgatgcccgctatttcgtccgggattatctggacaggggtttgtgcgatgtggtcatcggcgtcgataccggcgacccgcggatgctcaccagcagtccttattaccggtccggctacgtattcgtctaccgcaaggacacgggactgagcatccaagattggaacagcgcggcactgaagaccgtgaagcggatc

SEQ ID NO: 97 - DNA, 20 bp, synthetic construct
  misc_feature 1..20: note = pSL178 - Targeting Sequence
  source 1..20: mol_type = other DNA
tcaaccacgatacactccag

SEQ ID NO: 98 - DNA, 138 bp, synthetic construct; identical to SEQ ID NO: 86
  misc_feature 1..138: note = pSL178 - Full sgRNA sequence
  source 1..138: mol_type = other DNA

SEQ ID NO: 99 - DNA, 322 bp, synthetic construct
  misc_feature 1..322: note = pSL178 - Donor Sequence
  source 1..322: mol_type = other DNA
gtgttccctggaacacgccatcaatttcgatctcgtgctcaacaccgaccatctgccagccggtaacgctctgccgaccgtactcatggcggtacggcagttcggcttcgaaatcttcgatctcggtcagcgggaagcctcgtgagcccggcggtgggacagaccgcatcccgtctgctaacggtcgagatcgtcgacgtctgccgggagatattttccgggcgttgtagccgggtggtcgcgccagcggcagacggtgaggtcggcgttctgccccgtcatacgccgttcctgacccggctccggcccggcgagataaggc

SEQ ID NO: 100 - DNA, 20 bp, synthetic construct
  misc_feature 1..20: note = pSL179 - Targeting Sequence
  source 1..20: mol_type = other DNA
tcaaccacgatacactccag

SEQ ID NO: 101 - DNA, 138 bp, synthetic construct; identical to SEQ ID NO: 86
  misc_feature 1..138: note = pSL179 - Full sgRNA sequence
  source 1..138: mol_type = other DNA

SEQ ID NO: 102 - DNA, 2028 bp, synthetic construct
  misc_feature 1..2028: note = pSL179 - Donor sequence
  source 1..2028: mol_type = other DNA
gtgttccctggaacacgccatcaatttcgatctcgtgctcaacaccgaccatctgccagccggtaacgctctgccgaccgtactcatggcggtacggcagttcggcttcgaaatcttcgatctcggtcagcgggaagcctcgtgagcccggcggtgggacagaccgcatcccgtctgctaacggtcgagatcgtcgacgtctgccgggagatattttccgggcgttgtagccgggtggtcgcgccagcggcagacggtgaggtcggcgttctgccccgtcatacgccgttcctgacccggctccggcccggcgagataaggctcaggaccgaggcaggcgaagaccagtatttctacctctccgggggctacatggaggtgcagcgctgggaggtcagcatcctggccgaccaggtgctccgctcccaagagatcgaccgggaagcggccctggcggccaagcgcaacgcagagcggatgctccgcgagaaccggattcccggcgagcgtgaccgagcctatctggaattgctcaaggccctggcccagttgcgcctgctggaacggctgaatgctcgaaaaatgggccggaaccggctctgatcctgcagtgacaagcggcgaacgcgacagttcgaggagaccatcatgacgatgaaaaagcgtgtctacgccttctccgaaggcgacggcaagaacaaacgcctgctcggcggcaagggcgccaacctctgcgaaatgacgcagatcgggctcaacgtgccgccgggtttcgttattaccacggaagcctgcctcgaatacctggcagacaagaagctgccggccggcttgatggacgaagtccgggagcacatggcccggctcgaacgggctaccggcaagcgcttcggcgatcccgccaatccactcttggtttcggtgcgttccggttcggccctgtccatgccgggcatgatggataccattctcaacctcggcctcaaccacgatacactctttcctcgccgtcgtacgccgccataccggcaagcctttccccgaggacgtctacgagcagctcgagatcgcgatccgggcggtattcgactcctggatgggaaagcgcgcggtggattaccgccgcgaattccacatcacgcccgaccaggccaacggcacggcggtgaacgtggtgaccatggtgttcggcaacatgggcgacgactccgccaccggtgtcggcttcacccgcaatccgggtaccggtgagaacgagatgttcggcgagtatctggtcaacgcccagggtgaggatgtggtagccggaatccgcacgcccaagcccgtgcacgagatggcaaccgaaatgccggcgctttacgcccaactggtggaactgcgcgacaagctcgaagcccattaccacgaggtgcaggacttcgagtacaccatcgagaagggggtcttgtactgtctgcagacgcgcaacggcaagatgaacgcccaggcgatggtgcgcacctcggtcgagatgtgccgggaaggactgatcacgcgggatcaggccctcttgcgggtcaaccccgcccatctggaacagttactccatccctgcctcgacacctcgcacaaccccacgccgctggcgcaggggctgcctgcctcgcccggcgccgccagcggccgttgcgtgttcgatgcggatcaggccgaactgttgggacgggccggtgaaaaggtcatcctggtgcgtgaggagaccaagccggaagacatccacggcttcttcgcggcccagggaatcctcaccagtcgcggcggcaagacctcgcatgccgccgtggtcgcccgcggcatgggcaaggcctgcgtggccggggccgaaggcatcagggtggacagccgggcgcggctggcaacggtgggagaggtcacgttgcacgaaggtgacatcatcaccatcgacggcagcaccggccgtgtctatctcggcgcgatcccgacgatcgcgccgaccttctccgaacacctcaggacactgctgtc

SEQ ID NO: 103 - DNA, 20 bp, synthetic construct
  misc_feature 1..20: note = pSL162 - Targeting Sequence
  source 1..20: mol_type = other DNA
tcaaccacgatacactccag

SEQ ID NO: 104 - DNA, 138 bp, synthetic construct; identical to SEQ ID NO: 86
  misc_feature 1..138: note = pSL162 - Full sgRNA sequence
  source 1..138: mol_type = other DNA

SEQ ID NO: 105 - DNA, 199 bp, synthetic construct; identical to SEQ ID NO: 87
  misc_feature 1..199: note = pSL162 - Donor Sequence
  source 1..199: mol_type = other DNA

SEQ ID NO: 106 - DNA, 20 bp, synthetic construct
  misc_feature 1..20: note = pSL154 - Targeting Sequence
  source 1..20: mol_type = other DNA
tcaaccacgatacactccag

SEQ ID NO: 107 - DNA, 138 bp, synthetic construct; identical to SEQ ID NO: 86
  misc_feature 1..138: note = pSL154 - Full sgRNA sequence
  source 1..138: mol_type = other DNA

SEQ ID NO: 108 - DNA, 1075 bp, synthetic construct
  misc_feature 1..1075: note = pSL154 - Donor Sequence
  source 1..1075: mol_type = other DNA
gtgaccgagcctatctggaattgctcaaggccctggcccagttgcgcctgctggaacggctgaatgctcgaaaaatgggccggaaccggctctgatcctgcagtgacaagcggcgaacgcgacagttcgaggagaccatcatgacgatgaaaaagcgtgtctacgccttctccgaaggcgacggcaagaacaaacgcctgctcggcggcaagggcgccaacctctgcgaaatgacgcagatcgggctcaacgtgccgccgggtttcgttattaccacggaagcctgcctcgaatacctggcagacaagaagctgccggccggcttgatggacgaagtccgggagcacatggcccggctcgaacgggctaccggcaagcgcttcggcgatcccgccaatccactcttggtttcggtgcgttccggttcggccctgtccatgccgggcatgatggataccattctcaacctcggcctcaaccacgatacactctaagtgccagggcgtgcccttgggctccccgggcgcggggttgatccggcagaccggcaacgagcgcttcggtcacgatgcctaccggcggttcatccagttgttcggcaaggttgccctcggtgttcccgacgagctgttcgacgccgaattcgaagccatcaagcaccaggccggggtcgccgccgacatcggcctgagcgccgtccatctcgccgacatcggcgaacgtttcctcgccgtcgtacgccgccataccggcaagcctttccccgaggacgtctacgagcagctcgagatcgcgatccgggcggtattcgactcctggatgggaaagcgcgcggtggattaccgccgcgaattccacatcacgcccgaccaggccaacggcacggcggtgaacgtggtgaccatggtgttcggcaacatgggcgacgactccgccaccggtgtcggcttcacccgcaatccgggtaccggtgagaacgagatgttcggcgagtatctggtcaacgcccagggtgaggatgtggtagccggaatccgcacgcccaagcccgtgcacgagatggcaaccgaaatgc

SEQ ID NO: 109 - DNA, 20 bp, synthetic construct
  misc_feature 1..20: note = pSL175 - Targeting Sequence
  source 1..20: mol_type = other DNA
tactacggagcaagttcccg

SEQ ID NO: 110 - DNA, 138 bp, synthetic construct
  misc_feature 1..138: note = pSL175 - Full sgRNA sequence
  source 1..138: mol_type = other DNA
ttgacggctagctcagtcctaggtatagtgctagctactacggagcaagttcccggttttagagctagaaatagcaagttaaaataaggctagtccgttatcaacttgaaaaagtggcaccgagtcggtgcttttttt

SEQ ID NO: 111 - DNA, 1045 bp, synthetic construct
  misc_feature 1..1045: note = pSL175 - Donor Sequence
  source 1..1045: mol_type = other DNA
gccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattagctcccccaactgagagaactcaaaggttaccccagttggggcgggtaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcataacttcgtatagcatacattatacgaagttattggcagagcattacgctgacttgaccagaggctgcatttccaccgctgattgcgattcggaaggtgcaggccggagggtccggaccgccgctccacccgttgttttc

SEQ ID NO: 112 - DNA, 20 bp, synthetic construct
  misc_feature 1..20: note = pSL163 - Targeting Sequence
  source 1..20: mol_type = other DNA
tcaaccacgatacactccag

SEQ ID NO: 113 - DNA, 138 bp, synthetic construct; identical to SEQ ID NO: 86
  misc_feature 1..138: note = pSL163 - Full sgRNA sequence
  source 1..138: mol_type = other DNA

SEQ ID NO: 114 - DNA, 1785 bp, synthetic construct
  misc_feature 1..1785: note = pSL163 - Donor Sequence
  source 1..1785: mol_type = other DNA
gtgaccgagcctatctggaattgctcaaggccctggcccagttgcgcctgctggaacggctgaatgctcgaaaaatgggccggaaccggctctgatcctgcagtgacaagcggcgaacgcgacagttcgaggagaccatcatgacgatgaaaaagcgtgtctacgccttctccgaaggcgacggcaagaacaaacgcctgctcggcggcaagggcgccaacctctgcgaaatgacgcagatcgggctcaacgtgccgccgggtttcgttattaccacggaagcctgcctcgaatacctggcagacaagaagctgccggccggcttgatggacgaagtccgggagcacatggcccggctcgaacgggctaccggcaagcgcttcggcgatcccgccaatccactcttggtttcggtgcgttccggttcggccctgtccatgccgggcatgatggataccattctcaacctcggcctcaaccacgatacactctaattgacggctagctcagtcctaggtatagtgctagcaaaaagctcaacgagaggaagttccatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagggttgatccggcagaccggcaacgagcgcttcggtcacgatgcctaccggcggttcatccagttgttcggcaaggttgccctcggtgttcccgacgagctgttcgacgccgaattcgaagccatcaagcaccaggccggggtcgccgccgacatcggcctgagcgccgtccatctcgccgacatcggcgaacgtttcctcgccgtcgtacgccgccataccggcaagcctttccccgaggacgtctacgagcagctcgagatcgcgatccgggcggtattcgactcctggatgggaaagcgcgcggtggattaccgccgcgaattccacatcacgcccgaccaggccaacggcacggcggtgaacgtggtgaccatggtgttcggcaacatgggcgacgactccgccaccggtgtcggcttcacccgcaatccgggtaccggtgagaacgagatgttcggcgagtatctggtcaacgcccagggtgaggatgtggtagccggaatccgcacgcccaagcccgtgcacgagatggcaaccgaaatgc

SEQ ID NO: 115 - DNA, 20 bp, synthetic construct
  misc_feature 1..20: note = pSL165 - Targeting Sequence
  source 1..20: mol_type = other DNA
tcaaccacgatacactccag

SEQ ID NO: 116 - DNA, 138 bp, synthetic construct; identical to SEQ ID NO: 86
  misc_feature 1..138: note = pSL165 - Full sgRNA sequence
  source 1..138: mol_type = other DNA

SEQ ID NO: 117 - DNA, 2184 bp, synthetic construct
  misc_feature 1..2184: note = pSL165 - Donor sequence
  source 1..2184: mol_type = other DNA
gtgaccgagcctatctggaattgctcaaggccctggcccagttgcgcctgctggaacggctgaatgctcgaaaaatgggccggaaccggctctgatcctgcagtgacaagcggcgaacgcgacagttcgaggagaccatcatgacgatgaaaaagcgtgtctacgccttctccgaaggcgacggcaagaacaaacgcctgctcggcggcaagggcgccaacctctgcgaaatgacgcagatcgggctcaacgtgccgccgggtttcgttattaccacggaagcctgcctcgaatacctggcagacaagaagctgccggccggcttgatggacgaagtccgggagcacatggcccggctcgaacgggctaccggcaagcgcttcggcgatcccgccaatccactcttggtttcggtgcgttccggttcggccctgtccatgccgggcatgatggataccattctcaacctcggcctcaaccacgatacactctaattgacggctagctcagtcctaggtatagtgctagcaaaaagctcaacgagaggaagttccatgagctatcccgagaagttcgaggggatcgccatccagagccacgaggactggaagaacccgaaaaagaccaagtatgatccgaagcccttctacgatcacgacatcgacatcaagatcgaggcctgcggcgtctgcggcagcgatatccattgtgcggctggccactggggcaacatgaagatgccgttggtcgtcggccacgagatcgtgggcaaggtcgtgaagttaggcccgaaaagcaacagcggcttgaaggtgggccagcgcgtgggtgtgggtgcgcaggtcttcagctgtctggagtgcgaccgttgcaagaacgacaacgaaccgtactgcaccaagttcgtcaccacctactcgcagccctacgaggacggctacgtctcgcagggcggttacgccaactatgtccgagtccacgaacacttcgtggtgcccatcccggaaaatatccccagccatctggcggctcccctgctgtgcggtggcttgaccgtctacagccccctcgtccgcaatggctgcggtcccggcaagaaggtgggtatcgtgggcctcggcggtataggctctatgggcacgctgatctcgaaagcgatgggcgcagaaacgtacgtgatctcgcgttcctcgcgcaagcgcgaggatgcgatgaagatgggtgcggaccactacatcgccacgctggaggagggtgactggggtgagaagtacttcgacacgttcgacctcatcgtggtgtgcgcgagttccctgacggacatcgacttcaatatcatgcccaaggcgatgaaggtcggagggcgcatcgtctccatctcgatcccggagcagcacgaaatgctgtcgctgaagccctacggcctgaaagccgtctccattagctactcggcgctcggtagtatcaaggagctcaaccagctgttgaagttggtttccgaaaaggacatcaagatctgggtggaaacgctcccggtgggcgaagccggtgtgcacgaggcctttgagcggatggagaagggggatgtccgttatcggtttacactcgtcggctacgataaagagttctcggattaagggttgatccggcagaccggcaacgagcgcttcggtcacgatgcctaccggcggttcatccagttgttcggcaaggttgccctcggtgttcccgacgagctgttcgacgccgaattcgaagccatcaagcaccaggccggggtcgccgccgacatcggcctgagcgccgtccatctcgccgacatcggcgaacgtttcctcgccgtcgtacgccgccataccggcaagcctttccccgaggacgtctacgagcagctcgagatcgcgatccgggcggtattcgactcctggatgggaaagcgcgcggtggattaccgccgcgaattccacatcacgcccgaccaggccaacggcacggcggtgaacgtggtgaccatggtgttcggcaacatgggcgacgactccgccaccggtgtcggcttcacccgcaatccgggtaccggtgagaacgagatgttcggcgagtatctggtcaacgcccagggtgaggatgtggtagccggaatccgcacgcccaagcccgtgcacgagatggcaaccgaaatgc

SEQ ID NO: 118 - DNA, 4107 bp, synthetic construct
  misc_feature 1..4107: note = dCas9
  source 1..4107: mol_type = other DNA
atggacaagaagtattcgatcggcctggccatcggcaccaacagcgtcggctgggcggtcatcacggatgagtacaaggtgccgtcgaagaagttcaaggtgctgggcaataccgaccgccatagcatcaagaagaatctcatcggcgcactgctgttcgactccggcgaaaccgccgaagcgacccgcctcaagcgcacggcccggcggcgctatacgcgccggaagaaccgcatctgctacctccaggaaatcttctccaacgagatggccaaggtggatgactccttcttccatcgcctggaagaatccttcctggtcgaagaagataagaaacatgagcgccaccccatcttcggcaatatcgtggacgaggtggcgtatcacgagaaatacccgaccatctatcacctgcggaaaaagctggtggactcgacggacaaagccgacctgcgcctcatctatctggccctggcccacatgatcaagttccggggccatttcctgatcgaaggcgacctgaaccccgataacagcgacgtggacaagctcttcatccagctcgtccagacctataaccagctgttcgaggagaaccccatcaacgcctcgggcgtggacgccaaggccatcctgagcgcacggctctccaagtcgcgccgcctggaaaacctgatcgcgcagctgccgggcgaaaagaaaaacggcctgttcggcaacctgatcgccctgtccctcggcctcaccccgaacttcaagtccaacttcgacctggccgaggacgcgaagctccagctgtcgaaagacacctacgatgacgacctggacaacctcctggcgcagatcggcgaccagtacgccgacctcttcctcgcggccaagaatctgtcggacgccatcctgctgtcggatatcctgcgggtgaatacggagatcacgaaggcccccctctcggcctcgatgatcaagcgctacgacgagcaccatcaggacctgacgctgctcaaggccctcgtccggcagcagctgccggagaagtataaagagatcttcttcgaccagtccaagaacggctacgcgggctacatcgacggcggcgcgtcgcaggaggagttctataaattcatcaagccgatcctggagaaaatggacggcaccgaagaactcctcgtcaagctgaaccgggaggatctgctccgcaagcagcgcaccttcgacaatggctccatcccgcaccagatccatctcggcgagctgcacgccatcctgcgccgccaggaggacttctaccccttcctcaaagacaaccgggagaaaatcgagaagatcctgacgttccgcatcccctactacgtgggccccctcgcccgcggcaactcgcggttcgcgtggatgacccggaagagcgaggagacgatcaccccgtggaatttcgaggaggtcgtcgataaaggcgcgtcggcgcagtcgttcatcgagcgcatgaccaacttcgataaaaatctgccgaacgaaaaagtcctgcccaagcatagcctgctgtacgagtacttcacggtctacaacgagctgacgaaagtgaaatatgtcacggagggcatgcgcaaaccggccttcctgtccggcgagcagaaaaaggccatcgtggatctgctgttcaagacgaaccggaaggtcaccgtgaaacagctgaaggaagattacttcaagaaaatcgagtgcttcgattccgtcgaaatcagcggcgtggaggaccgcttcaatgcctcgctgggcacctatcacgatctcctcaagatcatcaaggacaaggacttcctggacaacgaagagaacgaggacatcctggaagacatcgtcctcaccctgaccctgttcgaggaccgcgaaatgatcgaagagcgcctgaagacctacgcccacctgttcgacgacaaggtcatgaagcagctcaagcgccgccggtacaccggctggggccgcctgtcccggaagctgatcaacggcatccgcgataagcagagcggcaagacgatcctggacttcctcaagagcgacggcttcgccaatcggaatttcatgcagctcatccacgacgatagcctgaccttcaaagaggatatccagaaggcgcaggtgtccggccagggcgacagcctgcacgaacatatcgccaacctggcgggctcccccgcgatcaagaaaggcatcctccagacggtcaaagtcgtggacgagctggtcaaggtgatgggccgccacaaaccggagaatatcgtcatcgagatggcacgcgagaaccagaccacgcagaagggccagaagaacagccgggaacgcatgaaacggatcgaagagggcatcaaggaactgggctcgcagatcctgaaggagcaccccgtcgaaaacacgcagctccagaacgagaagctgtatctgtactatctccagaacggccgggacatgtatgtcgatcaggaactggatatcaaccgcctctccgattacgatgtggacgccatcgtgccgcagagcttcctgaaagacgactcgatcgacaacaaggtcctgacccggtcggacaagaaccgcggcaagtcggataacgtgccgtcggaagaagtcgtgaaaaagatgaagaactactggcggcagctcctgaacgcgaagctcatcacgcagcgcaagttcgacaatctgaccaaggccgagcgcggcggcctctcggaactcgacaaggcgggcttcatcaaacggcagctcgtcgagacgcgccagatcaccaaacacgtggcccagatcctggacagccggatgaacaccaaatacgacgaaaacgacaagctgatccgcgaagtcaaggtcatcacgctgaagagcaagctggtgtcggatttccgcaaggatttccagttctacaaggtgcgcgagatcaacaattaccatcacgcgcacgatgcctatctcaatgcggtcgtgggcaccgccctgatcaaaaagtacccgaaactggagtccgagttcgtctacggcgactataaggtctatgatgtccgcaagatgatcgccaaatcggagcaggagatcggcaaggcgaccgcgaaatatttcttctactcgaacatcatgaatttcttcaagaccgagatcacgctggcgaacggcgaaatccgcaagcggcccctgatcgaaaccaatggcgagaccggcgagatcgtgtgggacaaaggccgggatttcgccaccgtccgcaaggtcctctcgatgccgcaggtgaacatcgtcaagaagacggaggtccagaccggcggcttcagcaaagaaagcatcctccccaagcggaatagcgacaaactgatcgcccggaagaaggactgggacccgaagaagtatggcggcttcgatagccccaccgtcgcctattccgtcctggtggtggcgaaggtggagaaaggcaaaagcaagaaactgaagagcgtgaaggagctgctgggcatcaccatcatggaacgcagcagcttcgagaagaacccgatcgacttcctggaagccaaaggctataaggaagtgaagaaggacctcatcatcaaactcccgaagtattcgctgttcgagctggaaaatggccgcaaacggatgctcgcctccgcgggcgaactccagaagggcaacgaactggcgctgccgtccaaatacgtcaacttcctctatctggccagccattacgaaaagctgaagggctcgcccgaagataacgagcagaaacagctgttcgtcgagcagcacaagcactacctcgacgagatcatcgagcagatcagcgagttctccaagcgggtgatcctcgcggacgccaacctggacaaggtgctgtcggcgtacaacaaacatcgggataagccgatccgcgagcaggccgaaaatatcatccacctgttcaccctgacgaacctcggcgcccccgccgccttcaagtatttcgataccaccatcgaccggaagcgctatacctccaccaaagaggtcctggatgccaccctcatccaccagtccatcacgggcctgtacgagacccgcatcgacctgtcgcagctgggcggcgactaa

SEQ ID NO: 119 - DNA, 20 bp, synthetic construct
  misc_feature 1..20: note = oriV_dcas9_gRNA3 - Targeting Sequence
  source 1..20: mol_type = other DNA
ataatgtgtggaattgtgag

SEQ ID NO: 120 - DNA, 138 bp, synthetic construct
  misc_feature 1..138: note = oriV_dcas9_gRNA3 - Full sgRNA sequence
  source 1..138: mol_type = other DNA
tttatagctagctcagcccttggtacaatgctagcataatgtgtggaattgtgaggttttagagctagaaatagcaagttaaaataaggctagtccgttatcaacttgaaaaagtggcaccgagtcggtgcttttttt

SEQ ID NO: 121 - DNA, 72 bp, synthetic construct
  misc_feature 1..72: note = Ptrc_laxZ - Promoter
  source 1..72: mol_type = other DNA
ttgacaattaatcatccggctcgtataatgtgtggaattgtgagcggataacaattaacgagaggaagttcc

SEQ ID NO: 122 - DNA, 3075 bp, synthetic construct
  misc_feature 1..3075: note = Ptrc_laxZ - LacZ sequence used in Vector
  source 1..3075: mol_type = other DNA
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcccgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 123 - DNA, 4077 bp, Methylococcus capsulatus
  source 1..4077: mol_type = genomic DNA
atggcttactcgtttaccgagaaaaaacgaattcgcaagagtttcggaaagcgtcaggacgtgctggaggtcccctatcttctggcgacacaggttgactcgtaccggcggtttttgcaactcgacaagcagcccgccgcccggagcgacgaaggtctgcatgcggcgttgaagtcggttttcccgatcaagagccattccggcaacatcgtactcgaatatgtgagttaccggctgggcgacccggtgttcgacgtcaaggaatgccagcagcgtggaaccacctacgccgccccgctgcgcgtgctcgtccgtctggtggtctacgacaaggatgcgccggtgggcgccaaggtcgtcagagacatcaaggagcaggagatctacatgggcgaaattccgctcatgaccgacaacggcacgttcgtcatcaacggtaccgagcgggtgatcgtttcccaactgcaccgttcgcccggcgtatttttcgaccatgatcggggaaagacccattcttcgggaaagctgctgttcaatgccagaatcatcccctaccgcgggtcctggctggacttcgaattcgaccacaaggactgcgtttacgtccgtatcgacaggcgtcgcaagctgcccgccacggtgttgctgcgagccctgggctatgacaacgagcagatcatcgccgagtttttcgacaccaaccgtttcctgctctcatccgccggcattcagctggaactgatacccgagcggctgcgcggcgatatcgccagcttcgacattcggcttggcgatcagatcgtggtcgagaaggaccaccggatcacggcgcgtcacatccggatgctgcaaaaggagaacgtgaacctcctcgacgtccccaaggactacctgtacggcaagattcttgcccacaacgtcgtggatacttcgacgggcgaactgatcgccaaggtgaatcaggagatcacggaggatgtctatgcgcgtctcgtggctgcggccatccccgagatccgtacgctctatgtgaacgatctcgaccgcggtccgtacatttccaataccatgcgcatcgatctgaccgagacgcagctggatgccctggtggaaatctaccggatgatgcggccgggcgaaccgccgaccaaggaggccgcccagaccctgttcgaaaatctgtttttctcggccgaacgctatgatctgtcggctgtcggccggatgaagttcaaccggcgcctggggcggaccgatcctaccggccccggcgtgctggaaaacgatgacatcatcgcggtgctgaaggaactgatcaacatccgtaacggggggggcacggtcgacgacatcgaccatctgggtaaccgccgcgtccggtccgtgggggagatggtggagaatcagttcaggctcgggctggtccgggtcgaacgggccgtgaaggagcgtctgtcgctgcccgacgccgatggtctgatgccacaggagatcatcaacgccaaaccggtggccgcttccatcaaggaattcttcggttcgagccagctttcgcagttcatggaccagaacaatccgttgtcggaggtcactcacaagcgccgtgtctcggctctggggccgggggggctggcgcgtgagcgcgccggcttcgaagtgcgcgacgtgcataccacccactacggccgtgtctgtccgatcgaaacgcccgaaggtccgaacatcggcctgatcaactcgctcgcggtctactcgcgcaccaacgaatacggctttctggagacgccgtatcgaaaggtgatcgacggccgggtgaccgatcagatcgagtacctgtcggctatcgaggagggccagtactacattgctcaggccagcgcctcggtcgatgaaaacggcatgctcaaggatgaactggtgtcgtgtcgccacaaggatgaattcaccctggcgtcgcgggaaaacatcaactacatggatgtgtcgtccaaacagatcgtgtcggtcgcggcctcgctgatccccttcctcgaacacgatgatgccaaccgcgccctgatgggctcgaacatgcagcggcaggccgtcccgacgctgcgtacggagaagcctctggtgggcacgggcatggaacgcatcgtcgcccgcgattccggcgtggcggtcgtcgccaagcgcggtggcacggtcgagttcgtggacgccagccgcatcgtggtccgggtgaacgacgaggagaccgaggcgggggttccgggtgtggatatctacaacctgaccaaatacacccgttccaaccagaacacctgtattaaccagaggccgttggttaagccgggcgacgtcgtggcccgcaacgacgtgctggcggatggtccttcgaccgacatgggcgaactggcgctgggccagaatctgctggtcgcgttcatgccctggaacggttacaacttcgaagactcgatcctgatttcggaacgcgtggtgcaggacgaccgtttcacgaccatccacatcgaggagaagacctgcgtcgctcgagacaccaaactggggcccgaggaaatcaccgccgacattccgaacgttggggaggccgcgctgtccaagctggacgagtcgggtatcgtctacatcggcgccgaggtcaaggccggcgacatcctggtgggcaaggtgacgcccaagggcgaaacccagctcaccccggaggagaagctgctgcgagcgatcttcggtgagaaggcctcggatgtgaaggacacttcgctgcgcgtgccttcgggtatggacggcacggtcatagacgtgcaggtcttcacccgcgacggcgtgaagaaggatgaacgcgcccgtcagatcgaggaagccgagattgagagggttcgcaaggatctgaacgatcagctccgtatcatcgagaaggacttttaccagcgggccgagcagatggttctgggcaaggtcgccgacatggggccgggcggtctgaagcgcggcgccaccatcacccgggaatacctggattcgatcaagcccgcccagtggctggagatccggttgcaggacgaagacgtcaatcttcagatcgaagcgatcgccgagcagatcgcccagcagcgcgaggaaatcgcgaagcggctcgaagagaagcgccgcaagatcaccatgggtgacgacctcgctccgggtgtgctcaagatggtaaaggtgtacctggccgtcaagcgccgtattcagccgggcgacaagatggccggccgccacgggaacaaaggtgtgatttcccgcatcgtaccggtggaagacatgccgtattcggctgacggtacgccggtcgacatcgtgttgaacccactgggcgtgccttcgcgcatgaacgtaggccaggtgctcgagacccatctcggctgggcggccaagggtgtgggcctgaagatcgggcggatgctggaggccaaggccaagatcgaggagatccggtctttcctcactcaggtgtacaacgtgagtggccgccaggaggacatcgccagtctgagcgacgccgaggtgctggagctcgccggtaatctgcaggacggcgtgccgatggcgactcccgtgttcgatggagccaccgaggaagacatcaaggccatgctgcggctggccgatcttcccgaaagcggccaggcaactttgttcgacggacgtaccggcgacgtgttcgaccgtccggtgaccgtcggttacatgtacatgctcaaactcaatcatctggtcgacgacaagatgcacgcacggtccaccggtccgtacagtctggtgacccagcagccgctgggcggcaaggcgcaattcggtggacagcgtttcggcgaaatggaggtgtgggcgttggaggcctacggtgccgcctatactctgcaggagatgctgacggtgaaatctgacgacgtgaacggcaggaccaagatgtacaagaacatcgtcgacggtgatcaccgtatggaagccgccatgcccgagtcgttcaacgtcctgatcaaagagatccgctcgctcggaatcaacatcgagctcgagcaggactga

SEQ ID NO: 124 - DNA, 20 bp, synthetic construct
  misc_feature 1..20: note = RPOB gRNA targeting sequence
  source 1..20: mol_type = other DNA
ggaccagaacaatccgttgt

SEQ ID NO: 125 - DNA, 103 bp, synthetic construct
  misc_feature 1..103: note = RPOB gRNA with Handle and Terminator
  source 1..103: mol_type = other DNA
ggaccagaacaatccgttgtgttttagagctagaaatagcaagttaaaataaggctagtccgttatcaacttgaaaaagtggcaccgagtcggtgcttttttt

SEQ ID NO: 126 - DNA, 1044 bp, synthetic construct; identical to SEQ ID NO: 90
  misc_feature 1..1044: note = RpoB Donor DNA
  source 1..1044: mol_type = other DNA